SEQUENCE LISTING (939 sequences)

SEQ ID NO: 1 - DNA, 2210 bp, Artificial Sequence
  Synthetic Sequence
  misc_feature (187)..(187): n is a, c, g, or t
  misc_feature (210)..(210): n is a, c, g, or t
  misc_feature (1088)..(1088): n is a, c, g, or t
  misc_feature (2190)..(2190): n is a, c, g, or t
cccatttcctgtttgccccgagaatactcaccagcggcacttgcagctgcagcgtttaccccgagataactcgycgattacagtcctaaccttacccccaaagtttgccatgaaatatctcgcttttattattattttcgcatcgctctagtatatcgatagtctttggaaacaaatgacatcattntatttacagcattctgtttttantagtggtatttccatttacaaaatatagtaattttctatcgctgaaaatgtcaaatcctagaaaacgtagcattcctacatgtgatgttaacttcgttctcgaacagttgttagccgaagattcatttgatgaatccgatttttccgaaatagacgattctgatgatttttcggatagtgcttcggaagactatacggtcaggcctccgtccgattcggaatctgatggaaatagccctacatcagctgactcgggtcgcgctctgaaatggtcaactcgtgttatgattccacgtcaaaggtatgactttaccggcacacctggcagaaaagttgatgtcagtgataccactgacccactgcagtattttgaactgttctttactgaggaattagtttcaaaaattaccagtgaaatgaatgcccaagctgccttgttggcttcaaagccacctggtccgaaaggattttcgcgaatggataaatggaaagacactgacaatgatgaactgaaagtcttttttgcagtaatgttactgcaaggtattgtgcagaaacctgagctggagatgttttggtcgacaaggcctcttttggatataccttatctcaggcaaattatgactggtgaaagatttttacttttgcttcggtgcctgcattttgtcaacaattcttccatatccgctggtcaatcaaaggcccagatttcattgcagaagatcaaacctgtgttcgactttcttgtaaataagttttcaactgtatatactccaaacagaaacattgcagtcgatgaatcactgatgctgttcaaggggcggttagctatgaagcagtacatcccgacgaaatgtgcacgatttggtctcaagctntatgtactttgtgaaagtcaatctggttacgtgtggaatgcgcttgttcacacagggcccagtatgaatttgaaagattcagctgatggtctgaaatcgtcatgcattgttcttaccttggtcaatgaccttcttggccaaggatattgtgtcttcctcaataacttttatacatctcccatgcttttcagagaattacatcaaaacaggactgatgcagttgggacagctcgtttgaacagaaaacagatgccaaatgatctgaaaaaaaggattgcaaaggggacgactgtagccagattctgtggtgaacttatggcactgaaatggtgtgacaagaaggaggtgacaatgttgtcaacattccacaatgatactgtgattgaagtagacaacagaaatggaaagaaaactaagaagccatgtgtcattgtggattataacgagaatatgggagcagtggactcggctgatcagatgctcacttcttatccaactgagcgcaaaaggcacaagttttggtataagaaattctttcgccaccttctaaacattacagtgctgaactcctacatcctgttcaagaaggacaatcctgagcacacgatcagccatgtaaacttcagactgacgttgattgaaagaatgctggaaaagcatcacaagccagggcagcaacgtcttcgaggtcgtccgtgctctgatgatgtcacacctcttcgcctgtctggaagacatttccccaagagcataccaccaacatcagggaaacagaatccaactggtcgctgcaaagtttgctgctcgcacgacaaggatggcaagaagatccggagagaaacgttatatttttgtgcggaatgtgatgttccgctttgtgttgttccgtgctttgaaatttaccacacgaaaaaaaattattaaatactgatcatcatatacatttctgttacattaggattagagacaagttctgtttagaaataactccaagaacagtttttatattttattttcacattgaaaaccagtcagatttgcttcagcctcaaagagcatgtttatgtaaaattaaattaacgctggcagcgagctgcacttnttttctaaacgggaaatggg

SEQ ID NO: 2 - protein, 584 aa, Artificial Sequence
  Synthetic Sequence
MSNPRKRSIPTCDVNFVLEQLLAEDSFDESDFSEIDDSDDFSDSASEDYTVRPPSDSESDGNSPTSADSGRALKWSTRVMIPRQRYDFTGTPGRKVDVSDTTDPLQYFELFFTEELVSKITSEMNAQAALLASKPPGPKGFSRMDKWKDTDNDELKVFFAVMLLQGIVQKPELEMFWSTRPLLDIPYLRQIMTGERFLLLLRCLHFVNNSSISAGQSKAQISLQKIKPVFDFLVNKFSTVYTPNRNIAVDESLMLFKGRLAMKQYIPTKCARFGLKLYVLCESQSGYVWNALVHTGPSMNLKDSADGLKSSCIVLTLVNDLLGQGYCVFLNNFYTSPMLFRELHQNRTDAVGTARLNRKQMPNDLKKRIAKGTTVARFCGELMALKWCDKKEVTMLSTFHNDTVIEVDNRNGKKTKKPCVIVDYNENMGAVDSADQMLTSYPTERKRHKFWYKKFFRHLLNITVLNSYILFKKDNPEHTISHVNFRLTLIERMLEKHHKPGQQRLRGRPCSDDVTPLRLSGRHFPKSIPPTSGKQNPTGRCKVCCSHDKDGKKIRRETLYFCAECDVPLCVVPCFEIYHTKKNY

SEQ ID NO: 3 - protein, 585 aa, Artificial Sequence
  Synthetic Sequence
MSNPRKRSIPMRDSNTGLEQLLAEDSFDESDFSEIDDSDNFSDSALEADKIRPLSHLESDGKSSTSSDSGRSMKWSARAMIPRQRYDFTGTPGRKVDVSDITDPLQYFELFFTEELVSKITRETNAQAALLASKPPGPKGFSRMDKWKDTDNDELKVFFAVMLLQGIVQKPELEMFWSTRPLLDTPYLRQIMTGERFLLLFRCLHFVNNSSISAGQSKAQISLQKIKPVFDFLVNKFSTVYTPNRNIAVDESLMLFKGPLAMKQYLPTKRVRFGLKLYVLCESQSGYVWNALVHTGPGMNLKDSADGLKSSRIVLTLVNDLLGQGYCVFLDNFNISPMLFRELHQNRTDAVGTARLNRKQIPNDLKKRIAKGTTVARFCGELMALKWCDGKEVTMLSTFHNDTVIEVNNRNGKKTKRPRVIVDYNENMGAVDSADQMLTSYPSERKRHKVWYKKFFHHLLHITVLNSYILFKKDNPEHTMSHINFRLALIERMLEKHHKPGQQHLRGRPCSDDVTPLRLSGRHFPKSIPATSGKQNPTGRCKICCSQYDKDGKKIRKETRYFCAECDVPLCVVPCFEIYHTKKNY

SEQ ID NO: 4 - protein, 585 aa, Artificial Sequence
  Synthetic Sequence
MSNPRKRPIPMRDSNTRLEQLLAEDSFDESDFSEIDDSDNFSDSALEADKIRPLSHLESDGKSSTSSDSGRSMKWSARAMIPRQRYDFTGTPGRKVDVSDITDPLQYFELFFTEELVSKITRETNAQAALLASKPPGPKGFSRMDKWKDTDNDELKVFFAVMLLQGIVQKPELEMFWSTRPLLDTPYLRQIMTGERFLLLFRCLHFVNNSSISAGQSKAQISLQKIKPVFDFLVNKFSTVYTPNRNIAVDESLMLFKGPLAMKQYLPTKRVRFGLKLYVLCESQSGYVWNALVHTGPGMNLKDSADGLKSSRIVLTLVNDLLGQGYCVFLDNFNISPMLFRELHQNRTDAVGTARLNRKQIPNDLKKRIAKGTTVARFCGELMALKWCDGKEVTMLSTFHNDTVIEVNNRNGKKTKRPRVIVDYNENMGAVDSADQMLTSYPSERKRHKVWYKKFFHHLLHITVLNSYILFKKDNPEHTMSHINFRLALIERMLEKHHKPGQQHLRGRPCSDDVTPLRLSGRHFPKSIPATSGKQNPTGRCKICCSQYDKDGKKIRKETRYFCAECDVPLCVVPCFEIYHTKKNY

SEQ ID NO: 5 - DNA, 1758 bp, Artificial Sequence
  Synthetic Sequence
atgtcaaatcctagaaaacgtcccattcctatgcgtgatagtaatacccgtctcgaacagttgttggctgaagattcatttgatgaatctgatttttcggaaatagatgattctgataatttttcggatagtgctttagaagccgataagatcaggcctctgtcccatttagaatctgatggaaagagctctacatcaagtgactcagggcgctccatgaaatggtcagctcgtgctatgattccacgtcaaaggtatgactttaccggcacacctggcagaaaagtcgatgtcagtgatatcactgacccattgcagtattttgaactgttctttactgaggaattagtttcaaaaattactagagaaacaaatgcccaagctgccttgttggcttcaaagccaccgggtccgaaaggattttcgcgaatggataaatggaaagacactgacaatgacgagctcaaagtcttttttgcagtaatgttactgcaaggtattgtgcagaaacctgagctggagatgttttggtcaacaaggcctcttttggatacaccttatctcaggcaaattatgactggtgaaagatttttacttttgtttcggtgcctgcattttgtcaacaattcttctatatctgctggtcaatcaaaggcccagatttcattgcagaagatcaaacctgtgttcgactttcttgtaaataaattttccactgtatatactccaaacagaaacattgcagttgatgaatcactgatgctgttcaaggggccattagctatgaagcagtacctcccgacaaaacgagtacgatttggtctgaagctatatgtactttgtgaaagtcagtctggttatgtgtggaatgcgcttgttcacacagggcctggcatgaatttgaaagattcagcggatggcctgaaatcatcacgcattgttcttaccttggtcaatgaccttcttggccaagggtattgtgtcttcctcgataactttaatatatctcccatgcttttcagagaattacatcaaaataggactgatgcagttgggacagctcgtttgaacagaaaacagattccaaatgatctgaaaaaaaggattgcaaaggggacgactgtagccagattctgtggtgaacttatggcactgaaatggtgtgacggcaaggaggtgacaatgttgtcaacattccacaatgatactgtgattgaagtaaacaatagaaatggaaagaaaactaaaaggccacgtgtcattgtggattataacgagaatatgggagcagtggactcggctgatcaaatgcttacttcttatccatctgagcgcaaaagacacaaggtttggtataagaaattctttcaccatcttctacacattacagtgctgaactcctacatcctgttcaagaaggataatcctgagcacacgatgagccatataaacttcagactggcattgattgaaagaatgctggaaaagcatcacaagccagggcagcaacatcttcgaggtcgtccttgctccgatgatgtcacacctcttcgtctgtctggaagacatttccccaagagcataccagcaacgtccgggaaacagaatccaactggtcgctgcaaaatttgctgctcccaatacgacaaggatggcaagaagatccggaaagaaacgcgctatttttgtgccgaatgtgatgttccgctttgtgttgttccgtgctttgaaatttaccacacgaaaaaaaattattaa

SEQ ID NO: 6 - protein, 809 aa, Artificial Sequence
  Synthetic Sequence
MYEALPGPAPENEDGLVKVKEEDPTWEQVCNSQEGSSHTQEICRLRFRHFCYQEAHGPQEALAQLRELCHQWLRPEMHTKEQIMELLVLEQFLTILPKELQPCVKTYPLESGEEAVTVLENLETGSGDTGQQASVYIQGQDMHPMVAEYQGVSLECQSLQLLPGITTLKCEPPQRPQGNPQEVSGPVPHGSAHLQEKNPRDKAVVPVFNPVRSQTLVKTEEETAQAVAAEKWSHLSLTRRNLCGNSAQETVMSLSPMTEEIVTKDRLFKAKQETSEEMEQSGEASGKPNRECAPQIPCSTPIATERTVAHLNTLKDRHPGDLWARMHISSLEYAAGDITRKGRKKDKARVSELLQGLSFSGDSDVEKDNEPEIQPAQKKLKVSCFPEKSWTKRDIKPNFPSWSALDSGLLNLKSEKLNPVELFELFFDDETFNLIVNETNNYASQKNVSLEVTVQEMRCVFGVLLLSGFMRHPRREMYWEVSDTDQNLVRDAIRRDRFELIFSNLHFADNGHLDQKDKFTKLRPLIKQMNKNFLLYAPLEEYYCFDKSMCECFDSDQFLNGKPIRIGYKIWCGTTTQGYLVWFEPYQEESTMKVDEDPDLGLGGNLVMNFADVLLERGQYPYHLCFDSFFTSVKLLSALKKKGVRATGTIRENRTEKCPLMNVEHMKKMKRGYFDFRIEENNEIILCRWYGDGIISLCSNAVGIEPVNEVSCCDADNEEIPQISQPSIVKVYDECKEGVAKMDQIISKYRVRIRSKKWYSILVSYMIDVAMNNAWQLHRACNPGASLDPLDFRRFVAHFYLEHNAHLSD

SEQ ID NO: 7 - protein, 592 aa, Artificial Sequence
  Synthetic Sequence
MASTSRDVIAGRGIHSKVKSAKLLEVLNAMEEEESNNNREEIFIAPPDNAAGEFTDEDSGDEDSQRGAHLPGSVLHASVLCEDSGTGEDNDDLELQPAKKRQKAVVKPQRIWTKRDIRPDFGSWTASDPHIEDLKSQELSPVGLFELFFDEGTINFIVNETNRYAWQKNVNLSLTAQELKCVLGILILSGYISYPRRRMFWETSPDSHHHLVADAIRRDRFELIFSYLHFADNNELDASDRFAKVRPLIIRMNCNFQKHAPLEEFYSFGESMCEYFGHRGSKQLHRGKPVRLGYKIWCGTTSRGYLVWFEPSQGTLFTKPDRSLDLGGSMVIKFVDALQERGFLPYHIFFDKVFTSVKLMSILRKKGVKATGTVREYRTERCPLKDPKELKKMKRGSFDYKVDESEEIIVCRWHDSSVVNICSNAVGIEPVRLTSRHSGAAKTRTQVHQPSLVKLYQEKVGGVGRMDQNIAKYKVKIRGMKWYSSFIGYVIDAALNNAWQLHRICCQDAQVDLLAFRRYIACVYLESNADTTSQGRRSRRLETESRFDMIGHWIIHQDKRTRCALCHSQTNTRCEKCQKGVHAKCFREYHIR

SEQ ID NO: 8 - protein, 593 aa, Artificial Sequence
  Synthetic Sequence
MPRTLSLHEITDLLETDDSIEASAIVIQPPENATAPVSDEESGDEEGGTINNLPGSLLHTAAYLIQDGSDAESDSDDPSYAPKDDSPDEVPSTFTVQQPPPSRRRKMTKILCKWKKADLTVQPVAGRVTAPPNDFFTVMRTPTEILELFLDDEVIELIVKYSNLYACSKGVHLGLTSSEFKCFLGIIFLSGYVSVPRRRMFWEQRTDVHNVLVSAAMRRDRFETIFSNLHVADNANLDPVDKFSKLRPLISKLNERCMKFVPNETYFSFDEFMVPYFGRHGCKQFIRGKPIRFGYKFWCGATCLGYICWFQPYQGKNPNTKHEEYGVGASLVLQFSEALTEAHPGQYHFVFNNFFTSIALLDKLSSMGHQATGTVRKDHIDRVPLESDVALKKKERGTFDYRIDGKGNIVCRWNDNSVVTVASSGAGIHPLCLVSRYSQKLKKKIQVQQPNMIKVYNQFMGGVDRADENIDKYRASIRGKKWYSSPLLFCFELVLQNAWQLHKTYDEKPVDFLEFRRRVVCHYLETHGHPPEPGQKGRPQKRNIDSRYDGINHVIVKQGKQTRCAECHKNTTFRCEKCDVALHVKCSVEYHTE

SEQ ID NO: 9 - protein, 524 aa, Artificial Sequence
  Synthetic Sequence
MAEGGGGARRRAPALLEAARARYESLHISDDVFGESGPDSGGNPFYSTSAASRSSSAASSDDEREPPGPPGAAPPPPRAPDAQEPEEDEAGAGWSAALRDRPPPRFEDTGGPTRKMPPSASAVDFFQLFVPDNVLKNMVVQTNMYAKKFQERFGSDGAWVEVTLTEMKAFLGYMISTSISHCESVLSIWSGGFYSNRSLALVMSQARFEKILKYFHVVAFRSSQTTHGLYKVQPFLDSLQNSFDSAFRPSQTQVLHEPLIDEDPVFIATCTERELRKRKKRKFSLWVRQCSSTGFIIQIYVHLKEGGGPDGLDALKNKPQLHSMVARSLCRNAAGKNYIIFTGPSITSLTLFEEFEKQGIYCCGLLRARKSDCTGLPLSMLTNPATPPARGQYQIKMKGNMSLICWYNKGHFRFLTNAYSPVQQGVIIKRKSGEIPCPLAVEAFAAHLSYICRYDDKYSKYFISHKPNKTWQQVFWFAISIAINNAYILYKMSDAYHVKRYSRAQFGERLVRELLGLEDASPTH

SEQ ID NO: 10 - protein, 572 aa, Artificial Sequence
  Synthetic Sequence
MSQHSDYPDDEFRADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQKRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 11 - DNA, 381 bp, Artificial Sequence
  Synthetic Sequence
ttaacccatttcctgtttgccccgagaatactcaccagcggcacttgcagctgcagcgtttaccccgagataactcgtcgattacagtcctaaccttacccccaaagtttgccatgaaatatctcgcttttattattattttcgcatcgctctagtatatcgatagtctttggaaacaaatgacatcattctatttacagcattctgtttttagtagtggtatttccatttacaaaatatagtaattttctatcgctgaaaatgtcaaatcctagaaaacgtagcattcctacatgtgatgttaacttcgttctcgaacagttgttagccgaagattcatttgatgaatccgatttttccgaaatagacgattctgatgat

SEQ ID NO: 12 - DNA, 373 bp, Artificial Sequence
  Synthetic Sequence
ttaactcatttctccttagccccgagattacgcgctgctgtgcctgcgactgcagcgtttacgccgagataactcgtggattacagtgccaaccttactcccaaagtttgccacgaaatatctcgcttctgttattttcgcatggttctggtatattgacttttgaaacaaaagacatcattctgtttatagcattctgtttttagtagtgggatttccatctacaaaatatagtaattctcgatcgctgaaatgtcaaatcctagaaaacgtagcattcctatgcgtgatagtaataccggtctcgaacagttgttggctgaagattcatttgatgaatctgatttttcggaaatagatgattctgataatt

SEQ ID NO: 13 - DNA, 344 bp, Artificial Sequence
  Synthetic Sequence
ttaacccttttcccgtttgccccgagaatactcgccggcggcgcttgcggctgcagcgtttaccccgagataactttgccacgaaatatctcgcttttattattattttcgcatcgctctagtatatcgactttggaaacaaaagacatcattctatttatagcattctgtttttagtagtggtatttccatttacaaaatatagtaattctcgatcgctgaaaatgtcaaatcctagaaaacgtagcattcctacgcgtgatgttaacatcgttctcgaacagttgttggccgaagattcatttgatgaatccgatttttccgaaatagacgattctgatgat

SEQ ID NO: 14 - DNA, 91 bp, Artificial Sequence
  Synthetic Sequence
ttaacccatttcccgtttgccccgagaatactcttgtctctaatcctaatgtaacatcatatacatttctgttacattaggattagagaca

SEQ ID NO: 15 - DNA, 32 bp, Artificial Sequence
  Synthetic Sequence
ttaacccatttcccgtttgccccgagaatact

SEQ ID NO: 16 - DNA, 171 bp, Artificial Sequence
  Synthetic Sequence
taggattagagacaagttctgtttagaaataactccaagaacagtttttatattttattttcacattgaaaaccagtcagatttgcttcagcctcaaagagcatgtttatgtaaaattaaattaacgctggcagcgagctgcactttttttctaaacgggaaatgggttaa

SEQ ID NO: 17 - DNA, 176 bp, Artificial Sequence
  Synthetic Sequence
cctgggattataggcatgagccactgcgcctagcaccaagaacagtttttatattttattttcacattgaaaatcagtcagatttgcttcagcctcaaagagggtgtttatgtaaaactaaatgagtgcaggcagcgagctacacttttttttttcctaaatggaaaatgggttaa

SEQ ID NO: 18 - DNA, 178 bp, Artificial Sequence
  Synthetic Sequence
tcagacgattctgatgttagttctgtttagaaataactccaagaacagtttttatattttattttcacattgaaaatcagtcagatttgcttcagcctcaaagagcgtgtttatgtaaaattaaatgagcgctggcagcgagctgcacttttttttttctaaacgggaaaagggttaa

SEQ ID NO: 19 - DNA, 160 bp, Artificial Sequence
  Synthetic Sequence
agttctgtttagaaataactccaagaacagtttttatattttattttcacattgaaaatcagtcagatttgcttcagcctcaaagagcgtgtttatgtaaaattaaatgagcgctggcagcgagctgcacttttttttttctaaacgggaaaagggttaa

SEQ ID NO: 20 - DNA, 46 bp, Artificial Sequence
  Synthetic Sequence
cgctggcagcgagctgcactttttttctaaacgggaaatgggttaa

SEQ ID NO: 21 - protein, 1379 aa, Artificial Sequence
  Synthetic Sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 22 - DNA, 4137 bp, Artificial Sequence
  Synthetic Sequence
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctggaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtg

SEQ ID NO: 23 - DNA, 19 bp, Artificial Sequence
  Synthetic Sequence
tggccggcctgaccactgg

SEQ ID NO: 24 - DNA, 19 bp, Artificial Sequence
  Synthetic Sequence
tgaaggcctggccggcctg

SEQ ID NO: 25 - DNA, 19 bp, Artificial Sequence
  Synthetic Sequence
tgagcactgaaggcctggc

SEQ ID NO: 26 - DNA, 19 bp, Artificial Sequence
  Synthetic Sequence
tccactgagcactgaaggc

SEQ ID NO: 27 - DNA, 19 bp, Artificial Sequence
  Synthetic Sequence
tggtttccactgagcactg

SEQ ID NO: 28 - DNA, 19 bp, Artificial Sequence
  Synthetic Sequence
tggggaaaatgacccaaca

SEQ ID NO: 29 - DNA, 19 bp, Artificial Sequence
  Synthetic Sequence
taggacagtggggaaaatg

SEQ ID NO: 30 - DNA, 19 bp, Artificial Sequence
  Synthetic Sequence
tccagggacacggtgctag

SEQ ID NO: 31 - DNA, 19 bp, Artificial Sequence
  Synthetic Sequence
tcagagccaggagtcctgg

SEQ ID NO: 32 - DNA, 19 bp, Artificial Sequence
  Synthetic Sequence
tccttcagagccaggagtc

SEQ ID NO: 33 - DNA, 19 bp, Artificial Sequence
  Synthetic Sequence
tcctccttcagagccagga

SEQ ID NO: 34 - DNA, 19 bp, Artificial Sequence
  Synthetic Sequence
tccagcccctcctccttca

SEQ ID NO: 35 - DNA, 19 bp, Artificial Sequence
  Synthetic Sequence
tccgagcttgacccttgga

SEQ ID NO: 36 - DNA, 19 bp, Artificial Sequence
  Synthetic Sequence
tggtttccgagcttgaccc

SEQ ID NO: 37 - DNA, 19 bp, Artificial Sequence
  Synthetic Sequence
tggggtggtttccgagctt

SEQ ID NO: 38 - DNA, 19 bp, Artificial Sequence
  Synthetic Sequence
tctgctggggtggtttccg

SEQ ID NO: 39 - DNA, 19 bp, Artificial Sequence
  Synthetic Sequence
tgcagagtatctgctgggg

SEQ ID NO: 40 - DNA, 14 bp, Artificial Sequence
  Synthetic Sequence
ccaatcccctcagt

SEQ ID NO: 41 - DNA, 15 bp, Artificial Sequence
  Synthetic Sequence
cagtgctcagtggaa

SEQ ID NO: 42 - DNA, 18 bp, Artificial Sequence
  Synthetic Sequence
gaaacatccggcgactca

SEQ ID NO: 43 - DNA, 19 bp, Artificial Sequence
  Synthetic Sequence
tcgcccctcaaatcttaca

SEQ ID NO: 44 - DNA, 19 bp, Artificial Sequence
  Synthetic Sequence
tcaaatcttacagctgctc

SEQ ID NO: 45 - DNA, 19 bp, Artificial Sequence
  Synthetic Sequence
tcttacagctgctcactcc

SEQ ID NO: 46 - DNA, 19 bp, Artificial Sequence
  Synthetic Sequence
tacagctgctcactcccct

SEQ ID NO: 47 - DNA, 19 bp, Artificial Sequence
  Synthetic Sequence
tgctcactcccctgcaggg

SEQ ID NO: 48 - DNA, 19 bp, Artificial Sequence
  Synthetic Sequence
tcccctgcagggcaacgcc

SEQ ID NO: 49 - DNA, 19 bp, Artificial Sequence
  Synthetic Sequence
tgcagggcaacgcccaggg

SEQ ID NO: 50 - DNA, 19 bp, Artificial Sequence
  Synthetic Sequence
tctcgattatgggcgggat

SEQ ID NO: 51 - DNA, 19 bp, Artificial Sequence
  Synthetic Sequence
tcgcttctcgattatgggc

SEQ ID NO: 52 - DNA, 19 bp, Artificial Sequence
  Synthetic Sequence
tgtcgagtcgcttctcgat

SEQ ID NO: 53 - DNA, 19 bp, Artificial Sequence
  Synthetic Sequence
tccatgtcgagtcgcttct

SEQ ID NO: 54 - DNA, 19 bp, Artificial Sequence
  Synthetic Sequence
tcgcctccatgtcgagtcg

SEQ ID NO: 55 - DNA, 19 bp, Artificial Sequence
  Synthetic Sequence
tcgtcatcgcctccatgtc

SEQ ID NO: 56 - DNA, 19 bp, Artificial Sequence
  Synthetic Sequence
tgatctcgtcatcgcctcc

SEQ ID NO: 57 - DNA, 14 bp, Artificial Sequence
  Synthetic Sequence
gcttcagcttccta

SEQ ID NO: 58 - DNA, 14 bp, Artificial Sequence
  Synthetic Sequence
ctgtgatcatgcca

SEQ ID NO: 59 - DNA, 16 bp, Artificial Sequence
  Synthetic Sequence
acagtggtacacacct

SEQ ID NO: 60 - DNA, 15 bp, Artificial Sequence
  Synthetic Sequence
ccaccccccactaag

SEQ ID NO: 61 - DNA, 14 bp, Artificial Sequence
  Synthetic Sequence
cattggccgggcac

SEQ ID NO: 62 - DNA, 16 bp, Artificial Sequence
  Synthetic Sequence
gcttgaacccaggaga

SEQ ID NO: 63 - DNA, 17 bp, Artificial Sequence
  Synthetic Sequence
acacccgatccactggg

SEQ ID NO: 64 - DNA, 14 bp, Artificial Sequence
  Synthetic Sequence
gctgcatcaacccc

SEQ ID NO: 65 - DNA, 16 bp, Artificial Sequence
  Synthetic Sequence
gccacaaacagaaata

SEQ ID NO: 66 - DNA, 15 bp, Artificial Sequence
  Synthetic Sequence
ggtggctcatgcctg

SEQ ID NO: 67 - DNA, 16 bp, Artificial Sequence
  Synthetic Sequence
gatttgcacagctcat

SEQ ID NO: 68 - DNA, 15 bp, Artificial Sequence
  Synthetic Sequence
aagctctgaggagca

SEQ ID NO: 69 - DNA, 13 bp, Artificial Sequence
  Synthetic Sequence
ccctagctgtccc

SEQ ID NO: 70 - DNA, 14 bp, Artificial Sequence
  Synthetic Sequence
gcctagcatgctag

SEQ ID NO: 71 - DNA, 15 bp, Artificial Sequence
  Synthetic Sequence
atgggcttcacggat

SEQ ID NO: 72 - DNA, 14 bp, Artificial Sequence
  Synthetic Sequence
gaaactatgcctgc

SEQ ID NO: 73 - DNA, 14 bp, Artificial Sequence
  Synthetic Sequence
gcaccattgctccc

SEQ ID NO: 74 - DNA, 14 bp, Artificial Sequence
  Synthetic Sequence
gacatgcaactcag

SEQ ID NO: 75 - DNA, 14 bp, Artificial Sequence
  Synthetic Sequence
acaccactaggggt

SEQ ID NO: 76 - DNA, 14 bp, Artificial Sequence
  Synthetic Sequence
gtctgctagacagg

SEQ ID NO: 77 - DNA, 15 bp, Artificial Sequence
  Synthetic Sequence
ggcctagacaggctg

SEQ ID NO: 78 - DNA, 15 bp, Artificial Sequence
  Synthetic Sequence
gaggcattcttatcg

SEQ ID NO: 79 - DNA, 15 bp, Artificial Sequence
  Synthetic Sequence
gcctggaaacgttcc

SEQ ID NO: 80 - DNA, 14 bp, Artificial Sequence
  Synthetic Sequence
gtgctctgacaata

SEQ ID NO: 81 - DNA, 14 bp, Artificial Sequence
  Synthetic Sequence
gttttgcagcctcc

SEQ ID NO: 82 - DNA, 15 bp, Artificial Sequence
  Synthetic Sequence
acagctgtggaacgt

SEQ ID NO: 83 - DNA, 15 bp, Artificial Sequence
  Synthetic Sequence
ggctctcttcctcct

SEQ ID NO: 84 - DNA, 15 bp, Artificial Sequence
  Synthetic Sequence
ctatcccaaaactct

SEQ ID NO: 85 - DNA, 14 bp, Artificial Sequence
  Synthetic Sequence
gaaaaactatgtat

SEQ ID NO: 86 - DNA, 15 bp, Artificial Sequence
  Synthetic Sequence
aggcaggctggttga

SEQ ID NO: 87 - DNA, 14 bp, Artificial Sequence
  Synthetic Sequence
caatacaaccacgc

SEQ ID NO: 88 - DNA, 15 bp, Artificial Sequence
  Synthetic Sequence
atgacggactcaact

SEQ ID NO: 89 - DNA, 14 bp, Artificial Sequence
  Synthetic Sequence
cacaacatttgtaa

SEQ ID NO: 90 - DNA, 14 bp, Artificial Sequence
  Synthetic Sequence
atttccagtgcaca

SEQ ID NO: 91 - DNA, 20 bp, Artificial Sequence
  Synthetic Sequence
gtttagctcacccgtgagcc

SEQ ID NO: 92 - DNA, 20 bp, Artificial Sequence
  Synthetic Sequence
cccaatattattgttctctg

SEQ ID NO: 93 - DNA, 20 bp, Artificial Sequence
  Synthetic Sequence
ggggtgggataggggatacg

SEQ ID NO: 94 - DNA, 20 bp, Artificial Sequence
  Synthetic Sequence
ggatccccctctacatttaa

SEQ ID NO: 95 - DNA, 20 bp, Artificial Sequence
  Synthetic Sequence
gtgatcttgtacaaatcatt

SEQ ID NO: 96 - DNA, 20 bp, Artificial Sequence
  Synthetic Sequence
ctacacagaatctgttagaa

SEQ ID NO: 97 - DNA, 20 bp, Artificial Sequence
  Synthetic Sequence
taagctagagaatagatctc

SEQ ID NO: 98 - DNA, 20 bp, Artificial Sequence
  Synthetic Sequence
tcaatacacttaatgattta

SEQ ID NO: 99 - DNA, 25 bp, Artificial Sequence
  Synthetic Sequence
caccgggagccacgaaaacagatcc

SEQ ID NO: 100 - DNA, 25 bp, Artificial Sequence
  Synthetic Sequence
caccgcgaaaacagatccagggaca

SEQ ID NO: 101 - DNA, 25 bp, Artificial Sequence
  Synthetic Sequence
caccgagatccagggacacggtgct

SEQ ID NO: 102 - DNA, 25 bp, Artificial Sequence
  Synthetic Sequence
caccggacacggtgctaggacagtg

SEQ ID NO: 103 - DNA, 25 bp, Artificial Sequence
  Synthetic Sequence
caccggaaaatgacccaacagcctc

SEQ ID NO: 104 - DNA, 25 bp, Artificial Sequence
  Synthetic Sequence
caccggcctggccggcctgaccact

SEQ ID NO: 105 - DNA, 25 bp, Artificial Sequence
  Synthetic Sequence
caccgctgagcactgaaggcctggc

SEQ ID NO: 106 - DNA, 25 bp, Artificial Sequence
  Synthetic Sequence
caccgtggtttccactgagcactga

SEQ ID NO: 107 - DNA, 25 bp, Artificial Sequence
  Synthetic Sequence
caccggatagccaggagtcctttcg

SEQ ID NO: 108 - DNA, 25 bp, Artificial Sequence
  Synthetic Sequence
caccggcgcttccagtgctcagact

SEQ ID NO: 109 - DNA, 25 bp, Artificial Sequence
  Synthetic Sequence
caccgcagtgctcagactagggaag

SEQ ID NO: 110 - DNA, 25 bp, Artificial Sequence
  Synthetic Sequence
caccggcccctcctccttcagagcc

SEQ ID NO: 111 - DNA, 25 bp, Artificial Sequence
  Synthetic Sequence
caccgtccttcagagccaggagtcc

SEQ ID NO: 112 - DNA, 25 bp, Artificial Sequence
  Synthetic Sequence
caccgtggtttccgagcttgaccct

SEQ ID NO: 113 - DNA, 25 bp, Artificial Sequence
  Synthetic Sequence
caccgctgcagagtatctgctgggg

SEQ ID NO: 114 - DNA, 25 bp, Artificial Sequence
  Synthetic Sequence
caccgcgttcctgcagagtatctgc

SEQ ID NO: 115-130 - skipped (16 entries)

SEQ ID NO: 131 - DNA, 20 bp, Artificial Sequence
  Synthetic Sequence
tcccctcccagaaagacctg

SEQ ID NO: 132 - DNA, 20 bp, Artificial Sequence
  Synthetic Sequence
tgggctccaagcaatcctgg

SEQ ID NO: 133 - DNA, 20 bp, Artificial Sequence
  Synthetic Sequence
gtggctcaggaggtacctgg

SEQ ID NO: 134 - DNA, 20 bp, Artificial Sequence
  Synthetic Sequence
gagccacgaaaacagatcca

SEQ ID NO: 135 - DNA, 20 bp, Artificial Sequence
  Synthetic Sequence
aagtgaacggggaagggagg

SEQ ID NO: 136 - DNA, 20 bp, Artificial Sequence
  Synthetic Sequence
gacaaaagccgaagtccagg

SEQ ID NO: 137 - DNA, 20 bp, Artificial Sequence
  Synthetic Sequence
gtggttgataaacccacgtg

SEQ ID NO: 138 - DNA, 20 bp, Artificial Sequence
  Synthetic Sequence
tgggaacagccacagcaggg

SEQ ID NO: 139 - DNA, 20 bp, Artificial Sequence
  Synthetic Sequence
gcaggggaacggggatgcag

SEQ ID NO: 140 - DNA, 20 bp, Artificial Sequence
  Synthetic Sequence
gagatggtggacgaggaagg

SEQ ID NO: 141 - DNA, 20 bp, Artificial Sequence
  Synthetic Sequence
gagatggctccaggaaatgg

SEQ ID NO: 142 - DNA, 20 bp, Artificial Sequence
  Synthetic Sequence
taaggaatctgcctaacagg

SEQ ID NO: 143 - DNA, 20 bp, Artificial Sequence
  Synthetic Sequence
tcaggagactaggaaggagg

SEQ ID NO: 144 - DNA, 20 bp, Artificial Sequence
  Synthetic Sequence
tataaggtggtcccagctcg

SEQ ID NO: 145 - DNA, 20 bp, Artificial Sequence
  Synthetic Sequence
ctggaagatgccatgacagg

SEQ ID NO: 146 - DNA, 20 bp, Artificial Sequence
  Synthetic Sequence
gcacagactagagaggtaag

SEQ ID NO: 147 - DNA, 20 bp, Artificial Sequence
  Synthetic Sequence
acagactagagaggtaaggg

SEQ ID NO: 148 - DNA, 20 bp, Artificial Sequence
  Synthetic Sequence
gagaggtgacccgaatccac

SEQ ID NO: 149 - DNA, 20 bp, Artificial Sequence
  Synthetic Sequence
gcacaggccccagaaggaga

SEQ ID NO: 150 - DNA, 20 bp, Artificial Sequence
  Synthetic Sequence
ccggagaggacccagacacg

SEQ ID NO: 151 - DNA, 20 bp, Artificial Sequence
  Synthetic Sequence
gagaggacccagacacgggg

SEQ ID NO: 152 - DNA, 20 bp, Artificial Sequence
  Synthetic Sequence
gcaacacagcagagagcaag

SEQ ID NO: 153 - DNA, 20 bp, Artificial Sequence
  Synthetic Sequence
gaagagggagtggaggaaga

SEQ ID NO: 154 - DNA, 20 bp, Artificial Sequence
  Synthetic Sequence
aagacggaacctgaaggagg

SEQ ID NO: 155 - DNA, 20 bp, Artificial Sequence
  Synthetic Sequence
agaaagcggcacaggcccag

SEQ ID NO: 156 - DNA, 20 bp, Artificial Sequence
  Synthetic Sequence
gggaaacagtgggccagagg

SEQ ID NO: 157 - DNA, 20 bp, Artificial Sequence
  Synthetic Sequence
gtccggactcaggagagaga

SEQ ID NO: 158 - DNA, 20 bp, Artificial Sequence
  Synthetic Sequence
ggcacagcaagggcactcgg

SEQ ID NO: 159 - DNA, 20 bp, Artificial Sequence
  Synthetic Sequence
gaagaggggaagtcgaggga

SEQ ID NO: 160 - DNA, 20 bp, Artificial Sequence
  Synthetic Sequence
gggaatggtaaggaggcctg

SEQ ID NO: 161 - DNA, 20 bp, Artificial Sequence
  Synthetic Sequence
gcagagtggtcagcacagag

SEQ ID NO: 162 - DNA, 20 bp, Artificial Sequence
  Synthetic Sequence
gcacagagtggctaagccca

SEQ ID NO: 163 - DNA, 20 bp, Artificial Sequence
  Synthetic Sequence
gacggggtgtcagcataggg

SEQ ID NO: 164 - DNA, 20 bp, Artificial Sequence
  Synthetic Sequence
gcccagggccaggaacgacg

SEQ ID NO: 165 - DNA, 20 bp, Artificial Sequence
  Synthetic Sequence
ggtggagtccagcacggcgc

SEQ ID NO: 166 - DNA, 20 bp, Artificial Sequence
  Synthetic Sequence
acaggccgccaggaactcgg

SEQ ID NO: 167 - DNA, 20 bp, Artificial Sequence
  Synthetic Sequence
actaggaagtgtgtagcacc

SEQ ID NO: 168 - DNA, 20 bp, Artificial Sequence
  Synthetic Sequence
atgaatagcagactgccccg

SEQ ID NO: 169 - DNA, 20 bp, Artificial Sequence
  Synthetic Sequence
acacccctaaaagcacagtg

SEQ ID NO: 170 - DNA, 20 bp, Artificial Sequence
  Synthetic Sequence
caaggagttccagcaggtgg

SEQ ID NO: 171 - DNA, 20 bp, Artificial Sequence
  Synthetic Sequence
aaggagttccagcaggtggg

SEQ ID NO: 172 - DNA, 20 bp, Artificial Sequence
  Synthetic Sequence
tggaaagaggagggaagagg

SEQ ID NO: 173 - DNA, 20 bp, Artificial Sequence
  Synthetic Sequence
tcgaattcctaactgccccg

SEQ ID NO: 174 - DNA, 20 bp, Artificial Sequence
  Synthetic Sequence
gacctgcccagcacaccctg

SEQ ID NO: 175 - DNA, 20 bp, Artificial Sequence
  Synthetic Sequence
ggagcagctgcggcagtggg

SEQ ID NO: 176 - DNA, 20 bp, Artificial Sequence
  Synthetic Sequence
gggagggagagcttggcagg

SEQ ID NO: 177 - DNA, 20 bp, Artificial Sequence
  Synthetic Sequence
gttacgtggccaagaagcag

SEQ ID NO: 178 - DNA, 20 bp, Artificial Sequence
  Synthetic Sequence
gctgaacagagaagagctgg

SEQ ID NO: 179 - DNA, 20 bp, Artificial Sequence
  Synthetic Sequence
tctgagggtggagggactgg

SEQ ID NO: 180 - DNA, 20 bp, Artificial Sequence
  Synthetic Sequence
ggagaggtgagggacttggg

SEQ ID NO: 181 - DNA, 20 bp, Artificial Sequence
  Synthetic Sequence
gtgaaccaggcagacaacga

SEQ ID NO: 182 - DNA, 20 bp, Artificial Sequence
  Synthetic Sequence
caggtacctcctgagccacg

SEQ ID NO: 183 - DNA, 20 bp, Artificial Sequence
  Synthetic Sequence
gggggagtaggggcatgcag

SEQ ID NO: 184 - DNA, 20 bp, Artificial Sequence
  Synthetic Sequence
gcaaatggccagcaagggtg

SEQ ID NO: 185 - DNA, 25 bp, Artificial Sequence
  Synthetic Sequence
caccgaatcgagaagcgactcgaca

SEQ ID NO: 186 - DNA, 25 bp, Artificial Sequence
  Synthetic Sequence
caccggtccctgggcgttgccctgc

SEQ ID NO: 187 - DNA, 25 bp, Artificial Sequence
  Synthetic Sequence
caccgccctgggcgttgccctgcag

SEQ ID NO: 188 - DNA, 25 bp, Artificial Sequence
  Synthetic Sequence
caccgccgtgggaagataaactaat

SEQ ID NO: 189 - DNA, 25 bp, Artificial Sequence
  Synthetic Sequence
caccgtcccctgcagggcaacgccc

SEQ ID NO: 190 - DNA, 25 bp, Artificial Sequence
  Synthetic Sequence
caccggtcgagtcgcttctcgatta

SEQ ID NO: 191 - DNA, 25 bp, Artificial Sequence
  Synthetic Sequence
caccgctgctgcctcccgtcttgta

SEQ ID NO: 192 - DNA, 25 bp, Artificial Sequence
  Synthetic Sequence
caccggagtgccgcaatacctttat

SEQ ID NO: 193 - DNA, 25 bp, Artificial Sequence
  Synthetic Sequence
caccgacactttggtggtgcagcaa

SEQ ID NO: 194 - DNA, 25 bp, Artificial Sequence
  Synthetic Sequence
caccgtctcaaatggtataaaactc

SEQ ID NO: 195 - DNA, 25 bp, Artificial Sequence
  Synthetic Sequence
caccgaatcccgcccataatcgaga

SEQ ID NO: 196 - DNA, 25 bp, Artificial Sequence
  Synthetic Sequence
caccgtcccgcccataatcgagaag

SEQ ID NO: 197 - DNA, 25 bp, Artificial Sequence
  Synthetic Sequence
caccgcccataatcgagaagcgact

SEQ ID NO: 198 - DNA, 25 bp, Artificial Sequence
  Synthetic Sequence
caccggagaagcgactcgacatgga

SEQ ID NO: 199 - DNA, 25 bp, Artificial Sequence
  Synthetic Sequence
caccggaagcgactcgacatggagg

SEQ ID NO: 200 - DNA, 25 bp, Artificial Sequence
  Synthetic Sequence
caccggcgactcgacatggaggcga

SEQ ID NO: 201 - DNA, 25 bp, Artificial Sequence
  Synthetic Sequence
aaactgtcgagtcgcttctcgattc

SEQ ID NO: 202 - DNA, 25 bp, Artificial Sequence
  Synthetic Sequence
aaacgcagggcaacgcccagggacc

SEQ ID NO: 203 - DNA, 25 bp, Artificial Sequence
  Synthetic Sequence
aaacctgcagggcaacgcccagggc

SEQ ID NO: 204 - DNA, 25 bp, Artificial Sequence
  Synthetic Sequence
aaacattagtttatcttcccacggc

SEQ ID NO: 205 - DNA, 25 bp, Artificial Sequence
  Synthetic Sequence
aaacgggcgttgccctgcaggggac

SEQ ID NO: 206 - DNA, 25 bp, Artificial Sequence
  Synthetic Sequence
aaactaatcgagaagcgactcgacc

SEQ ID NO: 207 - DNA, 25 bp, Artificial Sequence
  Synthetic Sequence
aaactacaagacgggaggcagcagc

SEQ ID NO: 208 - DNA, 25 bp, Artificial Sequence
  Synthetic Sequence
aaacataaaggtattgcggcactcc

SEQ ID NO: 209 - DNA, 25 bp, Artificial Sequence
  Synthetic Sequence
aaacttgctgcaccaccaaagtgtc

SEQ ID NO: 210 - DNA, 25 bp, Artificial Sequence
  Synthetic Sequence
aaacgagttttataccatttgagac

SEQ ID NO: 211 - DNA, 25 bp, Artificial Sequence
  Synthetic Sequence
aaactctcgattatgggcgggattc

SEQ ID NO: 212 - DNA, 25 bp, Artificial Sequence
  Synthetic Sequence
aaaccttctcgattatgggcgggac

SEQ ID NO: 213 - DNA, 25 bp, Artificial Sequence
  Synthetic Sequence
aaacagtcgcttctcgattatgggc

SEQ ID NO: 214 - DNA, 25 bp, Artificial Sequence
  Synthetic Sequence
aaactccatgtcgagtcgcttctcc

SEQ ID NO: 215 - DNA, 25 bp, Artificial Sequence
  Synthetic Sequence
aaaccctccatgtcgagtcgcttcc

SEQ ID NO: 216 - DNA, 25 bp, Artificial Sequence
  Synthetic Sequence
aaactcgcctccatgtcgagtcgcc

SEQ ID NO: 217 - DNA, 25 bp, Artificial Sequence
  Synthetic Sequence
caccgacagggttaatgtgaagtcc

SEQ ID NO: 218 - DNA, 25 bp, Artificial Sequence
  Synthetic Sequence
caccgtccccctctacatttaaagt

SEQ ID NO: 219 - DNA, 25 bp, Artificial Sequence
  Synthetic Sequence
caccgcatttaaagttggtttaagt

SEQ ID NO: 220 - DNA, 25 bp, Artificial Sequence
  Synthetic Sequence
caccgttagaaaatataaagaataa

SEQ ID NO: 221 - DNA, 25 bp, Artificial Sequence
  Synthetic Sequence
caccgtaaatgcttactggtttgaa

SEQ ID NO: 222 - DNA, 25 bp, Artificial Sequence
  Synthetic Sequence
caccgtcctgggtccagaaaaagat

SEQ ID NO: 223 - DNA, 25 bp, Artificial Sequence
  Synthetic Sequence
caccgttgggtggtgagcatctgtg

SEQ ID NO: 224 - DNA, 25 bp, Artificial Sequence
  Synthetic Sequence
caccgcggggagagtggagaaaaag

SEQ ID NO: 225 - DNA, 25 bp, Artificial Sequence
  Synthetic Sequence
caccggttaaaactctttagacaac

SEQ ID NO: 226 - DNA, 25 bp, Artificial Sequence
  Synthetic Sequence
caccggaaaatccccactaagatcc

SEQ ID NO: 227 - DNA, 25 bp, Artificial Sequence
  Synthetic Sequence
aaacggacttcacattaaccctgtc

SEQ ID NO: 228 - DNA, 25 bp, Artificial Sequence
  Synthetic Sequence
aaacactttaaatgtagagggggac

SEQ ID NO: 229 - DNA, 25 bp, Artificial Sequence
  Synthetic Sequence
aaacacttaaaccaactttaaatgc

SEQ ID NO: 230 - DNA, 25 bp, Artificial Sequence
  Synthetic Sequence
aaacttattctttatattttctaac

SEQ ID NO: 231 - DNA, 25 bp, Artificial Sequence
  Synthetic Sequence
aaacttcaaaccagtaagcatttac

SEQ ID NO: 232 - DNA, 25 bp, Artificial Sequence
  Synthetic Sequence
aaacatctttttctggacccaggac

SEQ ID NO: 233 - DNA, 25 bp, Artificial Sequence
  Synthetic Sequence
aaaccacagatgctcaccacccaac

SEQ ID NO: 234 - DNA, 25 bp, Artificial Sequence
  Synthetic Sequence
aaacctttttctccactctccccgc

SEQ ID NO: 235 - DNA, 25 bp, Artificial Sequence
  Synthetic Sequence
aaacgttgtctaaagagttttaacc

SEQ ID NO: 236 - DNA, 25 bp, Artificial Sequence
  Synthetic Sequence
aaacggatcttagtggggattttcc

SEQ ID NO: 237 - DNA, 20 bp, Artificial Sequence
  Synthetic Sequence
agtagcagtaatgaagctgg

SEQ ID NO: 238 - DNA, 20 bp, Artificial Sequence
  Synthetic Sequence
atacccagacgagaaagctg

SEQ ID NO: 239 - DNA, 20 bp, Artificial Sequence
  Synthetic Sequence
tacccagacgagaaagctga

SEQ ID NO: 240 - DNA, 20 bp, Artificial Sequence
  Synthetic Sequence
ggtggtgagcatctgtgtgg

SEQ ID NO: 241 - DNA, 20 bp, Artificial Sequence
  Synthetic Sequence
aaatgagaagaagaggcaca

SEQ ID NO: 242 - DNA, 20 bp, Artificial Sequence
  Synthetic Sequence
cttgtggcctgggagagctg

SEQ ID NO: 243 - DNA, 20 bp, Artificial Sequence
  Synthetic Sequence
gctgtagaaggagacagagc

SEQ ID NO: 244 - DNA, 20 bp, Artificial Sequence
  Synthetic Sequence
gagctggttgggaagacatg

SEQ ID NO: 245 - DNA, 20 bp, Artificial Sequence
  Synthetic Sequence
ctggttgggaagacatgggg

SEQ ID NO: 246 - DNA, 20 bp, Artificial Sequence
  Synthetic Sequence
cgtgaggatgggaaggaggg

SEQ ID NO: 247 - DNA, 20 bp, Artificial Sequence
  Synthetic Sequence
atgcagagtcagcagaactg

SEQ ID NO: 248 - DNA, 20 bp, Artificial Sequence
  Synthetic Sequence
aagacatcaagcacagaagg

SEQ ID NO: 249 - DNA, 20 bp, Artificial Sequence
  Synthetic Sequence
tcaagcacagaaggaggagg

SEQ ID NO: 250 - DNA, 20 bp, Artificial Sequence
  Synthetic Sequence
aaccgtcaataggcaaaggg

SEQ ID NO: 251 - DNA, 20 bp, Artificial Sequence
  Synthetic Sequence
ccgtatttcagactgaatgg

SEQ ID NO: 252 - DNA, 20 bp, Artificial Sequence
  Synthetic Sequence
gagaggacaggtgctacagg

SEQ ID NO: 253 - DNA, 20 bp, Artificial Sequence
  Synthetic Sequence
aaccaaggaagggcaggagg

SEQ ID NO: 254 - DNA, 20 bp, Artificial Sequence
  Synthetic Sequence
gacctctgggtggagacaga

SEQ ID NO: 255 - DNA, 20 bp, Artificial Sequence
  Synthetic Sequence
cagatgaccatgacaagcag

SEQ ID NO: 256 - DNA, 20 bp, Artificial Sequence
  Synthetic Sequence
aacaccagtgagtagagcgg

SEQ ID NO: 257 - DNA, 20 bp, Artificial Sequence
  Synthetic Sequence
aggaccttgaagcacagaga

SEQ ID NO: 258 - DNA, 20 bp, Artificial Sequence
  Synthetic Sequence
tacagaggcagactaaccca

SEQ ID NO: 259 - DNA, 20 bp, Artificial Sequence
  Synthetic Sequence
acagaggcagactaacccag

SEQ ID NO: 260 - DNA, 20 bp, Artificial Sequence
  Synthetic Sequence
taaatgacgtgctagacctg

SEQ ID NO: 261 - DNA, 20 bp, Artificial Sequence
  Synthetic Sequence
agtaaccactcaggacaggg

SEQ ID NO: 262 - DNA, 20 bp, Artificial Sequence
  Synthetic Sequence
accacaaaacagaaacacca

SEQ ID NO: 263 - DNA, 20 bp, Artificial Sequence
  Synthetic Sequence
gtttgaagacaagcctgagg

SEQ ID NO: 264 - DNA, 20 bp, Artificial Sequence
  Synthetic Sequence
gctgaaccccaaaagacagg

SEQ ID NO: 265 - DNA, 20 bp, Artificial Sequence
  Synthetic Sequence
gcagctgagacacacaccag

SEQ ID NO: 266 - DNA, 20 bp, Artificial Sequence
  Synthetic Sequence
aggacaccccaaagaagctg

SEQ ID NO: 267 - DNA, 20 bp, Artificial Sequence
  Synthetic Sequence
ggacaccccaaagaagctga

SEQ ID NO: 268 - DNA, 20 bp, Artificial Sequence
  Synthetic Sequence
ccagtgcaatggacagaaga

SEQ ID NO: 269 - DNA, 20 bp, Artificial Sequence
  Synthetic Sequence
agaagagggagcctgcaagt

SEQ ID NO: 270 - DNA, 20 bp, Artificial Sequence
  Synthetic Sequence
gtgtttgggccctagagcga

SEQ ID NO: 271 - DNA, 20 bp, Artificial Sequence
  Synthetic Sequence
catgtgcctggtgcaatgca

SEQ ID NO: 272 - DNA, 20 bp, Artificial Sequence
  Synthetic Sequence
tacaaagaggaagataagtg

SEQ ID NO: 273 - DNA, 20 bp, Artificial Sequence
  Synthetic Sequence
gtcacagaatacaccactag

SEQ ID NO: 274 - DNA, 20 bp, Artificial Sequence
  Synthetic Sequence
gggttaccctggacatggaa

SEQ ID NO: 275 - DNA, 20 bp, Artificial Sequence
  Synthetic Sequence
catggaagggtattcactcg

SEQ ID NO: 276 - DNA, 20 bp, Artificial Sequence
  Synthetic Sequence
agagtggcctagacaggctg

SEQ ID NO: 277 - DNA, 20 bp, Artificial Sequence
  Synthetic Sequence
catgctggacagctcggcag

SEQ ID NO: 278 - DNA, 20 bp, Artificial Sequence
  Synthetic Sequence
agtgaaagaagagaaaattc

SEQ ID NO: 279 - DNA, 20 bp, Artificial Sequence
  Synthetic Sequence
tggtaagtctaagaaaccta

SEQ ID NO: 280 - DNA, 20 bp, Artificial Sequence
  Synthetic Sequence
cccacagcctaaccacccta

SEQ ID NO: 281 - DNA, 20 bp, Artificial Sequence
  Synthetic Sequence
aatatttcaaagccctaggg

SEQ ID NO: 282 - DNA, 20 bp, Artificial Sequence
  Synthetic Sequence
gcactcggaacagggtctgg

SEQ ID NO: 283 - DNA, 20 bp, Artificial Sequence
  Synthetic Sequence
agataggagctccaacagtg

SEQ ID NO: 284 - DNA, 20 bp, Artificial Sequence
  Synthetic Sequence
aagttagagcagccaggaaa

SEQ ID NO: 285 - DNA, 20 bp, Artificial Sequence
  Synthetic Sequence
tagagcagccaggaaaggga

SEQ ID NO: 286 - DNA, 20 bp, Artificial Sequence
  Synthetic Sequence
tgaatacccttccatgtcca

SEQ ID NO: 287 - DNA, 20 bp, Artificial Sequence
  Synthetic Sequence
cctgcattgcaccaggcaca

SEQ ID NO: 288 - DNA, 20 bp, Artificial Sequence
  Synthetic Sequence
tctagggcccaaacacacct

SEQ ID NO: 289 - DNA, 20 bp, Artificial Sequence
  Synthetic Sequence
tccctccatctatcaaaagg

SEQ ID NO: 290 - DNA, 20 bp, Artificial Sequence
  Synthetic Sequence
agccctgagacagaagcagg

SEQ ID NO: 291 - DNA, 20 bp, Artificial Sequence
  Synthetic Sequence
gccctgagacagaagcaggt

SEQ ID NO: 292 - DNA, 20 bp, Artificial Sequence
  Synthetic Sequence
aggagatgcagtgatacgca

SEQ ID NO: 293 - DNA, 20 bp, Artificial Sequence
  Synthetic Sequence
acaataccaagggtatccgg

SEQ ID NO: 294 - DNA, 20 bp, Artificial Sequence
  Synthetic Sequence
tgataaagaaaacaaagtga

SEQ ID NO: 295 - DNA, 20 bp, Artificial Sequence
  Synthetic Sequence
aaagaaaacaaagtgaggga

SEQ ID NO: 296 - DNA, 20 bp, Artificial Sequence
  Synthetic Sequence
gtggcaagtggagaaattga

SEQ ID NO: 297 - DNA, 20 bp, Artificial Sequence
  Synthetic Sequence
caagtggagaaattgaggga

SEQ ID NO: 298 - DNA, 20 bp, Artificial Sequence
  Synthetic Sequence
gtggtgatgattgcagctgg

SEQ ID NO: 299 - DNA, 20 bp, Artificial Sequence
  Synthetic Sequence
ctatgtgcctgacacacagg

SEQ ID NO: 300 - DNA, 20 bp, Artificial Sequence
  Synthetic Sequence
gggttggaccaggaaagagg

SEQ ID NO: 301 - DNA, 20 bp, Artificial Sequence
  Synthetic Sequence
gatgcctggaaaaggaaaga

SEQ ID NO: 302 - DNA, 20 bp, Artificial Sequence
  Synthetic Sequence
tagtatgcacctgcaagagg

SEQ ID NO: 303 - DNA, 20 bp, Artificial Sequence
  Synthetic Sequence
tatgcacctgcaagaggcgg

SEQ ID NO: 304 - DNA, 20 bp, Artificial Sequence
  Synthetic Sequence
aggggaagaagagaagcaga

SEQ ID NO: 305 - DNA, 20 bp, Artificial Sequence
  Synthetic Sequence
gctgaatcaagagacaagcg

SEQ ID NO: 306 - DNA, 20 bp, Artificial Sequence
  Synthetic Sequence
aagcaaataaatctcctggg

SEQ ID NO: 307 - DNA, 20 bp, Artificial Sequence
  Synthetic Sequence
agatgagtgctagagactgg

SEQ ID NO: 308 - DNA, 20 bp, Artificial Sequence
  Synthetic Sequence
ctgatggttgagcacagcag

SEQ ID NO: 309 - DNA, 20 bp, Artificial Sequence
  Synthetic Sequence
caaatggccagcaagggtgg

SEQ ID NO: 310 - DNA, 20 bp, Artificial Sequence
  Synthetic Sequence
gcagaacctgaggatatgga

SEQ ID NO: 311 - DNA, 20 bp, Artificial Sequence
  Synthetic Sequence
aatacacagaatgaaaatag

SEQ ID NO: 312 - DNA, 20 bp, Artificial Sequence
  Synthetic Sequence
ctggtgactagaataggcag

SEQ ID NO: 313 - DNA, 20 bp, Artificial Sequence
  Synthetic Sequence
tggtgactagaataggcagt

SEQ ID NO: 314 - DNA, 20 bp, Artificial Sequence
  Synthetic Sequence
taaaagaatgtgaaaagatg

SEQ ID NO: 315 - DNA, 20 bp, Artificial Sequence
  Synthetic Sequence
tcaggagttcaagaccaccc

SEQ ID NO: 316 - DNA, 20 bp, Artificial Sequence
  Synthetic Sequence
tgtagtcccagttatgcagg

SEQ ID NO: 317 - DNA, 20 bp, Artificial Sequence
  Synthetic Sequence
gggttcacaccacaaatgca

SEQ ID NO: 318 - DNA, 20 bp, Artificial Sequence
  Synthetic Sequence
ggcaaatggccagcaagggt

SEQ ID NO: 319 - DNA, 20 bp, Artificial Sequence
  Synthetic Sequence
agaaaccaatcccaaagcaa

SEQ ID NO: 320 - DNA, 20 bp, Artificial Sequence
  Synthetic Sequence
gccaaggacaccaaaaccca

SEQ ID NO: 321 - DNA, 20 bp, Artificial Sequence
  Synthetic Sequence
agtggtgataaggcaacagt

SEQ ID NO: 322 - DNA, 20 bp, Artificial Sequence
  Synthetic Sequence
cctgagacagaagtattaag

SEQ ID NO: 323 - DNA, 20 bp, Artificial Sequence
  Synthetic Sequence
aaggtcacacaatgaatagg

SEQ ID NO: 324 - DNA, 20 bp, Artificial Sequence
  Synthetic Sequence
caccatactagggaagaaga

SEQ ID NO: 325 - DNA, 20 bp, Artificial Sequence
  Synthetic Sequence
aataccctgcccttagtggg

SEQ ID NO: 326 - DNA, 20 bp, Artificial Sequence
  Synthetic Sequence
ttagtggggggtggagtggg

SEQ ID NO: 327 - DNA, 20 bp, Artificial Sequence
  Synthetic Sequence
caataccctgcccttagtgg

SEQ ID NO: 328 - DNA, 20 bp, Artificial Sequence
  Synthetic Sequence
gtggggggtggagtgggggg

SEQ ID NO: 329 - DNA, 20 bp, Artificial Sequence
  Synthetic Sequence
ggggggtggagtggggggtg

SEQ ID NO: 330 - DNA, 20 bp, Artificial Sequence
  Synthetic Sequence
ggggtggagtggggggtggg

SEQ ID NO: 331 - DNA, 20 bp, Artificial Sequence
  Synthetic Sequence
gggtggagtggggggtgggg

SEQ ID NO: 332 - DNA, 20 bp, Artificial Sequence
  Synthetic Sequence
gggggtggggaaagacatcg

SEQ ID NO: 333 - DNA, 20 bp, Artificial Sequence
  Synthetic Sequence
gcagctgtgaattctgatag

SEQ ID NO: 334 - DNA, 20 bp, Artificial Sequence
  Synthetic Sequence
gagatcagagaaaccagatg

SEQ ID NO: 335 - DNA, 20 bp, Artificial Sequence
  Synthetic Sequence
tctatactgattgcagccag

SEQ ID NO: 336-347 - skipped (12 entries)

SEQ ID NO: 348 - protein, 6 aa, Artificial Sequence
  Synthetic Sequence
  MISC_FEATURE (1)..(6): Xaa can be any naturally occurring amino acid; SEQ ID NO: 348 corresponds to the sequence K(K/R)X(K/R)
XXXXXX

SEQ ID NO: 349 - protein, 7 aa, Artificial Sequence
  Synthetic Sequence
  MISC_FEATURE (1)..(7): Xaa can be any naturally occurring amino acid; SEQ ID NO: 349 corresponds to the sequence (K/R)(K/R)X10-12(K/R) 3/5
XXXXXXX

SEQ ID NO: 350 - protein, 9 aa, Artificial Sequence
  Synthetic Sequence
PAAKRVKLD

SEQ ID NO: 351 - protein, 16 aa, Artificial Sequence
  Synthetic Sequence
KRPAATKKAGQAKKKK

SEQ ID NO: 352 - protein, 7 aa, Artificial Sequence
  Synthetic Sequence
PKKKRKV

SEQ ID NO: 353 - protein, 24 aa, Artificial Sequence
  Synthetic Sequence
DPKKKRKVDPKKKRKVDPKKKRKV

SEQ ID NO: 354 - skipped

SEQ ID NO: 355 - protein, 36 aa, Artificial Sequence
  Synthetic Sequence
NHNHHDHDNHNHHDHDNGNHNIHDHDNIHDNGNHNH

SEQ ID NO: 356 - protein, 36 aa, Artificial Sequence
  Synthetic Sequence
NHNININHNHHDHDNGNHNHHDHDNHNHHDHDNGNH

SEQ ID NO: 357 - protein, 36 aa, Artificial Sequence
  Synthetic Sequence
NHNINHHDNIHDNGNHNININHNHHDHDNGNHNHHD

SEQ ID NO: 358 - protein, 36 aa, Artificial Sequence
  Synthetic Sequence
HDHDNIHDNGNHNINHHDNIHDNGNHNININHNHHD

SEQ ID NO: 359 - protein, 36 aa, Artificial Sequence
  Synthetic Sequence
NHNHNGNGNGHDHDNIHDNGNHNINHHDNIHDNGNH

SEQ ID NO: 360 - protein, 36 aa, Artificial Sequence
  Synthetic Sequence
NHNHNHNHNININININGNHNIHDHDHDNINIHDNI

SEQ ID NO: 361 - protein, 36 aa, Artificial Sequence
  Synthetic Sequence
NINHNHNIHDNINHNGNHNHNHNHNININININGNH

SEQ ID NO: 362 - protein, 36 aa, Artificial Sequence
  Synthetic Sequence
HDHDNINHNHNHNIHDNIHDNHNHNGNHHDNGNINH

SEQ ID NO: 363 - protein, 36 aa, Artificial Sequence
  Synthetic Sequence
HDNINHNINHHDHDNINHNHNINHNGHDHDNGNHNH

SEQ ID NO: 364 - protein, 36 aa, Artificial Sequence
  Synthetic Sequence
HDHDNGNGHDNINHNINHHDHDNINHNHNINHNGHD

SEQ ID NO: 365 - protein, 36 aa, Artificial Sequence
  Synthetic Sequence
HDHDNGHDHDNGNGHDNINHNINHHDHDNINHNHNI

SEQ ID NO: 366 - protein, 36 aa, Artificial Sequence
  Synthetic Sequence
HDHDNINHHDHDHDHDNGHDHDNGHDHDNGNGHDNI

SEQ ID NO: 367 - protein, 36 aa, Artificial Sequence
  Synthetic Sequence
HDHDNHNINHHDNGNGNHNIHDHDHDNGNGNHNHNI

SEQ ID NO: 368 - protein, 36 aa, Artificial Sequence
  Synthetic Sequence
NHNHNGNGNGHDHDNHNINHHDNGNGNHNIHDHDHD

SEQ ID NO: 369 - protein, 36 aa, Artificial Sequence
  Synthetic Sequence
NHNHNHNHNGNHNHNGNGNGHDHDNHNINHHDNGNG

SEQ ID NO: 370 - protein, 36 aa, Artificial Sequence
  Synthetic Sequence
HDNGNHHDNGNHNHNHNHNGNHNHNGNGNGHDHDNH

SEQ ID NO: 371 - protein, 36 aa, Artificial Sequence
  Synthetic Sequence
NHHDNINHNINHNGNINGHDNGNHHDNGNHNHNHNH

SEQ ID NO: 372 - protein, 28 aa, Artificial Sequence
  Synthetic Sequence
HDHDNININGHDHDHDHDNGHDNINHNG

SEQ ID NO: 373 - protein, 30 aa, Artificial Sequence
  Synthetic Sequence
HDNINHNGNHHDNGHDNINHNGNHNHNINI

SEQ ID NO: 374 - protein, 36 aa, Artificial Sequence
  Synthetic Sequence
NHNININIHDNINGHDHDNHNHHDNHNIHDNGHDNI

SEQ ID NO: 375 - protein, 36 aa, Artificial Sequence
  Synthetic Sequence
HDNHHDHDHDHDNGHDNINININGHDNGNGNIHDNI

SEQ ID NO: 376 - protein, 36 aa, Artificial Sequence
  Synthetic Sequence
HDNINININGHDNGNGNIHDNINHHDNGNHHDNGHD

SEQ ID NO: 377 - protein, 36 aa, Artificial Sequence
  Synthetic Sequence
HDNGNGNIHDNINHHDNGNHHDNGHDNIHDNGHDHD

SEQ ID NO: 378 - protein, 36 aa, Artificial Sequence
  Synthetic Sequence
NIHDNINHHDNGNHHDNGHDNIHDNGHDHDHDHDNG

SEQ ID NO: 379 - protein, 36 aa, Artificial Sequence
  Synthetic Sequence
NHHDNGHDNIHDNGHDHDHDHDNGNHHDNINHNHNH

SEQ ID NO: 380 - protein, 36 aa, Artificial Sequence
  Synthetic Sequence
HDHDHDHDNGNHHDNINHNHNHHDNINIHDNHHDHD

SEQ ID NO: 381 - protein, 36 aa, Artificial Sequence
  Synthetic Sequence
NHHDNINHNHNHHDNINIHDNHHDHDHDNINHNHNH

SEQ ID NO: 382 - protein, 36 aa, Artificial Sequence
  Synthetic Sequence
HDNGHDNHNINGNGNINGNHNHNHHDNHNHNHNING

SEQ ID NO: 383 - protein, 36 aa, Artificial Sequence
  Synthetic Sequence
HDNHHDNGNGHDNGHDNHNINGNGNINGNHNHNHHD

SEQ ID NO: 384 - protein, 36 aa, Artificial Sequence
  Synthetic Sequence
NHNGHDNHNINHNGHDNHHDNGNGHDNGHDNHNING

SEQ ID NO: 385 - protein, 36 aa, Artificial Sequence
  Synthetic Sequence
HDHDNINGNHNGHDNHNINHNGHDNHHDNGNGHDNG

SEQ ID NO: 386 - protein, 36 aa, Artificial Sequence
  Synthetic Sequence
HDNHHDHDNGHDHDNINGNHNGHDNHNINHNGHDNH

SEQ ID NO: 387 - protein, 36 aa, Artificial Sequence
  Synthetic Sequence
HDNHNGHDNINGHDNHHDHDNGHDHDNINGNHNGHD

SEQ ID NO: 388 - protein, 36 aa, Artificial Sequence
  Synthetic Sequence
NHNINGHDNGHDNHNGHDNINGHDNHHDHDNGHDHD

SEQ ID NO: 389 - protein, 28 aa, Artificial Sequence
  Synthetic Sequence
NHHDNGNGHDNINHHDNGNGHDHDNGNI

SEQ ID NO: 390 - protein, 28 aa, Artificial Sequence
  Synthetic Sequence
HDNGNKNGNHNINGHDNINGNHHDHDNI

SEQ ID NO: 391 - protein, 32 aa, Artificial Sequence
  Synthetic Sequence
NIHDNINNNGNNNNNGNIHDNIHDNIHDHDNG

SEQ ID NO: 392 - protein, 30 aa, Artificial Sequence
  Synthetic Sequence
HDHDNIHDHDHDHDHDHDNIHDNGNININN

SEQ ID NO: 393 - protein, 28 aa, Artificial Sequence
  Synthetic Sequence
HDNINGNGNNNNHDHDNNNNNNHDNIHD

SEQ ID NO: 394 - protein, 32 aa, Artificial Sequence
  Synthetic Sequence
NNHDNGNGNNNINIHDHDHDNINNNNNINNNI

SEQ ID NO: 395 - protein, 34 aa, Artificial Sequence
  Synthetic Sequence
NIHDNIHDHDHDNNNINGHDHDNIHDNGNNNNNN

SEQ ID NO: 396 - protein, 28 aa, Artificial Sequence
  Synthetic Sequence
NNHDNGNNHDNINGHDNINIHDHDHDHD

SEQ ID NO: 397 - protein, 34 aa, Artificial Sequence
  Synthetic Sequence
NNNNHDNIHDNNNININIHDNIHDHDHDNGHDHD

SEQ ID NO: 398 - protein, 30 aa, Artificial Sequence
  Synthetic Sequence
NNNNNGNNNNHDNGHDNINGNNHDHDNGNN

SEQ ID NO: 399 - protein, 32 aa, Artificial Sequence
  Synthetic Sequence
NNNINGNGNGNNHDNIHDNINNHDNGHDNING

SEQ ID NO: 400 - protein, 28 aa, Artificial Sequence
  Synthetic Sequence
NININHHDNGHDNGNHNINHNHNINHHD

SEQ ID NO: 401 - protein, 28 aa, Artificial Sequence
  Synthetic Sequence
HDHDHDNGNINKHDNGNHNGHDHDHDHD

SEQ ID NO: 402 - protein, 28 aa, Artificial Sequence
  Synthetic Sequence
NHHDHDNGNINHHDNINGNHHDNGNINH

SEQ ID NO: 403 - protein, 30 aa, Artificial Sequence
  Synthetic Sequence
NINGNHNHNHHDNGNGHDNIHDNHNHNING

SEQ ID NO: 404 - protein, 28 aa, Artificial Sequence
  Synthetic Sequence
NHNININIHDNGNINGNHHDHDNGNHHD

SEQ ID NO: 405 - protein, 28 aa, Artificial Sequence
  Synthetic Sequence
NHHDNIHDHDNINGNGNHHDNGHDHDHD

SEQ ID NO: 406 - protein, 28 aa, Artificial Sequence
  Synthetic Sequence
NHNIHDNINGNHHDNINIHDNGHDNINH

SEQ ID NO: 407 - protein, 28 aa, Artificial Sequence
  Synthetic Sequence
NIHDNIHDHDNIHDNGNINHNHNHNHNG

SEQ ID NO: 408 - protein, 28 aa, Artificial Sequence
  Synthetic Sequence
NHNGHDNGNHHDNGNINHNIHDNINHNH

SEQ ID NO: 409 - protein, 30 aa, Artificial Sequence
  Synthetic Sequence
NHNHHDHDNGNINHNIHDNINHNHHDNGNH

SEQ ID NO: 410 - protein, 30 aa, Artificial Sequence
  Synthetic Sequence
NHNINHNHHDNINGNGHDNGNGNINGHDNH

SEQ ID NO: 411 - protein, 30 aa, Artificial Sequence
  Synthetic Sequence
NNHDHDNGNNNNNININIHDNNNGNGHDHD

SEQ ID NO: 412 - protein, 28 aa, Artificial Sequence
  Synthetic Sequence
NNNGNNHDNGHDNGNNNIHDNININGNI

SEQ ID NO: 413 - protein, 28 aa, Artificial Sequence
  Synthetic Sequence
NNNGNGNGNGNNHDNINNHDHDNGHDHD

SEQ ID NO: 414 - protein, 30 aa, Artificial Sequence
  Synthetic Sequence
NIHDNINNHDNGNNNGNNNNNINIHDNNNG

SEQ ID NO: 415 - protein, 36 aa, Artificial Sequence
  Synthetic Sequence
HDNININNNIHDHDNNNINNHDNIHDNGNNHDNGNN

SEQ ID NO: 416 - protein, 30 aa, Artificial Sequence
  Synthetic Sequence
HDNGNINGHDHDHDNINININIHDNGHDNG

SEQ ID NO: 417 - protein, 28 aa, Artificial Sequence
  Synthetic Sequence
NHNININININIHDNGNINGNHNGNING

SEQ ID NO: 418 - protein, 30 aa, Artificial Sequence
  Synthetic Sequence
NINHNHHDNINHNHHDNGNHNHNGNGNHNI

SEQ ID NO: 419 - protein, 28 aa, Artificial Sequence
  Synthetic Sequence
HDNININGNIHDNINIHDHDNIHDNNHD

SEQ ID NO: 420 - protein, 30 aa, Artificial Sequence
  Synthetic Sequence
NINGNNNIHDNNNNNIHDNGHDNINIHDNG

SEQ ID NO: 421 - protein, 28 aa, Artificial Sequence
  Synthetic Sequence
HDNIHDNINIHDNINGNGNGNNNGNINI

SEQ ID NO: 422 - protein, 28 aa, Artificial Sequence
  Synthetic Sequence
NINGNGNGHDHDNINNNGNNHDNIHDNI

SEQ ID NO: 423-424 - skipped (2 entries)

SEQ ID NO: 425 - DNA, 20 bp, Artificial Sequence
  Synthetic Sequence
aatcgagaagcgactcgaca

SEQ ID NO: 426 - DNA, 20 bp, Artificial Sequence
  Synthetic Sequence
tgccctgcaggggagtgagc

SEQ ID NO: 427 - DNA, 20 bp, Artificial Sequence
  Synthetic Sequence
gaagcgactcgacatggagg

SEQ ID NO: 428 - DNA, 20 bp, Artificial Sequence
  Synthetic Sequence
cctgcaggggagtgagcagc

SEQ ID NO: 429-439 - skipped (11 entries)

SEQ ID NO: 440 - DNA, 4 bp, Artificial Sequence
  Synthetic Sequence
ttaa

SEQ ID NO: 441 - DNA, 20 bp, Artificial Sequence
  Synthetic Sequence
gtccctgggcgttgccctgc

SEQ ID NO: 442 - DNA, 20 bp, Artificial Sequence
  Synthetic Sequence
ccctgggcgttgccctgcag

SEQ ID NO: 443 - DNA, 20 bp, Artificial Sequence
  Synthetic Sequence
gagtgagcagctgtaagatt

SEQ ID NO: 444 - DNA, 20 bp, Artificial Sequence
  Synthetic Sequence
caggggagtgagcagctgta

SEQ ID NO: 445 - DNA, 20 bp, Artificial Sequence
  Synthetic Sequence
cgttgccctgcaggggagtg

SEQ ID NO: 446 - DNA, 20 bp, Artificial Sequence
  Synthetic Sequence
tgggcgttgccctgcagggg

SEQ ID NO: 447 - DNA, 20 bp, Artificial Sequence
  Synthetic Sequence
ttggtccctgggcgttgccc

SEQ ID NO: 448 - DNA, 20 bp, Artificial Sequence
  Synthetic Sequence
aagaatcccgcccataatcg

SEQ ID NO: 449 - DNA, 20 bp, Artificial Sequence
  Synthetic Sequence
aatcccgcccataatcgaga

SEQ ID NO: 450 - DNA, 20 bp, Artificial Sequence
  Synthetic Sequence
tcccgcccataatcgagaag

SEQ ID NO: 451 - DNA, 20 bp, Artificial Sequence
  Synthetic Sequence
cccataatcgagaagcgact

SEQ ID NO: 452 - DNA, 20 bp, Artificial Sequence
  Synthetic Sequence
gagaagcgactcgacatgga

SEQ ID NO: 453 - DNA, 20 bp, Artificial Sequence
  Synthetic Sequence
gcgactcgacatggaggcga

SEQ ID NO: 454 - DNA, 20 bp, Artificial Sequence
  Synthetic Sequence
ccgtgggaagataaactaat

SEQ ID NO: 455 - DNA, 20 bp, Artificial Sequence
  Synthetic Sequence
tcccctgcagggcaacgccc

SEQ ID NO: 456 - DNA, 20 bp, Artificial Sequence
  Synthetic Sequence
gtcgagtcgcttctcgatta

SEQ ID NO: 457 - DNA, 20 bp, Artificial Sequence
  Synthetic Sequence
cgacaccaactctagtccgt

SEQ ID NO: 458 - DNA, 20 bp, Artificial Sequence
  Synthetic Sequence
cagctgctcactcccctgca

SEQ ID NO: 459 - DNA, 20 bp, Artificial Sequence
  Synthetic Sequence
agtcgcttctcgattatggg

SEQ ID NO: 460 - DNA, 23 bp, Artificial Sequence
  Synthetic Sequence
acccttggaaggacctggctggg

SEQ ID NO: 461 - DNA, 20 bp, Artificial Sequence
  Synthetic Sequence
tccgagcttgacccttggaa

SEQ ID NO: 462 - DNA, 23 bp, Artificial Sequence
  Synthetic Sequence
ggagccacgaaaacagatccagg

SEQ ID NO: 463 - DNA, 23 bp, Artificial Sequence
  Synthetic Sequence
agatccagggacacggtgctagg

SEQ ID NO: 464 - DNA, 23 bp, Artificial Sequence
  Synthetic Sequence
gacacggtgctaggacagtgggg

SEQ ID NO: 465 - DNA, 23 bp, Artificial Sequence
  Synthetic Sequence
gaaaatgacccaacagcctctgg

SEQ ID NO: 466 - DNA, 23 bp, Artificial Sequence
  Synthetic Sequence
gcctggccggcctgaccactggg

SEQ ID NO: 467 - DNA, 23 bp, Artificial Sequence
  Synthetic Sequence
ctgagcactgaaggcctggccgg

SEQ ID NO: 468 - DNA, 23 bp, Artificial Sequence
  Synthetic Sequence
tggtttccactgagcactgaagg

SEQ ID NO: 469 - DNA, 23 bp, Artificial Sequence
  Synthetic Sequence
ggtgctttcctgaggaccgatag

SEQ ID NO: 470 - DNA, 23 bp, Artificial Sequence
  Synthetic Sequence
gcgcttccagtgctcagactagg

SEQ ID NO: 471 - DNA, 23 bp, Artificial Sequence
  Synthetic Sequence
cagtgctcagactagggaagagg

SEQ ID NO: 472 - DNA, 23 bp, Artificial Sequence
  Synthetic Sequence
gcccctcctccttcagagccagg

SEQ ID NO: 473 - DNA, 23 bp, Artificial Sequence
  Synthetic Sequence
tccttcagagccaggagtcctgg

SEQ ID NO: 474 - DNA, 23 bp, Artificial Sequence
  Synthetic Sequence
ccaagggtcaagctcggaaacca

SEQ ID NO: 475 - DNA, 23 bp, Artificial Sequence
  Synthetic Sequence
ctgcagagtatctgctggggtgg

SEQ ID NO: 476 - DNA, 23 bp, Artificial Sequence
  Synthetic Sequence
cgttcctgcagagtatctgctgg

SEQ ID NO: 477 - DNA, 20 bp, Artificial Sequence
  Synthetic Sequence
gtggggaaaatgacccaaca

SEQ ID NO: 478 - DNA, 20 bp, Artificial Sequence
  Synthetic Sequence
gaaggcctggccggcctgac

SEQ ID NO: 479 - DNA, 20 bp, Artificial Sequence
  Synthetic Sequence
actcctggctctgaaggagg

SEQ ID NO: 480 - DNA, 20 bp, Artificial Sequence
  Synthetic Sequence
gggctgggggccaggactcc

SEQ ID NO: 481 - DNA, 20 bp, Artificial Sequence
  Synthetic Sequence
gtccttccaagggtcaagct

SEQ ID NO: 482 - DNA, 20 bp, Artificial Sequence
  Synthetic Sequence
tcaagctcggaaaccacccc

SEQ ID NO: 483 - DNA, 23 bp, Artificial Sequence
  Synthetic Sequence
attgtcttcactaaacccgttgg

SEQ ID NO: 484 - DNA, 23 bp, Artificial Sequence
  Synthetic Sequence
taaacccgttgggaatacaatgg

SEQ ID NO: 485 - DNA, 23 bp, Artificial Sequence
  Synthetic Sequence
ttgtcttcactaaacccgttggg

SEQ ID NO: 486 - DNA, 23 bp, Artificial Sequence
  Synthetic Sequence
tgattcataggagtctattaagg

SEQ ID NO: 487 - DNA, 23 bp, Artificial Sequence
  Synthetic Sequence
ttacatatgcttcgagtttgtgg

SEQ ID NO: 488 - DNA, 23 bp, Artificial Sequence
  Synthetic Sequence
actcttaaggtaggactaattgg

SEQ ID NO: 489 - DNA, 23 bp, Artificial Sequence
  Synthetic Sequence
tatgtgtgcaatagcgttaaagg

SEQ ID NO: 490 - DNA, 23 bp, Artificial Sequence
  Synthetic Sequence
cgttgggaatacaatggcttagg

SEQ ID NO: 491 - DNA, 23 bp, Artificial Sequence
  Synthetic Sequence
tcacaatggaactctgcctttgg

SEQ ID NO: 492 - DNA, 23 bp, Artificial Sequence
  Synthetic Sequence
gaccacaaatcaatgcccaaagg

SEQ ID NO: 493 - DNA, 23 bp, Artificial Sequence
  Synthetic Sequence
ctaagccattgtattcccaacgg

SEQ ID NO: 494 - DNA, 23 bp, Artificial Sequence
  Synthetic Sequence
agcattctggagtgtcacaatgg

SEQ ID NO: 495 - DNA, 23 bp, Artificial Sequence
  Synthetic Sequence
caatagcccactttaatactagg

SEQ ID NO: 496 - DNA, 23 bp, Artificial Sequence
  Synthetic Sequence
ctttatccaagtgaatcctttgg

SEQ ID NO: 497 - DNA, 23 bp, Artificial Sequence
  Synthetic Sequence
ggcattgatttgtggtcatttgg

SEQ ID NO: 498 - DNA, 23 bp, Artificial Sequence
  Synthetic Sequence
taagccattgtattcccaacggg

SEQ ID NO: 499 - DNA, 23 bp, Artificial Sequence
  Synthetic Sequence
aatacaatcactcttaaggtagg

SEQ ID NO: 500 - DNA, 23 bp, Artificial Sequence
  Synthetic Sequence
gaagtacctttcactattttggg

SEQ ID NO: 501 - DNA, 23 bp, Artificial Sequence
  Synthetic Sequence
caagcaacaaatgacttctaagg

SEQ ID NO: 502 - DNA, 23 bp, Artificial Sequence
  Synthetic Sequence
tttgaatacaatcactcttaagg

SEQ ID NO: 503 - DNA, 23 bp, Artificial Sequence
  Synthetic Sequence
acaaacggactacgtaaacttgg

SEQ ID NO: 504 - DNA, 23 bp, Artificial Sequence
  Synthetic Sequence
acaagatgtgaacacgacgatgg

SEQ ID NO: 505 - DNA, 23 bp, Artificial Sequence
  Synthetic Sequence
gttgcaccgttgattccttcagg

SEQ ID NO: 506 - DNA, 23 bp, Artificial Sequence
  Synthetic Sequence
agtaatattgaattagggcgtgg

SEQ ID NO: 507 - DNA, 23 bp, Artificial Sequence
  Synthetic Sequence
cctgatgttggctcgacattagg

SEQ ID NO: 508 - DNA, 23 bp, Artificial Sequence
  Synthetic Sequence
ctttgttgggtcttagcttaagg

SEQ ID NO: 509 - DNA, 23 bp, Artificial Sequence
  Synthetic Sequence
tcggaacagctccttcctgaagg

SEQ ID NO: 510 - DNA, 23 bp, Artificial Sequence
  Synthetic Sequence
agtagtttctgaggtcatgttgg

SEQ ID NO: 511 - DNA, 23 bp, Artificial Sequence
  Synthetic Sequence
cttgaaaatacgatgatgtgagg

SEQ ID NO: 512 - DNA, 23 bp, Artificial Sequence
  Synthetic Sequence
gcattaatctagagagagggagg

SEQ ID NO: 513 - DNA, 23 bp, Artificial Sequence
  Synthetic Sequence
gggtcatgttagaattcatgtgg

SEQ ID NO: 514 - DNA, 23 bp, Artificial Sequence
  Synthetic Sequence
tgatgcattaatctagagagagg

SEQ ID NO: 515 - DNA, 23 bp, Artificial Sequence
  Synthetic Sequence
acatcatcgtattttcaagttgg

SEQ ID NO: 516 - DNA, 23 bp, Artificial Sequence
  Synthetic Sequence
ctagctgacaaacatgtgagtgg

SEQ ID NO: 517 - DNA, 23 bp, Artificial Sequence
  Synthetic Sequence
aacatgacccaagtgagtccagg

SEQ ID NO: 518 - DNA, 23 bp, Artificial Sequence
  Synthetic Sequence
gattccgtatttgctttgttggg

SEQ ID NO: 519 - DNA, 23 bp, Artificial Sequence
  Synthetic Sequence
tacgatgatgtgaggaaataagg

SEQ ID NO: 520 - DNA, 23 bp, Artificial Sequence
  Synthetic Sequence
gtaatatgtctaagtactgatgg

SEQ ID NO: 521 - DNA, 23 bp, Artificial Sequence
  Synthetic Sequence
gtaaagtgagctggttcattagg

SEQ ID NO: 522 - DNA, 23 bp, Artificial Sequence
  Synthetic Sequence
actagagtccttaagaagggggg

SEQ ID NO: 523 - DNA, 23 bp, Artificial Sequence
  Synthetic Sequence
ataacacgtgagccgtcctaagg

SEQ ID NO: 524 - DNA, 23 bp, Artificial Sequence
  Synthetic Sequence
ggaagacttttctctatacgagg

SEQ ID NO: 525 - DNA, 23 bp, Artificial Sequence
  Synthetic Sequence
gcattcctttcatccatggcagg

SEQ ID NO: 526 - DNA, 23 bp, Artificial Sequence
  Synthetic Sequence
gacatatggttataaaaatcagg

SEQ ID NO: 527 - DNA, 23 bp, Artificial Sequence
  Synthetic Sequence
ggagtgcagtccctgacatatgg

SEQ ID NO: 528 - DNA, 23 bp, Artificial Sequence
  Synthetic Sequence
gtgggttagggtggttaactggg

SEQ ID NO: 529 - DNA, 23 bp, Artificial Sequence
  Synthetic Sequence
aggtgcaaaaaggttgctgtggg

SEQ ID NO: 530 - DNA, 23 bp, Artificial Sequence
  Synthetic Sequence
cgtgacaaggcaaagtggcgtgg

SEQ ID NO: 531 - DNA, 23 bp, Artificial Sequence
  Synthetic Sequence
gaaggactgcccctgacgtcagg

SEQ ID NO: 532 - DNA, 23 bp, Artificial Sequence
  Synthetic Sequence
ctgcccctgacgtcaggagttgg

SEQ ID NO: 533 - DNA, 23 bp, Artificial Sequence
  Synthetic Sequence
tgtgggttagggtggttaactgg

SEQ ID NO: 534 - DNA, 23 bp, Artificial Sequence
  Synthetic Sequence
acccttttagagttttctgctgg

SEQ ID NO: 535 - DNA, 23 bp, Artificial Sequence
  Synthetic Sequence
aacttcctgccatggatgaaagg

SEQ ID NO: 536 - DNA, 23 bp, Artificial Sequence
  Synthetic Sequence
gcaaaaaggttgctgtgggttgg

SEQ ID NO: 537 - DNA, 23 bp, Artificial Sequence
  Synthetic Sequence
aatttgggggtagataggcatgg

SEQ ID NO: 538 - DNA, 23 bp, Artificial Sequence
  Synthetic Sequence
agaaaactctaaaagggtatagg

SEQ ID NO: 539 - DNA, 23 bp, Artificial Sequence
  Synthetic Sequence
attagcattcctttcatccatgg

SEQ ID NO: 540 - DNA, 23 bp, Artificial Sequence
  Synthetic Sequence
cccagcagaaaactctaaaaggg

SEQ ID NO: 541 - DNA, 23 bp, Artificial Sequence
  Synthetic Sequence
caggtgcaaaaaggttgctgtgg

SEQ ID NO: 542 - DNA, 23 bp, Artificial Sequence
  Synthetic Sequence
gcaagagatgaaattccatatgg

SEQ ID NO: 543 - DNA, 23 bp, Artificial Sequence
  Synthetic Sequence
gggctgttctaacgaagtctggg

SEQ ID NO: 544 - DNA, 23 bp, Artificial Sequence
  Synthetic Sequence
tgtccattcagcgaccctagagg

SEQ ID NO: 545 - DNA, 23 bp, Artificial Sequence
  Synthetic Sequence
ggctgttctaacgaagtctgggg

SEQ ID NO: 546 - DNA, 23 bp, Artificial Sequence
  Synthetic Sequence
gtccattcagcgaccctagaggg

SEQ ID NO: 547 - DNA, 23 bp, Artificial Sequence
  Synthetic Sequence
ggggctgttctaacgaagtctgg

SEQ ID NO: 548 - DNA, 23 bp, Artificial Sequence
  Synthetic Sequence
ggctgaatcagcatgcgaaaggg

SEQ ID NO: 549 - DNA, 23 bp, Artificial Sequence
  Synthetic Sequence
ttccaatggggggcatagcctgg

SEQ ID NO: 550 - DNA, 23 bp, Artificial Sequence
  Synthetic Sequence
taccctctagggtcgctgaatgg

SEQ ID NO: 551 - DNA, 23 bp, Artificial Sequence
  Synthetic Sequence
atcctcttgggccttataagagg

SEQ ID NO: 552 - DNA, 23 bp, Artificial Sequence
  Synthetic Sequence
ggccaggctatgccccccattgg

SEQ ID NO: 553 - DNA, 23 bp, Artificial Sequence
  Synthetic Sequence
ctagaggaccagaacaactctgg

SEQ ID NO: 554 - DNA, 23 bp, Artificial Sequence
  Synthetic Sequence
tccctcttataaggcccaagagg

SEQ ID NO: 555 - DNA, 23 bp, Artificial Sequence
  Synthetic Sequence
aggctgaatcagcatgcgaaagg

SEQ ID NO: 556 - DNA, 23 bp, Artificial Sequence
  Synthetic Sequence
ggaccagaacaactctggcctgg

SEQ ID NO: 557 - DNA, 23 bp, Artificial Sequence
  Synthetic Sequence
gggcttttatttggcccagcagg

SEQ ID NO: 558 - DNA, 23 bp, Artificial Sequence
  Synthetic Sequence
gtcgctgaatggacagactctgg

SEQ ID NO: 559 - DNA, 23 bp, Artificial Sequence
  Synthetic Sequence
ctcatgagttttaccctctaggg

SEQ ID NO: 560 - DNA, 23 bp, Artificial Sequence
  Synthetic Sequence
tcctcttgggccttataagaggg

SEQ ID NO: 561 - DNA, 23 bp, Artificial Sequence
  Synthetic Sequence
tcttgggccttataagagggagg

SEQ ID NO: 562 - DNA, 23 bp, Artificial Sequence
  Synthetic Sequence
tagaacagccccccacacagtgg

SEQ ID NO: 563 - DNA, 23 bp, Artificial Sequence
  Synthetic Sequence
gttacgttatgactaatctttgg

SEQ ID NO: 564 - DNA, 23 bp, Artificial Sequence
  Synthetic Sequence
tacgttatgactaatctttgggg

SEQ ID NO: 565 - DNA, 23 bp, Artificial Sequence
  Synthetic Sequence
ggaagtagtgttatgatgtatgg

SEQ ID NO: 566 - DNA, 23 bp, Artificial Sequence
  Synthetic Sequence
gttatgatgtatgggcataaagg

SEQ ID NO: 567 - DNA, 23 bp, Artificial Sequence
  Synthetic Sequence
gaagtagtgttatgatgtatggg

SEQ ID NO: 568 - DNA, 23 bp, Artificial Sequence
  Synthetic Sequence
atagctgctggcagtataactgg

SEQ ID NO: 569 - DNA, 23 bp, Artificial Sequence
  Synthetic Sequence
gcatcacaacattgacactgtgg

SEQ ID NO: 570 - DNA, 23 bp, Artificial Sequence
  Synthetic Sequence
aaggcgagtttctacaaagatgg

SEQ ID NO: 571 - DNA, 23 bp, Artificial Sequence
  Synthetic Sequence
ttacgttatgactaatctttggg

SEQ ID NO: 572 - DNA, 23 bp, Artificial Sequence
  Synthetic Sequence
caagactgattaagactgatggg

SEQ ID NO: 573 - DNA, 23 bp, Artificial Sequence
  Synthetic Sequence
agcagcaatgtattaaaggctgg

SEQ ID NO: 574 - DNA, 23 bp, Artificial Sequence
  Synthetic Sequence
ctacaggattgatgtaaacatgg

SEQ ID NO: 575 - DNA, 23 bp, Artificial Sequence
  Synthetic Sequence
tgggcataaagggttttaatggg

SEQ ID NO: 576 - DNA, 23 bp, Artificial Sequence
  Synthetic Sequence
acatcaatcctgtaggtgattgg

SEQ ID NO: 577 - DNA, 23 bp, Artificial Sequence
  Synthetic Sequence
attctagtcattatagctgctgg

SEQ ID NO: 578 - DNA, 23 bp, Artificial Sequence
  Synthetic Sequence
catcaatcctgtaggtgattggg

SEQ ID NO: 579 - DNA, 23 bp, Artificial Sequence
  Synthetic Sequence
gttataagatcaattctgagtgg

SEQ ID NO: 580 - DNA, 23 bp, Artificial Sequence
  Synthetic Sequence
ggcagactgtggatcaaaagtgg

SEQ ID NO: 581 - DNA, 23 bp, Artificial Sequence
  Synthetic Sequence
atggctgcccaatcacctacagg

SEQ ID NO: 582 - DNA, 23 bp, Artificial Sequence
  Synthetic Sequence
tcaaagcatgtacttagagttgg

SEQ ID NO: 583 - DNA, 20 bp, Artificial Sequence
  Synthetic Sequence
tcgcccctcaaatcttacag

SEQ ID NO: 584 - DNA, 20 bp, Artificial Sequence
  Synthetic Sequence
tcaaatcttacagctgctca

SEQ ID NO: 585 - DNA, 20 bp, Artificial Sequence
  Synthetic Sequence
tcttacagctgctcactccc

SEQ ID NO: 586 - DNA, 20 bp, Artificial Sequence
  Synthetic Sequence
tacagctgctcactcccctg

SEQ ID NO: 587 - DNA, 20 bp, Artificial Sequence
  Synthetic Sequence
tgctcactcccctgcagggc

SEQ ID NO: 588 - DNA, 20 bp, Artificial Sequence
  Synthetic Sequence
tgcagggcaacgcccaggga

SEQ ID NO: 589 - DNA, 20 bp, Artificial Sequence
  Synthetic Sequence
tctcgattatgggcgggatt

SEQ ID NO: 590 - DNA, 20 bp, Artificial Sequence
  Synthetic Sequence
tcgcttctcgattatgggcg

SEQ ID NO: 591 - DNA, 20 bp, Artificial Sequence
  Synthetic Sequence
tgtcgagtcgcttctcgatt

SEQ ID NO: 592 - DNA, 20 bp, Artificial Sequence
  Synthetic Sequence
tccatgtcgagtcgcttctc

SEQ ID NO: 593 - DNA, 20 bp, Artificial Sequence
  Synthetic Sequence
tcgcctccatgtcgagtcgc

SEQ ID NO: 594 - DNA, 20 bp, Artificial Sequence
  Synthetic Sequence
tcgtcatcgcctccatgtcg

SEQ ID NO: 595 - DNA, 20 bp, Artificial Sequence
  Synthetic Sequence
tgatctcgtcatcgcctcca

SEQ ID NO: 596 - protein, 38 aa, Artificial Sequence
  Synthetic Sequence
HDNHHDHDHDHDNGHDNINININGHDNGNGNIHDNINH

SEQ ID NO: 597 - protein, 38 aa, Artificial Sequence
  Synthetic Sequence
HDNINININGHDNGNGNIHDNINHHDNGNHHDNGHDNI

SEQ ID NO: 598 - protein, 38 aa, Artificial Sequence
  Synthetic Sequence
HDNGNGNIHDNINHHDNGNHHDNGHDNIHDNGHDHDHD

SEQ ID NO: 599 - protein, 38 aa, Artificial Sequence
  Synthetic Sequence
NIHDNINHHDNGNHHDNGHDNIHDNGHDHDHDHDNGNH

SEQ ID NO: 600 - protein, 38 aa, Artificial Sequence
  Synthetic Sequence
NHHDNGHDNIHDNGHDHDHDHDNGNHHDNINHNHNHHD

SEQ ID NO: 601 - protein, 38 aa, Artificial Sequence
  Synthetic Sequence
HDHDHDHDNGNHHDNINHNHNHHDNINIHDNHHDHDHD

SEQ ID NO: 602 - protein, 38 aa, Artificial Sequence
  Synthetic Sequence
NHHDNINHNHNHHDNINIHDNHHDHDHDNINHNHNHNI

SEQ ID NO: 603 - protein, 38 aa, Artificial Sequence
  Synthetic Sequence
HDNGHDNHNINGNGNINGNHNHNHHDNHNHNHNINGNG

SEQ ID NO: 604 - protein, 38 aa, Artificial Sequence
  Synthetic Sequence
HDNHHDNGNGHDNGHDNHNINGNGNINGNHNHNHHDNH

SEQ ID NO: 605 - protein, 38 aa, Artificial Sequence
  Synthetic Sequence
NHNGHDNHNINHNGHDNHHDNGNGHDNGHDNHNINGNG

SEQ ID NO: 606 - protein, 38 aa, Artificial Sequence
  Synthetic Sequence
HDHDNINGNHNGHDNHNINHNGHDNHHDNGNGHDNGHD

SEQ ID NO: 607 - protein, 38 aa, Artificial Sequence
  Synthetic Sequence
HDNHHDHDNGHDHDNINGNHNGHDNHNINHNGHDNHHD

SEQ ID NO: 608 - protein, 38 aa, Artificial Sequence
  Synthetic Sequence
HDNHNGHDNINGHDNHHDHDNGHDHDNINGNHNGHDNH

SEQ ID NO: 609 - protein, 38 aa, Artificial Sequence
  Synthetic Sequence
NHNINGHDNGHDNHNGHDNINGHDNHHDHDNGHDHDNI

SEQ ID NO: 610 - DNA, 20 bp, Artificial Sequence
  Synthetic Sequence
tggccggcctgaccactggg

SEQ ID NO: 611 - DNA, 20 bp, Artificial Sequence
  Synthetic Sequence
tgaaggcctggccggcctga

SEQ ID NO: 612 - DNA, 20 bp, Artificial Sequence
  Synthetic Sequence
tgagcactgaaggcctggcc

SEQ ID NO: 613 - DNA, 20 bp, Artificial Sequence
  Synthetic Sequence
tccactgagcactgaaggcc

SEQ ID NO: 614 - DNA, 20 bp, Artificial Sequence
  Synthetic Sequence
tggtttccactgagcactga

SEQ ID NO: 615 - DNA, 20 bp, Artificial Sequence
  Synthetic Sequence
tggggaaaatgacccaacag

SEQ ID NO: 616 - DNA, 20 bp, Artificial Sequence
  Synthetic Sequence
taggacagtggggaaaatga

SEQ ID NO: 617 - DNA, 20 bp, Artificial Sequence
  Synthetic Sequence
tccagggacacggtgctagg

SEQ ID NO: 618 - DNA, 20 bp, Artificial Sequence
  Synthetic Sequence
tcagagccaggagtcctggc

SEQ ID NO: 619 - DNA, 20 bp, Artificial Sequence
  Synthetic Sequence
tccttcagagccaggagtcc

SEQ ID NO: 620 - DNA, 20 bp, Artificial Sequence
  Synthetic Sequence
tcctccttcagagccaggag

SEQ ID NO: 621 - DNA, 20 bp, Artificial Sequence
  Synthetic Sequence
tccagcccctcctccttcag

SEQ ID NO: 622 - DNA, 20 bp, Artificial Sequence
  Synthetic Sequence
tggggtggtttccgagcttg

SEQ ID NO: 623 - DNA, 20 bp, Artificial Sequence
  Synthetic Sequence
tctgctggggtggtttccga

SEQ ID NO: 624 - DNA, 20 bp, Artificial Sequence
  Synthetic Sequence
tgcagagtatctgctggggt

SEQ ID NO: 625 - protein, 38 aa, Artificial Sequence
  Synthetic Sequence
NHNHHDHDNHNHHDHDNGNHNIHDHDNIHDNGNHNHNH

SEQ ID NO: 626 - protein, 38 aa, Artificial Sequence
  Synthetic Sequence
NHNININHNHHDHDNGNHNHHDHDNHNHHDHDNGNHNI

SEQ ID NO: 627 - protein, 38 aa, Artificial Sequence
  Synthetic Sequence
NHNINHHDNIHDNGNHNININHNHHDHDNGNHNHHDHD

SEQ ID NO: 628 - protein, 38 aa, Artificial Sequence
  Synthetic Sequence
HDHDNIHDNGNHNINHHDNIHDNGNHNININHNHHDHD

SEQ ID NO: 629 - protein, 38 aa, Artificial Sequence
  Synthetic Sequence
NHNHNGNGNGHDHDNIHDNGNHNINHHDNIHDNGNHNI

SEQ ID NO: 630 - protein, 38 aa, Artificial Sequence
  Synthetic Sequence
NHNHNHNHNININININGNHNIHDHDHDNINIHDNINH

SEQ ID NO: 631 - protein, 38 aa, Artificial Sequence
  Synthetic Sequence
NINHNHNIHDNINHNGNHNHNHNHNININININGNHNI

SEQ ID NO: 632 - protein, 38 aa, Artificial Sequence
  Synthetic Sequence
HDHDNINHNHNHNIHDNIHDNHNHNGNHHDNGNINHNH

SEQ ID NO: 633 - protein, 38 aa, Artificial Sequence
  Synthetic Sequence
HDNINHNINHHDHDNINHNHNINHNGHDHDNGNHNHHD

SEQ ID NO: 634 - protein, 38 aa, Artificial Sequence
  Synthetic Sequence
HDHDNGNGHDNINHNINHHDHDNINHNHNINHNGHDHD

SEQ ID NO: 635 - protein, 38 aa, Artificial Sequence
  Synthetic Sequence
HDHDNGHDHDNGNGHDNINHNINHHDHDNINHNHNINH

SEQ ID NO: 636 - protein, 38 aa, Artificial Sequence
  Synthetic Sequence
HDHDNINHHDHDHDHDNGHDHDNGHDHDNGNGHDNINH

SEQ ID NO: 637 - protein, 38 aa, Artificial Sequence
  Synthetic Sequence
HDHDNHNINHHDNGNGNHNIHDHDHDNGNGNHNHNINI

SEQ ID NO: 638 - protein, 38 aa, Artificial Sequence
  Synthetic Sequence
NHNHNGNGNGHDHDNHNINHHDNGNGNHNIHDHDHDNG

SEQ ID NO: 639 - protein, 38 aa, Artificial Sequence
  Synthetic Sequence
NHNHNHNHNGNHNHNGNGNGHDHDNHNINHHDNGNGNH

SEQ ID NO: 640 - protein, 38 aa, Artificial Sequence
  Synthetic Sequence
HDNGNHHDNGNHNHNHNHNGNHNHNGNGNGHDHDNHNI

SEQ ID NO: 641 - protein, 38 aa, Artificial Sequence
  Synthetic Sequence
NHHDNINHNINHNGNINGHDNGNHHDNGNHNHNHNHNG

SEQ ID NO: 642 - DNA, 18 bp, Artificial Sequence
  Synthetic Sequence
tcttcctagtattaaagt

SEQ ID NO: 643 - DNA, 17 bp, Artificial Sequence
  Synthetic Sequence
tccttaatattaccagt

SEQ ID NO: 644 - DNA, 25 bp, Artificial Sequence
  Synthetic Sequence
taccaagctgaaatgacacaaaagt

SEQ ID NO: 645 - DNA, 25 bp, Artificial Sequence
  Synthetic Sequence
tggctgtgtcacataccagcagaat

SEQ ID NO: 646 - DNA, 22 bp, Artificial Sequence
  Synthetic Sequence
tgttaatttgaatacaatcact

SEQ ID NO: 647 - DNA, 21 bp, Artificial Sequence
  Synthetic Sequence
tgtgtcacataccagcagaat

SEQ ID NO: 648 - DNA, 16 bp, Artificial Sequence
  Synthetic Sequence
tggtaactactaattt

SEQ ID NO: 649 - DNA, 19 bp, Artificial Sequence
  Synthetic Sequence
tgtcacataccagcagaat

SEQ ID NO: 650 - DNA, 21 bp, Artificial Sequence
  Synthetic Sequence
tgtgacacagccatcaacaat

SEQ ID NO: 651 - DNA, 16 bp, Artificial Sequence
  Synthetic Sequence
tcctttgatgaacagt

SEQ ID NO: 652 - DNA, 29 bp, Artificial Sequence
  Synthetic Sequence
tgtgtgcaatagcgttaaaggaactacat

SEQ ID NO: 653 - DNA, 17 bp, Artificial Sequence
  Synthetic Sequence
tctttcaatagcccact

SEQ ID NO: 654 - DNA, 22 bp, Artificial Sequence
  Synthetic Sequence
tctcaaatgacaagagcacagt

SEQ ID NO: 655 - DNA, 18 bp, Artificial Sequence
  Synthetic Sequence
taccagttaattagcact

SEQ ID NO: 656 - DNA, 18 bp, Artificial Sequence
  Synthetic Sequence
tgttgtgacctaagccat

SEQ ID NO: 657 - DNA, 23 bp, Artificial Sequence
  Synthetic Sequence
tctcatgttttaaagtcaagaat

SEQ ID NO: 658 - DNA, 19 bp, Artificial Sequence
  Synthetic Sequence
tcctgaattcagaacagat

SEQ ID NO: 659 - DNA, 26 bp, Artificial Sequence
  Synthetic Sequence
tagcatgatgtttcatgttgtgacct

SEQ ID NO: 660 - DNA, 25 bp, Artificial Sequence
  Synthetic Sequence
tgtttcatgttgtgacctaagccat

SEQ ID NO: 661 - DNA, 18 bp, Artificial Sequence
  Synthetic Sequence
tacaacagtctatttcat

SEQ ID NO: 662 - protein, 34 aa, Artificial Sequence
  Synthetic Sequence
HDNGNGHDHDNGNINHNGNINGNGNINININHNG

SEQ ID NO: 663 - protein, 32 aa, Artificial Sequence
  Synthetic Sequence
HDHDNGNGNININGNINGNGNIHDHDNINHNG

SEQ ID NO: 664 - protein, 48 aa, Artificial Sequence
  Synthetic Sequence
NIHDHDNININHHDNGNHNINININGNHNIHDNIHDNININININHNG

SEQ ID NO: 665 - protein, 48 aa, Artificial Sequence
  Synthetic Sequence
NHNHHDNGNHNGNHNGHDNIHDNINGNIHDHDNINHHDNINHNINING

SEQ ID NO: 666 - protein, 42 aa, Artificial Sequence
  Synthetic Sequence
NHNGNGNININGNGNGNHNININGNIHDNININGHDNIHDNG

SEQ ID NO: 667 - protein, 40 aa, Artificial Sequence
  Synthetic Sequence
NHNGNHNGHDNIHDNINGNIHDHDNINHHDNINHNINING

SEQ ID NO: 668 - protein, 30 aa, Artificial Sequence
  Synthetic Sequence
NHNHNGNINIHDNGNIHDNGNININGNGNG

SEQ ID NO: 669 - protein, 36 aa, Artificial Sequence
  Synthetic Sequence
NHNGHDNIHDNINGNIHDHDNINHHDNINHNINING

SEQ ID NO: 670 - protein, 40 aa, Artificial Sequence
  Synthetic Sequence
NHNGNHNIHDNIHDNINHHDHDNINGHDNINIHDNINING

SEQ ID NO: 671 - protein, 30 aa, Artificial Sequence
  Synthetic Sequence
HDHDNGNGNGNHNINGNHNINIHDNINHNG

SEQ ID NO: 672 - protein, 56 aa, Artificial Sequence
  Synthetic Sequence
NHNGNHNGNHHDNININGNINHHDNHNGNGNINININHNHNINIHDNGNIHDNING

SEQ ID NO: 673 - protein, 32 aa, Artificial Sequence
  Synthetic Sequence
HDNGNGNGHDNININGNINHHDHDHDNIHDNG

SEQ ID NO: 674 - protein, 42 aa, Artificial Sequence
  Synthetic Sequence
HDNGHDNINININGNHNIHDNININHNINHHDNIHDNINHNG

SEQ ID NO: 675 - protein, 34 aa, Artificial Sequence
  Synthetic Sequence
NIHDHDNINHNGNGNININGNGNINHHDNIHDNG

SEQ ID NO: 676 - protein, 34 aa, Artificial Sequence
  Synthetic Sequence
NHNGNGNHNGNHNIHDHDNGNININHHDHDNING

SEQ ID NO: 677 - protein, 44 aa, Artificial Sequence
  Synthetic Sequence
HDNGHDNINGNHNGNGNGNGNINININHNGHDNININHNINING

SEQ ID NO: 678 - protein, 36 aa, Artificial Sequence
  Synthetic Sequence
HDHDNGNHNININGNGHDNINHNINIHDNINHNING

SEQ ID NO: 679 - protein, 50 aa, Artificial Sequence
  Synthetic Sequence
NINHHDNINGNHNINGNHNGNGNGHDNINGNHNGNGNHNGNHNIHDHDNG

SEQ ID NO: 680 - protein, 48 aa, Artificial Sequence
  Synthetic Sequence
NHNGNGNGHDNINGNHNGNGNHNGNHNIHDHDNGNININHHDHDNING

SEQ ID NO: 681 - protein, 34 aa, Artificial Sequence
  Synthetic Sequence
NIHDNINIHDNINHNGHDNGNINGNGNGHDNING

SEQ ID NO: 682 - DNA, 26 bp, Artificial Sequence
  Synthetic Sequence
tcttcctagtctcttctctacccagt

SEQ ID NO: 683 - DNA, 25 bp, Artificial Sequence
  Synthetic Sequence
tacactccagcctgggaaacagagt

SEQ ID NO: 684 - DNA, 19 bp, Artificial Sequence
  Synthetic Sequence
tcttttccttaggacggct

SEQ ID NO: 685 - DNA, 17 bp, Artificial Sequence
  Synthetic Sequence
tcgctcaggcctgtcat

SEQ ID NO: 686 - DNA, 16 bp, Artificial Sequence
  Synthetic Sequence
tccatatggaagactt

SEQ ID NO: 687 - DNA, 18 bp, Artificial Sequence
  Synthetic Sequence
tacccagttaaccaccct

SEQ ID NO: 688 - DNA, 27 bp, Artificial Sequence
  Synthetic Sequence
tggcgcatgcctgtaatcccagctact

SEQ ID NO: 689 - DNA, 26 bp, Artificial Sequence
  Synthetic Sequence
tatacgaggagaaaattagcattcct

SEQ ID NO: 690 - DNA, 23 bp, Artificial Sequence
  Synthetic Sequence
tctgcctcccaggttcacgcaat

SEQ ID NO: 691 - DNA, 22 bp, Artificial Sequence
  Synthetic Sequence
tgccttgtcacgttttcacagt

SEQ ID NO: 692 - DNA, 25 bp, Artificial Sequence
  Synthetic Sequence
tgtcaccttctgtatgtgcaaccat

SEQ ID NO: 693 - DNA, 17 bp, Artificial Sequence
  Synthetic Sequence
tctgtatgtgcaaccat

SEQ ID NO: 694 - DNA, 17 bp, Artificial Sequence
  Synthetic Sequence
tagtcaagcaacaggat

SEQ ID NO: 695 - DNA, 18 bp, Artificial Sequence
  Synthetic Sequence
tccaagataattccccat

SEQ ID NO: 696 - DNA, 16 bp, Artificial Sequence
  Synthetic Sequence
tctgcaagatcctttt

SEQ ID NO: 697 - DNA, 29 bp, Artificial Sequence
  Synthetic Sequence
tgctatgtaaggtagcaaaaaggtaacct

SEQ ID NO: 698 - DNA, 16 bp, Artificial Sequence
  Synthetic Sequence
tctctctcctcctgct

SEQ ID NO: 699 - DNA, 19 bp, Artificial Sequence
  Synthetic Sequence
tccaaatgctattctctct

SEQ ID NO: 700 - DNA, 17 bp, Artificial Sequence
  Synthetic Sequence
tgctgattcagcctcct

SEQ ID NO: 701 - DNA, 21 bp, Artificial Sequence
  Synthetic Sequence
tagaacagccccccacacagt

SEQ ID NO: 702 - protein, 50 aa, Artificial Sequence
  Synthetic Sequence
HDNGNGHDHDNGNINHNGHDNGHDNGNGHDNGHDNGNIHDHDHDNINHNG

SEQ ID NO: 703 - protein, 48 aa, Artificial Sequence
  Synthetic Sequence
NIHDNIHDNGHDHDNINHHDHDNGNHNHNHNININIHDNINHNINHNG

SEQ ID NO: 704 - protein, 36 aa, Artificial Sequence
  Synthetic Sequence
HDNGNGNGNGHDHDNGNGNINHNHNIHDNHNHHDNG

SEQ ID NO: 705 - protein, 32 aa, Artificial Sequence
  Synthetic Sequence
HDNHHDNGHDNINHNHHDHDNGNHNGHDNING

SEQ ID NO: 706 - protein, 30 aa, Artificial Sequence
  Synthetic Sequence
HDHDNINGNINGNHNHNININHNIHDNGNG

SEQ ID NO: 707 - protein, 34 aa, Artificial Sequence
  Synthetic Sequence
NIHDHDHDNINHNGNGNINIHDHDNIHDHDHDNG

SEQ ID NO: 708 - protein, 52 aa, Artificial Sequence
  Synthetic Sequence
NHNHHDNHHDNINGNHHDHDNGNHNGNININGHDHDHDNINHHDNGNIHDNG

SEQ ID NO: 709 - protein, 50 aa, Artificial Sequence
  Synthetic Sequence
NINGNIHDNHNINHNHNINHNININININGNGNINHHDNINGNGHDHDNG

SEQ ID NO: 710 - protein, 44 aa, Artificial Sequence
  Synthetic Sequence
HDNGNHHDHDNGHDHDHDNINHNHNGNGHDNIHDNHHDNINING

SEQ ID NO: 711 - protein, 42 aa, Artificial Sequence
  Synthetic Sequence
NHHDHDNGNGNHNGHDNIHDNHNGNGNGNGHDNIHDNINHNG

SEQ ID NO: 712 - protein, 48 aa, Artificial Sequence
  Synthetic Sequence
NHNGHDNIHDHDNGNGHDNGNHNGNINGNHNGNHHDNINIHDHDNING

SEQ ID NO: 713 - protein, 32 aa, Artificial Sequence
  Synthetic Sequence
HDNGNHNGNINGNHNGNHHDNINIHDHDNING

SEQ ID NO: 714 - protein, 32 aa, Artificial Sequence
  Synthetic Sequence
NINHNGHDNININHHDNINIHDNINHNHNING

SEQ ID NO: 715 - protein, 34 aa, Artificial Sequence
  Synthetic Sequence
HDHDNININHNINGNININGNGHDHDHDHDNING

SEQ ID NO: 716 - protein, 30 aa, Artificial Sequence
  Synthetic Sequence
HDNGNHHDNININHNINGHDHDNGNGNGNG

SEQ ID NO: 717 - protein, 56 aa, Artificial Sequence
  Synthetic Sequence
NHHDNGNINGNHNGNININHNHNGNINHHDNINININININHNHNGNINIHDHDNG

SEQ ID NO: 718 - protein, 30 aa, Artificial Sequence
  Synthetic Sequence
HDNGHDNGHDNGHDHDNGHDHDNGNHHDNG

SEQ ID NO: 719 - protein, 36 aa, Artificial Sequence
  Synthetic Sequence
HDHDNINININGNHHDNGNINGNGHDNGHDNGHDNG

SEQ ID NO: 720 - protein, 32 aa, Artificial Sequence
  Synthetic Sequence
NHHDNGNHNINGNGHDNINHHDHDNGHDHDNG

SEQ ID NO: 721 - protein, 40 aa, Artificial Sequence
  Synthetic Sequence
NINHNINIHDNINHHDHDHDHDHDHDNIHDNIHDNINHNG

SEQ ID NO: 722 - DNA, 18 bp, Artificial Sequence
  Synthetic Sequence
tttagcagatgcatcagc

SEQ ID NO: 723 - DNA, 20 bp, Artificial Sequence
  Synthetic Sequence
tgaccaggggcatgtcctgg

SEQ ID NO: 724 - DNA, 21 bp, Artificial Sequence
  Synthetic Sequence
tggtccacctacctgaaaatg

SEQ ID NO: 725 - DNA, 21 bp, Artificial Sequence
  Synthetic Sequence
tgtcccacaggtattacgggc

SEQ ID NO: 726 - DNA, 21 bp, Artificial Sequence
  Synthetic Sequence
tacgggccaacctgacaatac

SEQ ID NO: 727 - DNA, 21 bp, Artificial Sequence
  Synthetic Sequence
tgagctttggggactgaaaga

SEQ ID NO: 728 - DNA, 21 bp, Artificial Sequence
  Synthetic Sequence
ctggcataatcttttccccca

SEQ ID NO: 729 - DNA, 21 bp, Artificial Sequence
  Synthetic Sequence
ccagcctcctggccatgtgca

SEQ ID NO: 730 - protein, 21 aa, Artificial Sequence
  Synthetic Sequence
GGCCATGTGCACAGGGGCTGA

SEQ ID NO: 731 - DNA, 21 bp, Artificial Sequence
  Synthetic Sequence
ctgatatgtgaaggtttagca

SEQ ID NO: 732 - DNA, 21 bp, Artificial Sequence
  Synthetic Sequence
tgaccaggcgtggtggctcac

SEQ ID NO: 733 - DNA, 16 bp, Artificial Sequence
  Synthetic Sequence
tatagacattttcact

SEQ ID NO: 734 - DNA, 21 bp, Artificial Sequence
  Synthetic Sequence
tctacatttaactatcaacct

SEQ ID NO: 735 - DNA, 17 bp, Artificial Sequence
  Synthetic Sequence
tcgtgcaaacgtttgat

SEQ ID NO: 736 - DNA, 18 bp, Artificial Sequence
  Synthetic Sequence
tacatcaatcctgtaggt

SEQ ID NO: 737 - DNA, 20 bp, Artificial Sequence
  Synthetic Sequence
tctattttagtgacccaagt

SEQ ID NO: 738 - DNA, 19 bp, Artificial Sequence
  Synthetic Sequence
tagagtcaaagcatgtact

SEQ ID NO: 739 - DNA, 18 bp, Artificial Sequence
  Synthetic Sequence
tcctacccataagctcct

SEQ ID NO: 740 - DNA, 17 bp, Artificial Sequence
  Synthetic Sequence
tccccatccccatcagt

SEQ ID NO: 741 - DNA, 29 bp, Artificial Sequence
  Synthetic Sequence
tctttaattcaagcaagactttaacaagt

SEQ ID NO: 742 - DNA, 16 bp, Artificial Sequence
  Synthetic Sequence
tgcagtcccctttctt

SEQ ID NO: 743 - DNA, 21 bp, Artificial Sequence
  Synthetic Sequence
tctgcacaaatccccaaagat

SEQ ID NO: 744 - DNA, 16 bp, Artificial Sequence
  Synthetic Sequence
tacatgctttgactct

SEQ ID NO: 745 - DNA, 29 bp, Artificial Sequence
  Synthetic Sequence
tggccagttatactgccagcagctataat

SEQ ID NO: 746 - protein, 34 aa, Artificial Sequence
  Synthetic Sequence
NGNGNINHHDNINHNINGNHHDNINGHDNINHHD

SEQ ID NO: 747 - protein, 38 aa, Artificial Sequence
  Synthetic Sequence
NHNIHDHDNINHNHNHNHHDNINGNHNGHDHDNGNHNH

SEQ ID NO: 748 - protein, 40 aa, Artificial Sequence
  Synthetic Sequence
HDNININHNHNINHNGNGHDNGNHNHHDNGNHNHNHNGHD

SEQ ID NO: 749 - protein, 40 aa, Artificial Sequence
  Synthetic Sequence
NHNGHDHDHDNIHDNINHNHNGNINGNGNIHDNHNHNHHD

SEQ ID NO: 750 - protein, 40 aa, Artificial Sequence
  Synthetic Sequence
NIHDNHNHNHHDHDNINIHDHDNGNHNIHDNININGNIHD

SEQ ID NO: 751 - protein, 40 aa, Artificial Sequence
  Synthetic Sequence
NHNINHHDNGNGNGNHNHNHNHNIHDNGNHNINININHNI

SEQ ID NO: 752 - protein, 40 aa, Artificial Sequence
  Synthetic Sequence
NHNHNHNHNHNININININHNINGNGNINGNHHDHDNINH

SEQ ID NO: 753 - protein, 40 aa, Artificial Sequence
  Synthetic Sequence
NHHDNIHDNINGNHNHHDHDNINHNHNINHNHHDNGNHNH

SEQ ID NO: 754 - protein, 40 aa, Artificial Sequence
  Synthetic Sequence
HDNINHHDHDHDHDNGNHNGNHHDNIHDNINGNHNHHDHD

SEQ ID NO: 755 - protein, 40 aa, Artificial Sequence
  Synthetic Sequence
NHHDNGNININIHDHDNGNGHDNIHDNINGNINGHDNINH

SEQ ID NO: 756 - protein, 40 aa, Artificial Sequence
  Synthetic Sequence
NHNIHDHDNINHNHHDNHNGNHNHNGNHNHHDNGHDNIHD

SEQ ID NO: 757 - protein, 30 aa, Artificial Sequence
  Synthetic Sequence
NINGNINHNIHDNINGNGNGNGHDNIHDNG

SEQ ID NO: 758 - protein, 40 aa, Artificial Sequence
  Synthetic Sequence
HDNGNIHDNINGNGNGNINIHDNGNINGHDNINIHDHDNG

SEQ ID NO: 759 - protein, 32 aa, Artificial Sequence
  Synthetic Sequence
HDNHNGNHHDNININIHDNHNGNGNGNHNING

SEQ ID NO: 760 - protein, 34 aa, Artificial Sequence
  Synthetic Sequence
NIHDNINGHDNININGHDHDNGNHNGNINHNHNG

SEQ ID NO: 761 - protein, 38 aa, Artificial Sequence
  Synthetic Sequence
HDNGNINGNGNGNGNINHNGNHNIHDHDHDNININHNG

SEQ ID NO: 762 - protein, 36 aa, Artificial Sequence
  Synthetic Sequence
NINHNINHNGHDNINININHHDNINGNHNGNIHDNG

SEQ ID NO: 763 - protein, 34 aa, Artificial Sequence
  Synthetic Sequence
HDHDNGNIHDHDHDNINGNININHHDNGHDHDNG

SEQ ID NO: 764 - protein, 32 aa, Artificial Sequence
  Synthetic Sequence
HDHDHDHDNINGHDHDHDHDNINGHDNINHNG

SEQ ID NO: 765 - protein, 56 aa, Artificial Sequence
  Synthetic Sequence
HDNGNGNGNININGNGHDNININHHDNININHNIHDNGNGNGNINIHDNININHNG

SEQ ID NO: 766 - protein, 30 aa, Artificial Sequence
  Synthetic Sequence
NHHDNINHNGHDHDHDHDNGNGNGHDNGNG

SEQ ID NO: 767 - protein, 30 aa, Artificial Sequence; identical to SEQ ID NO: 766
  Synthetic Sequence

SEQ ID NO: 768 - protein, 30 aa, Artificial Sequence; identical to SEQ ID NO: 766
  Synthetic Sequence

SEQ ID NO: 769 - protein, 56 aa, Artificial Sequence
  Synthetic Sequence
NHNHHDHDNINHNGNGNINGNIHDNGNHHDHDNINHHDNINHHDNGNINGNINING

SEQ ID NO: 770 - DNA, 15 bp, Artificial Sequence
  Synthetic Sequence
tgggaagataaacta

SEQ ID NO: 771 - DNA, 18 bp, Artificial Sequence
  Synthetic Sequence
actcccctgcagggcaac

SEQ ID NO: 772 - DNA, 18 bp, Artificial Sequence
  Synthetic Sequence
cccctgcagggcaacgcc

SEQ ID NO: 773 - DNA, 18 bp, Artificial Sequence
  Synthetic Sequence
ctgcagggcaacgcccag

SEQ ID NO: 774 - DNA, 18 bp, Artificial Sequence
  Synthetic Sequence
cagggcaacgcccaggga

SEQ ID NO: 775 - DNA, 18 bp, Artificial Sequence
  Synthetic Sequence
ggcaacgcccagggacca

SEQ ID NO: 776 - DNA, 18 bp, Artificial Sequence
  Synthetic Sequence
aacgcccagggaccaagt

SEQ ID NO: 777 - DNA, 18 bp, Artificial Sequence
  Synthetic Sequence
gcccagggaccaagttag

SEQ ID NO: 778 - DNA, 18 bp, Artificial Sequence
  Synthetic Sequence
cagggaccaagttagccc

SEQ ID NO: 779 - DNA, 15 bp, Artificial Sequence
  Synthetic Sequence
gcctaggcaaaagaa

SEQ ID NO: 780 - DNA, 18 bp, Artificial Sequence
  Synthetic Sequence
cgcgaggaggaaaggagg

SEQ ID NO: 781 - DNA, 18 bp, Artificial Sequence
  Synthetic Sequence
gaggaggaaaggagggag

SEQ ID NO: 782 - DNA, 18 bp, Artificial Sequence
  Synthetic Sequence
gaggaaaggagggagggc

SEQ ID NO: 783 - protein, 148 aa, Artificial Sequence
  Synthetic Sequence
LEPGEKPYKCPECGKSFSQNSTLTEHQRTHTGEKPYKCPECGKSFSQRANLRAHQRTHTGEKPYKCPECGKSFSTSGNLVRHQRTHTGEKPYKCPECGKSFSQSSNLVRHQRTHTGEKPYKCPECGKSFSRSDHLTTHQRTHTGKKTS

SEQ ID NO: 784 - protein, 176 aa, Artificial Sequence
  Synthetic Sequence
LEPGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGEKPYKCPECGKSFSSKKHLAEHQRTHTGEKPYKCPECGKSFSTHLDLIRHQRTHTGKKTS

SEQ ID NO: 785 - protein, 176 aa, Artificial Sequence
  Synthetic Sequence
LEPGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGEKPYKCPECGKSFSSKKHLAEHQRTHTGKKTS

SEQ ID NO: 786 - protein, 176 aa, Artificial Sequence
  Synthetic Sequence
LEPGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGKKTS

SEQ ID NO: 787 - protein, 176 aa, Artificial Sequence
  Synthetic Sequence
LEPGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGKKTS

SEQ ID NO: 788 - protein, 176 aa, Artificial Sequence
  Synthetic Sequence
LEPGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGKKTS

SEQ ID NO: 789 - protein, 176 aa, Artificial Sequence
  Synthetic Sequence
LEPGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGKKTS

SEQ ID NO: 790 - protein, 176 aa, Artificial Sequence
  Synthetic Sequence
LEPGEKPYKCPECGKSFSREDNLHTHQRTHTGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGKKTS

SEQ ID NO: 791 - protein, 176 aa, Artificial Sequence
  Synthetic Sequence
LEPGEKPYKCPECGKSFSSKKHLAEHQRTHTGEKPYKCPECGKSFSREDNLHTHQRTHTGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGKKTS

SEQ ID NO: 792 - protein, 148 aa, Artificial Sequence
  Synthetic Sequence
LEPGEKPYKCPECGKSFSQSSNLVRHQRTHTGEKPYKCPECGKSFSQRANLRAHQRTHTGEKPYKCPECGKSFSQSGDLRRHQRTHTGEKPYKCPECGKSFSREDNLHTHQRTHTGEKPYKCPECGKSFSDCRDLARHQRTHTGKKTS

SEQ ID NO: 793 - protein, 176 aa, Artificial Sequence
  Synthetic Sequence
LEPGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGEKPYKCPECGKSFSRSDNLVRHQRTHTGEKPYKCPECGKSFSHTGHLLEHQRTHTGKKTS

SEQ ID NO: 794 - protein, 176 aa, Artificial Sequence
  Synthetic Sequence
LEPGEKPYKCPECGKSFSRSDNLVRHQRTHTGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGEKPYKCPECGKSFSRSDNLVRHQRTHTGKKTS

SEQ ID NO: 795 - protein, 176 aa, Artificial Sequence
  Synthetic Sequence
LEPGEKPYKCPECGKSFSDPGHLVRHQRTHTGEKPYKCPECGKSFSRSDNLVRHQRTHTGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGKKTS

SEQ ID NO: 796 - protein, 30 aa, Artificial Sequence
  Synthetic Sequence
TAGGACAGTGGGGAAAATGACCCAACAGCC

SEQ ID NO: 797 - DNA, 21 bp, Artificial Sequence
  Synthetic Sequence
agagggagccacgaaaacaga

SEQ ID NO: 798 - DNA, 15 bp, Artificial Sequence
  Synthetic Sequence
gcagatagccaggag

SEQ ID NO: 799 - DNA, 18 bp, Artificial Sequence
  Synthetic Sequence
agatagccaggagtcctt

SEQ ID NO: 800 - DNA, 24 bp, Artificial Sequence
  Synthetic Sequence
cccagtggtcaggccggccaggcc

SEQ ID NO: 801 - DNA, 18 bp, Artificial Sequence
  Synthetic Sequence
ggccggccaggccttcag

SEQ ID NO: 802 - DNA, 27 bp, Artificial Sequence
  Synthetic Sequence
agtgctcagtggaaaccacgaaaggac

SEQ ID NO: 803 - DNA, 21 bp, Artificial Sequence
  Synthetic Sequence
tggcccccagcccctcctgcc

SEQ ID NO: 804 - DNA, 27 bp, Artificial Sequence
  Synthetic Sequence
agagccaggagtcctggcccccagccc

SEQ ID NO: 805 - DNA, 24 bp, Artificial Sequence
  Synthetic Sequence
gcaggaggggctgggggccaggac

SEQ ID NO: 806 - DNA, 24 bp, Artificial Sequence
  Synthetic Sequence
atagccctgggcccacggcttcgt

SEQ ID NO: 807 - DNA, 15 bp, Artificial Sequence
  Synthetic Sequence
gaaggacctggctgg

SEQ ID NO: 808 - DNA, 27 bp, Artificial Sequence
  Synthetic Sequence
gcaggaacgaagccgtgggcccagggc

SEQ ID NO: 809 - DNA, 18 bp, Artificial Sequence
  Synthetic Sequence
ggaaaccaccccagcaga

SEQ ID NO: 810 - DNA, 33 bp, Artificial Sequence
  Synthetic Sequence
aagggtcaagctcggaaaccaccccagcagata

SEQ ID NO: 811 - protein, 288 aa, Artificial Sequence
  Synthetic Sequence
LEPGEKPYKCPECGKSFSDCRDLARHQRTHTGEKPYKCPECGKSFSSPADLTRHQRTHTGEKPYKCPECGKSFSTSHSLTEHQRTHTGEKPYKCPECGKSFSDPGNLVRHQRTHTGEKPYKCPECGKSFSTTGNLTVHQRTHTGEKPYKCPECGKSFSQSSNLVRHQRTHTGEKPYKCPECGKSFSRSDKLVRHQRTHTGEKPYKCPECGKSFSHRTTLTNHQRTHTGEKPYKCPECGKSFSDPGNLVRHQRTHTGEKPYKCPECGKSFSREDNLHTHQRTHTGKKTS

SEQ ID NO: 812 - protein, 204 aa, Artificial Sequence
  Synthetic Sequence
LEPGEKPYKCPECGKSFSQLAHLRAHQRTHTGEKPYKCPECGKSFSDSGNLRVHQRTHTGEKPYKCPECGKSFSQSSNLVRHQRTHTGEKPYKCPECGKSFSSKKALTEHQRTHTGEKPYKCPECGKSFSERSHLREHQRTHTGEKPYKCPECGKSFSRSDKLVRHQRTHTGEKPYKCPECGKSFSQLAHLRAHQRTHTGKKTS

SEQ ID NO: 813 - protein, 148 aa, Artificial Sequence
  Synthetic Sequence
LEPGEKPYKCPECGKSFSRSDNLVRHQRTHTGEKPYKCPECGKSFSRADNLTEHQRTHTGEKPYKCPECGKSFSERSHLREHQRTHTGEKPYKCPECGKSFSTSGNLVRHQRTHTGEKPYKCPECGKSFSQSGDLRRHQRTHTGKKTS

SEQ ID NO: 814 - protein, 176 aa, Artificial Sequence
  Synthetic Sequence
LEPGEKPYKCPECGKSFSTTGALTEHQRTHTGEKPYKCPECGKSFSDPGALVRHQRTHTGEKPYKCPECGKSFSQRAHLERHQRTHTGEKPYKCPECGKSFSTSHSLTEHQRTHTGEKPYKCPECGKSFSREDNLHTHQRTHTGEKPYKCPECGKSFSQLAHLRAHQRTHTGKKTS

SEQ ID NO: 815 - protein, 232 aa, Artificial Sequence
  Synthetic Sequence
LEPGEKPYKCPECGKSFSDCRDLARHQRTHTGEKPYKCPECGKSFSRADNLTEHQRTHTGEKPYKCPECGKSFSDPGHLVRHQRTHTGEKPYKCPECGKSFSDCRDLARHQRTHTGEKPYKCPECGKSFSRADNLTEHQRTHTGEKPYKCPECGKSFSTSGHLVRHQRTHTGEKPYKCPECGKSFSHRTTLTNHQRTHTGEKPYKCPECGKSFSSKKHLAEHQRTHTGKKTS

SEQ ID NO: 816 - protein, 176 aa, Artificial Sequence
  Synthetic Sequence
LEPGEKPYKCPECGKSFSRADNLTEHQRTHTGEKPYKCPECGKSFSTTGALTEHQRTHTGEKPYKCPECGKSFSDPGHLVRHQRTHTGEKPYKCPECGKSFSTSHSLTEHQRTHTGEKPYKCPECGKSFSRSDKLTEHQRTHTGEKPYKCPECGKSFSDPGHLVRHQRTHTGKKTS

SEQ ID NO: 817 - protein, 260 aa, Artificial Sequence
  Synthetic Sequence
LEPGEKPYKCPECGKSFSDPGNLVRHQRTHTGEKPYKCPECGKSFSRKDNLKNHQRTHTGEKPYKCPECGKSFSQSGHLTEHQRTHTGEKPYKCPECGKSFSTSHSLTEHQRTHTGEKPYKCPECGKSFSQRANLRAHQRTHTGEKPYKCPECGKSFSRSDHLTTHQRTHTGEKPYKCPECGKSFSRADNLTEHQRTHTGEKPYKCPECGKSFSTSGELVRHQRTHTGEKPYKCPECGKSFSHRTTLTNHQRTHTGKKTS

SEQ ID NO: 818 - protein, 204 aa, Artificial Sequence
  Synthetic Sequence
LEPGEKPYKCPECGKSFSDCRDLARHQRTHTGEKPYKCPECGKSFSTKNSLTEHQRTHTGEKPYKCPECGKSFSTKNSLTEHQRTHTGEKPYKCPECGKSFSDCRDLARHQRTHTGEKPYKCPECGKSFSTSHSLTEHQRTHTGEKPYKCPECGKSFSSKKHLAEHQRTHTGEKPYKCPECGKSFSRSDHLTTHQRTHTGKKTS

SEQ ID NO: 819 - protein, 260 aa, Artificial Sequence
  Synthetic Sequence
LEPGEKPYKCPECGKSFSSKKHLAEHQRTHTGEKPYKCPECGKSFSRADNLTEHQRTHTGEKPYKCPECGKSFSSKKHLAEHQRTHTGEKPYKCPECGKSFSDPGHLVRHQRTHTGEKPYKCPECGKSFSTKNSLTEHQRTHTGEKPYKCPECGKSFSHRTTLTNHQRTHTGEKPYKCPECGKSFSRSDHLTNHQRTHTGEKPYKCPECGKSFSDCRDLARHQRTHTGEKPYKCPECGKSFSQLAHLRAHQRTHTGKKTS

SEQ ID NO: 820 - protein, 232 aa, Artificial Sequence
  Synthetic Sequence
LEPGEKPYKCPECGKSFSDPGNLVRHQRTHTGEKPYKCPECGKSFSRADNLTEHQRTHTGEKPYKCPECGKSFSDPGHLVRHQRTHTGEKPYKCPECGKSFSRSDKLVRHQRTHTGEKPYKCPECGKSFSTSGELVRHQRTHTGEKPYKCPECGKSFSRSDKLVRHQRTHTGEKPYKCPECGKSFSQRAHLERHQRTHTGEKPYKCPECGKSFSQSGDLRRHQRTHTGKKTS

SEQ ID NO: 821 - protein, 231 aa, Artificial Sequence
  Synthetic Sequence
LEPGEKPYKCPECGKSFSSRRTCRAHQRTHTGEKPYKCPECGKSFSTTGALTEHQRTHTGEKPYKCPECGKSFSRSDKLTEHQRTHTGEKPYKCPECGKSFSTSHSLTEHQRTHTGEKPYKCPECGKSFSDPGHLVRHQRTHTGEKPYKCPECGKSFSRNDALTEHQRTHTGEKPYKCPECGKSFSDCRDLARHQRTHTGEKPYKCPECGKSFSQKSSLIAHQRTHTGKKT

SEQ ID NO: 822 - protein, 148 aa, Artificial Sequence
  Synthetic Sequence
LEPGEKPYKCPECGKSFSRSDHLTTHQRTHTGEKPYKCPECGKSFSDPGHLVRHQRTHTGEKPYKCPECGKSFSTKNSLTEHQRTHTGEKPYKCPECGKSFSQRAHLERHQRTHTGEKPYKCPECGKSFSQSSNLVRHQRTHTGKKTS

SEQ ID NO: 823 - protein, 260 aa, Artificial Sequence
  Synthetic Sequence
LEPGEKPYKCPECGKSFSDPGHLVRHQRTHTGEKPYKCPECGKSFSRADNLTEHQRTHTGEKPYKCPECGKSFSDCRDLARHQRTHTGEKPYKCPECGKSFSRSDHLTTHQRTHTGEKPYKCPECGKSFSRNDTLTEHQRTHTGEKPYKCPECGKSFSRKDNLKNHQRTHTGEKPYKCPECGKSFSRTDTLRDHQRTHTGEKPYKCPECGKSFSQRAHLERHQRTHTGEKPYKCPECGKSFSQSGDLRRHQRTHTGKKTS

SEQ ID NO: 824 - protein, 176 aa, Artificial Sequence
  Synthetic Sequence
LEPGEKPYKCPECGKSFSQLAHLRAHQRTHTGEKPYKCPECGKSFSERSHLREHQRTHTGEKPYKCPECGKSFSSKKHLAEHQRTHTGEKPYKCPECGKSFSSKKALTEHQRTHTGEKPYKCPECGKSFSDSGNLRVHQRTHTGEKPYKCPECGKSFSQRAHLERHQRTHTGKKTS

SEQ ID NO: 825 - protein, 316 aa, Artificial Sequence
  Synthetic Sequence
LEPGEKPYKCPECGKSFSQKSSLIAHQRTHTGEKPYKCPECGKSFSRADNLTEHQRTHTGEKPYKCPECGKSFSRADNLTEHQRTHTGEKPYKCPECGKSFSSKKHLAEHQRTHTGEKPYKCPECGKSFSTSHSLTEHQRTHTGEKPYKCPECGKSFSQRANLRAHQRTHTGEKPYKCPECGKSFSRSDKLTEHQRTHTGEKPYKCPECGKSFSTSGELVRHQRTHTGEKPYKCPECGKSFSQSGNLTEHQRTHTGEKPYKCPECGKSFSTSGHLVRHQRTHTGEKPYKCPECGKSFSRKDNLKNHQRTHTGKKTS

SEQ ID NO: 826 - DNA, 18 bp, Artificial Sequence
  Synthetic Sequence
ctttgatgaacagtcaca

SEQ ID NO: 827 - DNA, 18 bp, Artificial Sequence
  Synthetic Sequence
cttccaattagtcctacc

SEQ ID NO: 828 - DNA, 21 bp, Artificial Sequence
  Synthetic Sequence
atactaggaagaaatacaata

SEQ ID NO: 829 - DNA, 18 bp, Artificial Sequence
  Synthetic Sequence
gctcttgtcatttgagat

SEQ ID NO: 830 - DNA, 33 bp, Artificial Sequence
  Synthetic Sequence
ccaagctgaaatgacacaaaagttaaaacaaag

SEQ ID NO: 831 - DNA, 21 bp, Artificial Sequence
  Synthetic Sequence
cttataccagttaattagcac

SEQ ID NO: 832 - DNA, 24 bp, Artificial Sequence
  Synthetic Sequence
aacgctattgcacacatagttaca

SEQ ID NO: 833 - DNA, 21 bp, Artificial Sequence
  Synthetic Sequence
tgaattcaggaacaaagtata

SEQ ID NO: 834 - DNA, 27 bp, Artificial Sequence
  Synthetic Sequence
gctggtatgtgacacagccatcaacaa

SEQ ID NO: 835 - protein, 176 aa, Artificial Sequence
  Synthetic Sequence
LEPGEKPYKCPECGKSFSSPADLTRHQRTHTGEKPYKCPECGKSFSDPGALVRHQRTHTGEKPYKCPECGKSFSSPADLTRHQRTHTGEKPYKCPECGKSFSQAGHLASHQRTHTGEKPYKCPECGKSFSQAGHLASHQRTHTGEKPYKCPECGKSFSTTGALTEHQRTHTGKKTS

SEQ ID NO: 836 - protein, 176 aa, Artificial Sequence
  Synthetic Sequence
LEPGEKPYKCPECGKSFSDKKDLTRHQRTHTGEKPYKCPECGKSFSTKNSLTEHQRTHTGEKPYKCPECGKSFSHRTTLTNHQRTHTGEKPYKCPECGKSFSHKNALQNHQRTHTGEKPYKCPECGKSFSTSHSLTEHQRTHTGEKPYKCPECGKSFSTTGALTEHQRTHTGKKTS

SEQ ID NO: 837 - protein, 204 aa, Artificial Sequence
  Synthetic Sequence
LEPGEKPYKCPECGKSFSQKSSLIAHQRTHTGEKPYKCPECGKSFSSPADLTRHQRTHTGEKPYKCPECGKSFSTTGNLTVHQRTHTGEKPYKCPECGKSFSQLAHLRAHQRTHTGEKPYKCPECGKSFSQRAHLERHQRTHTGEKPYKCPECGKSFSQNSTLTEHQRTHTGEKPYKCPECGKSFSQKSSLIAHQRTHTGKKTS

SEQ ID NO: 838 - protein, 176 aa, Artificial Sequence
  Synthetic Sequence
LEPGEKPYKCPECGKSFSTSGNLVRHQRTHTGEKPYKCPECGKSFSQAGHLASHQRTHTGEKPYKCPECGKSFSHKNALQNHQRTHTGEKPYKCPECGKSFSDPGALVRHQRTHTGEKPYKCPECGKSFSTTGALTEHQRTHTGEKPYKCPECGKSFSTSGELVRHQRTHTGKKTS

SEQ ID NO: 839 - protein, 316 aa, Artificial Sequence
  Synthetic Sequence
LEPGEKPYKCPECGKSFSRKDNLKNHQRTHTGEKPYKCPECGKSFSSPADLTRHQRTHTGEKPYKCPECGKSFSQRANLRAHQRTHTGEKPYKCPECGKSFSTSGSLVRHQRTHTGEKPYKCPECGKSFSQRANLRAHQRTHTGEKPYKCPECGKSFSSPADLTRHQRTHTGEKPYKCPECGKSFSDPGNLVRHQRTHTGEKPYKCPECGKSFSTTGNLTVHQRTHTGEKPYKCPECGKSFSQAGHLASHQRTHTGEKPYKCPECGKSFSERSHLREHQRTHTGEKPYKCPECGKSFSTSHSLTEHQRTHTGKKTS

SEQ ID NO: 840 - protein, 204 aa, Artificial Sequence
  Synthetic Sequence
LEPGEKPYKCPECGKSFSSKKALTEHQRTHTGEKPYKCPECGKSFSREDNLHTHQRTHTGEKPYKCPECGKSFSTTGNLTVHQRTHTGEKPYKCPECGKSFSTSGSLVRHQRTHTGEKPYKCPECGKSFSTSHSLTEHQRTHTGEKPYKCPECGKSFSQKSSLIAHQRTHTGEKPYKCPECGKSFSTTGALTEHQRTHTGKKTS

SEQ ID NO: 841 - protein, 232 aa, Artificial Sequence
  Synthetic Sequence
LEPGEKPYKCPECGKSFSSPADLTRHQRTHTGEKPYKCPECGKSFSTSGSLVRHQRTHTGEKPYKCPECGKSFSQKSSLIAHQRTHTGEKPYKCPECGKSFSSKKALTEHQRTHTGEKPYKCPECGKSFSQSGDLRRHQRTHTGEKPYKCPECGKSFSHKNALQNHQRTHTGEKPYKCPECGKSFSTSGELVRHQRTHTGEKPYKCPECGKSFSDSGNLRVHQRTHTGKKTS

SEQ ID NO: 842 - protein, 204 aa, Artificial Sequence
  Synthetic Sequence
LEPGEKPYKCPECGKSFSQKSSLIAHQRTHTGEKPYKCPECGKSFSHRTTLTNHQRTHTGEKPYKCPECGKSFSQSGNLTEHQRTHTGEKPYKCPECGKSFSQSSNLVRHQRTHTGEKPYKCPECGKSFSRADNLTEHQRTHTGEKPYKCPECGKSFSHKNALQNHQRTHTGEKPYKCPECGKSFSQAGHLASHQRTHTGKKTS

SEQ ID NO: 843 - protein, 260 aa, Artificial Sequence
  Synthetic Sequence
LEPGEKPYKCPECGKSFSQSGNLTEHQRTHTGEKPYKCPECGKSFSQSGNLTEHQRTHTGEKPYKCPECGKSFSTSGNLTEHQRTHTGEKPYKCPECGKSFSERSHLREHQRTHTGEKPYKCPECGKSFSSKKALTEHQRTHTGEKPYKCPECGKSFSQAGHLASHQRTHTGEKPYKCPECGKSFSRRDELNVHQRTHTGEKPYKCPECGKSFSTSGHLVRHQRTHTGEKPYKCPECGKSFSTSGELVRHQRTHTGKKTS

SEQ ID NO: 844 - DNA, 24 bp, Artificial Sequence
  Synthetic Sequence
cttcctgaaagcaagagatgaaat

SEQ ID NO: 845 - DNA, 24 bp, Artificial Sequence
  Synthetic Sequence
ctgaaagcaagagatgaaattcca

SEQ ID NO: 846 - DNA, 21 bp, Artificial Sequence
  Synthetic Sequence
atacgaggagaaaattagcat

SEQ ID NO: 847 - DNA, 36 bp, Artificial Sequence
  Synthetic Sequence
catccatggcaggaagttgaagccaaaataaatctg

SEQ ID NO: 848 - DNA, 27 bp, Artificial Sequence
  Synthetic Sequence
atggcaggaagttgaagccaaaataaa

SEQ ID NO: 849 - DNA, 33 bp, Artificial Sequence
  Synthetic Sequence
gaaaagaagactcaaggaaacagagccaaacac

SEQ ID NO: 850 - DNA, 24 bp, Artificial Sequence
  Synthetic Sequence
aggaaacagagccaaacacttaca

SEQ ID NO: 851 - DNA, 60 bp, Artificial Sequence
  Synthetic Sequence
atgcagatttggacacagagtagtaaactgtgaaaacgtgacaaggcaaagtggcgtggg

SEQ ID NO: 852 - DNA, 18 bp, Artificial Sequence
  Synthetic Sequence
ggacacagagtagtaaac

SEQ ID NO: 853 - DNA, 18 bp, Artificial Sequence
  Synthetic Sequence
aaagctagcagcatggca

SEQ ID NO: 854 - DNA, 24 bp, Artificial Sequence
  Synthetic Sequence
cctcttataaggcccaagaggata

SEQ ID NO: 855 - DNA, 21 bp, Artificial Sequence
  Synthetic Sequence
caacatccttgacttaatcac

SEQ ID NO: 856 - DNA, 18 bp, Artificial Sequence
  Synthetic Sequence
ggtagcaaaaaggtaacc

SEQ ID NO: 857 - DNA, 36 bp, Artificial Sequence
  Synthetic Sequence
tggggtgcaagaggccaggccagagttgttctggtc

SEQ ID NO: 858 - DNA, 24 bp, Artificial Sequence
  Synthetic Sequence
cgcatgctgattcagcctcctgac

SEQ ID NO: 859 - DNA, 18 bp, Artificial Sequence
  Synthetic Sequence
agtcaagcaacaggatga

SEQ ID NO: 860 - DNA, 30 bp, Artificial Sequence
  Synthetic Sequence
gtcaagcaacaggatgatccaaatgctatt

SEQ ID NO: 861 - protein, 232 aa, Artificial Sequence
  Synthetic Sequence
LEPGEKPYKCPECGKSFSTTGNLTVHQRTHTGEKPYKCPECGKSFSQAGHLASHQRTHTGEKPYKCPECGKSFSQLAHLRAHQRTHTGEKPYKCPECGKSFSRKDNLKNHQRTHTGEKPYKCPECGKSFSERSHLREHQRTHTGEKPYKCPECGKSFSQSSNLVRHQRTHTGEKPYKCPECGKSFSTKNSLTEHQRTHTGEKPYKCPECGKSFSTTGALTEHQRTHTGKKTS

SEQ ID NO: 862 - protein, 232 aa, Artificial Sequence
  Synthetic Sequence
LEPGEKPYKCPECGKSFSTSHSLTEHQRTHTGEKPYKCPECGKSFSHKNALQNHQRTHTGEKPYKCPECGKSFSQSSNLVRHQRTHTGEKPYKCPECGKSFSTSGNLVRHQRTHTGEKPYKCPECGKSFSQLAHLRAHQRTHTGEKPYKCPECGKSFSQSGDLRRHQRTHTGEKPYKCPECGKSFSQRANLRAHQRTHTGEKPYKCPECGKSFSRNDALTEHQRTHTGKKTS

SEQ ID NO: 863 - protein, 204 aa, Artificial Sequence
  Synthetic Sequence
LEPGEKPYKCPECGKSFSTSGNLTEHQRTHTGEKPYKCPECGKSFSREDNLHTHQRTHTGEKPYKCPECGKSFSTTGNLTVHQRTHTGEKPYKCPECGKSFSQSSNLVRHQRTHTGEKPYKCPECGKSFSQRAHLERHQRTHTGEKPYKCPECGKSFSQSGHLTEHQRTHTGEKPYKCPECGKSFSQKSSLIAHQRTHTGKKTS

SEQ ID NO: 864 - protein, 344 aa, Artificial Sequence
  Synthetic Sequence
LEPGEKPYKCPECGKSFSRNDALTEHQRTHTGEKPYKCPECGKSFSTTGNLTVHQRTHTGEKPYKCPECGKSFSQKSSLIAHQRTHTGEKPYKCPECGKSFSQRANLRAHQRTHTGEKPYKCPECGKSFSDCRDLARHQRTHTGEKPYKCPECGKSFSQSSNLVRHQRTHTGEKPYKCPECGKSFSTSGSLVRHQRTHTGEKPYKCPECGKSFSQSSNLVRHQRTHTGEKPYKCPECGKSFSRADNLTEHQRTHTGEKPYKCPECGKSFSRSDHLTTHQRTHTGEKPYKCPECGKSFSTSHSLTEHQRTHTGEKPYKCPECGKSFSTSGNLTEHQRTHTGKKTS

SEQ ID NO: 865 - protein, 260 aa, Artificial Sequence
  Synthetic Sequence
LEPGEKPYKCPECGKSFSQRANLRAHQRTHTGEKPYKCPECGKSFSTTGNLTVHQRTHTGEKPYKCPECGKSFSQSGNLTEHQRTHTGEKPYKCPECGKSFSERSHLREHQRTHTGEKPYKCPECGKSFSQAGHLASHQRTHTGEKPYKCPECGKSFSHRTTLTNHQRTHTGEKPYKCPECGKSFSQRAHLERHQRTHTGEKPYKCPECGKSFSQSGDLRRHQRTHTGEKPYKCPECGKSFSRRDELNVHQRTHTGKKTS

SEQ ID NO: 866 - protein, 316 aa, Artificial Sequence
  Synthetic Sequence
LEPGEKPYKCPECGKSFSSKKALTEHQRTHTGEKPYKCPECGKSFSQRANLRAHQRTHTGEKPYKCPECGKSFSDCRDLARHQRTHTGEKPYKCPECGKSFSQLAHLRAHQRTHTGEKPYKCPECGKSFSDSGNLRVHQRTHTGEKPYKCPECGKSFSQRAHLERHQRTHTGEKPYKCPECGKSFSQSGNLTEHQRTHTGEKPYKCPECGKSFSTHLDLIRHQRTHTGEKPYKCPECGKSFSRKDNLKNHQRTHTGEKPYKCPECGKSFSRKDNLKNHQRTHTGEKPYKCPECGKSFSQSSNLVRHQRTHTGKKTS

SEQ ID NO: 867 - protein, 232 aa, Artificial Sequence
  Synthetic Sequence
LEPGEKPYKCPECGKSFSSPADLTRHQRTHTGEKPYKCPECGKSFSTTGALTEHQRTHTGEKPYKCPECGKSFSSPADLTRHQRTHTGEKPYKCPECGKSFSQSGNLTEHQRTHTGEKPYKCPECGKSFSERSHLREHQRTHTGEKPYKCPECGKSFSRADNLTEHQRTHTGEKPYKCPECGKSFSQRANLRAHQRTHTGEKPYKCPECGKSFSRSDHLTNHQRTHTGKKTS

SEQ ID NO: 868 - protein, 568 aa, Artificial Sequence
  Synthetic Sequence
LEPGEKPYKCPECGKSFSRSDKLVRHQRTHTGEKPYKCPECGKSFSSRRTCRAHQRTHTGEKPYKCPECGKSFSRSDHLTTHQRTHTGEKPYKCPECGKSFSRKDNLKNHQRTHTGEKPYKCPECGKSFSQSGDLRRHQRTHTGEKPYKCPECGKSFSRKDNLKNHQRTHTGEKPYKCPECGKSFSDPGNLVRHQRTHTGEKPYKCPECGKSFSSRRTCRAHQRTHTGEKPYKCPECGKSFSQRANLRAHQRTHTGEKPYKCPECGKSFSQAGHLASHQRTHTGEKPYKCPECGKSFSRNDALTEHQRTHTGEKPYKCPECGKSFSQRANLRAHQRTHTGEKPYKCPECGKSFSHRTTLTNHQRTHTGEKPYKCPECGKSFSHRTTLTNHQRTHTGEKPYKCPECGKSFSRADNLTEHQRTHTGEKPYKCPECGKSFSSPADLTRHQRTHTGEKPYKCPECGKSFSRSDHLTTHQRTHTGEKPYKCPECGKSFSHKNALQNHQRTHTGEKPYKCPECGKSFSRADNLTEHQRTHTGEKPYKCPECGKSFSRRDELNVHQRTHTGKKTS

SEQ ID NO: 869 - protein, 176 aa, Artificial Sequence
  Synthetic Sequence
LEPGEKPYKCPECGKSFSDSGNLRVHQRTHTGEKPYKCPECGKSFSQSSSLVRHQRTHTGEKPYKCPECGKSFSQSSSLVRHQRTHTGEKPYKCPECGKSFSQLAHLRAHQRTHTGEKPYKCPECGKSFSSKKALTEHQRTHTGEKPYKCPECGKSFSQRAHLERHQRTHTGKKTS

SEQ ID NO: 870 - protein, 176 aa, Artificial Sequence; identical to SEQ ID NO: 869
  Synthetic Sequence

SEQ ID NO: 871 - protein, 232 aa, Artificial Sequence
  Synthetic Sequence
LEPGEKPYKCPECGKSFSQKSSLIAHQRTHTGEKPYKCPECGKSFSRSDHLTNHQRTHTGEKPYKCPECGKSFSRKDNLKNHQRTHTGEKPYKCPECGKSFSSKKHLAEHQRTHTGEKPYKCPECGKSFSRSDHLTNHQRTHTGEKPYKCPECGKSFSQKSSLIAHQRTHTGEKPYKCPECGKSFSTTGALTEHQRTHTGEKPYKCPECGKSFSTKNSLTEHQRTHTGKKTS

SEQ ID NO: 872 - protein, 204 aa, Artificial Sequence
  Synthetic Sequence
LEPGEKPYKCPECGKSFSSKKALTEHQRTHTGEKPYKCPECGKSFSTTGNLTVHQRTHTGEKPYKCPECGKSFSTTGALTEHQRTHTGEKPYKCPECGKSFSQAGHLASHQRTHTGEKPYKCPECGKSFSTKNSLTEHQRTHTGEKPYKCPECGKSFSTSGNLTEHQRTHTGEKPYKCPECGKSFSQSGNLTEHQRTHTGKKTS

SEQ ID NO: 873 - protein, 176 aa, Artificial Sequence
  Synthetic Sequence
LEPGEKPYKCPECGKSFSDKKDLTRHQRTHTGEKPYKCPECGKSFSQSSSLVRHQRTHTGEKPYKCPECGKSFSRKDNLKNHQRTHTGEKPYKCPECGKSFSQRANLRAHQRTHTGEKPYKCPECGKSFSERSHLREHQRTHTGEKPYKCPECGKSFSTSGHLVRHQRTHTGKKTS

SEQ ID NO: 874 - protein, 344 aa, Artificial Sequence
  Synthetic Sequence
LEPGEKPYKCPECGKSFSDPGALVRHQRTHTGEKPYKCPECGKSFSRNDALTEHQRTHTGEKPYKCPECGKSFSTSGSLVRHQRTHTGEKPYKCPECGKSFSTSGSLVRHQRTHTGEKPYKCPECGKSFSQLAHLRAHQRTHTGEKPYKCPECGKSFSDCRDLARHQRTHTGEKPYKCPECGKSFSRADNLTEHQRTHTGEKPYKCPECGKSFSDPGHLVRHQRTHTGEKPYKCPECGKSFSQLAHLRAHQRTHTGEKPYKCPECGKSFSQSGDLRRHQRTHTGEKPYKCPECGKSFSTSGHLVRHQRTHTGEKPYKCPECGKSFSRSDHLTTHQRTHTGKKTS

SEQ ID NO: 875 - protein, 232 aa, Artificial Sequence
  Synthetic Sequence
LEPGEKPYKCPECGKSFSDPGNLVRHQRTHTGEKPYKCPECGKSFSTKNSLTEHQRTHTGEKPYKCPECGKSFSTKNSLTEHQRTHTGEKPYKCPECGKSFSRADNLTEHQRTHTGEKPYKCPECGKSFSHKNALQNHQRTHTGEKPYKCPECGKSFSRNDALTEHQRTHTGEKPYKCPECGKSFSRRDELNVHQRTHTGEKPYKCPECGKSFSHTGHLLEHQRTHTGKKTS

SEQ ID NO: 876 - protein, 176 aa, Artificial Sequence
  Synthetic Sequence
LEPGEKPYKCPECGKSFSQAGHLASHQRTHTGEKPYKCPECGKSFSQRAHLERHQRTHTGEKPYKCPECGKSFSSPADLTRHQRTHTGEKPYKCPECGKSFSQSGDLRRHQRTHTGEKPYKCPECGKSFSQSGNLTEHQRTHTGEKPYKCPECGKSFSHRTTLTNHQRTHTGKKTS

SEQ ID NO: 877 - protein, 288 aa, Artificial Sequence
  Synthetic Sequence
LEPGEKPYKCPECGKSFSHKNALQNHQRTHTGEKPYKCPECGKSFSTSGELVRHQRTHTGEKPYKCPECGKSFSTTGNLTVHQRTHTGEKPYKCPECGKSFSTSHSLTEHQRTHTGEKPYKCPECGKSFSTSGNLVRHQRTHTGEKPYKCPECGKSFSTSGNLVRHQRTHTGEKPYKCPECGKSFSRADNLTEHQRTHTGEKPYKCPECGKSFSQSGNLTEHQRTHTGEKPYKCPECGKSFSRKDNLKNHQRTHTGEKPYKCPECGKSFSDPGALVRHQRTHTGKKTS

SEQ ID NO: 878 - DNA, 18 bp, Artificial Sequence
  Synthetic Sequence
gtagaaactcgccttatg

SEQ ID NO: 879 - DNA, 21 bp, Artificial Sequence
  Synthetic Sequence
tgaatgagtcctgtccatctt

SEQ ID NO: 880 - DNA, 21 bp, Artificial Sequence
  Synthetic Sequence
aagattagaacaaatgtccag

SEQ ID NO: 881 - DNA, 18 bp, Artificial Sequence
  Synthetic Sequence
actctaagcagcaatgta

SEQ ID NO: 882 - DNA, 18 bp, Artificial Sequence
  Synthetic Sequence
tgggatagtgaaaatgtc

SEQ ID NO: 883 - DNA, 27 bp, Artificial Sequence
  Synthetic Sequence
aaaacttgggtcactaaaatagatgat

SEQ ID NO: 884 - DNA, 27 bp, Artificial Sequence
  Synthetic Sequence
aaacatggaaaaggtcaaaaacttggg

SEQ ID NO: 885 - DNA, 24 bp, Artificial Sequence
  Synthetic Sequence
aatgactagaatgaagtcctactg

SEQ ID NO: 886 - protein, 176 aa, Artificial Sequence
  Synthetic Sequence
LEPGEKPYKCPECGKSFSRRDELNVHQRTHTGEKPYKCPECGKSFSTTGALTEHQRTHTGEKPYKCPECGKSFSHTGHLLEHQRTHTGEKPYKCPECGKSFSTHLDLIRHQRTHTGEKPYKCPECGKSFSQSSNLVRHQRTHTGEKPYKCPECGKSFSQSSSLVRHQRTHTGKKTS

SEQ ID NO: 887 - protein, 204 aa, Artificial Sequence
  Synthetic Sequence
LEPGEKPYKCPECGKSFSTTGALTEHQRTHTGEKPYKCPECGKSFSTSGNLTEHQRTHTGEKPYKCPECGKSFSDPGALVRHQRTHTGEKPYKCPECGKSFSTKNSLTEHQRTHTGEKPYKCPECGKSFSHRTTLTNHQRTHTGEKPYKCPECGKSFSRRDELNVHQRTHTGEKPYKCPECGKSFSQAGHLASHQRTHTGKKTS

SEQ ID NO: 888 - protein, 204 aa, Artificial Sequence
  Synthetic Sequence
LEPGEKPYKCPECGKSFSRADNLTEHQRTHTGEKPYKCPECGKSFSDPGALVRHQRTHTGEKPYKCPECGKSFSTTGNLTVHQRTHTGEKPYKCPECGKSFSSPADLTRHQRTHTGEKPYKCPECGKSFSQLAHLRAHQRTHTGEKPYKCPECGKSFSHKNALQNHQRTHTGEKPYKCPECGKSFSRKDNLKNHQRTHTGKKTS

SEQ ID NO: 889 - protein, 176 aa, Artificial Sequence
  Synthetic Sequence
LEPGEKPYKCPECGKSFSQSSSLVRHQRTHTGEKPYKCPECGKSFSTTGNLTVHQRTHTGEKPYKCPECGKSFSERSHLREHQRTHTGEKPYKCPECGKSFSERSHLREHQRTHTGEKPYKCPECGKSFSQNSTLTEHQRTHTGEKPYKCPECGKSFSTHLDLIRHQRTHTGKKTS

SEQ ID NO: 890 - protein, 176 aa, Artificial Sequence
  Synthetic Sequence
LEPGEKPYKCPECGKSFSDPGALVRHQRTHTGEKPYKCPECGKSFSTTGNLTVHQRTHTGEKPYKCPECGKSFSQSSNLVRHQRTHTGEKPYKCPECGKSFSHRTTLTNHQRTHTGEKPYKCPECGKSFSTSGNLVRHQRTHTGEKPYKCPECGKSFSRSDHLTTHQRTHTGKKTS

SEQ ID NO: 891 - protein, 260 aa, Artificial Sequence
  Synthetic Sequence
LEPGEKPYKCPECGKSFSTSGNLVRHQRTHTGEKPYKCPECGKSFSTSGNLVRHQRTHTGEKPYKCPECGKSFSQKSSLIAHQRTHTGEKPYKCPECGKSFSQRANLRAHQRTHTGEKPYKCPECGKSFSTHLDLIRHQRTHTGEKPYKCPECGKSFSDPGALVRHQRTHTGEKPYKCPECGKSFSRSDHLTTHQRTHTGEKPYKCPECGKSFSTHLDLIRHQRTHTGEKPYKCPECGKSFSQRANLRAHQRTHTGKKTS

SEQ ID NO: 892 - protein, 260 aa, Artificial Sequence
  Synthetic Sequence
LEPGEKPYKCPECGKSFSRSDKLVRHQRTHTGEKPYKCPECGKSFSTTGALTEHQRTHTGEKPYKCPECGKSFSQRANLRAHQRTHTGEKPYKCPECGKSFSQSGNLTEHQRTHTGEKPYKCPECGKSFSTSGHLVRHQRTHTGEKPYKCPECGKSFSQRANLRAHQRTHTGEKPYKCPECGKSFSQRAHLERHQRTHTGEKPYKCPECGKSFSTSGNLTEHQRTHTGEKPYKCPECGKSFSQRANLRAHQRTHTGKKTS

SEQ ID NO: 893 - protein, 232 aa, Artificial Sequence
  Synthetic Sequence
LEPGEKPYKCPECGKSFSRNDALTEHQRTHTGEKPYKCPECGKSFSQNSTLTEHQRTHTGEKPYKCPECGKSFSDPGALVRHQRTHTGEKPYKCPECGKSFSQSSNLVRHQRTHTGEKPYKCPECGKSFSTTGNLTVHQRTHTGEKPYKCPECGKSFSREDNLHTHQRTHTGEKPYKCPECGKSFSDPGNLVRHQRTHTGEKPYKCPECGKSFSTTGNLTVHQRTHTGKKTS

SEQ ID NO: 894 - protein, 34 aa, Artificial Sequence; identical to SEQ ID NO: 395
  Synthetic Sequence

SEQ ID NO: 895 - protein, 28 aa, Artificial Sequence
  Synthetic Sequence
NNHDNGNNHDNINGHDNINIHDHDHDHD

SEQ ID NO: 896 - protein, 34 aa, Artificial Sequence; identical to SEQ ID NO: 397
  Synthetic Sequence

SEQ ID NO: 897 - protein, 30 aa, Artificial Sequence; identical to SEQ ID NO: 398
  Synthetic Sequence

SEQ ID NO: 898 - protein, 30 aa, Artificial Sequence
  Synthetic Sequence
NNNINGNGNGNNHDNIHDNINNHDNGHDNI

SEQ ID NO: 899 - protein, 28 aa, Artificial Sequence
  Synthetic Sequence
HDHDNININGHDHDHDHDNGHDNINHNG

SEQ ID NO: 900 - protein, 30 aa, Artificial Sequence; identical to SEQ ID NO: 373
  Synthetic Sequence

SEQ ID NO: 901 - protein, 36 aa, Artificial Sequence; identical to SEQ ID NO: 374
  Synthetic Sequence

SEQ ID NO: 902 - protein, 28 aa, Artificial Sequence
  Synthetic Sequence
NHHDNGNGHDNINHHDNGNGHDHDNGNI

SEQ ID NO: 903 - protein, 28 aa, Artificial Sequence
  Synthetic Sequence
HDNGNKNGNHNINGHDNINGNHHDHDNI

SEQ ID NO: 904 - protein, 32 aa, Artificial Sequence; identical to SEQ ID NO: 391
  Synthetic Sequence

SEQ ID NO: 905 - protein, 30 aa, Artificial Sequence; identical to SEQ ID NO: 392
  Synthetic Sequence

SEQ ID NO: 906 - protein, 28 aa, Artificial Sequence
  Synthetic Sequence
HDNINGNGNNNNHDHDNNNNNNHDNIHD

SEQ ID NO: 907 - protein, 32 aa, Artificial Sequence; identical to SEQ ID NO: 394
  Synthetic Sequence

SEQ ID NO: 908 - protein, 28 aa, Artificial Sequence
  Synthetic Sequence
NININHHDNGHDNGNHNINHNHNINHHD

SEQ ID NO: 909 - protein, 28 aa, Artificial Sequence
  Synthetic Sequence
HDHDHDNGNINKHDNGNHNGHDHDHDHD

SEQ ID NO: 910 - protein, 28 aa, Artificial Sequence
  Synthetic Sequence
NHHDHDNGNINHHDNINGNHHDNGNINH

SEQ ID NO: 911 - protein, 30 aa, Artificial Sequence; identical to SEQ ID NO: 403
  Synthetic Sequence

SEQ ID NO: 912 - protein, 28 aa, Artificial Sequence
  Synthetic Sequence
NHNININIHDNGNINGNHHDHDNGNHHD

SEQ ID NO: 913 - protein, 28 aa, Artificial Sequence
  Synthetic Sequence
NHHDNIHDHDNINGNGNHHDNGHDHDHD

SEQ ID NO: 914 - protein, 28 aa, Artificial Sequence
  Synthetic Sequence
NHNIHDNINGNHHDNINIHDNGHDNINH

SEQ ID NO: 915 - protein, 28 aa, Artificial Sequence
  Synthetic Sequence
NIHDNIHDHDNIHDNGNINHNHNHNHNG

SEQ ID NO: 916 - protein, 28 aa, Artificial Sequence
  Synthetic Sequence
NHNGHDNGNHHDNGNINHNIHDNINHNH

SEQ ID NO: 917 - protein, 30 aa, Artificial Sequence; identical to SEQ ID NO: 409
  Synthetic Sequence

SEQ ID NO: 918 - protein, 30 aa, Artificial Sequence; identical to SEQ ID NO: 410
  Synthetic Sequence

SEQ ID NO: 919 - protein, 30 aa, Artificial Sequence; identical to SEQ ID NO: 411
  Synthetic Sequence

SEQ ID NO: 920 - protein, 28 aa, Artificial Sequence
  Synthetic Sequence
NNNGNNHDNGHDNGNNNIHDNININGNI

SEQ ID NO: 921 - protein, 28 aa, Artificial Sequence
  Synthetic Sequence
NNNGNGNGNGNNHDNINNHDHDNGHDHD

SEQ ID NO: 922 - protein, 30 aa, Artificial Sequence; identical to SEQ ID NO: 414
  Synthetic Sequence

SEQ ID NO: 923 - protein, 36 aa, Artificial Sequence; identical to SEQ ID NO: 415
  Synthetic Sequence

SEQ ID NO: 924 - protein, 30 aa, Artificial Sequence; identical to SEQ ID NO: 416
  Synthetic Sequence

SEQ ID NO: 925 - protein, 28 aa, Artificial Sequence
  Synthetic Sequence
NHNININININIHDNGNINGNHNGNING

SEQ ID NO: 926 - protein, 30 aa, Artificial Sequence; identical to SEQ ID NO: 418
  Synthetic Sequence

SEQ ID NO: 927 - protein, 28 aa, Artificial Sequence
  Synthetic Sequence
HDNININGNIHDNINIHDHDNIHDNNHD

SEQ ID NO: 928 - protein, 30 aa, Artificial Sequence; identical to SEQ ID NO: 420
  Synthetic Sequence

SEQ ID NO: 929 - protein, 28 aa, Artificial Sequence
  Synthetic Sequence
HDNIHDNINIHDNINGNGNGNNNGNINI

SEQ ID NO: 930 - protein, 28 aa, Artificial Sequence
  Synthetic Sequence
NINGNGNGHDHDNINNNGNNHDNIHDNI

SEQ ID NO: 931 - DNA, 10065 bp, Artificial Sequence
  Synthetic Sequence
gtgcagtgactagacatggggcagctgtttaaagacaaaaaggccccaaaaaggagggatggcacgaaacaccctccaatatgggcatggagtctagagtgacaaagtgatcaaaagttcatttcctatggggtgtccgaatgtacttaataataaaaagagaacaagagccatgcaaactgagagggacaaagtagaaagagtagcagacaccaagcaactaagtcacagcatgataagctgctagcttgttgtcattattgtatccagaacaacatttcatttaaatgctgaagaatttcccatgggtccccactttcttgtgaatccttgggctgaacccccctgtcctgagtggttactagaacacacctctggaccagaaacacaaaagtggagtaacgcacactgcaaagctgtgcttccttgtttcagcctgtgaatcctcaccttgtttcccatctagcctatatttttcaaactaacttggccatagaatcatgtagtatttagggtggaagctgccccaggtctagcacgtcatttaacagatgaggaaatggaagcttgggcagtggaagtatcttgccgaggtcacacagcaagtcagcagcacagcgtgtgtgactccgagcctgctccgctagcccacattgccctctgggggtgagtatgtcttcacatcctccaataccctaatgacagacaaacagaacatggcaaagcctcagctctgcatggtgaaagtaagaaccagcaattgccacaaacagaaatacagtgttggtccggcagcctccgggggttctgcacaagtggattaccagtgaatacaaggctatctatctttcgaaaaaccaaagttgtatttatgctatctattttctataaaattttatattaatttatttgttacctatttttgaactctttcaaaagcacactttatatttccctgcttaaacagtcccccgagggtgggtgcccaaaaggctctacacttgttatcattccctctccaccacaggcatattgagtaagtttgtatttgggtttttttaaaacctccactctacagttaagaaaactaaggcacagagcttcaataatttggtcagagccaagtagcagtaatgaagctggaggttaaacccagcagcatgactgcagttcttaatcaatgccttttgaattgcacatatgggatgaactagaacattttctcgatgattcgctgtccttgttatgattatgttactgagctctgttgtagcacagacatatgtccctatatggggcgggggtgggggtgtcttgatcgctgggctatttctatactgttctggcttttcccaagcagtcatttctttctattctccaagcaccagcaattagctttaccttttcagcttctagtttgctgaaactaatctgctatagacagagactccggtgaaccaattttattaggatttgatcaaataaactctctctgacaaaggactgctgaaagagtaactaagagtttgatgtttactgagtgcatagtatgtgctagatgctggccgtggatgcctcatagaatcctcccaacaactcatgaaatgactactgtcattcagcccaatacccagacgagaaagctgagggtaagacaggtttcaagcttggcagtctgactacagaggccactggcttagcccctgggttagtctgcctctgtaggattgggggcacgtaattttgctgtttggggtctcatttgccttcttagagatcacaagccaaagctttttattctagagccaaggtcacggaagcccagagggcatcttgtggctcgggagtagctctctgctgtcttctcagctctgctgacaatacttgagattttcagatgtcaccaaccgccaagagagcttgatatgactgtatatagtatagtcataaagaacctgaacttgaccatatacttatgtcatgtggaaaatttctcatagcttcagatagattatatctggagtgaagaatcctgccacctatgtatctggcatagtgtgagtcctcataaatgcttactggtttgaagggcaacaaaatagtgaacagagtgaaaatccccactaagatcctgggtccagaaaaagatgggaaacctgtttagctcacccgtgagcccatagttaaaactctttagacaacaggttgtttccgtttacagagaacaataatattgggtggtgagcatctgtgtgggggttggggtgggataggggatacggggagagtggagaaaaaggggacacagggttaatgtgaagtccaggatccccctctacatttaaagttggtttaagttggctttaattaatagcaactcttaagataatcagaattttcttaaccttttagccttactgttgaaaagccctgtgatcttgtacaaatcatttgcttcttggatagtaatttcttttactaaaatgtgggcttttgactagatgaatgtaaatgttcttctagctctgatatcctttattctttatattttctaacagattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccggtaagtaacctctcagctgcttggcctgttagttagcttctgagatgagtaaaagactttacaggaaacccatagaagacatttggcaaacaccaagtgctcatacaattatcttaaaatataatctttaagataaggaaagggtcacagtttggaatgagtttcagacggttataacatcaaagatacaaaacatgattgtgagtgaaagactttaaagggagcaatagtattttaataactaacaatccttacctctcaaaagaaagatttgcagagagatgagtcttagctgaaatcttgaaatcttatcttctgctaaggagaactaaaccctctccagtgagatgccttctgaatatgtgcccacaagaagttgtgtctaagtctggttctcttttttctttttcctccagacaagagggaagcctaaaaatggtcaaaattaatattaaattacaaacgccaaataaaattttcctctaatatatcagtttcatggcacagttagtatataattctttatggttcaaaattaaaaatgagcttttctaggggcttctctcagctgcctagtctaaggtgcagggagtttgagactcacagggtttaataagagaaaattctcagctagagcagctgaacttaaatagactaggcaagacagctggttataagactaaactacccagaatgcatgacattcatctgtggtggcagacgaaacattttttattatattatttcttgggtatgtatgacaactcttaattgtggcaactcagaaactacaaacacaaacttcacagaaaatgtgaggattttacaattggctgttgtcatctatgaccttccctgggacttgggcacccggccatttcactctgactacatcatgtcaccaaacatctgatggtcttgccttttaattctcttttcgaggactgagagggagggtagcatggtagttaagagtgcaggcttcccgcattcaaaatcggttgcttactagctgtgtggctttgagcaagttactcaccctctctgtgcttcaaggtccttgtctgcaaaatgtgaaaaatatttcctgcctcataaggttgccctaaggattaaatgaatgaatgggtatgatgcttagaacagtgattggcatccagtatgtgccctcgaggcctcttaattattactggcttgctcatagtgcatgttctttgtgggctaactctagcgtcaataaaaatgttaagactgagttgcagccgggcatggtggctcatgcctgtaatcccagcattctaggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataattcattatattcttgctctttcagtcaagtgtacatttagagaatagcacataaaactgccagagcattttataagcagctgttttcttccttagtgtgtgtgcatgtgtgtgtgatgtatacaaagagagagataattgtatttttgtattttcttttaaataatttttaaaattgacccttttcctgagacaaattgccagaatagtttgtatttagagatggtacctctaagagtaaggttgctggttgctgagcaattgacttgaaaacttttaaaattcaaattttaattccactactcaaaagaattgccatgttttaaaaaagagaattggtgccataagttagttgtctatgtttgaaaatgaagaagatatgcaacgtcatggcctggtcacttacccgcagccctgagttgtaggcacatcatatgtgagaatgaggatgcttttctttcatttaaaatccctccccaaaacttggctctaattgcagtcatgacaatcatgtacatttggatttatgtgcacgagtctcttaccctgagagaggacaggtgctacaggtggaggggacccgtctgggtcacgttcacattttgaacatgctggttttcagtcactgcacactcatctcccagcacaggtcatgggcagcagatgcaaaagctgcccgtggtcctatttggaggtgcatgaaatgagcagaagacagaacagcttgatctgactagaagggcagcttgtccctaccaagacttgaaggattgcctttcatctgttagggtaaaaggtagaatgaaccaaggaagggcaggagggggctggggttagggtagaaggaaggggccatggagaagggagatccatcccataggaggaaggcagtgcggcagggaggtttgaaggtatcagcttttgtggctgacatacatgcagtcatgtcaattgctcgtttttccttttccatcttattaaatgtcttccaacgttagcacgaagaaaagctatttgcagtgttgccagcctttccagagcccgtccccattacctccccaggcccatgcctttactccttggagtttcaactcacgaccttcaggatctgactttattcaccaactctggggtgaacgtaccttctgtctccacccagaggtctctatcaaagaggagattgcatgccatggataaagtcaaagtagaggtgactgtccttaggaagagtaatgtgaaaattcataaactgggattctgtttacattttgtactccaggggttcttagtttaaatcgctctgaataaattaagatgcaatggcatttcaactgttatgattaaatttacaaatcatttattttctatcacggggagagatagagctccaaatgcaaacataactgctcaagtgttaacacttataatgaaaacataagaattaccaccaactaccctgggggctagaagcagaaatgtgaaccagaaaacaaatcatgaactttcctttttttttttgagatggagtctcgctctgttgcccaggctggagtgcaatggtgcgatctcggctcactgcaaccactgcctcccgggttcaagcaattctcctgcctcagcctcctgagtagctgggactacaggcatgcaccaccacgcctgggtaattttttgtatttttagtagagacagggtttcaccgtattagccaggatgctctcgatctcctgacctcgtgatctgcccgcctcggcctcccaccgaagtgctgggattacaggcatgagccactgtgcccggccaacaaatcatgaactttctaactgcagttccttgtagcttgttaacacatccacttacttattgtcagagtacgtggagattttccacaaccctcggggataaggctgaacagaagaggcaaaaacgtgaaaacatttcgatagctcctatactttgaaataaaattcactgtaaaagttgcttgtatttttccaaaac

SEQ ID NO: 932 - DNA, 8111 bp, Artificial Sequence
  Synthetic Sequence
tgggaggcaagcccaggactttagaagacatgctggggacccagggatcctgtgtggccatcacacacctgctagaagcaacaccccttccctagaaaaatgaacctggccagacacagtttcaggggctcctggaccccatatgaaggctcctggggtacgatgagtctcagaggacagggagaccagtccctggcaacccctgcatgcccctactcccccccaagtaaacgcatcccctcccagaaagacctgcggcagggttccagcaatcctacctaacgcactcctgggtgagtccctccacccacccaccccgccccggcattccaatcccctcagttactcagggggattgacagtcccagttggtaggggtttgagttctcatcctgtgcgtgtgctgggctccaagcaatcctggaggcaggcccctacacccccatttcacaggtgaggaaactgaggcacaaagaaagacaatcctaggaagcagggtcagcctcggtgcccaggccgcctctaaagctctcagcctggagaccacgggtgaggaggtgtcccccaccccacacacagcacaccagagcccacctcggcgggcgatctccgccttcagcagcccctccatggcgtccgactcggccaggtccaggggcaggtccccgtcactgttgacggcggcgatgttggccccgtggctcaggaggtacctgggggtgggggctggtcagggctgagggtccaggcccccaccccgaccaggtcctgcccggccctcccttgcctcacctggcgatatctaggtagccacaggaggcggccacgtgcagtggcgtccagccctcgttgtctgcctggttcacagtggcgccctgctccaccaagaagcgcaccacctccaggttctcatcaatgcaggcctgggggtgggaaacagccgtcagccgcacctaccccagccacggtgtcccagcaagtcgggactccagagggagccacgaaaacagatccagggacacggtgctaggacagtggggaaaatgacccaacagcctctggccactggctgtttaagatacgcctggttgcccagtggtcaggccggccaggccttcagtgctcagtggaaaccacgaaaggactcctggctatctgcaaacaggaagtgaacggggaagggagggggcttctcatctgggtgcgggaaccccacatggtacctgttagacacggcaaaacccccgtcaccacccacaggtggcgcttccagtgctcagactagggaagaggttccagcccctcctccttcagagccaggagtcctggcccccagcccctcctgccttaaacccagccaggtccttccaagggtcaagctcggaaaccaccccagcagatactctgcaggaacgaagccgtgggcccagggctatgcagggtggaggaaggccaccctgtgctgggacagactcaggggcctgggcgggactcccagaggggtgagacagctgcacacctgtgtgcctgggccccaggctgtcacactccagttcactgaggccccctctgcacggggccctgcagccaggggctgacacgggccaccgtttctcattcttcccttaggggtccaaaacttggggggacaaaagccgaagtccagggggtcggaggagggacttgccccaggccttgtggacactgggtgggctccgggacctgaactggagctgaggaaggagtgaagctaaactcctagatccacgggataaattaccccccaagtccctcacctctccaaagctgcccatctggaggaggcgggagggagctacgagggccaagagcatgaggtcatggaaactcgggctgtgaaggggccgcacgtgccctgggaacgggatgaactcggctcgtttatttccacccagttgtcatggcgataggggaggggggcaaggagagcaatgggcctttccctttcaaggacctgcccagtacaggcatccctgtgaaagatgcctgaggcctgggcaccagggactccagagtccaggcccaacccctccccattcaacccaggaggccaggccccagcccttccgccctcagatgaaggagtccaggcccccagcctctccccattcagacccaggggtccaggcccagccccgcctccctaagacccagaagtccaggcccccagcccctcctccctcagacccacgagtccaggccccagcccctcctccctcggacccaggagtccaggcccccagtccctccaccctcagacccaggagtccaggccccagcccctcctccctcggacccaggagtccaggccccagcccctcctctctcaaacccaggagcccaggcccccagctcttctctgttcagccctaagaatcctggctccagcccctcctactctagcccccaaccccctagccactaaggcaattggggtgcaggaatgggggcagggtaccagcctcaccaagtggttgataaacccacgtggggtaccctaagaacttgggaacagccacagcaggggggcgatgcttggggacctgcctggagaaggatgcaggacgagaaacacagccccaggtggagaaactggccgggaatcaagagtcacccagagacagtgaccaaccatccctgttttcctaggactgagggtttcagtgctaaaactaggctgtcctgggcaaacagcataagctggtcaccccacacccagacctgacccaaacccagctcccctgcttcttggccacgtaacctgagaagggaatccctcctctctgaaccccagcccaccccaatgctccaggcctcctgggataccccgaagagtgagtttgccaagcagtcaccccacagttggaggagaatccacccaaaaggcagcctggtagacagggctggggtggcctctcgtggggtccaggccaagtaggtggcctggggcctctgggggatgcaggggaagggggatgcaggggaacggggatgcaggggaacggggctcagtctgaagagcagagccaggaacccctgtagggaaggggcaggagagccaggggcatgagatggtggacgaggaagggggacagggaagcctgagcgcctctcctgggcttgccaaggactcaaacccagaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccaatcctgtccctagtggccccactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggcagggaaggatctgggccagccgtagaggtgacccaggccacaagctgcagacagaaagcggcacaggcccaggggagagaatgcaggtcagagaaagcaggacctgcctgggaaggggaaacagtgggccagaggcggcgcagaagccagtagagctcaaagtggtccggactcaggagagagacggcagcgttagagggcagagttccggcggcacagcaagggcactcgggggcgagaggagggcagcgcaaagtgacaatggccagggccaggcagatagaccagactgagctatgggagctggctcaggttcaggagagggcagggcagggaaggagacaaagtccaggaccggctggaggggctcaacatcggaagaggggaagtcgagggagggatggtaaggaggactgcatgggtcagcacaggctgccaaagccagggccagttaaagcgactccaatgcggaagagagtaggtcgaaggggaatggtaaggaggcctggggcagagtggtcagcacagagtggctaagcccagggccagttgaagcggctccaattcggaagtggggtggtcgaaggggaatggtaagggggactgggacggggtgtcagcatagggtggcaaagcccagggccaggaacgacggggcggatcgagactggcaacggggaaggaggatgccccaggtggcgcagcagagggtggacctggccccgggagacgccgggcggggggcgctgacctggtgcagggcgctgataccgtcggcgttggtggagtccagcacggcgcgggcgggcggcggcgcggcggggtcgagctcggcgccggggccagggtcggcggcgcgcagcatcagacgcgcctcgtccaggtcgccgcccgcacaggccgccaggaactcggcggcgcgctcgaagcggacggtgcgggcgcggcgctctccggggccaggctcggcgcccgcccgcgccccccactgccgcagctgctcccgtcgccgctcccgggcagccgccgccgccgcccccgggccagccgccgggccatcctctccggacatcgcaccgcccgcccgcccagcgagcgagcgagcgccgagccccaaccgccgccaccacccgcccgcccgcccgccccgggggccgccgggaactgccgctggccccccaccgccccaaggatctcccggtccccgcccggcgtgctgacgtcacggcgctgccccagggtgtgctgggcaggtcgcggggagcgctgggaaatggagtccattagcagaagtggcccttggccacttccaggagtcgctgtgccccgatgcacactgggaagtccgcagctccgaggcgcccagtggaaatcgccagatgagggcctcctccggggaatgctgggaaatggagtctacaggccggaggggtgccccacggcatactaggaagtgtgtagcaccgggtaaaggggatgaatagcagactgccccggggcagttaggaattcgactggacagccgcgtgggagggagtgcggggagaggcagagttgttttgttattgttgttttattttgttttctttgttttgagacggagtctcgctctgtcgccacgctggagttcagtggcgcgatatcggctcgctgcaacctccgcttcccaggttcaagcaattctggctcagtccccagagtagctgggataacaggcgcgcgccaccccgccctgctaatttttatatttttagtagagacgggatttcaccatgttggccatgatggtcttgatctcttgacctcatgatccgcccgcctcggcctgtaatcctgctgggatgacgagcgtaagccaccatgcccagctgggttttatttattttggtttttttcctgaccccttaactagaaataagctccacgagagcgggatcttttgtcttctgtgcactacttgtcctcggttcttagaacagaacctgagagaacctgatcgcaaatatttttggaatgaatgaatgaatgggttcaccagggcaccatgggaaactgagtccgcaacctagaagccatgaaagacagtccacttccaagcttccctgggtgacctcgcagggcatgctgggaaatgaaatttgcggtgaaaaggtcaggaccacgatcctagggcacgctgggaaatgtagcccacagggccacacccctaaaagcacagtggggtgcaaggcagggccccaaggcatttagggctcggggcaagagaaatccacactccactccctaatggtaatccctgagccacaccgagtaaaggaacccaagacacagtgtccacagggacagggctctcagagctttcactggcccgcgcttctcctgcgcccacccggacctcctgggaaccgcccaggccctcgcgcgctctcaaggcatgctgggattggtggtcccgggcaaggagttccagcaggtggggggcgaatcacctttcagcgggcccaagcgatgagcacaccttgatcttcaccttacggatcccgcgcccaactcaagattgggaaggtggctggcactttgtgacaggaagagtcccataaaaatcatacagaaaagggccaaaatcgggacagagactacagactgtttcccaagcgctgtgggagtttcccacccactctgaagtccttgggtttgcgcggagacgtaaactgcgcatcccacgaggcctgtttctttccctctctctttctcttttgttgttgttgttgctgctgctgttacgaaaatttttgtggttttattgtatcatgaggcattgaaacatccggcgactcaatgtctaggcggtgaggcagccgctttctccttcactttctttgggttaagtagagcaacttgtcagtagttttgtttttttttgttgttgttgttgtttttgagacggaggcttgctctgtcgcccaggctggagtgcagtggcgcgatgtcggctcactgcaagctctgcctcccgggttcaagcgattctcctgcctcagcctcctgagtagctgggactacaggcgtgctccaccacgcccggctaatttttgttttttagtagagacggggtttcaccatgttggccaggctggtctcgaattcctgacctcgtgatccgactgcctcggcctcccaaaatgctgggattacaggcgtgagccgccgcgaccggccagattttttttttttttaaaggacaaccttttgcattacttaagtctttccaaggcatgcgctggtacaacacaaacttttcccattacatgcagctagtctagtgtccagacgtcatgcacaacacctccgtggcatcagcgcactgcgcccactcccactgcgaccctgctcatttgtgcatcatatctggaggagtggcaatagttctggaaagaggagggaagaggaggcagcgtgagggcccggtggagaggaggtcagctgaagttgtgcagagcaagcctgcatat

SEQ ID NO: 933 - DNA, 5040 bp, Artificial Sequence
  Synthetic Sequence
ttatatttggctgatagtttgggtataaaatttggagttaggctttctttccttaagaatattgtagtcagtgttcattctcttttgtaaaagatttatttttttagacaagatcttgctctgttgtctaagctggagtgcattggcgtgatcatagctcattggagccttgacctcccaggctcaagcagtcctcccatctcagcttcctgagtagctgggactacaggcacgtgccaccatgcccagttaatttttgtatttttttgtggagacagagttttgccatgttgcccagtctggtctcaaactcctaagttcaagtgatccacttgcttcagcttcctaaagtgctgggattacaggcatgagccaccatatccagcccagactttttttcttgtcattatttcccaaacaatacagtataacaactatttccatagcatttacattgtatcaggtatcataggtaagatagagatgatttaagttactcttgagaggatgtgtgtaggtcatatgtagacactataccattttatatgagggacttttgagcatctttgggttttggtgtctttggcggtcctggaaccagtgtcccccacggataccaactgtccataagcaaagtagaggtttatttgtttgattatagtggtgagaaatgaatgaatttctttctctgtcctttacaggaagaagttctaaaggattttgaagacttggctggaaaactcccatggtcaaaccccttaaaagtgtggaaaattaatgccagttttaaaaagaaaaaagcaaagcgcaaaaagataaatcagaattcaagtaaagagaagattaataatggacaagaagtcaaaatcgatcagagaaatgttaaaaaagagttcactagccatgctttagattctcatatcttagattattatgaaaatccagccatcaaagaggatgtatcaacattaataggtgatgatttggcatcttgcaaagatgagactgatgaaagctcaaaagaagaaactgagcctcaagtgctgaagtttagagtcacatgcaacagggcaggagagaaacattgctttacctcaaatgaggctgcaagagattttgggggtgctgttcaagattattttaagtggaaggccgacatgaccaactttgatgtggaggtaggtataggctctgactgtggtgattgaagaatgctgtcacagatttgtagaatttccagactaacccaatgttatgcacaattaaactgaggaaatagtagggtgtggttaagaagagcataggctatggagacagatttagatccaaattttaactccacaactgtataactttggactagatagtctctgagcctctgtttcaaatggagccaataatattatgtacctttaaggttatcctgaaggataaatgaggtacatttaaaatatttggtgcaataccttgcatacactaagtgctccatagatggaagccatcaaggttgagttgtctgttttctttttctttttcgagacagagtcttgctctgtcacccaggctagagtgcagtggtacgatctcagctcactgcagcctccaccttcctggttcaagtgattcttgtgctcagcctcccaagtagctggggctacaggcacatgccaccacacctggctaatttttgtattttttaatagacatggggtttcaccctgttggccaggctggtctcaaactcctgtagtgatctgcctgtcttggcctcccaaagtgctgggattatagctgcacctggcctgatttctaatagactactcttttactatattatatggactttcatattataaaattcaaccagaattaaaatagttattttgtttttgtgtgtcttggttgcagagctttgctactgaaaaaataaaataaggctgggcgcagtggctcacgcctgtaatcccagcactttgggagggaggccgaggcaggtggatcacttgaggtcaggaatttgagaccagcgtggccaatatggtgaaaccctgtctctactaaaaatacaaaaattggcccgtcatgatggcacactcctgtggtcccagctactggggaggctgaggcaggagaatcacttgaaccagggaggtggaggttgcagtgagccaagatcatgccactgcacttcagcgtgggtgacagagcaagactccatctccaaaaaaaaaaaaagtgcattttttaaacaaaccctgaccttctcaaaccaggacatcattgctaatactcatcctagagtcagctctcctctttcttaaattctgctagatttccaaagccaggtacctcattttagtatatttactttccttggtcttaatcctcttttatctgggtgattgacatgtgaaccataggaactaaagtatttaagtctctgacaccttaaaacatctctagcagctgatatatttaccaaagagtcaatttccgttgcttttgggacacttgaatctggggcaagtcactgattctaacttttttgtgcatgtcaaatcaggagtctaattccccattcctttttttcctcatacactgccttgcaaatgaatacgtggctcacacctgtaattccagtactttgggaggccttggcaggcagatcgcttgagcccaagaatttgagaccagcctgggcaaaatagtgagaccctgtctccactaaaaataaaataaatattttaaaaaataaaatgaataggttaaccagaacagtgatgtctaaaagttagcaaggtgattcagaggagtttctggagccgtgctgcattggtttaaatcccagaggaggctgggtgtggtgacacacacctgtaatcccagcactttgggaggtcaagattggaggattgtttcaggccaggagttctagaccagcctaggcgacatagggagaccctgtctctacaaaaaataaaaaaaaaattagctgagtatgatgacagacacctatagtcccaggtacttgggaggctgaagtaagagattgcttgagcccaggaagtccaggccacagtgatctgtgatcatgccactgcactcaatcctgggcgacagagtgagaccctgtctcaaaaagggaattcctgaggagctgtctttgtgcctcagtatcctcatttataaagggaataatacctagcttaggggatgtgaatatgagatgaggccatgtatgtaaaatgtgtagaatagtgctttgcattaggaagtgctgtatgagtatcagctgtgcttctgctgtggttaagctcaggaagtctttcagtgaaagattggggaacattttggtttagtttagttcacttttgagacagggtctcgctgtatcacccagactggagtgcagtggtgcaatcatggctcactaaagccttgacctcccaggctcaggctgtcctcccacctcagcctcccgagtagctgggactgtgggcctgtgccactgcactcagtgtaataagactttttatttgagactttaccaatttttaaaacttttttgttgagatacactttgcaccccttaagtacacaaatcttaagttttcagtaagtttttacttatatacacgatgtgtaaacagcatccagatagagatacaaactatttccagtacctcgggctccccttgccctttcccagtcagtacccactcaaggcaatcactaatctgactgctaccaccaaaaattcgttttgccaatttttgaacttcatataaatgaaatcatatagtatatagtttttttcgttcattttaatatctattattttcttttaattcactatgtttattggctacaaccagatttcaccccaatttttataacacaaacatattttgaaggcttagaggttttttaatatgtattttttaaatgtcttaactttttagacagttctgggctgtggtgcgctatgccaatcaggtgtctgcactaagtttggcatcaatatggtgacctcccaggagtaggggaccaacaggttgcctaaggaggggtgaaccggcccaggtcagaaaagtcttattactaagcgcagtttgaacaatcctaaaactattttgtcttgttccactttgctataggttcttttgaacatccatgataatgaagtcattgtgggcattgcattgactgaagagagtctccaccgaagaaatataacacattttggacctacaactcttagatcaactcttgcctatgggatgctcaggtaagaaaatgagtttgccatccagcttaaagtggcaccttggtttgaatgttcccttcaaaatactgatttgtgtgtatgtgtgtttgaggtactgtgttggaatagagtggataaagaatagaatggaaagctaatgatctatttctttcccttctatccaatattgactccttgtgatctgaaaatactgcttaataggtggctcagttttctttatttctttttttgagacaaagtctcactctgtcgcccagactggactgcagtggcgagatctcagctcactgtaacctctgcctcccgggttcaagcgattctcctgtctcagccgaatagctgggattacaggtgcctgccaccacgcccagataatttttgtatttttttaatagagacgacatttcactatgttggcgaggctggtctcaaatacctgacctcaggtgatccacccgcctcggcctcccaaagtgctgggattacaggtgtaagccaccgcgcccggttttcttgattttaaaatgagagtaatagttttcacctgcctcttgaatgaggttaccacatttctgtttagcaaggatatgtgtgttgggagataagagtaaattttgctgtttcatttctgtgcagtgaaaaagatgaaagcagagatttttatgtgtataacgtgtaaatgaacagaaagagcttttacttatatttttaagctttatactcacagcaactcaggaatcatcctagaattt

SEQ ID NO: 934 - DNA, 2020 bp, Artificial Sequence
  Synthetic Sequence
cattttttaaaatgttccctagctactaaatacgtaccacaatggcatctaacaaccatcaggggctttttgaaactttgtcctcttagagttagattctattaagctctgaggagcagcctaatggttttcagattccagacttcaaccatacctctctagagtgttacagtgtcttactctccaagaatttgtttggatctttaattaattaagacatattattcccacagtgatgtggatgttactacataaaagctaaagaatacaaattgataacttatgcatatgcatgagtagtaatggtttacctgcttgcagacataatgtagaccacaaaacagaaacaccaggggaaagcattatggtggaatgagggacaaaactgagaaaaaataatgaaggtaatactgtcattatgggagaagataaaaaataaaataattgcaaaataaattttatttaattggatatagcatttctggctttatccacatttctacatagatattttacaatttcatggactagagtattattatcctagtttagggtatttcttccccctcaggcttgtcttcaaactctagaacattattaatttttctggcaaacaaggctgtcttagaaattctactttaaggatcttgctcagatatctcatcctttacagaactattgttgaccagcccagtaaaagaggatttctatcaacccataggcttcctacaatccttatcatcatttacttcttgacattctctaaatgtttcagtttttctttttgcatgaacatgtcaaatctcttaaaatattacgtattctgtggaatattgatgctatttctttgtagtctcctaagcactcaatatagagactttcaaagagtacctattttggccttagttagcttggctcttggctagtaagctggatattgctgaatatctgagagaaagaagagtcttttccagtttactcaatagtagtaactgaagcaaaaatgtggtgagacatttttcatacataagcgtcccaacccaacttagggatttcttaaatcaatctgctgatcaagaaagacatatagattatttattaacctaaggttgtgagggaattctctgaaaggaagtgattgacccctacttaattccatagaaattggagatgactcagatgcctagcatgctagggattcgtctaccaattgtttagcaattccctagctgtcccctcaaagggatggaactgtagaatcaatttttccatttccctctcacccaaattatgctgtgcaggcaactggacggggttccactgctaggctgtaatcatttagttcaccagaagtataaatgcactaatattttatccattatatatgtctatatacagagagagaatatgggcttcacggatcctggttttccaacaaagaccaattaaatcatccaattaaaagggagttaaaataacttttacaaaccatgttaaatacaaagttcacactagtgatcaaagaatatggccccctattatcttttggtgagtcttaaatttataaggcactcagtcaccacaatctctgtgggtcactatagttctctctgtaaccattgcttaatcctgccatgcacataattgcaagcattatacagctcctacatgttttgtctctaaattcttgaaatattacttgcaaacataaaaaaatgcttatgtccaggtacttcctttgttgctgttttattaccaaagaaaagcctcacatttcacttaacacattgtgtctcatgcaaataggatgcacaaatatccattaaaaatgtagactctaatataagaagaaatatttaaaatgtgtcttaaagaggcaatttggatagctctatatactccccattagacacattttatttgtttggttgtttgtttgcttatttgttttcagcttggaacatcaaccaccagagtttatctttgcatgcaaatgataatagattgcaagtgctctctc

SEQ ID NO: 935 - DNA, 2020 bp, Artificial Sequence
  Synthetic Sequence
gcctcccaaagtgctgagattatgggcatgagccaccgcacctggccctgacaagaacctttgagttaggtataatggttcaccccaatttatagataatgaacccaagtcacaggggaagtgaagtcagttgcctaaggtcagacagcagtaaatggttctctgaccctaactccactgcctccctctcataaaaacactgggtggttacagtgggcccacctggagaagtcaagctattctctccatctcaagaacattaatttaatcatcctttttaccatataagataacatcttcacaggttctgaggatgagaatgttgacatctttgggtggtcgttattcagcctatcacaggtatccagggaagaaaaaaggaatttccaaaaagagaaaatacgaacattgggaaggctaattacagatggtgactactgaagggttagtcagaagcataatggaggcagtgatgagatgacagcacagatgcatgactctagtcccagcaactcctaaaaggtaaagaaatgtatcctgccaccctcagcttctttggggtgtcctcataaaagagaggcagtaaagcagaatcagagtcagatagagaggttgtaagaagagaagcagaggtgagtaagctgtgtttcaaacccagagtcaaggctcttgcccctctgcggtgctgccgaagcccagggtgggtggggactgacatgcaactcaggtactgtgtggcagactttgtgccttggcatgaaactatgcctgcccacaggaaggggcaccattttctcattagctcaaagagacttctgctggccaattcctgtcttctcaatactgcagctctccagagacaacactgttctctattctcctgtaagtgaggcagagcctggcagtaccctctatgccacctctcactagtacaggttagcactcagggtggcccactggtgtgtgtctcagctgctggtgtgcgtgctggtgcaggtacaaaccgtcgtgatacattttactcccactcctactaattacagcttgtgtgtccttcagtcattcacttcccttcacatgaccagcccagcagaaatgaactaccaggaacatgagctcagagcgatgggctggccacctgccaagcacctctgaatggaaagagcagaattttgcattgcctgccatgccacgtggagcaggccctgggtggctctttaggggatgggtgtggactcccacaacaaaaccaagggccatattcaaagttaaaagctctgccatagatggtatttgttgaggctgtgtgtggtagctcatgcatgtatgcccaacactttaggaggctgaggtgggaagatcacttgaggctgggagttcaagtctagcctaggcaagatagtgagatcccttctctaaaaaagataaaatattaactgggcatcatggacgtgcctgtagccccagctactggggaggctgaggcaggaggatggcttgagtccaggagtttgagactgcagtgagctgtgattgcaccattgctccctagcccgggtgacagaacaagacttttatttctttaaaaaaaaaaaaaaaaaagaaggtgtttactgcagttgctttattaaaaaaaaagtaaatgaatgttctgactgttctacttttgaaaataagtggcaaggaattagaactgtatctttcagcaacaaaatgtacactgtggttccatgtcacagccaggaatggagtcagatgtctcagaccagaatcacagctctgccacctcctgtgacatggacttgctaagctaccttgactctctggagctcactatgcccatcaataacaagaaataaataaatccgtcctgtaaggttgtcaggagaaacaaatgaggcactatatgtggaagttcctggaatagtgaccagcacagaggacgtctcaaagaaagatttgctgaaccccaaaagacaggaggactggaggaacaacaaagagacaggaaagctagcat

SEQ ID NO: 936 - DNA, 2020 bp, Artificial Sequence
  Synthetic Sequence
tacatgtgtaaacagttttagcgtagatttcctcgcacttttaaattttggattcttaatttccctgtcccccctgccccccccccaaaaaaaacctgctaacgtttaaacgaacacagtttgggaaatctgcgttaagtccttcgtgggagtggggttgctcagctcacagtaggccacgaacctgaattttctcttgtctgctgcccccttttgatagatggagggaagagcaggcttccagtgcaatggacagaagagggagcctgcaagttggtaacagagtctattagggaaagagagagtcacttgaatcctcagagctgctcctgtcaactgctttgtgcagtttttgtgacttattagctgcttgtttgcactctatctacgcctgcccaggtgtgtttgggccctagagcgaagggagcacaggcgttcatttagaaacttatccctccgtccaaatattggatgcttaccatgtgcctggtgcaatgcagggtgatacaaagaggaagataagtgaggcattcttatcgaaggaccagacactcttccagcctgactatattcattacactcgtgcctgacctttctttgactctaagattcttcctttctaaatgtgaatcttaaagactgaagtctttgatctaagactgctttcttatcacatcacatccaacaaccaacttttcacagcttcccagatcccaaattctgtttagcaaggacacttggatttttttgttttttgttataaatgacctcttcaggttcatattttcactatgtccagaattcttattttattctgttttgtgctgacattggaggcagagtctgtgtcacagaatacaccactaggggttaccctggacatggaagggtattcactcggggaagaaattttaatggaatttttaatatctagagctgtcattatcctgtgatggttcacaagaaatggaacacttaaaaatttctacagaaaaaaaggaaaatgtcatgaggcagtttttagtacttccatccctttcctggctgctctaactttacaggtacttgtaagtggcaattaagcactttttcctaattccagagtcttgccccacttcagagcaacatagagtggcctagacaggctgaggtactttgccgcttcagtcatcattaatctatggtatttactgatgagaagtaaagtggtagaagaaaaaaaaattttctgttatcctgggcacttggaaatgaatgtattctcacaatctgttctcaaaacaacttactgattctggggttctggaagctctgatgtgcaggtgagccttttaaattcctcactgttggagctcctatctaggactcactggctggatgaaaacggttctttttattgctttctgaatgtctgctagacaggcgtaagcaacaccttatatctgccttctgaaaaaggtaaaagaactgggacccatccaccatgctggacagctcggcagtggcagtggcctcccccagaccctgttccgagtgctccaccaacaaactcaccagcagtcagagtctagcctctccccaaacttcaccttcatcacaattcattttaagcccttccacaacccaatcaactctagatctacttaatggataataatttgatctcatgcaaactgcactttcctcttctcagaatgatccttctaccccttaattaaacatttgagagtgaaagaagagaaaattcgggttcaaagattggtaagtctaagaaacctaaggaaaaggagttagtaaacatgttaatcaaagagtgagcacttttcggaagcgcaacattcagatacctttcttgattggattccagaagactatttctgggaagaggagatttgcatttttctaaagtcttctacccacagcctaaccaccctagggctttgaaatattttttttctgatgtgcagtcataattgaataaataaaatgattcctgatcatttcttctcttcagctttat

SEQ ID NO: 937 - DNA, 2020 bp, Artificial Sequence
  Synthetic Sequence
cccaccccctaccctagtctctttaaaaaaaaaaaagaaaaagaaaaaaaaaccccacagcttctgccttaggctttcttctgcagctatagctacagctctttccagattccagcatctgcttcattcccttatctcttcaggtacaggtgtgctaatcattctcccactacttttatctgtggggtgcttcaccatcccttgttgttttcccttacctctgtctacaactacgtaaatagctttcatttacgtagttcattaatcttttctcaattaccattccaccgtgccacctctttcctggtccaaccctgtttagtacaagtacattgagagaaagcatttcaagtatgggaagggatgtagaggaaaacaaaaccaaaaggcagaaagaactaagaattatgctaatggcaacaaagaattttggctaaaatagaatggaagatgcttgttaagaagcaataacaaacaatattgaacatgtagaatggaccagatcatgaaagctctggaaagccaattaaagaagcctgggacttgatttggtaggcaaaggagaattatttatcatttcttctacatataccaagtacctcctgtgtgtcaggcacatagaagttaaatcagtatcttagtaaacttagtctggcaaaaagaatgaataaaaacttgagaaaagaacactggctaagaggctattatattaatccaggcatgagatgatgagtgtctatagtattgaaaaatggaaagtaaaaaatatgagacattctaaaaaaaaatataatagactgaacaaagagaataaaggaaaaagatgagtcaaaaatgagtcaaagatgattctagagtgactaagtgaaactggaagaaaggaaaaggggaccatgaaatgcgaaaggaagaaacacaaaaatggaaaggaaatagaaagtgtgcttcactgatggaagagacaaaaagtatatttaaaattacacaaaataagagacagaaaatgtcctgagacagattctttcaaaagctaaaaaaatacaaattattaagcctgaaccataggttaactattctcaaaaaagtccttcatttacatttttttcaaattacaaaaggctgaacatgaggaattccctcaaataaagtttctatgtatcagtcatgattcaccttcacaggcagcaagatagcttcctttgaaaggagagttgcatgaattttttttttcagaggtagagtggatgggagagtgaagaacccaatgtgtgacctgttaaccctgaagttacaataagtcacaaaaggagatgatctacaggtagttgaacatatagaaccagagattaagagaaatgtgagattgaaaaactatgtatgccgtcatgcacatgaagtgaatggctgactaaagtctgagctcactgaaacaaagactttgagaaaagcaaaactctatcccaaaactctatccttacagagttttggaagaatggaggaaaaagataatcagggagtcagaggaatttattttaagagtagacgaccttggaaaagttaactatgacataaatgtcataaaccaatcattttatgttttaaacatccataaatttgtacaaaacacagtatttggtaatgaaaaacacaaagtgtcagtaacacttaggatgctttagctataaatatcagaaaaccataactaaaccacaaaataatcaggaaatttactatcctataacagggaccacaaagttaggcaggctggttgacctggatactcgatatagttcaatatagactagttattttcatgtctccactttccattcttgggcctcttccttagactattaacaagaggccaattcaggtattacatggcaattgtatcattctgggactcattaaagagaaagaaaatgtttccttgaactacctcttgcttgccagaattggatcacatgctcattcctgaaccaaacactgccatgaggaatagagtaatattaattggctt

SEQ ID NO: 938 - DNA, 4001 bp, Artificial Sequence
  Synthetic Sequence
atcagtgtctgttctttatcagccgctcagtccgtggcattttgttttagcagcaccaacagactaagacacacaccagagctgcagccccaagatgggagtgtagaactgtaaacaaagtcacaatatgtttttaaaaataaacaaaaacccaccaccactttaggagagcagcaattcccaatgagtcatccaaggaccaggaatgcaaatttgcaaagatgcacattttgggcttgtcccacacccactgatggggacgccctagggtggagcccccatggcccgtgtttgagcaagccccaggtgattctggtgcaggagaagcttcagaaccagagccctgagacagaagcaggtgggagcagagcccagtgaggccctcagtggtgatcagatcagcagcaaccccagatgacaactggagacctgacgagggccgcaggtgtagagatagaggctggactccagaggagacgcagagctcagagcaggagaaagagcagtcagttgttttcacagcagggtgggtgacgttcgttgcagaaaggatgtcacatcatcagccattcttctcgaagctccatgccttaagttttctcctcctcttcctcatcaattattttggcctccttcccattagtcatgaagcattaccttaagtttgtcctttatttcattcttttatcaggatcctctctctgggagccctcatctacctccagctgcaatcatcaccacaggagtgagagagagacttggacctgagctggaccctccagccctccttcgctgaactaccttccaaccttatgttcctactgatgtttaccaggttattcaataaatattgtatgttctgtggcagtggccaccagtcattttcaaatacctgttttcccttcttccttaccaaaaacacccatggtttttagctggacacctggttgcccaaaataaacactgcatttgcagcctcccttggagattggtgtggaattaaattaagttcaggccagagggacataagctaaagtggcaacttcccagagccttccttaaaaggaaccttgtacacaactcttgccccattttcaaacttttcctcttcttgttggccttttctccttcttgttagcaggaatgcagatgtgatagctggatctgggcaatcattttggagccatgaggtgaccatgggcatggagcccaatatagtggagaaagtagagagaaggaggccacgtcaacttcaaccctgacttattgagtttttggttcttttcagccaacctcaatataaaggatgtaagtttcagctatgagcaaggcactatgacatcataggagatgcagtgatacgcagggtaagcatggattcctcaatgaatttacatccaacaaggaaaacggacggtagacgagaccattgtgatgtgacgaatgtcaccaaggaagaagcaaagtgctgccctctgctcaatcattctgttgctgcttgtcttagtattgtcatcctgagacttgtgcatgcagtgtggggcagggtcaacatttcaaagtctaattctgcattcccacatcttgatgtcaatgagaggagatacagtgtaataaagaacaggtcaaataaaacaatacccctgcagtgagaactcagggctctggagaaatggccaacatcaggtcaagcacagggcatggaccacctaagcctagataccctgtcattcatgatggtgaggaacttaccaaagacgacaaggattaaacagaatgactcaaaagccaccatgaggaggctcccactggacagagatgagacaatttgaacatcagaagagaaaataattcaaaatattgaaacacatcaaaatgtctaactctatgagtttataatgacattaaataaaaaccaattggccatctttagaggatgatagagaactaatttactacactgagaactagttcatacagggaacagatcaagtatttacaataccaagggtatccggtggctcatcaaatagtagaaaccacctccttattaaattattctgatttataaagaaaaagaaagtgataaaattacagtatcactattttaaagctcctggccccgcaatgaattggttgatgcaggactaggaattgagcattggctgctgatgacatcatgaaaggataggcagtcaggtgttttgcagcctccaatggaagacactgggatgcctgcagccttgcaaacgtggtggagcctgagtgcagcccagcctagttttcagatgcatctgccaatctgtaaaatacacagtgggcagcaaagtggtcaagctgcatcctgagtgtgagatcgccaaaatccagatgccaagagactctgggtcaaacagccccggtgcttcaataaataactgataaagaaaacaaagtgagggaggggaagctgtggattgtaagacacttgaaactatctcaaaactttaaataaaaaagagggggggaggcaagtctaagcaatagttcacgtttagacgtgtggttcaaggttcacgtttagacgacatcattatttttaaaacacacaaagtgattattataagtcagcctaatggccactaatgggagaaggaaggagctggactaggataagcgggacccagcaactggatgggcttctcagacgtgtggccaaattccacttctggacgtgaggctcctttccctacaatagttcattagaccgcacattcattttgtgtgtttttctgcatctttgttttatgttacaataaaatgatttttagaaactatggcagggtcttggtaacctatgcaaaattctcagattccttgtacatgtcccccagaaacatgcaacctgcctaactgcgacctttcagccctgcattcctttccacagctgtgctctgacaatattccataggtgagctccacacccaggtggggcactggctctcttcctcctgaagtcaggactacagcagtgctcggtcttgaacaaaaaggtttttgtttgtgttacgagtcagcgtagtcaatccgttcccttttatgcctggaaacgttcccattttcccaggggaagaagaactcaggctacagctgtggaacgtgtgtttcttccaaccagtgaagaccggacgccattccttcatgctactgggttcttgcagctgtaatttacaacacagctgtcatatctgtgacatctgggccagctcctctgtgcccaacctcagccctgttcagtttgcccctgtacccccaattctccccagtagccatccctgaactgcactttctaactgcgggattgcccttgagttgggctgctggatcctccctcacccttgcctgacgcaggaagacagtcatcctcacagaagacgaagttcagttccacagctcggcctcagatcacaagggcgtgaggattaaagagaggataccacagtcggtaagaggcagatactagacatttacctttttaataaaagagtaacaaattatttggggtacggttgggtgacaattcaagtgaaagcttgcagaaaaccaaaaaattgaaaagtgacaaactggcacgtagctgtgacggcgagagcgctctggtttaagtcattgttgtgaactgcccgtgaccactgcgcctgacttctaggcccagtcataattacgcgtggcagtaaggtcagacctaactcattgctaatgtgattacactggtaaagtctgtgttcattgcaccaaggacagagccgtcggctcaagaagcatctgtccctgcaaaccacttttagaaaaatagaaggacccgcgccggacccgcgcctgccctgcgtctaggagccaaggcaattttctaaatgtggcaagtggagaaattgagggacggagaggcttagatggcttcccagaggtcatgtaagtctttgccttacaaatagagcattcacttgtaggtgtagagcaca

SEQ ID NO: 939 - DNA, 4001 bp, Artificial Sequence
  Synthetic Sequence
tggagtccagctcatccaagacccaccataagactccagctttaaattttctaagccatccaggattacattctggaatttgaggtttcatttccattatatattatagcttctgataaaatttctaaaacactttttcaactttttatgagattcttatcaaattattacttgtcatcttgctctgaattcaactgccctgttctcttttgtggctgctcattaaattatctaaccagttttcttagttgtttacctttataaccctttgaagggtcttgtatgaggagcaggtagctgaattttgtgttttaaaacattacatctacaatattataaatggtcaatattgtaaatgtaatgaccaatggtttggtcatatcttaaccatttaatcccatgctttctgttgtgacccctttttgatggttttcttagtttctctaagctctgacaccaccctccaaaaatgctaatttagtataacttactttctccacccaccaggtcacaacaagcaaatacacacataaacactctatatcaatattagtcctccaggaagcagacaccaagatagaactagaagtccaagagattgattggatgaaagacaaaggggaaatgaagcagtgaaatgtattttaatacaggtctgatgcctggaaaaggaaagagggaagaaaggcctgggcaggaagggcttctgactgcaatgcagctttgaaaatgtctcagccagcccatggggaaaccatagaacaaagagaactgaattgggcagaaaaggcccgactctcatcctctgctgtgctcaaccatcagctgggagtagcctagggagagcatggcttcatgtaaatgctgcaatagatcccaaagacatgcatgcatacacgtgacacttccaaattttgacctgatttatttattttgcattttaaaaatattcaggctgggtatgatggctcatgctataatctcagtgcttcaggaggctgaggtgggagattcacttgaggctgggagttcaaaaccagcctgggcaatatggtgagaccccatctctacaaaaaatagaaaaatgagctgggcatggtagtatgcacctgcaagaggcggaggcaggaggatcactgaagcccaggaattcaaggctgcagtgagttatgaagacaccactgcactctggcctgggcaacaggacaagatcctgtatctaataaaataaaataattcagtttggctgttgcatcttttcacacatacatacatacatgcatatataaggaatttactgagtttctaccctcatctgtattacatgaaagaagtacctggttctaattcctatgtccagccttgatactatgtttctctatcaacttcacttacctctaatattgctagactgaatctaagcagttttatcaaagagttcatatcctctatatttccatatttcagaaaatctttctgttgcttttgctcgtgagtggctatatggcatgttatgaaatacttgaatattgtctattaccaccccaaagtctccaagaattccaggattatctcgtagtgtaaagtattactaaggagaaagggcttgcttttgttcctttgtacataatctaagttttctgcttgaacattaagagaatctttaagaatctttgaaatacaaacattttgtcagtgtacatttatatatgagccctcctcttttagtgtgttttcatatgaatcttacttggatgtttttaatagtctcattatttatagttttgagcttctcttttaaaagtggagattattttatatttccaataatcgaaaaatcacttctgtatccattgtttggttccattgcctagatacaacagctttagcactattgctttttttctgaaagttcactggaaataatctaatggcttccagtgtcagtgttcataggttaaaaaatcccaagagctacttttctagccatactgctaaatcctacttttaaagaatcatcctaaaatttgtcaccccattcctttacttctattccccaccagtctctagcactcatctgggaagcagaaagaggtctcttaatcatctgtattttattaagtgtctaatatactataggagaagccaattctctgtttttccaacatttcaactgagtatcagaatttgcggccaaatgaattgcagcaaaccattttctaatagaagaaaaaaatgcctcaggagttctataatgtcccaatcagagcaaacaaaccttgggagtttagggtacttatccaaacaactctcccaccataaactaagtgcaactcaatttatctcaaaaaagacgaaagggtgagtcaacgtggctgtaatttgaactttacagttctagggagtctccccatttccaacctgagacttagaccactaagctatccccccaggagatttatttgcttctttaatttccttggttaaaaatatctgagtcaccataaaccaaatactgcaggaatcttaacttttaaaagagcattaaaaccacactgattgatttggcgagatgttggaaactgttttgttctctgacaattatgacggactcaacttggctcttggaataccaatgaaggagaatacaaatgcaatatcatcttccacatctgagcctgagaaagctcactgttcctcttcattatttcttaaatggacctttccagctgctcctaaatatgtacacatgtcaagcatttgagaaaagtgaccaaggttttacctacagtatttcttatgagagtattgaggacagcattcatttattcacttattcatcaaaaacgtattaagtgcctactatgtttattatttccagtgcacatgatacaaagatgaacaagacctaggcccttgaggacaatcaaaatatattattgtcataataatgacaaagaaggggaagaagagaagcagaagggaaatattatttcgccattttacagatgttgaaacacaggcagagagagataaaggaaagtgccttagctcacaacatttgtaatcagcaggacccaaattctaaggcttcagttggatgagtgcacaatcacctcatgtactattgtattattccatcttcatactgctatgaagaaatacctgagactgggtaatttataaaggaaaaaaggtttaatggactcagttccacatggctggggaggcctcacaatcatggtggaaggtgaaggaggaacaaaggcacatcttacatggtggcagatgtgagagcgtgtgcaggggaaccacgctttataaaaccatcagctcttgtgaggcttattcactataatgagaacagtatgagaaaaacctgcccccatgattcaattacctccaactgggtccctcccatgacatgtggggattatgggagctacagtttaagatagatttgggtggggacacagccaaaccatatcacctattgtctcaatagaagactgattaagtacatttaaaaggaactcaatacaaccacgcttgtctcttgattcagctggagtcacagtcgccaccttgatccaagagttctatcctgtagacttgagcttttccctttttaagtctctctctgttacttttgatggtttcaagagtgtttgacatgtgctcagacacaattttttcttgttcctcagaggctgacataacatctgtttatgacatggctgaaataaggagggtgtggagtcagatacattaagtgtgtactttggtgaagaaaatgatatgatcataatagctgtgagaaactttattcattcagcacacatgaaacacattctagcatgtgaaaagcatgctactgagcataaatcaaatgccattctgactcctgagaaacacacagcctagggagaaagaggcaccttaataccaatattatagtacaaactcaatgcactaagatgatcattcattctcgatcactt

(19) United States
(12) Patent Application Publication
(10) Pub. No.: US 2024/0002818 A1
(43) Pub. Date: Jan. 4, 2024

(54) MAMMALIAN MOBILE ELEMENT COMPOSITIONS, SYSTEMS AND THERAPEUTIC APPLICATIONS

(71) Applicant: Saliogen Therapeutics, Inc., Lexington, MA (US)

(72) Inventors: Joseph J. HIGGINS, Lexington, MA (US); Ray TABIBIAZAR, Lexington, MA (US)

(21) Appl. No.: 18/037,937

(22) PCT Filed: Nov. 24, 2021
(86) PCT No.: PCT/US21/60783

§ 371 Date: May 19, 2023

Related U.S. Application Data
(60) Provisional application No. 63/117,733, filed on Nov. 24, 2020.

(51) Int. Cl. C12N 9/12; C12N 15/63; C12N 9/22

(52) U.S. Cl. CPC C12N 9/1241; C12N 15/63; C12N 9/22; C12N 2310/20

(57) ABSTRACT

Recombinant mammalian helper enzymes for targeted transposition are described. The mammalian helper enzymes and corresponding donor DNAs can be used, e.g., for gene therapy.

### CROSS-REFERENCE TO RELATED APPLICATIONS

This application claims the benefit of and priority to U.S. Provisional Patent Application No. 63/117,733, filed Nov. 24, 2020, the contents of which are hereby incorporated by reference in their entirety.

### FIELD

The present disclosure relates to recombinant mammalian mobile element systems and uses thereof.

### DESCRIPTION OF THE TEXT FILE SUBMITTED ELECTRONICALLY

This application contains a Sequence Listing in ASCII format submitted electronically herewith via EFS-Web. Said ASCII copy, created on Nov. 23, 2021, is named SAL-004PC_SequenceListing_ST25.txt and is 446,464 bytes in size. The Sequence Listing is incorporated herein by reference in its entirety.

### BACKGROUND

Mobile elements are genetic sequences that are found, with small exceptions, in all living organisms. Mammalian, including human, genomes include DNA sequences that are mobile, transposable elements that are theoretically able to move from one location to another within the genome. Mobile elements have deep evolutionary origins and diversification and have an astonishing variety of forms and shapes. See Bourque et al., Genome Biol 19, 199 (2018).

A mobile element movement to a new location in the human genome is performed by the action of a helper enzyme that binds to an “end sequence” and inserts a donor DNA sequence at a specific DNA sequence such as the tetranucleotide, TTAA, by a “cut and paste” mechanism. No active DNA transposases have been identified in mammals, except in bats. Most mammalian genomes include only a handful of decayed transposable elements. In mammals, mobile elements are thought to have ceased their activity over 35 to 40 million years ago (See Pace et al., Genome Res 2007, 17: 422-432. 10.1101/gr.5826307; Pagan et al., Genome Biol Evol 2010; 2:293-303). The exception is the little brown bat, Myotis lucifugus, which contains thousands of active elements. Ray et al., Genome Res 2008; 18:717-28.

DNA donors, which are mobile elements that use a “cut-and-paste” mechanism, include donor DNA that is flanked by two large (greater than 150 base pair) end sequences in the case of mammals (e.g., Myotis lucifugus) and humans, or Inverted terminal inverted repeats (ITRs) in other living organisms such as insects (e.g., Trichnoplusia ni) or amphibians (Xenopus species). Genomic DNA is excised by double strand cleavage at the host's donor site and the donor DNA is integrated at this site.

The piggyBac transposon, from the looper moth, Trichnoplusa ni, is a bioengineered movable genetic element that transposes between vectors and human chromosomes through a “cut-and-paste” mechanism. Zhao et al., Translational lung cancer research vol. 5, 1 (2016): 120-5. doi:10.3978/j.issn.2218-6751.2016.01.05. During transposition, a helper enzyme (e.g., piggyBac) recognizes small (13 bp and 19 bp) ITR sequences located on both ends of the donor DNA vector, and then integrates the donor DNA into TTAA chromosomal sites.

In general, usage of mobile elements, including piggyBac, in mammals has long been limited due to the lack of an efficient transposition system and risk of mutagenesis. See Kim et al., Mol Cell Biochem 2011; 354:301-9. Mobile elements with protein domains similar to piggyBac have been identified in fungi, protozoa, plants, insects, crustaceans, echinoderms, urochordates, hemichordates, fish, amphibia, and mammals (e.g., bats). See Sarkar et al., Mol Genet Genomics 2003, 270: 173-180. Some human mobile elements, such as, e.g., the Cockayne syndrome Group B (CSB)-piggyBac transposable element derived (PGBD) domain 3 fusion protein (CSB-PGBD3), retain site-specific DNA binding but gain new functions by fusion with upstream coding exons. See Newman et al., PLoS Genet 2008; 4:e1000031. PLoS Genet 4(3): e1000031.; Bailey et al., DNA Repair (Amst) 2012; 11:488-501; Gray et al., PLoS Genet 8(9): e1002972.

There is a need for novel mobile elements (donors) and/or helper enzymes (e.g., transposases) that are suitable for use in humans and that efficiently target human genome with reduced risk of off-target effects.

### SUMMARY

Accordingly, the present disclosure provides, in aspects and embodiments, compositions comprising recombinant mammalian helper enzymes and/or ends that are suitable for recognition by such enzymes. In aspects such enzymes (or helpers) are bioengineered for use in humans, e.g., having increased integration efficiency (hyperactivity), enhanced or increased gene cleavage activity (e.g., being excision positive (Exc+)) and/or diminished or reduced integration activity (e.g., integration deficient (Int−)) and/or enhanced or increased integration activity (integration efficient (Int+)). Without wishing to be bound by theory, the present disclosure, inter alia, is based on the discovery of helper enzymes and related end sequences that have been evolutionarily silenced in humans and other mammals, and an engineering approach to reconstruct or revive their biological activity, e.g., for use in therapies.

In aspects, there is provided a composition comprising (a) a recombinant helper enzyme, or a nucleotide sequence encoding the same, having gene cleavage (Exc) and/or gene integration (Int) activity and at least about 90% (e.g. at least about 95%, or at least about 96%, at least about 97%, at least about 98%, at least about 99%) identity to the amino acid sequence of SEQ ID NO: 2, and/or (b) a gene transfer construct comprises a vector comprising a donor DNA comprising left and right end sequences recognized by the recombinant helper enzyme, the left and right end sequences having at least about 90% (e.g. at least about 95%, or at least about 96%, at least about 97%, at least about 98%, at least about 99%) identity to the nucleotide sequences of SEQ ID NO: 11 and SEQ ID NO: 16.

In embodiments, the recombinant helper enzyme has the nucleotide sequence having at least about 90% (e.g., at least about 95%, or at least about 96%, at least about 97%, at least about 98%, at least about 99%) identity to SEQ ID NO: 1 or a codon-optimized form thereof.

In embodiments, there is provided a system for genomic alteration comprising a helper enzyme, having gene cleavage (Exc) and/or gene integration (Int) activity, and at least about 90% (e.g. at least about 95%, or at least about 96%, at least about 97%, at least about 98%, at least about 99%) identity to an amino acid sequence of SEQ ID NO: 2, SEQ ID NO: 3, SEQ ID NO: 4, SEQ ID NO: 6, SEQ ID NO: 7, SEQ ID NO: 8, SEQ ID NO: 9, or SEQ ID NO: 10, or a nucleotide sequence encoding the same, and a gene transfer construct comprises a vector comprising a donor DNA comprising left and right end sequences recognized by the recombinant helper enzyme, the left and right end sequences having at least about 90% (e.g. at least about 95%, or at least about 96%, at least about 97%, at least about 98%, at least about 99%) identity to one or more (e.g. two) nucleotide sequences of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

In embodiments, the helper enzyme has one or more mutations which confer hyperactivity. In embodiments, the helper enzyme has an amino acid sequence having mutations at positions which correspond to at least one of S8P, C13R, and N125K mutations relative to the amino acid sequence of SEQ ID NO: 10 (Myotis lucifugus) or a functional equivalent thereof.

In embodiments, the helper enzyme has an amino acid sequence having mutations in at least one of positions 8, 17, and 134, relative to the amino acid sequence of SEQ ID NO: 2 or a functional equivalent thereof.

In embodiments, the helper enzyme is included in the gene transfer construct. In embodiments, the composition comprises a nucleic acid binding component of a gene-editing system. In embodiments, the gene-editing system is included in the gene transfer construct.

The gene-editing system targets the helper enzyme to a locus of interest. In embodiments, the nucleic acid binding component of the gene-editing system can be, for example, a DNA binding domain (DBD), such as a transcription activator-like effector protein (TALE). In embodiments, the gene-editing system comprises Cas9, or a variant thereof. In embodiments, the gene-editing system comprises a nuclease-deficient dCas9. In embodiments, the gene-editing system comprises Cas12, or a variant thereof. For example, the gene-editing system comprises a nuclease-deficient dCas12. In embodiments, the gene-editing system comprises Cas12j, such as, for example, nuclease-deficient dCas12j.

In embodiments, the helper enzyme is capable of inserting a donor DNA at a TA dinucleotide site or a TTAA tetranucleotide site in a genomic safe harbor site (GSHS) of a nucleic acid molecule.

In embodiments, a helper construct comprises an RNA or DNA fused or linked to a DNA binding domain (DBD), such as a transcription activator-like effector protein (TALE), zing finger (ZnF), or inactive Cas protein (dCas9) programmed by a guide RNA (gRNA), or a dimer enhanced construct as shown in FIGS. 13A-E. Another Cas protein such as, e.g., inactive dCas12a or dCas12j can be used in the helper construct shown in FIGS. 13A-E or in a similar helper construct. In embodiments, a donor DNA construct comprises DNA with recognition sites called ends or ITRs (both herein called “donor”) fused or linked via to insulators, promoters, genes of interest, or miRNA (sense, loop, antisense) as shown in FIGS. 14A-E.

In aspects, a nucleic acid encoding a recombinant mammalian helper enzyme or various ends in accordance with embodiments of the present disclosure is provided. In embodiments, the nucleic acid is DNA or RNA. In embodiments, the nucleic acid is RNA that has a 5′-m7G cap (cap 0, cap1, or cap2) with pseudouride substitution (e.g., without limitation n-methyl-pseudouridine), and a poly-A tail of or about 30, or about 50, or about 100, of about 150 nucleotides in length.

In aspects, a host cell comprising the nucleic acid in accordance with embodiments of the present disclosure is provided.

In aspects, a method for inserting a gene into the genome of a cell is provided that comprises contacting a cell with a recombinant mammalian helper enzyme and/or end sequences in accordance with embodiments of the present disclosure. The method can be in vivo or ex vivo method. In embodiments, the cell is contacted with a nucleic acid encoding the helper enzyme. In embodiments, the nucleic acid further comprises a donor DNA having a gene. In embodiments, the cell is contacted with a construct comprising a donor DNA having a gene and/or end sequences in accordance with embodiments of the present disclosure. In embodiments, the cell is contacted with an RNA encoding the helper enzyme. In embodiments, the cell is contacted with a DNA encoding the donor DNA. In embodiments, the donor DNA is flanked by one or more end sequences, such as left and right end sequences. In embodiments, the donor DNA can be under control of a tissue-specific promoter. In embodiments, the donor DNA is a gene encoding a complete polypeptide. In embodiments, the donor DNA is a gene which is defective or substantially absent in a disease state. In embodiments, the method is used to treat an inherited or acquired disease in a patient in need thereof.

In embodiments, the present method, which makes use of a recombinant mammalian helpers (inclusive of chimeric helpers, described herein) and/or ends, provides reduced insertional mutagenesis or oncogenesis as compared to a method with a non-chimeric helper or as compared to non-mammalian helper enzyme. Because the recombinant helper enzyme is from a mammalian genome, the mammalian helper enzyme is safer and more efficient than transposases from, e.g., plants and insects.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF DRAWINGS

FIG. 1 depicts an amino acid alignment and reconstruction of mammalian helper enzymes including human helper enzymes (PGBD1, PGBD2, PGBD3, PGBD4, and PGBD5), based on homology with Pteropus vampyrus nuclease. Red (bolded and underlined S, G, and K amino acids) indicates regions that were mutated in Myotis lucifugus (S8P, C13R, and N125K) that caused increased (hyperactive) transposition in HEK293 cells. Magenta (bolded and underlined D amino acids, starting in the rows that start at position 207 of Pteropus vampyrus) indicates the essential acidic amino acids of the RNaseH DD E/D motif at the active site, and green (bolded and underlined C amino acids, starting in the rows that start at position 538 of Pteropus vampyrus) indicates the Zn finger motifs. Twenty-six amino acids were added to the C-terminus of Pteropus vampyrus based on a single nucleotide base pair substitution of the published stop codon G1933T (SEQ ID NO: 1).

FIG. 2 depicts an amino acid alignment and reconstruction of mammalian helper enzymes including human helper enzyme (PGBD4), Pan troglodytes, and Pteropus vampyrus and Myotis lucifugus. Red (bolded and underlined amino acids in the rows starting at position 1 for all four sequences, and in the rows starting at positions 68, 68, 68, and 65 for PGBD4Hu, Pan Troglodytes, Pteropus vampyrus, and Myotis lucifugus, respectively) indicates regions that were mutated in Myotis lucifugus (S8P, C13R, and N125K) that caused increased (hyperactive) transposition in HEK293 cells. Magenta (bolded and underlined D amino acids, starting at the rows that start at positions 206, 206, 206, 197 for PGBD4Hu, Pan Troglodytes, Pteropus vampyrus, and Myotis lucifugus, respectively) indicates the essential acidic amino acids of the RNaseH DD E/D motif at the active site, and green (bolded and underlined C amino acids in the rows starting at positions 538, 538, 538, 531 for PGBD4Hu, Pan Troglodytes, Pteropus vampyrus, and Myotis lucifugus, respectively) indicates the Zn finger motifs. Twenty-six amino acids were added to the C-terminus of Pteropus vampyrus based on a single nucleotide base pair substitution of the stop codon G1933T (SEQ ID NO: 1).

FIG. 3A depicts an extended edited nucleotide sequence of Pteropus vampyrus helper enzyme.

FIG. 3B depicts an extended edited amino acid sequence of Pteropus vampyrus helper enzyme.

FIG. 4A depicts an amino acid sequence of human (PGBD4) helper enzyme.

FIG. 4B depicts a hyperactive mutant form of an amino acid sequence of human (PGBD4) helper enzyme.

FIG. 4C depicts a hyperactive mutant form of a nucleotide sequence of human (PGBD4) helper enzyme.

FIG. 5 depicts the amino acid sequence of human (PGBD1) helper enzyme.

FIG. 6 depicts the amino acid sequence of human (PGBD2) helper enzyme.

FIG. 7 depicts the amino acid sequence of human (PGBD3) helper enzyme.

FIG. 8 depicts the amino acid sequence of human (PGBD5) helper enzyme.

FIG. 9 depicts hyperactive mutant forms of an amino acid sequence of Myotis lucifugus helper enzyme.

FIG. 10A depicts a left end nucleotide sequence from Pteropus vampyrus.

FIG. 10B depicts a left end nucleotide sequence from PGBD4.

FIG. 10C depicts a left end nucleotide sequence from MER75.

FIG. 10D depicts a left end nucleotide sequence from MER75B.

FIG. 10E depicts a left end nucleotide sequence from MER75A.

FIG. 11A depicts a right end nucleotide sequence from Pteropus vampyrus.

FIG. 11B depicts a right end nucleotide sequence from PGBD4.

FIG. 11C depicts a right end nucleotide sequence from MER75.

FIG. 11D depicts a right end nucleotide sequence from MER75B.

FIG. 11E depicts a right end nucleotide sequence from MER75A.

FIG. 12A depicts an alignment used to identify right end sequences of a donor DNA. Sequence logo has 50% CG base composition, consensus threshold is greater than 50%. Bases that do not match the consensus sequence are shown in boxes.

FIG. 12B depicts an alignment used to identify left end sequences of a donor DNA. Sequence logo has 50% CG base composition, consensus threshold is greater than 50%. Bases that do not match the consensus sequence are shown in boxes.

FIGS. 13A-E depict representations of RNA or DNA helper enzymes that are designed to target human GSHS or endogeneous genes using TALE, ZnF, Cas9/guide RNA DNA binders, and enhanced dimerization. FIG. 13A. included the core construct with flanking UTRs and polyA tail. FIG. 13B include TALE(s) nuclear localization signals (NLS) and an activation domain (AD) to function as transcriptional activators. The DNA binding domain has approximately 16.5 repeats of 33-34 amino acids with a residual variable di-residue (RVD) at position 12-13. RVDs have specificity for one or several nucleotides. FIG. 13C includes ZnF as the DNA binder linked to the helper enzyme. FIG. 13D includes dCas as the DNA binder linked to the helper enzyme. FIG. 13E includes a N-terminus dimerization domain (e.g., SH3, rapamycin complex) to enhance monomer interaction at the target site. The chimeric helper enzymes form dimers or tetramers at open chromatin to insert donor DNA at TTAA recognition sites near DNA binding regions targeted by TALEs, ZnF, or dCas9/gRNA. Binding of the TALE, ZnF or Cas9/gRNA to GSHS physically sequesters the helper enzyme as a monomer or dimer to the same location and promotes transposition to the nearby TTAA sequences (See underlined and bolded TTAA regions in FIG. 16B, FIG. 17B, FIG. 18B, FIG. 19B, FIG. 20B, FIG. 21B, FIG. 22B, FIG. 23B, or FIG. 24B near repeat variable di-residues (RVD) nucleotide sequences.

FIGS. 14A-E depict representations of DNA donor comprising DNA with recognition sites called ends or ITRs fused or linked to insulators, promoters, genes of interest, or miRNA (sense, loop, antisense). The inverted terminal repeat (ITR) recognition sequences are included at the 5′- and 3′-ends and are illustrated in each figure. FIG. 14A depicts an illustrative core donor construct that is contained in a replication backbone (e.g., plasmid or miniplasmid) with a promoter driving a gene of interest (GOI) with a polyA tail flanked by two insulators and ITRs. FIG. 14B depicts an illustrative core donor construct that is contained in a replication backbone (e.g., plasmid or miniplasmid) with a splice acceptor site for exon 2 and other exons of a gene of interest (GOI) followed by a polyA tail and flanked by ITRs. FIG. 14C depicts an illustrative core donor construct that is contained in a replication backbone (e.g., plasmid or miniplasmid) with tandem promoters to affect expression in different tissues (e.g., without limitation, liver specific promoter, cardiac specific promoter) and a gene (s) of interest (GOI) followed by a polyA tail and flanked by ITRs. FIG. 14D depicts an illustrative core donor construct that is contained in a replication backbone (e.g., plasmid or miniplasmid) with two or more genes of interest (GOI) linked by P2A “self-cleaving” peptides and followed by WPRE and a polyA tail. The construct is flanked by ITRs. FIG. 14E depicts an illustrative core donor construct that is contained in a replication backbone (e.g., plasmid or miniplasmid) with a promoter(s) driving the expression of two or more genes as in FIG. 14D and linked to a sequence consisting of a 5′-miRNA, a sense and antisense miRNA pair, and completed with the 3′-miRNA. The construct is followed by WPRE and flanked by ITRs.

FIGS. 15A and 15B depict DNA binding codes for human genomic safe harbor sites in areas of open chromatin. Genomic location for chromosomes 2, 4, 6, and 11 is adapted from Pellenz et al. (Hum Gene Ther 2019; 30:814-28) and chromosomes 10 and 17 from Papapetrou et al. (Nat Biotechnol 2011; 29:73-8). Sequences are downloaded from the UCSC Genome browser using hg18 or hg19 and evaluated with E-TALEN, a software tool to design and evaluate TALE DBD and WU-CRISPR, a software tool to design guide RNAs.

FIG. 16A depicts CCR5 (chr3:46409633-46419697) TALE.

FIG. 16B depicts CCR5 gene (chr3:46409633-46419697). Underlined and bolded nucleotides are donor DNA insertion sites and TALE binding sites.

FIG. 17A depicts AAVS1 (chr19:55623241-55631351) TALE.

FIG. 17B depicts AAVS1 gene (chr19:55623241-55631351). Underlined and bolded nucleotides are donor DNA insertion sites and TALE binding sites.

FIG. 18A depicts HROSA26 (chr3:9412043-9417082) TALE.

FIG. 18B depicts HROSA26 gene (chr3:9412043-9417082). Underlined and bolded nucleotides are donor DNA insertion sites and TALE binding sites.

FIG. 19A depicts Chr2 (chr2:77262930-77264949) TALE.

FIG. 19B depicts Chr2 gene (chr2:77262930-77264949). Underlined and bolded nucleotides are donor DNA insertion sites and TALE binding sites.

FIG. 20A depicts Chr4 (chr4:37768238-37770257) TALE.

FIG. 20B depicts Chr4 gene (chr4:37768238-37770257). Underlined and bolded nucleotides are donor DNA insertion sites and TALE binding sites.

FIG. 21A depicts Chr6 (chr6:134384946-134386965) TALE.

FIG. 21B depicts Chr6 gene (chr6:134384946-134386965). Underlined and bolded nucleotides are donor DNA insertion sites and TALE binding sites.

FIG. 22A depicts Chr11 (chr11:32679546-32681565) TALE.

FIG. 22B depicts Chr11 gene (chr11:32679546-32681565). Underlined and bolded nucleotides are donor DNA insertion sites and TALE binding sites.

FIG. 23A depicts Chr10 (chr10:3044320-3048320) TALE.

FIG. 23B depicts Chr10 gene (chr10:3044320-3048320). Underlined and bolded nucleotides are donor DNA insertion sites and TALE binding sites.

FIG. 24A depicts Chr17 (chr17:67326980-67330980) TALE.

FIG. 24B depicts Chr17 gene (chr17:67326980-67330980). Underlined and bolded nucleotides are donor DNA insertion sites and TALE binding sites.

### DETAILED DESCRIPTION

The present disclosure is based, in part, on the discovery of new recombinant mammalian helper enzymes and/or associated ends.

Humans have 5 inactive elements, designated PiggyBac domain (PGBD)1, PGBD2, PGBD3, PGBD4, and PGBD5. PGBD1, PGBD2, and PGBD3 have multiple coding exons, but in each case the mobile element-related sequence is encoded by a single uninterrupted 3′ terminal exon. Thus, PGBD1 and PGBD2 may resemble the PGBD3 helper RNA in which the helper enzyme ORF is flanked upstream by a 3′ splice site and downstream by a polyadenylation site. See Newman et al., PLoS Genet 2008; 4:e1000031. PLoS Genet 4(3): e1000031.; Gray et al., PLoS Genet 8(9): e1002972.

The PGBD5 inactive helper enzyme sequence belongs to the RNase H clan of Pfam structures, while PGBD3 has sustained only a single D to N mutation in the essential catalytic triad DDD(D) and retains the ability to bind the upstream piggyBac terminal inverted repeat. Bailey et al., DNA Repair (Amst) 2012; 11:488-501. The PGBD5 helper enzyme does not retain the catalytic DDD (D) motif found in active elements, and the helper enzyme is not only inactive but fails to associate with either DNA or chromatin in vivo. Pavelitz et al., Mob DNA 2013; 4:23. However, in vitro studies showed that it is transpositionally active in HEK293 cells. See Henssen et al., Elife 2015; 4. PGBD1 and PGBD2 are thought to be present in the common ancestor of mammals, while PGBD3 and PGBD4 are restricted to primates. See Sarkar et al., Mol Genet Genomics 2003; 270:173-80. The Pteropus vampyrus helper enzyme is related to PGBD4 and shares DDD catalytic domain and the C-terminal region that are involved in excision mechanisms. See Mitra et al., EMBO J 2008; 27: 1097-109.

In the present disclosure, the amino acid sequence of Pteropus vampyrus helper enzyme was aligned to PGBD1, PGBD2, PGBD3, PGBD4 (also referred to as PGBD4hu herein), and PGBD5 sequences to identify helper enzyme sequences that were used to construct a mammalian helper enzyme in accordance with embodiments, which has gene cleavage and/or gene integration activity. Also, mutations were identified that confer hyperactivity to a recombinant mammalian helper enzyme. The constructed recombinant helper enzymes are novel mammalian helper enzymes, which can have advantages over existing plant- or insect-derived helper enzymes. The recombinant mammalian helper enzymes are more efficient and safe, with reduced risk of insertional mutagenesis.

Helper Enzymes

In aspects, a composition comprising (a) a recombinant helper enzyme, or a nucleotide sequence encoding the same, having gene cleavage (Exc) and/or gene integration (Int) activity and at least about 90% identity to the amino acid sequence of SEQ ID NO: 2, and/or (b) a gene transfer construct comprises a vector comprising a donor DNA comprising left and right end sequences recognized by the recombinant helper enzyme, the left and right end sequences having at least about 90% identity to the nucleotide sequences of SEQ ID NO: 11 and SEQ ID NO: 16.

| SEQ ID NO: 2: Extended Pteropus vampyrus Amino Acid | |
|---|---|
| Sequence (584 Amino Acids). | |
| MSNPRKRSIP TCDVNFVLEQ LLAEDSFDES DFSEIDDSDD FSDSASEDYT VRPPSDSESD | 60 |
| GNSPTSADSG RALKWSTRVM IPRQRYDFTG TPGRKVDVSD TTDPLQYFEL FFTEELVSKI | 120 |
| TSEMNAQAAL LASKPPGPKG FSRMDKWKDT DNDELKVFFA VMLLQGIVQK PELEMFWSTR | 180 |
| PLLDIPYLRQ IMTGERFLLL LRCLHFVNNS SISAGQSKAQ ISLQKIKPVF DFLVNKFSTV | 240 |
| YTPNRNIAVD ESLMLFKGRL AMKQYIPTKC ARFGLKLYVL CESQSGYVWN ALVHTGPSMN | 300 |
| LKDSADGLKS SCIVLTLVND LLGQGYCVFL NNFYTSPMLF RELHQNRTDA VGTARLNRKQ | 360 |
| MPNDLKKRIA KGTTVARFCG ELMALKWCDK KEVTMLSTFH NDTVIEVDNR NGKKTKKPCV | 420 |
| IVDYNENMGA VDSADQMLTS YPTERKRHKF WYKKFFRHLL NITVINSYIL FKKDNPEHTI | 480 |
| SHVNFRLTLI ERMLEKHHKP GQQRLRGRPC SDDVTPLRLS GRHFPKSIPP TSGKQNPTGR | 540 |
| CKVCCSHDKD GKKIRRETLY FCAECDVPLC VVPCFEIYHT KKNY | |

In embodiments, the helper enzyme has at least about 95%, or at least about 96%, at least about 97%, at least about 98%, at least about 99% identity to the amino acid sequence SEQ ID NO: 2.

In embodiments, the helper enzyme does not comprise a truncation at the C terminal end of 26 amino acids. In embodiments, the helper enzyme has at least about 95%, or at least about 96%, at least about 97%, at least about 98%, at least about 99% identity to the amino acid sequence SEQ ID NO: 2, wherein the helper has at least about 560 amino acids, or at least about 565 amino acids, or at least about 570 amino acids, or at least about 575 amino acids, or at least about 580 amino acids.

In embodiments, the helper enzyme has one or more mutations which confer hyperactivity.

In embodiments, the helper enzyme has an amino acid sequence having mutations in at least one of positions 8, 17, and 134, relative to the amino acid sequence of SEQ ID NO: 2 or a functional equivalent thereof.

In embodiments, the helper enzyme has an amino acid sequence having mutations at positions which correspond to at least one of S8P and G17R mutations relative to the amino acid sequence of SEQ ID NO: 2 or a functional equivalent thereof.

In embodiments, the helper enzyme has the nucleotide sequence having at least about 90% identity to SEQ ID NO: 1 or a codon-optimized form thereof.

| SEQ ID NO: 1: Extended Pteropus vampyrus Nucleotide Sequence* (2210 bp). | |
|---|---|
| CCCATTTCCT GTTTGCCCCG AGAATACTCA CCAGCGGCAC TTGCAGCTGC AGCGTTTACC | 60 |
| CCGAGATAAC TCGYCGATTA CAGTCCTAAC CTTACCCCCA AAGTTTGCCA TGAAATATCT | 120 |
| CGCTTTTATT ATTATTTTCG CATCGCTCTA GTATATCGAT AGTCTTTGGA AACAAATGAC | 180 |
| ATCATTNTAT TTACAGCATT CTGTTTTTAN TAGTGGTATT TCCATTTACA AAATATAGTA | 240 |
| ATTTTCTATC GCTGAAAATG TCAAATCCTA GAAAACGTAG CATTCCTACA TGTGATGTTA | 300 |
| ACTTCGTTCT CGAACAGTTG TTAGCCGAAG ATTCATTTGA TGAATCCGAT TTTTCCGAAA | 360 |
| TAGACGATTC TGATGATTTT TCGGATAGTG CTTCGGAAGA CTATACGGTC AGGCCTCCGT | 420 |
| CCGATTCGGA ATCTGATGGA AATAGCCCTA CATCAGCTGA CTCGGGTCGC GCTCTGAAAT | 480 |
| GGTCAACTCG TGTTATGATT CCACGTCAAA GGTATGACTT TACCGGCACA CCTGGCAGAA | 540 |
| AAGTTGATGT CAGTGATACC ACTGACCCAC TGCAGTATTT TGAACTGTTC TTTACTGAGG | 600 |
| AATTAGTTTC AAAAATTACC AGTGAAATGA ATGCCCAAGC TGCCTTGTTG GCTTCAAAGC | 660 |
| CACCTGGTCC GAAAGGATTT TCGCGAATGG ATAAATGGAA AGACACTGAC AATGATGAAC | 720 |
| TGAAAGTCTT TTTTGCAGTA ATGTTACTGC AAGGTATTGT GCAGAAACCT GAGCTGGAGA | 780 |
| TGTTTTGGTC GACAAGGCCT CTTTTGGATA TACCTTATCT CAGGCAAATT ATGACTGGTG | 840 |
| AAAGATTTTT ACTTTTGCTT CGGTGCCTGC ATTTTGTCAA CAATTCTTCC ATATCCGCTG | 900 |
| GTCAATCAAA GGCCCAGATT TCATTGCAGA AGATCAAACC TGTGTTCGAC TTTCTTGTAA | 960 |
| ATAAGTTTTC AACTGTATAT ACTCCAAACA GAAACATTGC AGTCGATGAA TCACTGATGC | 1020 |
| TGTTCAAGGG GCGGTTAGCT ATGAAGCAGT ACATCCCGAC GAAATGtGCA CGATTTGGTC | 1080 |
| TCAAGCTNTA TGTACTTTGT GAAAGTCAAT CTGGTTACGT GTGGAATGCG CTTGTTCACA | 1140 |
| CAGGGCCCAG TATGAATTTG AAAGATTCAG CTGATGGTCT GAAATCGTCA TGCATTGTTC | 1200 |
| TTACCTTGGT CAATGACCTT CTTGGCCAAG GATATTGTGT CTTCCTCAAT AACTTTTATA | 1260 |
| CATCTCCCAT GCTTTTCAGA GAATTACATC AAAACAGGAC TGATGCAGTT GGGACAGCTC | 1320 |
| GTTTGAACAG AAAACAGATG CCAAATGATC TGAAAAAAAG GATTGCAAAG GGGACGACTG | 1380 |
| TAGCCAGATT CTGTGGTGAA CTTATGGCAC TGAAATGGTG TGACAAGAAG GAGGTGACAA | 1440 |
| TGTTGTCAAC ATTCCACAAT GATACTGTGA TTGAAGTAGA CAACAGAAAT GGAAAGAAAA | 1500 |
| CTAAGAAGCC ATGTGTCATT GTGGATTATA ACGAGAATAT GGGAGCAGTG GACTCGGCTG | 1560 |
| ATCAGATGCT CACTTCTTAT CCAACTGAGC GCAAAAGGCA CAAGTTTTGG TATAAGAAAT | 1620 |
| TCTTTCGCCA CCTTCTAAAC ATTACAGTGC TGAACTCCTA CATCCTGTTC AAGAAGGACA | 1680 |
| ATCCTGAGCA CACGATCAGC CATGTAAACT TCAGACTGAC GTTGATTGAA AGAATGCTGG | 1740 |
| AAAAGCATCA CAAGCCAGGG CAGCAACGTC TTCGAGGTCG TCCGTGCTCT GATGATGTCA | 1800 |
| CACCTCTTCG CCTGTCTGGA AGACATTTCC CCAAGAGCAT ACCACCAACA TCAGGGAAAC | 1860 |
| AGAATCCAAC TGGTCGCTGC AAAGTTTGCT GCTCGCACGA CAAGGATGGC AAGAAGATCC | 1920 |
| GGAGAGAAAC GTtATATTTT TGTGCGGAAT GTGATGTTCC GCTTTGTGTT GTTCCGTGCT | 1980 |
| TTGAAATTTA CCACACGAAA AAAAATTATT AAATACTGAT CATCATATAC ATTTCTGTTA | 2040 |
| CATTAGGATT AGAGACAAGT TCTGTTTAGA AATAACTCCA AGAACAGTTT TTATATTTTA | 2100 |
| TTTTCACATT GAAAACCAGT CAGATTTGCT TCAGCCTCAA AGAGCATGTT TATGTAAAAT | 2160 |
| TAAATTAACG CTGGCAGCGA GCTGCACTTN TTTTCTAAAC GGGAAATGGG | 2210 |

In embodiments, the nucleotide sequence comprises a thymine (T) at position 1933 of SEQ ID NO: 1, or a position corresponding thereto. In embodiments, the nucleotide sequence does not comprise a guanine (G) at position 1933 of SEQ ID NO: 1, or a position corresponding thereto.

In embodiments, the helper enzyme has at least about 95%, or at least about 96%, at least about 97%, at least about 98%, at least about 99% identity to the amino acid sequence SEQ ID NO: 6. In embodiments, the helper enzyme has an amino acid sequence having 183P and/or V118R mutation relative to the amino acid sequence of SEQ ID NO: 6 or a functional equivalent thereof.

| SEQ ID NO: 6: PGBD1 Amino Acid Sequence (809 Amino Acids). | |
|---|---|
| MYEALPGPAP ENEDGLVKVK EEDPTWEQVC NSQEGSSHTQ EICRLRFRHF CYQEAHGPQE | 60 |
| ALAQLRELCH QWLRPEMHTK EQIMELLVLE QFLTILPKEL QPCVKTYPLE SGEEAVTVLE | 120 |
| NLETGSGDTG QQASVYIQGQ DMHPMVAEYQ GVSLECQSLQ LLPGITTLKC EPPQRPQGNP | 180 |
| QEVSGPVPHG SAHLQEKNPR DKAVVPVFNP VRSQTLVKTE EETAQAVAAE KWSHLSLTRR | 240 |
| NLCGNSAQET VMSLSPMTEE IVTKDRLFKA KQETSEEMEQ SGEASGKPNR ECAPQIPCST | 300 |
| PIATERTVAH LNTLKDRHPG DLWARMHISS LEYAAGDITR KGRKKDKARV SELLQGLSFS | 360 |
| GDSDVEKDNE PEIQPAQKKL KVSCFPEKSW TKRDIKPNFP SWSALDSGLL NLKSEKLNPV | 420 |
| ELFELFFDDE TFNLIVNETN NYASQKNVSL EVTVQEMRCV FGVLLLSGFM RHPRREMYWE | 480 |
| VSDTDQNLVR DAIRRDRFEL IFSNLHFADN GHLDQKDKFT KLRPLIKQMN KNFLLYAPLE | 540 |
| EYYCFDKSMC ECFDSDQFLN GKPIRIGYKI WCGTTTQGYL VWFEPYQEES TMKVDEDPDL | 600 |
| GLGGNLVMNF ADVLLERGQY PYHLCFDSFF TSVKLLSALK KKGVRATGTI RENRTEKCPL | 660 |
| MNVEHMKKMK RGYFDFRIEE NNEIILCRWY GDGIISLCSN AVGIEPVNEV SCCDADNEEI | 720 |
| PQISQPSIVK VYDECKEGVA KMDQIISKYR VRIRSKKWYS ILVSYMIDVA MNNAWQLHRA | 780 |
| CNPGASLDPL DFRRFVAHFY LEHNAHLSD | 809 |

In embodiments, the helper enzyme has at least about 95%, or at least about 96%, at least about 97%, at least about 98%, at least about 99% identity to the amino acid sequence SEQ ID NO: 7. In embodiments, the helper enzyme has an amino acid sequence having S20P and/or A29R mutation relative to the amino acid sequence of SEQ ID NO: 7 or a functional equivalent thereof.

| SEQ ID NO: 7: PGBD2 Amino Acid Sequence (592 Amino Acids). | |
|---|---|
| MASTSRDVIA GRGIHSKVKS AKLLEVLNAM EEEESNNNRE EIFIAPPDNA AGEFTDEDSG | 60 |
| DEDSQRGAHL PGSVLHASVL CEDSGTGEDN DDLELQPAKK RQKAVVKPQR IWTKRDIRPD | 120 |
| FGSWTASDPH IEDLKSQELS PVGLFELFFD EGTINFIVNE TNRYAWQKNV NLSLTAQELK | 180 |
| CVLGILILSG YISYPRRRMF WETSPDSHHH LVADAIRRDR FELIFSYLHF ADNNELDASD | 240 |
| RFAKVRPLII RMNCNFQKHA PLEEFYSFGE SMCEYFGHRG SKQLHRGKPV RLGYKIWCGT | 300 |
| TSRGYLVWFE PSQGTLFTKP DRSLDLGGSM VIKFVDALQE RGFLPYHIFF DKVFTSVKLM | 360 |
| SILRKKGVKA TGTVREYRTE RCPLKDPKEL KKMKRGSFDY KVDESEEIIV CRWHDSSVVN | 420 |
| ICSNAVGIEP VRLTSRHSGA AKTRTQVHQP SLVKLYQEKV GGVGRMDQNI AKYKVKIRGM | 480 |
| KWYSSFIGYV IDAALNNAWQ LHRICCQDAQ VDLLAFRRYI ACVYLESNAD TTSQGRRSRR | 540 |
| LETESRFDMI GHWIIHQDKR TRCALCHSQT NTRCEKCQKG VHAKCFREYH IR | 592 |

In embodiments, the helper enzyme has at least about 95%, or at least about 96%, at least about 97%, at least about 98%, at least about 99% identity to the amino acid sequence SEQ ID NO: 9. In embodiments, the helper enzyme has an amino acid sequence having A12P and/or 128R mutation and/or R152K mutation relative to the amino acid sequence of SEQ ID NO: 9 or a functional equivalent thereof.

| SEQ ID NO: 9: PGBD5 Amino Acid Sequence (524 Amino Acids). | |
|---|---|
| MAEGGGGARR RAPALLEAAR ARYESLHISD DVFGESGPDS GGNPFYSTSA ASRSSSAASS | 60 |
| DDEREPPGPP GAAPPPPRAP DAQEPEEDEA GAGWSAALRD RPPPRFEDTG GPTRKMPPSA | 120 |
| SAVDFFQLFV PDNVLKNMVV QTNMYAKKFQ ERFGSDGAWV EVTLTEMKAF LGYMISTSIS | 180 |
| HCESVLSIWS GGFYSNRSLA LVMSQARFEK ILKYFHVVAF RSSQTTHGLY KVQPFLDSLQ | 240 |
| NSFDSAFRPS QTQVLHEPLI DEDPVFIATC TERELRKRKK RKFSLWVRQC SSTGFIIQIY | 300 |
| VHLKEGGGPD GLDALKNKPQ LHSMVARSLC RNAAGKNYII FTGPSITSLT LFEEFEKQGI | 360 |
| YCCGLLRARK SDCTGLPLSM LTNPATPPAR GQYQIKMKGN MSLICWYNKG HFRFLTNAYS | 420 |
| PVQQGVIIKR KSGEIPCPLA VEAFAAHLSY ICRYDDKYSK YFISHKPNKT WQQVFWFAIS | 480 |
| IAINNAYILY KMSDAYHVKR YSRAQFGERL VRELLGLEDA SPTH | 524 |

In embodiments, the helper enzyme has at least about 95%, or at least about 96%, at least about 97%, at least about 98%, at least about 99% identity to the amino acid sequence SEQ ID NO: 8. In embodiments, the helper enzyme has an amino acid sequence having T4P and/or L13R mutation relative to the amino acid sequence of SEQ ID NO: 8 or a functional equivalent thereof.

| SEQ ID NO: 8: PGBD3 Amino Acid Sequence (593 Amino Acids). | |
|---|---|
| MPRTLSLHEI TDLLETDDSI EASAIVIQPP ENATAPVSDE ESGDEEGGTI NNLPGSLLHT | 60 |
| AAYLIQDGSD AESDSDDPSY APKDDSPDEV PSTFTVQQPP PSRRRKMTKI LCKWKKADLT | 120 |
| VQPVAGRVTA PPNDFFTVMR TPTEILELFL DDEVIELIVK YSNLYACSKG VHLGLTSSEF | 180 |
| KCFLGIIFLS GYVSVPRRRM FWEQRTDVHN VLVSAAMRRD RFETIFSNLH VADNANLDPV | 240 |
| DKFSKLRPLI SKLNERCMKF VPNETYFSFD EFMVPYFGRH GCKQFIRGKP IRFGYKFWCG | 300 |
| ATCLGYICWF QPYQGKNPNT KHEEYGVGAS LVLQFSEALT EAHPGQYHFV FNNFFTSIAL | 360 |
| LDKLSSMGHQ ATGTVRKDHI DRVPLESDVA LKKKERGTFD YRIDGKGNIV CRWNDNSVVT | 420 |
| VASSGAGIHP LCLVSRYSQK LKKKIQVQQP NMIKVYNQFM GGVDRADENI DKYRASIRGK | 480 |
| KWYSSPLLFC FELVLQNAWQ LHKTYDEKPV DFLEFRRRVV CHYLETHGHP PEPGQKGRPQ | 540 |
| KRNIDSRYDG INHVIVKQGK QTRCAECHKN TTFRCEKCDV ALHVKCSVEY HTE | 593 |

Ends and Constructs

In embodiments, the composition comprises a gene transfer construct. In embodiments, the gene transfer construct comprises left and right end sequences recognized by the helper enzyme. In embodiments, the gene transfer construct comprises a vector comprising a donor DNA comprising left and right end sequences recognized by the helper enzyme. In embodiments, the end sequences are selected from ends from Pteropus vampyrus, MER75, MER75A, MER75B, and MER85.

In embodiments, the end sequences are selected from nucleotide sequences of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20, or a nucleotide sequence having at least about 90% identity thereto.

| SEQ ID NO: 11: Pteropus vampyrus Left End Nucleotide Sequence (381 bp). | |
|---|---|
| TTAACCCATT TCCTGTTTGC CCCGAGAATA CTCACCAGCG GCACTTGCAG CTGCAGCGTT | 60 |
| TACCCCGAGA TAACTCGTCG ATTACAGTCC TAACCTTACC CCCAAAGTTT GCCATGAAAT | 120 |
| ATCTCGCTTT TATTATTATT TTCGCATCGC TCTAGTATAT CGATAGTCTT TGGAAACAAA | 180 |
| TGACATCATT CTATTTACAG CATTCTGTTT TTAGTAGTGG TATTTCCATT TACAAAATAT | 240 |
| AGTAATTTTC TATCGCTGAA AATGTCAAAT CCTAGAAAAC GTAGCATTCC TACATGTGAT | 300 |
| GTTAACTTCG TTCTCGAACA GTTGTTAGCC GAAGATTCAT TTGATGAATC CGATTTTTCC | 360 |
| GAAATAGACG ATTCTGATGA T | 381 |
| SEQ ID NO: 12: PGBD4 Left End Nucleotide Sequence (373 bp). | |
| TTAACTCATT TCTCCTTAGC CCCGAGATTA CGCGCTGCTG TGCCTGCGAC TGCAGCGTTT | 60 |
| ACGCCGAGAT AACTCGTGGA TTACAGTGCC AACCTTACTC CCAAAGTTTG CCACGAAATA | 120 |
| TCTCGCTTCT GTTATTTTCG CATGGTTCTG GTATATTGAC TTTTGAAACA AAAGACATCA | 180 |
| TTCTGTTTAT AGCATTCTGT TTTTAGTAGT GGGATTTCCA TCTACAAAAT ATAGTAATTC | 240 |
| TCGATCGCTG AAATGTCAAA TCCTAGAAAA CGTAGCATTC CTATGCGTGA TAGTAATACC | 300 |
| GGTCTCGAAC AGTTGTTGGC TGAAGATTCA TTTGATGAAT CTGATTTTTC GGAAATAGAT | 360 |
| GATTCTGATA ATT | 373 |
| SEQ ID NO: 13: MER75 Left End Nucleotide Sequence (344 bp). | |
| TTAACCCTTT TCCCGTTTGC CCCGAGAATA CTCGCCGGCG GCGCTTGCGG CTGCAGCGTT | 60 |
| TACCCCGAGA TAACTTTGCC ACGAAATATC TCGCTTTTAT TATTATTTTC GCATCGCTCT | 120 |
| AGTATATCGA CTTTGGAAAC AAAAGACATC ATTCTATTTA TAGCATTCTG TTTTTAGTAG | 180 |
| TGGTATTTCC ATTTACAAAA TATAGTAATT CTCGATCGCT GAAAATGTCA AATCCTAGAA | 240 |
| AACGTAGCAT TCCTACGCGT GATGTTAACA TCGTTCTCGA ACAGTTGTTG GCCGAAGATT | 300 |
| CATTTGATGA ATCCGATTTT TCCGAAATAG ACGATTCTGA TGAT | 344 |
| SEQ ID NO: 14: MER75B Left End Nucleotide Sequence (91 bp). | |
| TTAACCCATT TCCCGTTTGC CCCGAGAATA CTCTTGTCTC TAATCCTAAT GTAACATCAT | 60 |
| ATACATTTCT GTTACATTAG GATTAGAGAC A | 91 |
| SEQ ID NO: 15: MER75A Left End Nucleotide Sequence (32 bp). | |
| TTAACCCATT TCCCGTTTGC CCCGAGAATA CT | 32 |
| SEQ ID NO: 16: Pteropus vampyrus Right End Nucleotide Sequence (171 bp). | |
| TAGGATTAGA GACAAGTTCT GTTTAGAAAT AACTCCAAGA ACAGTTTTTA TATTTTATTT | 60 |
| TCACATTGAA AACCAGTCAG ATTTGCTTCA GCCTCAAAGA GCATGTTTAT GTAAAATTAA | 120 |
| ATTAACGCTG GCAGCGAGCT GCACTTTTTT TCTAAACGGG AAATGGGTTA A | 171 |
| SEQ ID NO: 17: PGBD4 Right End Nucleotide Sequence (176 bp). | |
| CCTGGGATTA TAGGCATGAG CCACTGCGCC TAGCACCAAG AACAGTTTTT ATATTTTATT | 60 |
| TTCACATTGA AAATCAGTCA GATTTGCTTC AGCCTCAAAG AGGGTGTTTA TGTAAAACTA | 120 |
| AATGAGTGCA GGCAGCGAGC TACACTTTTT TTTTTCCTAA ATGGAAAATG GGTTAA | 176 |
| SEQ ID NO: 18: MER75 Right End Nucleotide Sequence (178 bp). | |
| TCAGACGATT CTGATGTTAG TTCTGTTTAG AAATAACTCC AAGAACAGTT TTTATATTTT | 60 |
| ATTTTCACAT TGAAAATCAG TCAGATTTGC TTCAGCCTCA AAGAGCGTGT TTATGTAAAA | 120 |
| TTAAATGAGC GCTGGCAGCG AGCTGCACTT TTTTTTTTCT AAACGGGAAA AGGGTTAA | 178 |
| SEQ ID NO: 19: MER75B Right End Nucleotide Sequence (160 bp). | |
| AGTTCTGTTT AGAAATAACT CCAAGAACAG TTTTTATATT TTATTTTCAC ATTGAAAATC | 60 |
| AGTCAGATTT GCTTCAGCCT CAAAGAGCGT GTTTATGTAA AATTAAATGA GCGCTGGCAG | 120 |
| CGAGCTGCAC TTTTTTTTTT CTAAACGGGA AAAGGGTTAA | 160 |
| SEQ ID NO: 20: MER75A Right End Nucleotide Sequence (46 bp). | |
| CGCTGGCAGC GAGCTGCACT TTTTTTCTAA ACGGGAAATG GGTTAA | 46 |

In embodiments, one or more of the end sequences are optionally flanked by a TTAA sequence.

In embodiments, the end sequences include at least one repeat from a nucleotide sequence having at least about 90% identity to the nucleotide sequence of SEQ ID NO: 11, and wherein the at least one repeat from the nucleotide sequence having at least about 90% identity to the nucleotide sequence of SEQ ID NO: 11 is positioned at the 5′ end of the donor DNA. In embodiments, the end sequences include at least one repeat from a nucleotide sequence having at least about 90% identity to the nucleotide sequence of SEQ ID NO: 16, and wherein the at least one repeat from the nucleotide sequence having at least about 90% identity to the nucleotide sequence of SEQ ID NO: 16 is positioned at the 3′ end of the donor DNA. In embodiments, the end sequences are optionally flanked by a TTAA sequence.

In embodiments, the end sequences include at least one repeat from a nucleotide sequence having at least about 90% identity to the nucleotide sequence of SEQ ID NO: 12, and wherein the at least one repeat from the nucleotide sequence having at least about 90% identity to the nucleotide sequence of SEQ ID NO: 12 is positioned at the 5′ end of the donor DNA. In embodiments, the end sequences include at least one repeat from a nucleotide sequence having at least about 90% identity to the nucleotide sequence of SEQ ID NO: 17, and wherein the at least one repeat from the nucleotide sequence having at least about 90% identity to the nucleotide sequence of SEQ ID NO: 17 is positioned at the 3′ end of the donor DNA. In embodiments, the end sequences are optionally flanked by a TTAA sequence.

In embodiments, the end sequences include at least one repeat from a nucleotide sequence having at least about 90% identity to the nucleotide sequence of SEQ ID NO: 13, wherein the at least one repeat from the nucleotide sequence having at least about 90% identity to the nucleotide sequence of SEQ ID NO: 13 is positioned at the 5′ end of the donor DNA. In embodiments, the end sequences include at least one repeat from a nucleotide sequence having at least about 90% identity to the nucleotide sequence of SEQ ID NO: 18, wherein the at least one repeat from the nucleotide sequence having at least about 90% identity to the nucleotide sequence of SEQ ID NO: 18 is positioned at the 3′ end of the donor DNA. In embodiments, the end sequences are optionally flanked by a TTAA sequence.

In embodiments, the end sequences include at least one repeat from a nucleotide sequence having at least about 90% identity to the nucleotide sequence of SEQ ID NO: 14, wherein the at least one repeat from the nucleotide sequence having at least about 90% identity to the nucleotide sequence of SEQ ID NO: 14 is positioned at the 5′ end of the donor DNA. In embodiments, the end sequences include at least one repeat from a nucleotide sequence having at least about 90% identity to the nucleotide sequence of SEQ ID NO: 19, wherein the at least one repeat from the nucleotide sequence having at least about 90% identity to the nucleotide sequence of SEQ ID NO: 19 is positioned at the 3′ end of the donor DNA. In embodiments, the end sequences are optionally flanked by a TTAA sequence.

In embodiments, the end sequences include at least one repeat from a nucleotide sequence having at least about 90% identity to the nucleotide sequence of SEQ ID NO: 15, wherein the at least one repeat from the nucleotide sequence having at least about 90% identity to the nucleotide sequence of SEQ ID NO: 15 is positioned at the 5′ end of the donor DNA. In embodiments, the end sequences include at least one repeat from a nucleotide sequence having at least about 90% identity to the nucleotide sequence of SEQ ID NO: 20, wherein the at least one repeat from the nucleotide sequence having at least about 90% identity to the nucleotide sequence of SEQ ID NO: 20 is positioned at the 3′ end of the donor DNA. The composition of claim 25 or claim 26, wherein the end sequences are optionally flanked by a TTAA sequence.

Other Mammalian Helper Enzymes and Pteropus vampyrus End Sequences

In aspects, a composition is provided comprising: (a) a recombinant helper enzyme, or a nucleotide sequence encoding the same, e.g., having gene cleavage (Exc) and/or gene integration (Int) activity and at least about 90% identity to the amino acid sequence of SEQ ID NO: 3, SEQ ID NO: 4, SEQ ID NO: 6, SEQ ID NO: 7, SEQ ID NO: 8, or SEQ ID NO: 9 (inclusive of various mutants, e.g. as described herein), and (b) a gene transfer construct comprises a vector comprising a donor DNA comprising left and right end sequences recognized by the recombinant helper enzyme, the end sequences having at least about 90% identity to the nucleotide sequences of SEQ ID NO: 11 and SEQ ID NO: 16.

The following helpers are used in the aspects and embodiments described herein:

In embodiments, the helper enzyme has an amino acid sequence having mutations in at least one of positions 8, 17, and 134, relative to the amino acid sequence of SEQ ID NO: 3 or SEQ ID NO.: 4 or a functional equivalent thereof.

| SEQ ID NO: 3: PGBD4 Acid Sequence (585 Amino Acids). | |
|---|---|
| MSNPRKRSIP MRDSNTGLEQ LLAEDSFDES DFSEIDDSDN FSDSALEADK | 50 |
| IRPLSHLESD GKSSTSSDSG RSMKWSARAM IPRQRYDFTG TPGRKVDVSD | 100 |
| ITDPLQYFEL FFTEELVSKI TRETNAQAAL LASKPPGPKG FSRMDKWKDT | 150 |
| DNDELKVFFA VMLLQGIVQK PELEMFWSTR PLLDTPYLRQ IMTGERFLLL | 200 |
| FRCLHFVNNS SISAGQSKAQ ISLQKIKPVF DFLVNKFSTV YTPNRNIAVD | 250 |
| ESLMLFKGPL AMKQYLPTKR VRFGLKLYVL CESQSGYVWN ALVHTGPGMN | 300 |
| LKDSADGLKS SRIVLTLVND LLGQGYCVFL DNFNISPMLF RELHQNRTDA | 350 |
| VGTARLNRKQ IPNDLKKRIA KGTTVARFCG ELMALKWCDG KEVTMLSTFH | 400 |
| NDTVIEVNNR NGKKTKRPRV IVDYNENMGA VDSADQMLTS YPSERKRHKV | 450 |
| WYKKFFHHLL HITVLNSYIL FKKDNPEHTM SHINFRLALI ERMLEKHHKP | 500 |
| GQQHLRGRPC SDDVTPLRLS GRHFPKSIPA TSGKQNPTGR CKICCSQYDK | 550 |
| DGKKIRKETR YFCAECDVPL CVVPCFEIYH TKKNY | 585 |
| SEQ ID NO: 4: PGBD4 Hyperactive Mutant (S8P, G17R, K134K) | |
| Amino Acid Sequence (585 Amino Acids). | |
| MSNPRKRPIP MRDSNTRLEQ LLAEDSFDES DFSEIDDSDN FSDSALEADK | 50 |
| IRPLSHLESD GKSSTSSDSG RSMKWSARAM IPRQRYDFTG TPGRKVDVSD | 100 |
| ITDPLQYFEL FFTEELVSKI TRETNAQAAL LASKPPGPKG FSRMDKWKDT | 150 |
| DNDELKVFFA VMLLQGIVQK PELEMFWSTR PLLDTPYLRQ IMTGERFLLL | 200 |
| FRCLHFVNNS SISAGQSKAQ ISLQKIKPVF DFLVNKFSTV YTPNRNIAVD | 250 |
| ESLMLFKGPL AMKQYLPTKR VRFGLKLYVL CESQSGYVWN ALVHTGPGMN | 300 |
| LKDSADGLKS SRIVLTLVND LLGQGYCVFL DNFNISPMLF RELHQNRTDA | 350 |
| VGTARLNRKQ IPNDLKKRIA KGTTVARFCG ELMALKWCDG KEVTMLSTFH | 400 |
| NDTVIEVNNR NGKKTKRPRV IVDYNENMGA VDSADQMLTS YPSERKRHKV | 450 |
| WYKKFFHHLL HITVLNSYIL FKKDNPEHTM SHINFRLALI ERMLEKHHKP | 500 |
| GQQHLRGRPC SDDVTPLRLS GRHFPKSIPA TSGKQNPTGR CKICCSQYDK | 550 |
| DGKKIRKETR YFCAECDVPL CVVPCFEIYH TKKNY | 585 |

In embodiments, the helper enzyme has an nucleotide acid sequence having at least about 90% identity to the nucleotide sequence of SEQ ID NO: 5.

| SEQ ID NO: 5: PGBD4 Hyperactive Mutant (S8P, G17R, K134K) | |
|---|---|
| Nucleotide Sequence (1758 bp). | |
| ATGTCAAATC CTAGAAAACG TCCCATTCCT ATGCGTGATA GTAATACCCG TCTCGAACAG | 60 |
| TTGTTGGCTG AAGATTCATT TGATGAATCT GATTTTTCGG AAATAGATGA TTCTGATAAT | 120 |
| TTTTCGGATA GTGCTTTAGA AGCCGATAAG ATCAGGCCTC TGTCCCATTT AGAATCTGAT | 180 |
| GGAAAGAGCT CTACATCAAG TGACTCAGGG CGCTCCATGA AATGGTCAGC TCGTGCTATG | 240 |
| ATTCCACGTC AAAGGTATGA CTTTACCGGC ACACCTGGCA GAAAAGTCGA TGTCAGTGAT | 300 |
| ATCACTGACC CATTGCAGTA TTTTGAACTG TTCTTTACTG AGGAATTAGT TTCAAAAATT | 360 |
| ACTAGAGAAA CAAATGCCCA AGCTGCCTTG TTGGCTTCAA AGCCACCGGG TCCGAAAGGA | 420 |
| TTTTCGCGAA TGGATAAATG GAAAGACACT GACAATGACG AGCTCAAAGT CTTTTTTGCA | 480 |
| GTAATGTTAC TGCAAGGTAT TGTGCAGAAA CCTGAGCTGG AGATGTTTTG GTCAACAAGG | 540 |
| CCTCTTTTGG ATACACCTTA TCTCAGGCAA ATTATGACTG GTGAAAGATT TTTACTTTTG | 600 |
| TTTCGGTGCC TGCATTTTGT CAACAATTCT TCTATATCTG CTGGTCAATC AAAGGCCCAG | 660 |
| ATTTCATTGC AGAAGATCAA ACCTGTGTTC GACTTTCTTG TAAATAAATT TTCCACTGTA | 720 |
| TATACTCCAA ACAGAAACAT TGCAGTTGAT GAATCACTGA TGCTGTTCAA GGGGCCATTA | 780 |
| GCTATGAAGC AGTACCTCCC GACAAAACGA GTACGATTTG GTCTGAAGCT ATATGTACTT | 840 |
| TGTGAAAGTC AGTCTGGTTA TGTGTGGAAT GCGCTTGTTC ACACAGGGCC TGGCATGAAT | 900 |
| TTGAAAGATT CAGCGGATGG CCTGAAATCA TCACGCATTG TTCTTACCTT GGTCAATGAC | 960 |
| CTTCTTGGCC AAGGGTATTG TGTCTTCCTC GATAACTTTA ATATATCTCC CATGCTTTTC | 1020 |
| AGAGAATTAC ATCAAAATAG GACTGATGCA GTTGGGACAG CTCGTTTGAA CAGAAAACAG | 1080 |
| ATTCCAAATG ATCTGAAAAA AAGGATTGCA AAGGGGACGA CTGTAGCCAG ATTCTGTGGT | 1140 |
| GAACTTATGG CACTGAAATG GTGTGACGGC AAGGAGGTGA CAATGTTGTC AACATTCCAC | 1200 |
| AATGATACTG TGATTGAAGT AAACAATAGA AATGGAAAGA AAACTAAAAG GCCACGTGTC | 1260 |
| ATTGTGGATT ATAACGAGAA TATGGGAGCA GTGGACTCGG CTGATCAAAT GCTTACTTCT | 1320 |
| TATCCATCTG AGCGCAAAAG ACACAAGGTT TGGTATAAGA AATTCTTTCA CCATCTTCTA | 1380 |
| CACATTACAG TGCTGAACTC CTACATCCTG TTCAAGAAGG ATAATCCTGA GCACACGATG | 1440 |
| AGCCATATAA ACTTCAGACT GGCATTGATT GAAAGAATGC TGGAAAAGCA TCACAAGCCA | 1500 |
| GGGCAGCAAC ATCTTCGAGG TCGTCCTTGC TCCGATGATG TCACACCTCT TCGTCTGTCT | 1560 |
| GGAAGACATT TCCCCAAGAG CATACCAGCA ACGTCCGGGA AACAGAATCC AACTGGTCGC | 1620 |
| TGCAAAATTT GCTGCTCCCA ATACGACAAG GATGGCAAGA AGATCCGGAA AGAAACGCGC | 1680 |
| TATTTTTGTG CCGAATGTGA TGTTCCGCTT TGTGTTGTTC CGTGCTTTGA AATTTACCAC | 1740 |
| ACGAAAAAAA ATTATTAA | 1758 |

In embodiments, the helper enzyme has an amino acid sequence having a mutation in positions 83, and 118, relative to the amino acid sequence of SEQ ID NO: 6 or a functional equivalent thereof. In embodiments, the helper enzyme has an amino acid sequence having a mutation in position 83 and/or position 118 relative to the amino acid sequence of SEQ ID NO: 6 or a functional equivalent thereof. In embodiments, the helper enzyme has an amino acid sequence having 183P mutation and/or V118R mutation relative to the amino acid sequence of SEQ ID NO: 6 or a functional equivalent thereof.

| SEQ ID NO: 6: PGBD1 Amino Acid Sequence (809 Amino Acids). | |
|---|---|
| MYEALPGPAP ENEDGLVKVK EEDPTWEQVC NSQEGSSHTQ EICRLRFRHF CYQEAHGPQE | 60 |
| ALAQLRELCH QWLRPEMHTK EQIMELLVLE QFLTILPKEL QPCVKTYPLE SGEEAVTVLE | 120 |
| NLETGSGDTG QQASVYIQGQ DMHPMVAEYQ GVSLECQSLQ LLPGITTLKC EPPQRPQGNP | 180 |
| QEVSGPVPHG SAHLQEKNPR DKAVVPVFNP VRSQTLVKTE EETAQAVAAE KWSHLSLTRR | 240 |
| NLCGNSAQET VMSLSPMTEE IVTKDRLFKA KQETSEEMEQ SGEASGKPNR ECAPQIPCST | 300 |
| PIATERTVAH LNTLKDRHPG DLWARMHISS LEYAAGDITR KGRKKDKARV SELLQGLSFS | 360 |
| GDSDVEKDNE PEIQPAQKKL KVSCFPEKSW TKRDIKPNFP SWSALDSGLL NLKSEKLNPV | 420 |
| ELFELFFDDE TFNLIVNETN NYASQKNVSL EVTVQEMRCV FGVLLLSGFM RHPRREMYWE | 480 |
| VSDTDQNLVR DAIRRDRFEL IFSNLHFADN GHLDQKDKFT KLRPLIKQMN KNFLLYAPLE | 540 |
| EYYCFDKSMC ECFDSDQFLN GKPIRIGYKI WCGTTTQGYL VWFEPYQEES TMKVDEDPDL | 600 |
| GLGGNLVMNF ADVLLERGQY PYHLCFDSFF TSVKLLSALK KKGVRATGTI RENRTEKCPL | 660 |
| MNVEHMKKMK RGYFDFRIEE NNEIILCRWY GDGIISLCSN AVGIEPVNEV SCCDADNEEI | 720 |
| PQISQPSIVK VYDECKEGVA KMDQIISKYR VRIRSKKWYS ILVSYMIDVA MNNAWQLHRA | 780 |
| CNPGASLDPL DFRRFVAHFY LEHNAHLSD | 809 |

In embodiments, the helper enzyme has an amino acid sequence having a mutation in positions 20, and 29, relative to the amino acid sequence of SEQ ID NO: 7 or a functional equivalent thereof. In embodiments, the helper enzyme has an amino acid sequence having a mutation in position 20 and/or position 29 relative to the amino acid sequence of SEQ ID NO: 7 or a functional equivalent thereof. In embodiments, the helper enzyme has an amino acid sequence having S20P mutation and/or A29R mutation relative to the amino acid sequence of SEQ ID NO: 7 or a functional equivalent thereof.

| SEQ ID NO: 7: PGBD2 Amino Acid Sequence (592 Amino Acids). | |
|---|---|
| MASTSRDVIA GRGIHSKVKS AKLLEVLNAM EEEESNNNRE EIFIAPPDNA AGEFTDEDSG | 60 |
| DEDSQRGAHL PGSVLHASVL CEDSGTGEDN DDLELQPAKK RQKAVVKPQR IWTKRDIRPD | 120 |
| FGSWTASDPH IEDLKSQELS PVGLFELFFD EGTINFIVNE TNRYAWQKNV NLSLTAQELK | 180 |
| CVLGILILSG YISYPRRRMF WETSPDSHHH LVADAIRRDR FELIFSYLHF ADNNELDASD | 240 |
| RFAKVRPLII RMNCNFQKHA PLEEFYSFGE SMCEYFGHRG SKQLHRGKPV RLGYKIWCGT | 300 |
| TSRGYLVWFE PSQGTLFTKP DRSLDLGGSM VIKFVDALQE RGFLPYHIFF DKVFTSVKLM | 360 |
| SILRKKGVKA TGTVREYRTE RCPLKDPKEL KKMKRGSFDY KVDESEEIIV CRWHDSSVVN | 420 |
| ICSNAVGIEP VRLTSRHSGA AKTRTQVHQP SLVKLYQEKV GGVGRMDQNI AKYKVKIRGM | 480 |
| KWYSSFIGYV IDAALNNAWQ LHRICCQDAQ VDLLAFRRYI ACVYLESNAD TTSQGRRSRR | 540 |
| LETESRFDMI GHWIIHQDKR TRCALCHSQT NTRCEKCQKG VHAKCFREYH IR | 592 |

In embodiments, the helper enzyme has an amino acid sequence having a mutation in positions 4, and 13, relative to the amino acid sequence of SEQ ID NO: 8 or a functional equivalent thereof. In embodiments, the helper enzyme has an amino acid sequence having a mutation in position 4 and/or position 13 relative to the amino acid sequence of SEQ ID NO: 8 or a functional equivalent thereof. In embodiments, the helper enzyme has an amino acid sequence having T4P mutation and/or L13R mutation relative to the amino acid sequence of SEQ ID NO: 8 or a functional equivalent thereof.

| SEQ ID NO: 8: PGBD3 Amino Acid Sequence (593 Amino Acids). | |
|---|---|
| MPRTLSLHEI TDLLETDDSI EASAIVIQPP ENATAPVSDE ESGDEEGGTI NNLPGSLLHT | 60 |
| AAYLIQDGSD AESDSDDPSY APKDDSPDEV PSTFTVQQPP PSRRRKMTKI LCKWKKADLT | 120 |
| VQPVAGRVTA PPNDFFTVMR TPTEILELFL DDEVIELIVK YSNLYACSKG VHLGLTSSEF | 180 |
| KCFLGIIFLS GYVSVPRRRM FWEQRTDVHN VLVSAAMRRD RFETIFSNLH VADNANLDPV | 240 |
| DKFSKLRPLI SKLNERCMKF VPNETYFSFD EFMVPYFGRH GCKQFIRGKP IRFGYKFWCG | 300 |
| ATCLGYICWF QPYQGKNPNT KHEEYGVGAS LVLQFSEALT EAHPGQYHFV FNNFFTSIAL | 360 |
| LDKLSSMGHQ ATGTVRKDHI DRVPLESDVA LKKKERGTFD YRIDGKGNIV CRWNDNSVVT | 420 |
| VASSGAGIHP LCLVSRYSQK LKKKIQVQQP NMIKVYNQFM GGVDRADENI DKYRASIRGK | 480 |
| KWYSSPLLFC FELVLQNAWQ LHKTYDEKPV DFLEFRRRVV CHYLETHGHP PEPGQKGRPQ | 540 |
| KRNIDSRYDG INHVIVKQGK QTRCAECHKN TTFRCEKCDV ALHVKCSVEY HTE | 593 |

In embodiments, the helper enzyme has an amino acid sequence having a mutation in positions 12, 28 and 152, relative to the amino acid sequence of SEQ ID NO: 9 or a functional equivalent thereof. In embodiments, the helper enzyme has an amino acid sequence having a mutation in position 12 and/or position 28 and/or position 152 relative to the amino acid sequence of SEQ ID NO: 9 or a functional equivalent thereof. In embodiments, the helper enzyme has an amino acid sequence having A12P mutation and/or 128R mutation and/or R152K mutation relative to the amino acid sequence of SEQ ID NO: 9 or a functional equivalent thereof.

| SEQ ID NO: 9: PGBD5 Amino Acid Sequence (524 Amino Acids). | |
|---|---|
| MAEGGGGARR RAPALLEAAR ARYESLHISD DVFGESGPDS GGNPFYSTSA ASRSSSAASS | 60 |
| DDEREPPGPP GAAPPPPRAP DAQEPEEDEA GAGWSAALRD RPPPRFEDTG GPTRKMPPSA | 120 |
| SAVDFFQLFV PDNVLKNMVV QTNMYAKKFQ ERFGSDGAWV EVTLTEMKAF LGYMISTSIS | 180 |
| HCESVLSIWS GGFYSNRSLA LVMSQARFEK ILKYFHVVAF RSSQTTHGLY KVQPFLDSLQ | 240 |
| NSFDSAFRPS QTQVLHEPLI DEDPVFIATC TERELRKRKK RKFSLWVRQC SSTGFIIQIY | 300 |
| VHLKEGGGPD GLDALKNKPQ LHSMVARSLC RNAAGKNYII FTGPSITSLT LFEEFEKQGI | 360 |
| YCCGLLRARK SDCTGLPLSM LTNPATPPAR GQYQIKMKGN MSLICWYNKG HFRFLTNAYS | 420 |
| PVQQGVIIKR KSGEIPCPLA VEAFAAHLSY ICRYDDKYSK YFISHKPNKT WQQVFWFAIS | 480 |
| IAINNAYILY KMSDAYHVKR YSRAQFGERL VRELLGLEDA SPTH | 524 |

Targeting Chimeric Constructs

In aspects, the present disclosure provides for targeted chimeras, e.g., in embodiments, the enzyme, without limitation, a helper enzyme, comprises a targeting element.

In embodiments, the enzyme, without limitation, a helper enzyme, associated with the targeting element, is capable of inserting the donor DNA comprising a transgene, optionally at a TA dinucleotide site or a TTAA (SEQ ID NO: 440) tetranucleotide site in a genomic safe harbor site (GSHS). In embodiments, the binding of a GSHS of a nucleic acid molecule in a mammalian cell is with high target specificity.

In embodiments, the targeting element is able to direct a transposition machinery to the GSHS of a nucleic acid molecule in a mammalian cell.

In embodiments, the enzyme, without limitation, a helper enzyme, associated with the targeting element has one or more mutations which confer hyperactivity.

In embodiments, the enzyme, without limitation, a helper enzyme, associated with the targeting element has gene cleavage (Exc+) and/or gene integration activity (Int-F).

In embodiments, the enzyme, without limitation, a helper enzyme, associated with the targeting element has gene cleavage (Exc+) and/or a lack of gene integration activity (Int−).

In embodiments, the targeting element comprises one or more proteins or nucleic acids that are capable of binding to a nucleic acid.

In embodiments, the targeting element comprises one or more of a of a gRNA, optionally associated with a Cas enzyme, which is optionally catalytically inactive, transcription activator-like effector (TALE), catalytically inactive Zinc finger, catalytically inactive transcription factor, nickase, a transcriptional activator, a transcriptional repressor, a recombinase, a DNA methyltransferase, a histone methyltransferase, paternally expressed gene 10 (PEG10), and TnsD.

In embodiments, the targeting element comprises a transcription activator-like effector (TALE) DNA binding domain (DBD).

TALE nucleases (TALENs) are a known tool for genome editing and introducing targeted double-stranded breaks. TALENs comprise endonucleases, such as FokI nuclease domain, fused to a customizable DBD. This DBD is composed of highly conserved repeats from TALEs, which are proteins secreted by Xanthomonas bacteria to alter transcription of genes in host plant cells. The DBD includes a repeated highly conserved 33-34 amino acid sequence with divergent 12th and 13th amino acids. These two positions, referred to as the RVD, are highly variable and show a strong correlation with specific base pair or nucleotide recognition. This straightforward relationship between amino acid sequence and DNA recognition has allowed for the engineering of specific DBDs by selecting a combination of repeat segments containing the appropriate RVDs. Boch et al. Nature Biotechnology. 2011; 29 (2): 135-6.

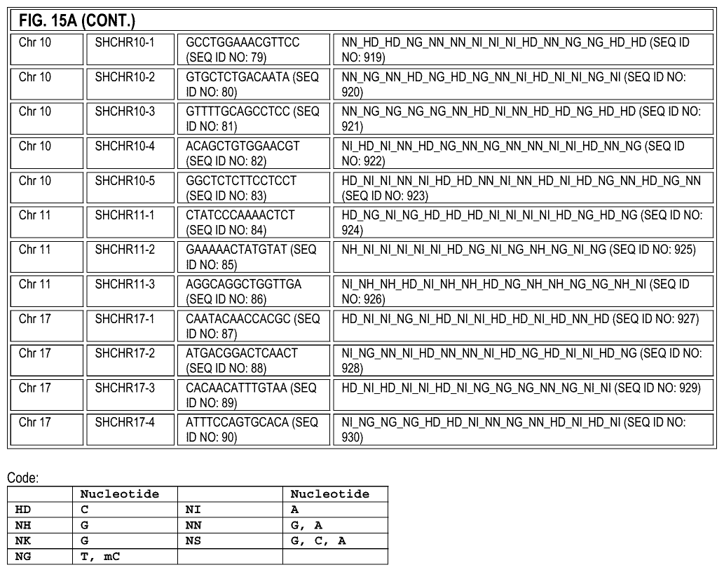

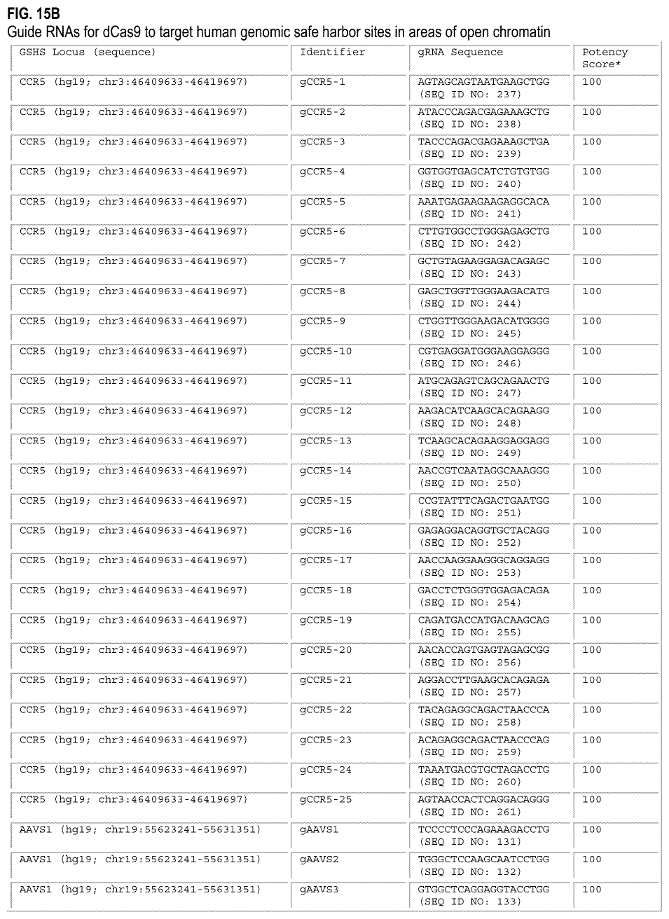

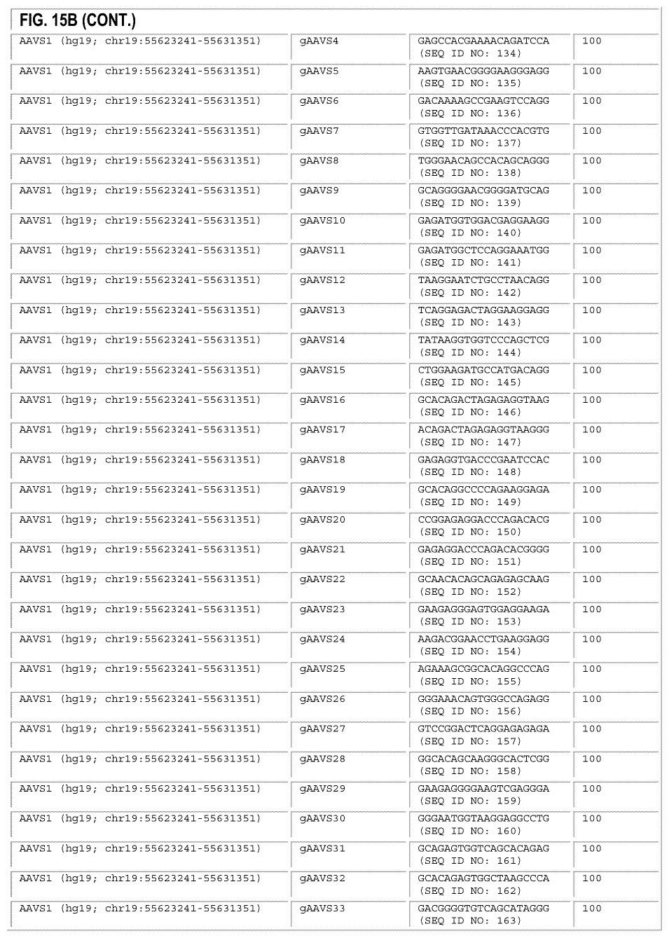

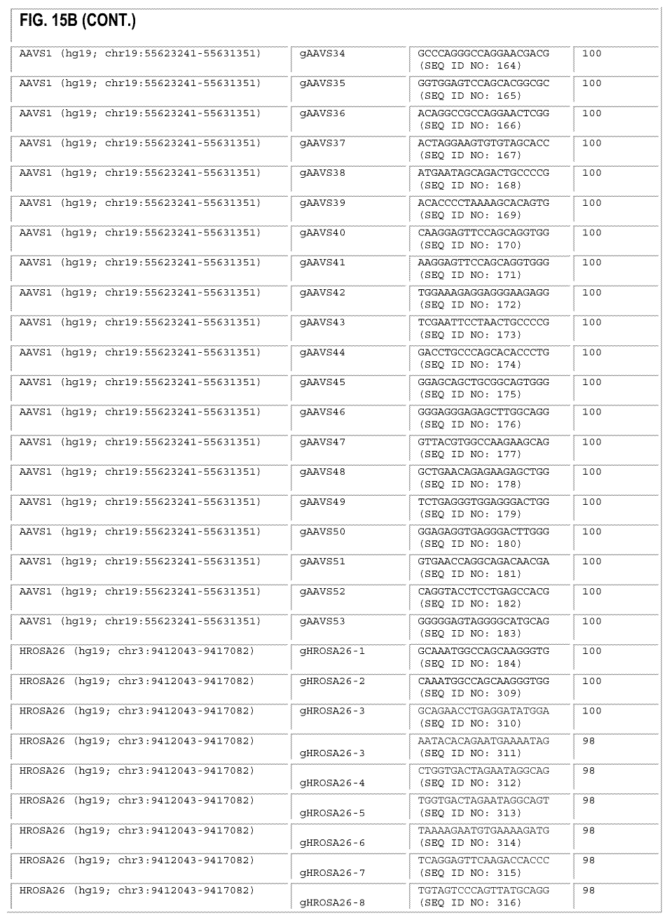

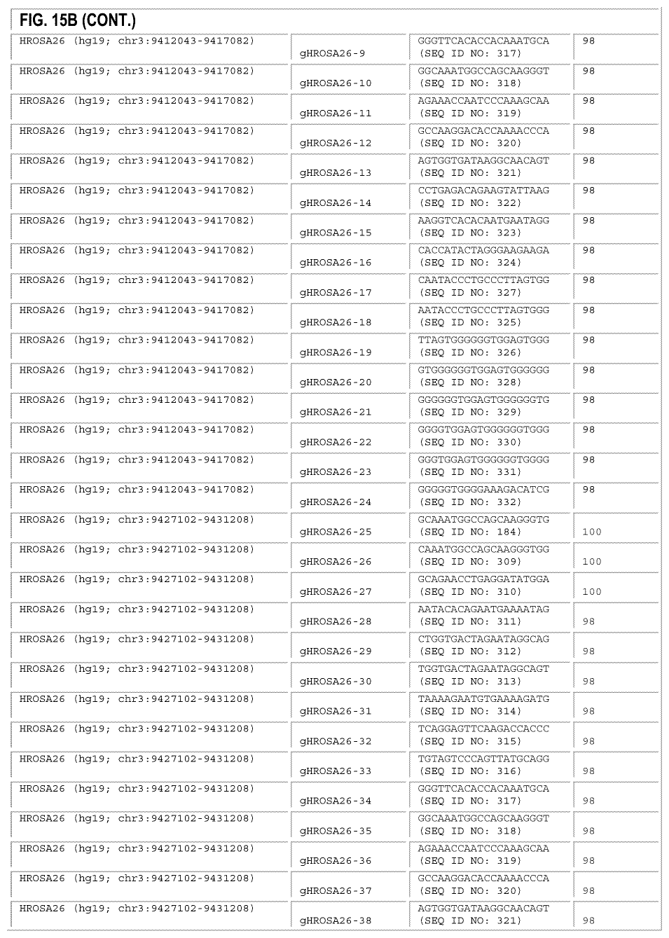

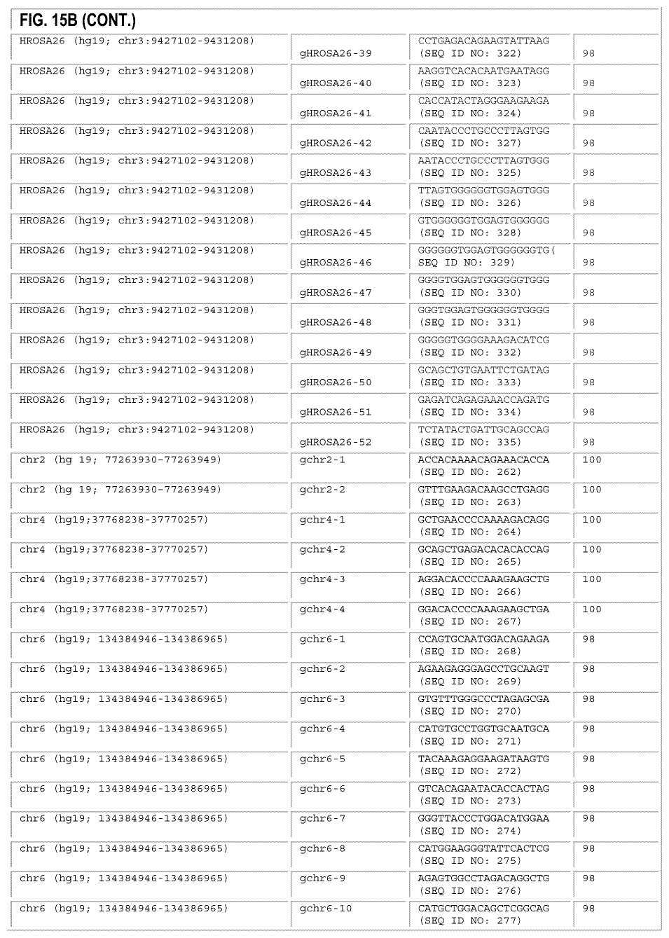

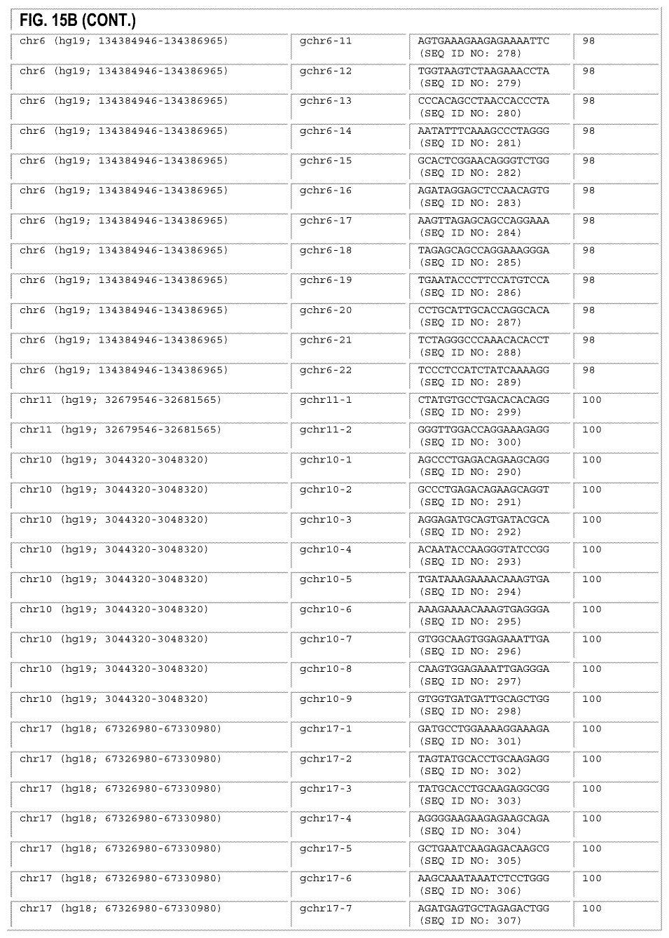

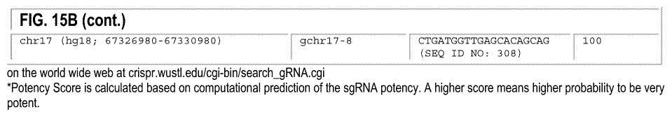

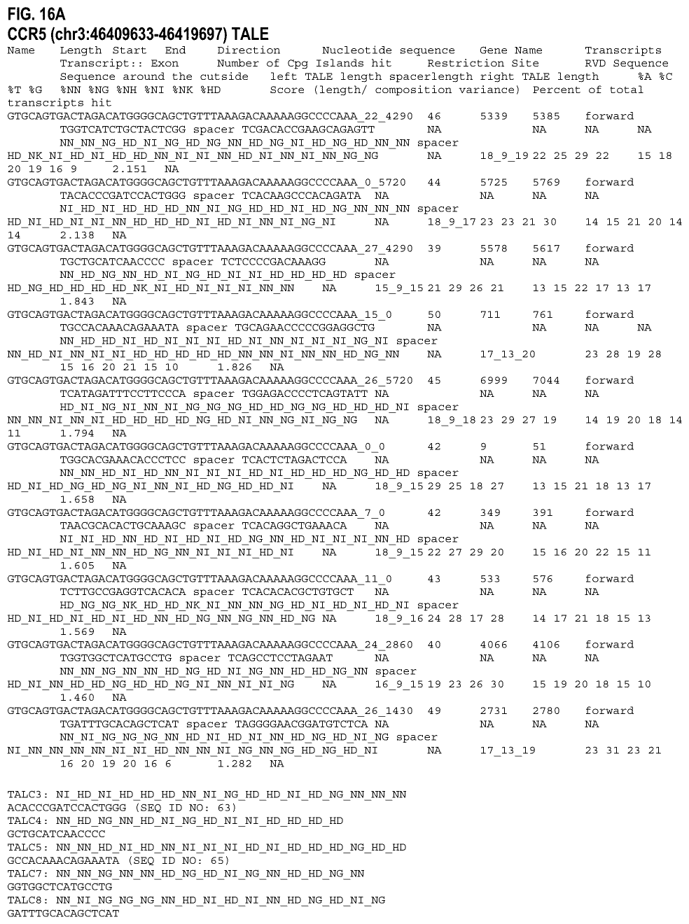

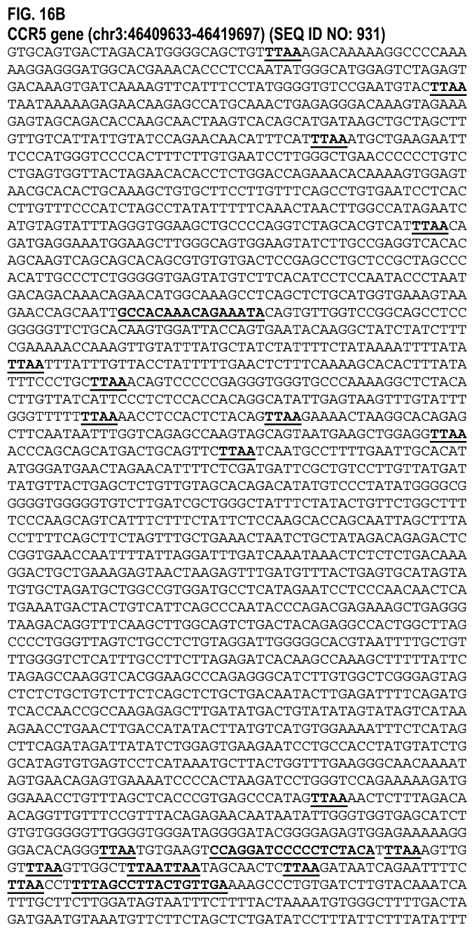

Accordingly, TALENs can be readily designed using a “protein-DNA code” that relates modular DNA-binding TALE repeat domains to individual bases in a target-binding site. See Joung et al. Nat Rev Mol Cell Biol. 2013; 14(1):49-55. doi:10.1038/nrm3486. FIG. 15A, for example, shows such code.

It has been demonstrated that TALENs can be used to target essentially any DNA sequence of interest in human cell. Miller et al. Nat Biotechnol. 2011; 29:143-148. Guidelines for selection of potential target sites and for use of particular TALE repeat domains (harboring NH residues at the hypervariable positions) for recognition of G bases have been proposed. See Streubel et al. Nat Biotechnol. 2012; 30:593-595.

In embodiments, the TALE DBD comprises one or more repeat sequences. In embodiments, the TALE DBD comprises about 14, or about 15, or about, 16, or about 17, or about 18, or about 18.5 repeat sequences. In embodiments, the TALE DBD repeat sequences comprise 33 or 34 amino acids. In embodiments, the TALE DBD repeat sequences comprise a repeat variable di-residue (RVD) at residue 12 or 13 of the 33 or 34 amino acids. In embodiments, the RVD recognizes one base pair in the nucleic acid molecule. In embodiments, the RVD recognizes a C residue in the nucleic acid molecule and is selected from HD, N(gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(gap), and IG. In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor, and human Rosa26 locus. In embodiments, the GSHS is an adeno-associated virus site 1 (AAVS1). In embodiments, the GSHS is a human Rosa26 locus. In embodiments, the GSHS is located on human chromosome 2, 4, 6, 10, 11, 17, 22, or X.

In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the targeting element comprises a Cas9 enzyme guide RNA complex. In embodiments, the Cas9 enzyme guide RNA complex comprises a nuclease-deficient dCas9 guide RNA complex. In embodiments, the targeting element comprises a Cas12 enzyme guide RNA complex. In embodiments, the targeting element comprises a nuclease-deficient dCas12 guide RNA complex, optionally dCas12j guide RNA complex or dCas12a guide RNA complex. In embodiments, the targeting element comprises a Cas12k enzyme guide RNA complex. In embodiments, the targeting element comprises a nuclease-deficient dCas12 guide RNA complex, optionally dCas12k guide RNA complex.

In embodiments, the targeting element comprises a Cas9 enzyme associated with a gRNA. In embodiments, the Cas9 enzyme associated with a gRNA comprises a catalytically inactive dCas9 associated with a gRNA.

In embodiments, the catalytically inactive dCas9 comprises at least about 90%, or at least about 95%, or at least about 96%, or at least about 97%, or at least about 98%, or at least about 99% identity to an amino acid sequence of SEQ ID NO: 21 or a nucleic acid comprising a nucleotide sequence of SEQ ID NO: 22 or a codon-optimized form thereof.

| SEQ ID NO: 21: Amino acid sequence of dead Cas9 protein | |
|---|---|
| (GENBANK ACC. No. MT882253.1) | |
| 1 | MDKKYSIGLA IGTNSVGWAV ITDEYKVPSK KFKVLGNTDR HSIKKNLIGA |
| 51 | LLFDSGETAE ATRLKRTARR RYTRRKNRIC YLQEIFSNEM AKVDDSFFHR |
| 101 | LEESFLVEED KKHERHPIFG NIVDEVAYHE KYPTIYHLRK KLVDSTDKAD |
| 151 | LRLIYLALAH MIKFRGHFLI EGDLNPDNSD VDKLFIQLVQ TYNQLFEENP |
| 201 | INASGVDAKA ILSARLSKSR RLENLIAQLP GEKKNGLFGN LIALSLGLTP |
| 251 | NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI |
| 351 | FFDQSKNGYA GYIDGGASQE EFYKFIKPIL EKMDGTEELL VKLNREDLLR |
| 401 | KQRTFDNGSI PHQIHLGELH AILRRQEDFY PFLKDNREKI EKILTFRIPY |
| 451 | YVGPLARGNS RFAWMTRKSE ETITPWNFEE VVDKGASAQS FIERMTNFDK |
| 501 | NLPNEKVLPK HSLLYEYFTV YNELTKVKYV TEGMRKPAFL SGEQKKAIVD |
| 551 | LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ |
| 651 | LKRRRYTGWG RLSRKLINGI RDKQSGKTIL DFLKSDGFAN RNFMQLIHDD |
| 701 | SLTFKEDIQK AQVSGQGDSL HEHIANLAGS PAIKKGILQT VKVVDELVKV |
| 751 | MGRHKPENIV IEMARENQTT QKGQKNSRER MKRIEEGIKE LGSQILKEHP |
| 801 | VENTQLQNEK LYLYYLQNGR DMYVDQELDI NRLSDYDVAA IVPQSFLKDD |
| 851 | SIDNKVLTRS DKARGKSDNV PSEEVVKKMK NYWRQLLNAK LITQRKFDNL |
| 901 | TKAERGGLSE LDKAGFIKRQ LVETRQITKH VAQILDSRMN TKYDENDKLI |
| 951 | REVKVITLKS KLVSDFRKDF QFYKVREINN YHHAHDAYLN AVVGTALIKK |
| 1001 | YPKLESEFVY GDYKVYDVRK MIAKSEQEIG KATAKYFFYS NIMNFFKTEI |
| 1051 | TLANGEIRKR PLIETNGETG EIVWDKGRDF ATVRKVLSMP QVNIVKKTEV |
| 1101 | QTGGFSKESI LPKRNSDKLI ARKKDWDPKK YGGFDSPTVA YSVLVVAKVE |
| 1151 | KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE |
| 1251 | DNEQKQLFVE QHKHYLDEII EQISEFSKRV ILADANLDKV LSAYNKHRDK |
| 1301 | PIREQAENII HLFTLTNLGA PAAFKYFDTT IDRKRYTSTK EVLDATLIHQ |
| 1351 | SITGLYETRI DLSQLGGDSR ADPKKKRKV |
| SEQ ID NO: 22: Nucleotide sequence of dead Cas9 protein | |
| (GENBANK ACC. NO. MT882253.1) | |
| 1 | ATGGACAAGA AGTACTCCAT TGGGCTCGCT ATCGGCACAA ACAGCGTCGG CTGGGCCGTC |
| 61 | ATTACGGACG AGTACAAGGT GCCGAGCAAA AAATTCAAAG TTCTGGGCAA TACCGATCGC |
| 121 | CACAGCATAA AGAAGAACCT CATTGGCGCC CTCCTGTTCG ACTCCGGGGA GACGGCCGAA |
| 181 | GCCACGCGGC TCAAAAGAAC AGCACGGCGC AGATATACCC GCAGAAAGAA TCGGATCTGC |
| 241 | TACCTGCAGG AGATCTTTAG TAATGAGATG GCTAAGGTGG ATGACTCTTT CTTCCATAGG |
| 301 | CTGGAGGAGT CCTTTTTGGT GGAGGAGGAT AAAAAGCACG AGCGCCACCC AATCTTTGGC |
| 361 | AATATCGTGG ACGAGGTGGC GTACCATGAA AAGTACCCAA CCATATATCA TCTGAGGAAG |
| 421 | AAGCTTGTAG ACAGTACTGA TAAGGCTGAC TTGCGGTTGA TCTATCTCGC GCTGGCGCAT |
| 481 | ATGATCAAAT TTCGGGGACA CTTCCTCATC GAGGGGGACC TGAACCCAGA CAACAGCGAT |
| 541 | GTCGACAAAC TCTTTATCCA ACTGGTTCAG ACTTACAATC AGCTTTTCGA AGAGAACCCG |
| 601 | ATCAACGCAT CCGGAGTTGA CGCCAAAGCA ATCCTGAGCG CTAGGCTGTC CAAATCCCGG |
| 661 | CGGCTCGAAA ACCTCATCGC ACAGCTCCCT GGGGAGAAGA AGAACGGCCT GTTTGGTAAT |
| 721 | CTTATCGCCC TGTCACTCGG GCTGACCCCC AACTTTAAAT CTAACTTCGA CCTGGCCGAA |
| 781 | GATGCCAAGC TTCAACTGAG CAAAGACACC TACGATGATG ATCTCGACAA TCTGCTGGCC |
| 841 | CAGATCGGCG ACCAGTACGC AGACCTTTTT TTGGCGGCAA AGAACCTGTC AGACGCCATT |
| 901 | CTGCTGAGTG ATATTCTGCG AGTGAACACG GAGATCACCA AAGCTCCGCT GAGCGCTAGT |
| 961 | ATGATCAAGC GCTATGATGA GCACCACCAA GACTTGACTT TGCTGAAGGC CCTTGTCAGA |
| 1021 | CAGCAACTGC CTGAGAAGTA CAAGGAAATT TTCTTCGATC AGTCTAAAAA TGGCTACGCC |
| 1081 | GGATACATTG ACGGCGGAGC AAGCCAGGAG GAATTTTACA AATTTATTAA GCCCATCTTG |
| 1141 | GAAAAAATGG ACGGCACCGA GGAGCTGCTG GTAAAGCTTA ACAGAGAAGA TCTGTTGCGC |
| 1201 | AAACAGCGCA CTTTCGACAA TGGAAGCATC CCCCACCAGA TTCACCTGGG CGAACTGCAC |
| 1261 | GCTATCCTCA GGCGGCAAGA GGATTTCTAC CCCTTTTTGA AAGATAACAG GGAAAAGATT |
| 1321 | GAGAAAATCC TCACATTTCG GATACCCTAC TATGTAGGCC CCCTCGCCCG GGGAAATTCC |
| 1381 | AGATTCGCGT GGATGACTCG CAAATCAGAA GAGACCATCA CTCCCTGGAA CTTCGAGGAA |
| 1441 | GTCGTGGATA AGGGGGCCTC TGCCCAGTCC TTCATCGAAA GGATGACTAA CTTTGATAAA |
| 1501 | AATCTGCCTA ACGAAAAGGT GCTTCCTAAA CACTCTCTGC TGTACGAGTA CTTCACAGTT |
| 1561 | TATAACGAGC TCACCAAGGT CAAATACGTC ACAGAAGGGA TGAGAAAGCC AGCATTCCTG |
| 1621 | TCTGGAGAGC AGAAGAAAGC TATCGTGGAC CTCCTCTTCA AGACGAACCG GAAAGTTACC |
| 1681 | GTGAAACAGC TCAAAGAAGA CTATTTCAAA AAGATTGAAT GTTTCGACTC TGTTGAAATC |
| 1741 | AGCGGAGTGG AGGATCGCTT CAACGCATCC CTGGGAACGT ATCACGATCT CCTGAAAATC |
| 1801 | ATTAAAGACA AGGACTTCCT GGACAATGAG GAGAACGAGG ACATTCTTGA GGACATTGTC |
| 1861 | CTCACCCTTA CGTTGTTTGA AGATAGGGAG ATGATTGAAG AACGCTTGAA AACTTACGCT |
| 1921 | CATCTCTTCG ACGACAAAGT CATGAAACAG CTCAAGAGGC GCCGATATAC AGGATGGGGG |
| 1981 | CGGCTGTCAA GAAAACTGAT CAATGGGATC CGAGACAAGC AGAGTGGAAA GACAATCCTG |
| 2041 | GATTTTCTTA AGTCCGATGG ATTTGCCAAC CGGAACTTCA TGCAGTTGAT CCATGATGAC |
| 2101 | TCTCTCACCT TTAAGGAGGA CATCCAGAAA GCACAAGTTT CTGGCCAGGG GGACAGTCTT |
| 2161 | CACGAGCACA TCGCTAATCT TGCAGGTAGC CCAGCTATCA AAAAGGGAAT ACTGCAGACC |
| 2221 | GTTAAGGTCG TGGATGAACT CGTCAAAGTA ATGGGAAGGC ATAAGCCCGA GAATATCGTT |
| 2281 | ATCGAGATGG CCCGAGAGAA CCAAACTACC CAGAAGGGAC AGAAGAACAG TAGGGAAAGG |
| 2341 | ATGAAGAGGA TTGAAGAGGG TATAAAAGAA CTGGGGTCCC AAATCCTTAA GGAACACCCA |
| 2401 | GTTGAAAACA CCCAGCTTCA GAATGAGAAG CTCTACCTGT ACTACCTGCA GAACGGCAGG |
| 2461 | GACATGTACG TGGATCAGGA ACTGGACATC AATCGGCTCT CCGACTACGA CGTGGCTGCT |
| 2521 | ATCGTGCCCC AGTCTTTTCT CAAAGATGAT TCTATTGATA ATAAAGTGTT GACAAGATCC |
| 2581 | GATAAAGCTA GAGGGAAGAG TGATAACGTC CCCTCAGAAG AAGTTGTCAA GAAAATGAAA |
| 2641 | AATTATTGGC GGCAGCTGCT GAACGCCAAA CTGATCACAC AACGGAAGTT CGATAATCTG |
| 2701 | ACTAAGGCTG AACGAGGTGG CCTGTCTGAG TTGGATAAAG CCGGCTTCAT CAAAAGGCAG |
| 2761 | CTTGTTGAGA CACGCCAGAT CACCAAGCAC GTGGCCCAAA TTCTCGATTC ACGCATGAAC |
| 2821 | ACCAAGTACG ATGAAAATGA CAAACTGATT CGAGAGGTGA AAGTTATTAC TCTGAAGTCT |
| 2881 | AAGCTGGTCT CAGATTTCAG AAAGGACTTT CAGTTTTATA AGGTGAGAGA GATCAACAAT |
| 2941 | TACCACCATG CGCATGATGC CTACCTGAAT GCAGTGGTAG GCACTGCACT TATCAAAAAA |
| 3001 | TATCCCAAGC TTGAATCTGA ATTTGTTTAC GGAGACTATA AAGTGTACGA TGTTAGGAAA |
| 3061 | ATGATCGCAA AGTCTGAGCA GGAAATAGGC AAGGCCACCG CTAAGTACTT CTTTTACAGC |
| 3121 | AATATTATGA ATTTTTTCAA GACCGAGATT ACACTGGCCA ATGGAGAGAT TCGGAAGCGA |
| 3181 | CCACTTATCG AAACAAACGG AGAAACAGGA GAAATCGTGT GGGACAAGGG TAGGGATTTC |
| 3241 | GCGACAGTCC GGAAGGTCCT GTCCATGCCG CAGGTGAACA TCGTTAAAAA GACCGAAGTA |
| 3301 | CAGACCGGAG GCTTCTCCAA GGAAAGTATC CTCCCGAAAA GGAACAGCGA CAAGCTGATC |
| 3361 | GCACGCAAAA AAGATTGGGA CCCCAAGAAA TACGGCGGAT TCGATTCTCC TACAGTCGCT |
| 3421 | TACAGTGTAC TGGTTGTGGC CAAAGTGGAG AAAGGGAAGT CTAAAAAACT CAAAAGCGTC |
| 3481 | AAGGAACTGC TGGGCATCAC AATCATGGAG CGATCAAGCT TCGAAAAAAA CCCCATCGAC |
| 3541 | TTTCTGGAGG CGAAAGGATA TAAAGAGGTC AAAAAAGACC TCATCATTAA GCTTCCCAAG |
| 3601 | TACTCTCTCT TTGAGCTTGA AAACGGCCGG AAACGAATGC TCGCTAGTGC GGGCGAGCTG |
| 3661 | CAGAAAGGTA ACGAGCTGGC ACTGCCCTCT AAATACGTTA ATTTCTTGTA TCTGGCCAGC |
| 3721 | CACTATGAAA AGCTCAAAGG GTCTCCCGAA GATAATGAGC AGAAGCAGCT GTTCGTGGAA |
| 3781 | CAACACAAAC ACTACCTTGA TGAGATCATC GAGCAAATAA GCGAATTCTC CAAAAGAGTG |
| 3841 | ATCCTCGCCG ACGCTAACCT CGATAAGGTG CTTTCTGCTT ACAATAAGCA CAGGGATAAG |
| 3901 | CCCATCAGGG AGCAGGCAGA AAACATTATC CACTTGTTTA CTCTGACCAA CTTGGGCGCG |
| 3961 | CCTGCAGCCT TCAAGTACTT CGACACCACC ATAGACAGAA AGCGGTACAC CTCTACAAAG |
| 4021 | GAGGTCCTGG ACGCCACACT GATTCATCAG TCAATTACGG GGCTCTATGA AACAAGAATC |
| 4081 | GACCTCTCTC AGCTCGGTGG AGACAGCAGG GCTGACCCCA AGAAGAAGAG GAAGGTG |

In embodiments, a targeting chimeric system or construct, having a DBD fused to a helper enzyme, directs binding of an enzyme capable of performing targeted genomic integration (e.g., without limitation, a helper enzyme) to a specific sequence (e.g., transcription activator-like effector proteins (TALE) repeat variable di-residues (RVD) or gRNA) near an enzyme recognition site. The enzyme is thus prevented from binding to random recognition sites. In embodiments, the targeting chimeric construct binds to human GSHS. In embodiments, dCas9 (i.e., deficient for nuclease activity) is programmed with gRNAs directed to bind at a desired sequence of DNA in GSHS.

In embodiments, TALEs described herein can physically sequester the enzyme such as, e.g., a helper enzyme, to GSHS and promote transposition to nearby TTAA (SEQ ID NO: 440) sequences in close proximity to the RVD TALE nucleotide sequences. GSHS in open chromatin sites are specifically targeted based on the predilection for helper enzymes to insert into open chromatin.

In embodiments, an enzyme capable of performing targeted genomic integration (e.g., without limitation, a recombinase, integrase, or a helper enzyme such as, without limitation, a mammalian helper enzyme) is linked to or fused with a TALE DNA binding domain (DBD) or a Cas-based gene-editing system, such as, e.g., Cas9 or a variant thereof.

In embodiments, the targeting element targets the enzyme to a locus of interest. In embodiments, the targeting element comprises CRISPR (Clustered Regularly Interspaced Short Palindromic Repeat) associated protein 9 (Cas9), or a variant thereof. A CRISPR/Cas9 tool only requires Cas9 nuclease for DNA cleavage and a single-guide RNA (sgRNA) for target specificity. See Jinek et al. (2012) Science 337, 816-821; Chylinski et al. (2014) Nucleic Acids Res 42, 6091-6105. The inactivated form of Cas9, which is a nuclease-deficient (or inactive, or “catalytically dead” Cas9, is typically denoted as “dCas9,” has no substantial nuclease activity. Qi, L. S. et al. (2013). Cell 152, 1173-1183. CRISPR/dCas9 binds precisely to specific genomic sequences through targeting of guide RNA (gRNA) sequences. See Dominguez et al., Nat Rev Mol Cell Biol. 2016; 17:5-15; Wang et al., Annu Rev Biochem. 2016; 85:227-64. dCas9 is utilized to edit gene expression when applied to the transcription binding site of a desired site and/or locus in a genome. When the dCas9 protein is coupled to guide RNA (gRNA) to create dCas9 guide RNA complex, dCas9 prevents the proliferation of repeating codons and DNA sequences that might be harmful to an organism's genome. Essentially, when multiple repeat codons are produced, it elicits a response, or recruits an abundance of dCas9 to combat the overproduction of those codons and results in the shut-down of transcription. Thus, dCas9 works synergistically with gRNA and directly affects the DNA polymerase II from continuing transcription.

In embodiments, the targeting element comprises a nuclease-deficient Cas enzyme guide RNA complex. In embodiments, the targeting element comprises a nuclease-deficient (or inactive, or “catalytically dead” Cas, e.g., Cas9, typically denoted as “dCas” or “dCas9”) guide RNA complex.

In embodiments, the dCas9/gRNA complex comprises a guide RNA selected from: GTTTAGCTCACCCGTGAGCC (SEQ ID NO: 91), CCCAATATTATTGTTCTCTG (SEQ ID NO: 92), GGGGTGGGATAGGGGATACG (SEQ ID NO: 93), GGATCCCCCTCTACATTTAA (SEQ ID NO: 94), GTGATCTTGTACAAATCATT (SEQ ID NO: 95), CTACACAGAATCTGTTAGAA (SEQ ID NO: 96), TAAGCTAGAGAATAGATCTC (SEQ ID NO: 97), and TCAATACACTTAATGATTTA (SEQ ID NO: 98), wherein the guide RNA directs the enzyme to a chemokine (C—C motif) receptor 5 (CCR5) gene.

In embodiments, the dCas9/gRNA complex comprises a guide RNA selected from:

(SEQ ID NO: 99)
CACCGGGAGCCACGAAAACAGATCC;
(SEQ ID NO: 100)
CACCGCGAAAACAGATCCAGGGACA;
(SEQ ID NO: 101)
CACCGAGATCCAGGGACACGGTGCT;
(SEQ ID NO: 102)
CACCGGACACGGTGCTAGGACAGTG;
(SEQ ID NO: 103)
CACCGGAAAATGACCCAACAGCCTC;
(SEQ ID NO: 104)
CACCGGCCTGGCCGGCCTGACCACT;
(SEQ ID NO: 105)
CACCGCTGAGCACTGAAGGCCTGGC;
(SEQ ID NO: 106)
CACCGTGGTTTCCACTGAGCACTGA;
(SEQ ID NO: 107)
CACCGGATAGCCAGGAGTCCTTTCG;
(SEQ ID NO: 108)
CACCGGCGCTTCCAGTGCTCAGACT;
(SEQ ID NO: 109)
CACCGCAGTGCTCAGACTAGGGAAG;
(SEQ ID NO: 110)
CACCGGCCCCTCCTCCTTCAGAGCC;
(SEQ ID NO: 111)
CACCGTCCTTCAGAGCCAGGAGTCC;
(SEQ ID NO: 112)
CACCGTGGTTTCCGAGCTTGACCCT;
(SEQ ID NO: 113)
CACCGCTGCAGAGTATCTGCTGGGG;
(SEQ ID NO: 114)
CACCGCGTTCCTGCAGAGTATCTGC;
(SEQ ID NO: 131)
TCCCCTCCCAGAAAGACCTG;
(SEQ ID NO: 132)
TGGGCTCCAAGCAATCCTGG;
(SEQ ID NO: 133)
GTGGCTCAGGAGGTACCTGG;
(SEQ ID NO: 134)
GAGCCACGAAAACAGATCCA;
(SEQ ID NO: 135)
AAGTGAACGGGGAAGGGAGG;
(SEQ ID NO: 136)
GACAAAAGCCGAAGTCCAGG;
(SEQ ID NO: 137)
GTGGTTGATAAACCCACGTG;
(SEQ ID NO: 138)
TGGGAACAGCCACAGCAGGG;
(SEQ ID NO: 139)
GCAGGGGAACGGGGATGCAG;
(SEQ ID NO: 140)
GAGATGGTGGACGAGGAAGG;
(SEQ ID NO: 141)
GAGATGGCTCCAGGAAATGG;
(SEQ ID NO: 142)
TAAGGAATCTGCCTAACAGG;
(SEQ ID NO: 143)
TCAGGAGACTAGGAAGGAGG;
(SEQ ID NO: 144)
TATAAGGTGGTCCCAGCTCG;
(SEQ ID NO: 145)
CTGGAAGATGCCATGACAGG;
(SEQ ID NO: 146)
GCACAGACTAGAGAGGTAAG;
(SEQ ID NO: 147)
ACAGACTAGAGAGGTAAGGG;
(SEQ ID NO: 148)
GAGAGGTGACCCGAATCCAC;
(SEQ ID NO: 149)
GCACAGGCCCCAGAAGGAGA;
(SEQ ID NO: 150)
CCGGAGAGGACCCAGACACG;
(SEQ ID NO: 151)
GAGAGGACCCAGACACGGGG;
(SEQ ID NO: 152)
GCAACACAGCAGAGAGCAAG;
(SEQ ID NO: 153)
GAAGAGGGAGTGGAGGAAGA;
(SEQ ID NO: 154)
AAGACGGAACCTGAAGGAGG;
(SEQ ID NO: 155)
AGAAAGCGGCACAGGCCCAG;
(SEQ ID NO: 156)
GGGAAACAGTGGGCCAGAGG;
(SEQ ID NO: 157)
GTCCGGACTCAGGAGAGAGA;
(SEQ ID NO: 158)
GGCACAGCAAGGGCACTCGG;
(SEQ ID NO: 159)
GAAGAGGGGAAGTCGAGGGA;
(SEQ ID NO: 160)
GGGAATGGTAAGGAGGCCTG;
(SEQ ID NO: 161)
GCAGAGTGGTCAGCACAGAG;
(SEQ ID NO: 162)
GCACAGAGTGGCTAAGCCCA;
(SEQ ID NO: 163)
GACGGGGTGTCAGCATAGGG;
(SEQ ID NO: 164)
GCCCAGGGCCAGGAACGACG;
(SEQ ID NO: 165)
GGTGGAGTCCAGCACGGCGC;
(SEQ ID NO: 166)
ACAGGCCGCCAGGAACTCGG;
(SEQ ID NO: 167)
ACTAGGAAGTGTGTAGCACC;
(SEQ ID NO: 168)
ATGAATAGCAGACTGCCCCG;
(SEQ ID NO: 169)
ACACCCCTAAAAGCACAGTG;
(SEQ ID NO: 170)
CAAGGAGTTCCAGCAGGTGG;
(SEQ ID NO: 171)
AAGGAGTTCCAGCAGGTGGG;
(SEQ ID NO: 172)
TGGAAAGAGGAGGGAAGAGG;
(SEQ ID NO: 173)
TCGAATTCCTAACTGCCCCG;
(SEQ ID NO: 174)
GACCTGCCCAGCACACCCTG;
(SEQ ID NO: 175)
GGAGCAGCTGCGGCAGTGGG;
(SEQ ID NO: 176)
GGGAGGGAGAGCTTGGCAGG;
(SEQ ID NO: 177)
GTTACGTGGCCAAGAAGCAG;
(SEQ ID NO: 178)
GCTGAACAGAGAAGAGCTGG;
(SEQ ID NO: 179)
TCTGAGGGTGGAGGGACTGG;
(SEQ ID NO: 180)
GGAGAGGTGAGGGACTTGGG;
(SEQ ID NO: 181)
GTGAACCAGGCAGACAACGA;
(SEQ ID NO: 182)
CAGGTACCTCCTGAGCCACG;
(SEQ ID NO: 183)
GGGGGAGTAGGGGCATGCAG;
(SEQ ID NO: 184)
GCAAATGGCCAGCAAGGGTG;
(SEQ ID NO: 309)
CAAATGGCCAGCAAGGGTGG;
(SEQ ID NO: 310)
GCAGAACCTGAGGATATGGA;
(SEQ ID NO: 311)
AATACACAGAATGAAAATAG;
(SEQ ID NO: 312)
CTGGTGACTAGAATAGGCAG;
(SEQ ID NO: 313)
TGGTGACTAGAATAGGCAGT;
(SEQ ID NO: 314)
TAAAAGAATGTGAAAAGATG;
(SEQ ID NO: 315)
TCAGGAGTTCAAGACCACCC;
(SEQ ID NO: 316)
TGTAGTCCCAGTTATGCAGG;
(SEQ ID NO: 317)
GGGTTCACACCACAAATGCA;
(SEQ ID NO: 318)
GGCAAATGGCCAGCAAGGGT;
(SEQ ID NO: 319)
AGAAACCAATCCCAAAGCAA;
(SEQ ID NO: 320)
GCCAAGGACACCAAAACCCA;
(SEQ ID NO: 321)
AGTGGTGATAAGGCAACAGT;
(SEQ ID NO: 322)
CCTGAGACAGAAGTATTAAG;
(SEQ ID NO: 323)
AAGGTCACACAATGAATAGG;
(SEQ ID NO: 324)
CACCATACTAGGGAAGAAGA;
(SEQ ID NO: 327)
CAATACCCTGCCCTTAGTGG;
(SEQ ID NO: 325)
AATACCCTGCCCTTAGTGGG;
(SEQ ID NO: 326)
TTAGTGGGGGGGGAGTGGG;
(SEQ ID NO: 328)
GTGGGGGGGGAGTGGGGGG;
(SEQ ID NO: 329)
GGGGGGTGGAGTGGGGGGTG;
(SEQ ID NO: 330)
GGGGTGGAGTGGGGGGTGGG;
(SEQ ID NO: 331)
GGGTGGAGTGGGGGGTGGGG;
(SEQ ID NO: 332)
GGGGGGGGGAAAGACATCG;
(SEQ ID NO: 333)
GCAGCTGTGAATTCTGATAG;
(SEQ ID NO: 334)
GAGATCAGAGAAACCAGATG;
(SEQ ID NO: 335)
TCTATACTGATTGCAGCCAG;
(SEQ ID NO: 185)
CACCGAATCGAGAAGCGACTCGACA;
(SEQ ID NO: 186)
CACCGGTCCCTGGGCGTTGCCCTGC;
(SEQ ID NO: 187)
CACCGCCCTGGGCGTTGCCCTGCAG;
(SEQ ID NO: 188)
CACCGCCGTGGGAAGATAAACTAAT;
(SEQ ID NO: 189)
CACCGTCCCCTGCAGGGCAACGCCC;
(SEQ ID NO: 190)
CACCGGTCGAGTCGCTTCTCGATTA;
(SEQ ID NO: 191)
CACCGCTGCTGCCTCCCGTCTTGTA;
(SEQ ID NO: 192)
CACCGGAGTGCCGCAATACCTTTAT;
(SEQ ID NO: 193)
CACCGACACTTTGGTGGTGCAGCAA;
(SEQ ID NO: 194)
CACCGTCTCAAATGGTATAAAACTC;
(SEQ ID NO: 195)
CACCGAATCCCGCCCATAATCGAGA;
(SEQ ID NO: 196)
CACCGTCCCGCCCATAATCGAGAAG;
(SEQ ID NO: 197)
CACCGCCCATAATCGAGAAGCGACT;
(SEQ ID NO: 198)
CACCGGAGAAGCGACTCGACATGGA;
(SEQ ID NO: 199)
CACCGGAAGCGACTCGACATGGAGG;
(SEQ ID NO: 200)
CACCGGCGACTCGACATGGAGGCGA;
(SEQ ID NO: 201)
AAACTGTCGAGTCGCTTCTCGATTC;
(SEQ ID NO: 202)
AAACGCAGGGCAACGCCCAGGGACC;
(SEQ ID NO: 203)
AAACCTGCAGGGCAACGCCCAGGGC;
(SEQ ID NO: 204)
AAACATTAGTTTATCTTCCCACGGC;
(SEQ ID NO: 205)
AAACGGGCGTTGCCCTGCAGGGGAC;
(SEQ ID NO: 206)
AAACTAATCGAGAAGCGACTCGACC;
(SEQ ID NO: 207)
AAACTACAAGACGGGAGGCAGCAGC;
(SEQ ID NO: 208)
AAACATAAAGGTATTGCGGCACTCC;
(SEQ ID NO: 209)
AAACTTGCTGCACCACCAAAGTGTC;
(SEQ ID NO: 210)
AAACGAGTTTTATACCATTTGAGAC;
(SEQ ID NO: 211)
AAACTCTCGATTATGGGGGGGATTC;
(SEQ ID NO: 212)
AAACCTTCTCGATTATGGGGGGGAC;
(SEQ ID NO: 213)
AAACAGTCGCTTCTCGATTATGGGC;
(SEQ ID NO: 214)
AAACTCCATGTCGAGTCGCTTCTCC;
(SEQ ID NO: 215)
AAACCCTCCATGTCGAGTCGCTTCC;
(SEQ ID NO: 216)
AAACTCGCCTCCATGTCGAGTCGCC;
(SEQ ID NO: 217)
CACCGACAGGGTTAATGTGAAGTCC;
(SEQ ID NO: 218)
CACCGTCCCCCTCTACATTTAAAGT;
(SEQ ID NO: 219)
CACCGCATTTAAAGTTGGTTTAAGT;
(SEQ ID NO: 220)
CACCGTTAGAAAATATAAAGAATAA;
(SEQ ID NO: 221)
CACCGTAAATGCTTACTGGTTTGAA;
(SEQ ID NO: 222)
CACCGTCCTGGGTCCAGAAAAAGAT;
(SEQ ID NO: 223)
CACCGTTGGGTGGTGAGCATCTGTG;
(SEQ ID NO: 224)
CACCGCGGGGAGAGTGGAGAAAAAG;
(SEQ ID NO: 225)
CACCGGTTAAAACTCTTTAGACAAC;
(SEQ ID NO: 226)
CACCGGAAAATCCCCACTAAGATCC;
(SEQ ID NO: 227)
AAACGGACTTCACATTAACCCTGTC;
(SEQ ID NO: 228)
AAACACTTTAAATGTAGAGGGGGAC;
(SEQ ID NO: 229)
AAACACTTAAACCAACTTTAAATGC;
(SEQ ID NO: 230)
AAACTTATTCTTTATATTTTCTAAC;
(SEQ ID NO: 231)
AAACTTCAAACCAGTAAGCATTTAC;
(SEQ ID NO: 232)
AAACATCTTTTTCTGGACCCAGGAC;
(SEQ ID NO: 233)
AAACCACAGATGCTCACCACCCAAC;
(SEQ ID NO: 234)
AAACCTTTTTCTCCACTCTCCCCGC;
(SEQ ID NO: 235)
AAACGTTGTCTAAAGAGTTTTAACC;
(SEQ ID NO: 236)
AAACGGATCTTAGTGGGGATTTTCC;
(SEQ ID NO: 237)
AGTAGCAGTAATGAAGCTGG;
(SEQ ID NO: 238)
ATACCCAGACGAGAAAGCTG;
(SEQ ID NO: 239)
TACCCAGACGAGAAAGCTGA;
(SEQ ID NO: 240)
GGTGGTGAGCATCTGTGTGG;
(SEQ ID NO: 241)
AAATGAGAAGAAGAGGCACA;
(SEQ ID NO: 242)
CTTGTGGCCTGGGAGAGCTG;
(SEQ ID NO: 243)
GCTGTAGAAGGAGACAGAGC;
(SEQ ID NO: 244)
GAGCTGGTTGGGAAGACATG;
(SEQ ID NO: 245)
CTGGTTGGGAAGACATGGGG;
(SEQ ID NO: 246)
CGTGAGGATGGGAAGGAGGG;
(SEQ ID NO: 247)
ATGCAGAGTCAGCAGAACTG;
(SEQ ID NO: 248)
AAGACATCAAGCACAGAAGG;
(SEQ ID NO: 249)
TCAAGCACAGAAGGAGGAGG;
(SEQ ID NO: 250)
AACCGTCAATAGGCAAAGGG;
(SEQ ID NO: 251)
CCGTATTTCAGACTGAATGG;
(SEQ ID NO: 252)
GAGAGGACAGGTGCTACAGG;
(SEQ ID NO: 253)
AACCAAGGAAGGGCAGGAGG;
(SEQ ID NO: 254)
GACCTCTGGGTGGAGACAGA;
(SEQ ID NO: 255)
CAGATGACCATGACAAGCAG;
(SEQ ID NO: 256)
AACACCAGTGAGTAGAGCGG;
(SEQ ID NO: 257)
AGGACCTTGAAGCACAGAGA;
(SEQ ID NO: 258)
TACAGAGGCAGACTAACCCA;
(SEQ ID NO: 259)
ACAGAGGCAGACTAACCCAG;
(SEQ ID NO: 260)
TAAATGACGTGCTAGACCTG;
(SEQ ID NO: 261)
AGTAACCACTCAGGACAGGG;
(SEQ ID NO: 262)
ACCACAAAACAGAAACACCA;
(SEQ ID NO: 263)
GTTTGAAGACAAGCCTGAGG;
(SEQ ID NO: 264)
GCTGAACCCCAAAAGACAGG;
(SEQ ID NO: 265)
GCAGCTGAGACACACACCAG;
(SEQ ID NO: 266)
AGGACACCCCAAAGAAGCTG;
(SEQ ID NO: 267)
GGACACCCCAAAGAAGCTGA;
(SEQ ID NO: 268)
CCAGTGCAATGGACAGAAGA;
(SEQ ID NO: 269)
AGAAGAGGGAGCCTGCAAGT;
(SEQ ID NO: 270)
GTGTTTGGGCCCTAGAGCGA;
(SEQ ID NO: 271)
CATGTGCCTGGTGCAATGCA;
(SEQ ID NO: 272)
TACAAAGAGGAAGATAAGTG;
(SEQ ID NO: 273)
GTCACAGAATACACCACTAG;
(SEQ ID NO: 274)
GGGTTACCCTGGACATGGAA;
(SEQ ID NO: 275)
CATGGAAGGGTATTCACTCG;
(SEQ ID NO: 276)
AGAGTGGCCTAGACAGGCTG;
(SEQ ID NO: 277)
CATGCTGGACAGCTCGGCAG;
(SEQ ID NO: 278)
AGTGAAAGAAGAGAAAATTC;
(SEQ ID NO: 279)
TGGTAAGTCTAAGAAACCTA;
(SEQ ID NO: 280)
CCCACAGCCTAACCACCCTA;
(SEQ ID NO: 281)
AATATTTCAAAGCCCTAGGG;
(SEQ ID NO: 282)
GCACTCGGAACAGGGTCTGG;
(SEQ ID NO: 283)
AGATAGGAGCTCCAACAGTG;
(SEQ ID NO: 284)
AAGTTAGAGCAGCCAGGAAA;
(SEQ ID NO: 285)
TAGAGCAGCCAGGAAAGGGA;
(SEQ ID NO: 286)
TGAATACCCTTCCATGTCCA;
(SEQ ID NO: 287)
CCTGCATTGCACCAGGCACA;
(SEQ ID NO: 288)
TCTAGGGCCCAAACACACCT;
(SEQ ID NO: 289)
TCCCTCCATCTATCAAAAGG;
(SEQ ID NO: 290)
AGCCCTGAGACAGAAGCAGG;
(SEQ ID NO: 291)
GCCCTGAGACAGAAGCAGGT;
(SEQ ID NO: 292)
AGGAGATGCAGTGATACGCA;
(SEQ ID NO: 293)
ACAATACCAAGGGTATCCGG;
(SEQ ID NO: 294)
TGATAAAGAAAACAAAGTGA;
(SEQ ID NO: 295)
AAAGAAAACAAAGTGAGGGA;
(SEQ ID NO: 296)
GTGGCAAGTGGAGAAATTGA;
(SEQ ID NO: 297)
CAAGTGGAGAAATTGAGGGA;
(SEQ ID NO: 298)
GTGGTGATGATTGCAGCTGG;
(SEQ ID NO: 299)
CTATGTGCCTGACACACAGG;
(SEQ ID NO: 300)
GGGTTGGACCAGGAAAGAGG;
(SEQ ID NO: 301)
GATGCCTGGAAAAGGAAAGA;
(SEQ ID NO: 302)
TAGTATGCACCTGCAAGAGG;
(SEQ ID NO: 303)
TATGCACCTGCAAGAGGGGG;
(SEQ ID NO: 304)
AGGGGAAGAAGAGAAGCAGA;
(SEQ ID NO: 305)
GCTGAATCAAGAGACAAGCG;
(SEQ ID NO: 306)
AAGCAAATAAATCTCCTGGG;
(SEQ ID NO: 307)
AGATGAGTGCTAGAGACTGG;
and
(SEQ ID NO: 308)
CTGATGGTTGAGCACAGCAG.

In embodiments, the guide RNAs are: AATCGAGAAGCGACTCGACA (SEQ ID NO: 425), and tgccctgcaggggagtgagc (SEQ ID NO: 426). In embodiments, the guide RNAs are gaagcgactcgacatggagg (SEQ ID NO: 427) and cctgcaggggagtgagcagc (SEQ ID NO: 428).

In embodiments, guide RNAs (gRNAs) for targeting human genomic safe harbor sites using any of the gRNA-based targeting elements, e.g., without limitation dCas, in areas of open chromatin are as shown in TABLE 3A-3F.

In embodiments, guide RNAs (gRNAs) for targeting human genomic safe harbor sites using any of the gRNA-based targeting elements, e.g., without limitation dCas, in areas of open chromatin are as shown in TABLE 3A.

TABLE 3A

| GSHS | Identifier | Sequence |
|---|---|---|
| AAVS1 | 14F | ggagccacgaaaacagatcc |
| | | (SEQ ID NO: 99) |
| AAVS1 | 15F | cgaaaacagatccagggaca (SEQ ID NO: 100) |
| AAVS1 | 16F | agatccagggacacggtgct (SEQ ID NO: 101) |
| AAVS1 | 17F | gacacggtgctaggacagtg (SEQ ID NO: 102) |
| AAVS1 | 18F | gaaaatgacccaacagcctc (SEQ ID NO: 103) |
| AAVS1 | 19F | gcctggccggcctgaccact (SEQ ID NO: 104) |
| AAVS1 | 20F | ctgagcactgaaggcctggc (SEQ ID NO: 105) |
| AAVS1 | 21F | tggtttccactgagcactga (SEQ ID NO: 106) |
| AAVS1 | 22F | gatagccaggagtcctttcg (SEQ ID NO: 107) |
| AAVS1 | 23F | gcgcttccagtgctcagact (SEQ ID NO: 108) |
| AAVS1 | 24F | cagtgctcagactagggaag (SEQ ID NO: 109) |
| AAVS1 | 25F | gcccctcctccttcagagcc (SEQ ID NO: 110) |
| AAVS1 | 26F | tccttcagagccaggagtcc (SEQ ID NO: 111) |
| AAVS1 | 27F | tggtttccgagcttgaccct (SEQ ID NO: 112) |
| AAVS1 | 28F | ctgcagagtatctgctgggg (SEQ ID NO: 113) |
| AAVS1 | 29F | cgttcctgcagagtatctgc (SEQ ID NO: 114) |
| AAVS1 | AAVS1 | TCCCCTCCCAGAAAGACCTG (SEQ ID NO: 131) |
| AAVS1 | gAAVS2 | TGGGCTCCAAGCAATCCTGG (SEQ ID NO: 132) |
| AAVS1 | gAAVS3 | GTGGCTCAGGAGGTACCTGG (SEQ ID NO: 133) |
| AAVS1 | gAAVS4 | GAGCCACGAAAACAGATCCA (SEQ ID NO: 134) |
| AAVS1 | gAAVS5 | AAGTGAACGGGGAAGGGAGG (SEQ ID NO: 135) |
| AAVS1 | gAAVS6 | GACAAAAGCCGAAGTCCAGG (SEQ ID NO: 136) |
| AAVS1 | gAAVS7 | GTGGTTGATAAACCCACGTG (SEQ ID NO: 137) |
| AAVS1 | gAAVS8 | TGGGAACAGCCACAGCAGGG (SEQ ID NO: 138) |
| AAVS1 | gAAVS9 | GCAGGGGAACGGGGATGCAG (SEQ ID NO: 139) |
| AAVS1 | gAAVS10 | GAGATGGTGGACGAGGAAGG (SEQ ID NO: 140) |
| AAVS1 | gAAVS11 | GAGATGGCTCCAGGAAATGG (SEQ ID NO: 141) |
| AAVS1 | gAAVS12 | TAAGGAATCTGCCTAACAGG (SEQ ID NO: 142) |
| AAVS1 | gAAVS13 | TCAGGAGACTAGGAAGGAGG (SEQ ID NO: 143) |
| AAVS1 | gAAVS14 | TATAAGGTGGTCCCAGCTCG (SEQ ID NO: 144) |
| AAVS1 | gAAVS15 | CTGGAAGATGCCATGACAGG (SEQ ID NO: 145) |
| AAVS1 | gAAVS16 | GCACAGACTAGAGAGGTAAG (SEQ ID NO: 146) |
| AAVS1 | gAAVS17 | ACAGACTAGAGAGGTAAGGG (SEQ ID NO: 147) |
| AAVS1 | gAAVS18 | GAGAGGTGACCCGAATCCAC (SEQ ID NO: 148) |
| AAVS1 | gAAVS19 | GCACAGGCCCCAGAAGGAGA (SEQ ID NO: 149) |
| AAVS1 | gAAVS20 | CCGGAGAGGACCCAGACACG (SEQ ID NO: 150) |
| AAVS1 | gAAVS21 | GAGAGGACCCAGACACGGGG (SEQ ID NO: 151) |
| AAVS1 | gAAVS22 | GCAACACAGCAGAGAGCAAG (SEQ ID NO: 152) |
| AAVS1 | gAAVS23 | GAAGAGGGAGTGGAGGAAGA (SEQ ID NO: 153) |
| AAVS1 | gAAVS24 | AAGACGGAACCTGAAGGAGG (SEQ ID NO: 154) |
| AAVS1 | gAAVS25 | AGAAAGCGGCACAGGCCCAG (SEQ ID NO: 155) |
| AAVS1 | gAAVS26 | GGGAAACAGTGGGCCAGAGG (SEQ ID NO: 156) |
| AAVS1 | gAAVS27 | GTCCGGACTCAGGAGAGAGA (SEQ ID NO: 157) |
| AAVS1 | gAAVS28 | GGCACAGCAAGGGCACTCGG (SEQ ID NO: 158) |
| AAVS1 | gAAVS29 | GAAGAGGGGAAGTCGAGGGA (SEQ ID NO: 159) |
| AAVS1 | gAAVS30 | GGGAATGGTAAGGAGGCCTG (SEQ ID NO: 160) |
| AAVS1 | gAAVS31 | GCAGAGTGGTCAGCACAGAG (SEQ ID NO: 161) |
| AAVS1 | gAAVS32 | GCACAGAGTGGCTAAGCCCA (SEQ ID NO: 162) |
| AAVS1 | gAAVS33 | GACGGGGTGTCAGCATAGGG (SEQ ID NO: 163) |
| AAVS1 | gAAVS34 | GCCCAGGGCCAGGAACGACG (SEQ ID NO: 164) |
| AAVS1 | gAAVS35 | GGTGGAGTCCAGCACGGCGC (SEQ ID NO: 165) |
| AAVS1 | gAAVS36 | ACAGGCCGCCAGGAACTCGG (SEQ ID NO: 166) |
| AAVS1 | gAAVS37 | ACTAGGAAGTGTGTAGCACC (SEQ ID NO: 167) |
| AAVS1 | gAAVS38 | ATGAATAGCAGACTGCCCCG (SEQ ID NO: 168) |
| AAVS1 | gAAVS39 | ACACCCCTAAAAGCACAGTG (SEQ ID NO: 169) |
| AAVS1 | gAAVS40 | CAAGGAGTTCCAGCAGGTGG (SEQ ID NO: 170) |
| AAVS1 | gAAVS41 | AAGGAGTTCCAGCAGGTGGG (SEQ ID NO: 171) |
| AAVS1 | gAAVS42 | TGGAAAGAGGAGGGAAGAGG (SEQ ID NO: 172) |
| AAVS1 | gAAVS43 | TCGAATTCCTAACTGCCCCG (SEQ ID NO: 173) |
| AAVS1 | gAAVS44 | GACCTGCCCAGCACACCCTG (SEQ ID NO: 174) |
| AAVS1 | gAAVS45 | GGAGCAGCTGCGGCAGTGGG (SEQ ID NO: 175) |
| AAVS1 | gAAVS46 | GGGAGGGAGAGCTTGGCAGG (SEQ ID NO: 176) |
| AAVS1 | gAAVS47 | GTTACGTGGCCAAGAAGCAG (SEQ ID NO: 177) |
| AAVS1 | gAAVS48 | GCTGAACAGAGAAGAGCTGG (SEQ ID NO: 178) |
| AAVS1 | gAAVS49 | TCTGAGGGTGGAGGGACTGG (SEQ ID NO: 179) |
| AAVS1 | gAAVS50 | GGAGAGGTGAGGGACTTGGG (SEQ ID NO: 180) |
| AAVS1 | gAAVS51 | GTGAACCAGGCAGACAACGA (SEQ ID NO: 181) |
| AAVS1 | gAAVS52 | CAGGTACCTCCTGAGCCACG (SEQ ID NO: 182) |
| AAVS1 | gAAVS53 | GGGGGAGTAGGGGCATGCAG (SEQ ID NO: 183) |
| hROSA26 | gHROSA26-1 | GCAAATGGCCAGCAAGGGTG (SEQ ID NO: 184) |
| hROSA26 | gHROSA26-2 | CAAATGGCCAGCAAGGGTGG (SEQ ID NO: 309) |
| hROSA26 | gHROSA26-3 | GCAGAACCTGAGGATATGGA (SEQ ID NO: 310) |
| hROSA26 | gHROSA26-3 | AATACACAGAATGAAAATAG (SEQ ID NO: 311) |
| hROSA26 | gHROSA26-4 | CTGGTGACTAGAATAGGCAG (SEQ ID NO: 312) |
| hROSA26 | gHROSA26-5 | TGGTGACTAGAATAGGCAGT (SEQ ID NO: 313) |
| hROSA26 | gHROSA26-6 | TAAAAGAATGTGAAAAGATG (SEQ ID NO: 314) |
| hROSA26 | gHROSA26-7 | TCAGGAGTTCAAGACCACCC (SEQ ID NO: 315) |
| hROSA26 | gHROSA26-8 | TGTAGTCCCAGTTATGCAGG (SEQ ID NO: 316) |
| hROSA26 | gHROSA26-9 | GGGTTCACACCACAAATGCA (SEQ ID NO: 317) |
| hROSA26 | gHROSA26-10 | GGCAAATGGCCAGCAAGGGT (SEQ ID NO: 318) |
| hROSA26 | gHROSA26-11 | AGAAACCAATCCCAAAGCAA (SEQ ID NO: 319) |
| hROSA26 | gHROSA26-12 | GCCAAGGACACCAAAACCCA (SEQ ID NO: 320) |
| hROSA26 | gHROSA26-13 | AGTGGTGATAAGGCAACAGT (SEQ ID NO: 321) |
| hROSA26 | gHROSA26-14 | CCTGAGACAGAAGTATTAAG (SEQ ID NO: 322) |
| hROSA26 | gHROSA26-15 | AAGGTCACACAATGAATAGG (SEQ ID NO: 323) |
| hROSA26 | gHROSA26-16 | CACCATACTAGGGAAGAAGA (SEQ ID NO: 324) |
| hROSA26 | gHROSA26-17 | CAATACCCTGCCCTTAGTGG (SEQ ID NO: 327) |
| hROSA26 | gHROSA26-18 | AATACCCTGCCCTTAGTGGG (SEQ ID NO: 325) |
| hROSA26 | gHROSA26-19 | TTAGTGGGGGGTGGAGTGGG (SEQ ID NO: 326) |
| hROSA26 | gHROSA26-20 | GTGGGGGGTGGAGTGGGGGG (SEQ ID NO: 328) |
| hROSA26 | gHROSA26-21 | GGGGGGTGGAGTGGGGGGTG (SEQ ID NO: 329) |
| hROSA26 | gHROSA26-22 | GGGGTGGAGTGGGGGGTGGG (SEQ ID NO: 330) |
| hROSA26 | gHROSA26-23 | GGGTGGAGTGGGGGGTGGGG (SEQ ID NO: 331) |
| hROSA26 | gHROSA26-24 | GGGGTGGGGGAAAGACATCG (SEQ ID NO: 332) |
| hROSA26 | gHROSA26-25 | GCAAATGGCCAGCAAGGGTG (SEQ ID NO: 184) |
| hROSA26 | gHROSA26-26 | CAAATGGCCAGCAAGGGTGG (SEQ ID NO: 309) |
| hROSA26 | gHROSA26-27 | GCAGAACCTGAGGATATGGA (SEQ ID NO: 310) |
| hROSA26 | gHROSA26-28 | AATACACAGAATGAAAATAG (SEQ ID NO: 311) |
| hROSA26 | gHROSA26-29 | CTGGTGACTAGAATAGGCAG (SEQ ID NO: 312) |
| hROSA26 | gHROSA26-30 | TGGTGACTAGAATAGGCAGT (SEQ ID NO: 313) |
| hROSA26 | gHROSA26-31 | TAAAAGAATGTGAAAAGATG (SEQ ID NO: 314) |
| hROSA26 | gHROSA26-32 | TCAGGAGTTCAAGACCACCC (SEQ ID NO: 315) |
| hROSA26 | gHROSA26-33 | TGTAGTCCCAGTTATGCAGG (SEQ ID NO: 316) |
| hROSA26 | gHROSA26-34 | GGGTTCACACCACAAATGCA (SEQ ID NO: 317) |
| hROSA26 | gHROSA26-35 | GGCAAATGGCCAGCAAGGGT (SEQ ID NO: 318) |
| hROSA26 | gHROSA26-36 | AGAAACCAATCCCAAAGCAA (SEQ ID NO: 319) |
| hROSA26 | gHROSA26-37 | GCCAAGGACACCAAAACCCA (SEQ ID NO: 320) |
| hROSA26 | gHROSA26-38 | AGTGGTGATAAGGCAACAGT (SEQ ID NO: 321) |
| hROSA26 | gHROSA26-39 | CCTGAGACAGAAGTATTAAG (SEQ ID NO: 322) |
| hROSA26 | gHROSA26-40 | AAGGTCACACAATGAATAGG (SEQ ID NO: 323) |
| hROSA26 | gHROSA26-41 | CACCATACTAGGGAAGAAGA (SEQ ID NO: 324) |
| hROSA26 | gHROSA26-42 | CAATACCCTGCCCTTAGTGG (SEQ ID NO: 327) |
| hROSA26 | gHROSA26-43 | AATACCCTGCCCTTAGTGGG (SEQ ID NO: 325) |
| hROSA26 | gHROSA26-44 | TTAGTGGGGGGTGGAGTGGG (SEQ ID NO: 326) |
| hROSA26 | gHROSA26-45 | GTGGGGGGTGGAGTGGGGGG (SEQ ID NO: 328) |
| hROSA26 | gHROSA26-46 | GGGGGGTGGAGTGGGGGGTG (SEQ ID NO: 329) |
| hROSA26 | gHROSA26-47 | GGGGTGGAGTGGGGGGTGGG (SEQ ID NO: 330) |
| hROSA26 | gHROSA26-48 | GGGTGGAGTGGGGGGTGGGG (SEQ ID NO: 331) |
| hROSA26 | gHROSA26-49 | GGGGGTGGGGAAAGACATCG (SEQ ID NO: 332) |
| hROSA26 | gHROSA26-50 | GCAGCTGTGAATTCTGATAG (SEQ ID NO: 333) |
| hROSA26 | gHROSA26-51 | GAGATCAGAGAAACCAGATG (SEQ ID NO: 334) |
| hROSA26 | gHROSA26-52 | TCTATACTGATTGCAGCCAG (SEQ ID NO: 335) |
| hROSA26 | gHROSA26-1 | GCAAATGGCCAGCAAGGGTG (SEQ ID NO: 184) |
| hROSA26 | 44F | CACCGAATCGAGAAGCGACTCGACA (SEQ ID NO: 185) |
| hROSA26 | 45F | CACCGGTCCCTGGGCGTTGCCCTGC (SEQ ID NO: 186) |
| hROSA26 | 46F | CACCGCCCTGGGCGTTGCCCTGCAG (SEQ ID NO: 187) |
| hROSA26 | 1nF | CACCGCCGTGGGAAGATAAACTAAT (SEQ ID NO: 188) |
| hROSA26 | 2nF | CACCGTCCCCTGCAGGGCAACGCCC (SEQ ID NO: 189) |
| hROSA26 | 3nF | CACCGGTCGAGTCGCTTCTCGATTA (SEQ ID NO: 190) |
| hROSA26 | 4nF | CACCGCTGCTGCCTCCCGTCTTGTA (SEQ ID NO: 191) |
| hROSA26 | 5nF | CACCGGAGTGCCGCAATACCTTTAT (SEQ ID NO: 192) |
| hROSA26 | 6nF | CACCGACACTTTGGTGGTGCAGCAA (SEQ ID NO: 193) |
| hROSA26 | 7nF | CACCGTCTCAAATGGTATAAAACTC (SEQ ID NO: 194) |
| hROSA26 | 8nF | CACCGCCGTGGGAAGATAAACTAAT (SEQ ID NO: 188) |
| hROSA26 | 9F | CACCGAATCCCGCCCATAATCGAGA (SEQ ID NO: 195) |
| hROSA26 | 10F | CACCGTCCCGCCCATAATCGAGAAG (SEQ ID NO: 196) |
| hROSA26 | 11F | CACCGCCCATAATCGAGAAGCGACT (SEQ ID NO: 197) |
| hROSA26 | 12F | CACCGGAGAAGCGACTCGACATGGA (SEQ ID NO: 198) |
| hROSA26 | 13F | CACCGGAAGCGACTCGACATGGAGG (SEQ ID NO: 199) |
| hROSA26 | 14F | CACCGGCGACTCGACATGGAGGCGA (SEQ ID NO: 200) |
| hROSA26 | 44F | AAACTGTCGAGTCGCTTCTCGATTC (SEQ ID NO: 201) |
| hROSA26 | 45F | AAACGCAGGGCAACGCCCAGGGACC (SEQ ID NO: 202) |
| hROSA26 | 46F | AAACCTGCAGGGCAACGCCCAGGGC (SEQ ID NO: 203) |
| hROSA26 | 1nR | AAACATTAGTTTATCTTCCCACGGC (SEQ ID NO: 204) |
| hROSA26 | 2nR | AAACGGGCGTTGCCCTGCAGGGGAC (SEQ ID NO: 205) |
| hROSA26 | 3nR | AAACTAATCGAGAAGCGACTCGACC (SEQ ID NO: 206) |
| hROSA26 | 4nR | AAACTACAAGACGGGAGGCAGCAGC (SEQ ID NO: 207) |
| hROSA26 | 5nR | AAACATAAAGGTATTGCGGCACTCC (SEQ ID NO: 208) |
| hROSA26 | 6nR | AAACTTGCTGCACCACCAAAGTGTC (SEQ ID NO: 209) |
| hROSA26 | 7nR | AAACGAGTTTTATACCATTTGAGAC (SEQ ID NO: 210) |
| hROSA26 | 8nR | AAACATTAGTTTATCTTCCCACGGC (SEQ ID NO: 204) |
| hROSA26 | 9R | AAACTCTCGATTATGGGCGGGATTC (SEQ ID NO: 211) |
| hROSA26 | 10R | AAACCTTCTCGATTATGGGCGGGAC (SEQ ID NO: 212) |
| hROSA26 | 11R | AAACAGTCGCTTCTCGATTATGGGC (SEQ ID NO: 213) |
| hROSA26 | 12R | AAACTCCATGTCGAGTCGCTTCTCC (SEQ ID NO: 214) |
| hROSA26 | 13R | AAACCCTCCATGTCGAGTCGCTTCC (SEQ ID NO: 215) |
| hROSA26 | 14R | AAACTCGCCTCCATGTCGAGTCGCC (SEQ ID NO: 216) |
| CCR5 | 1F | CACCGACAGGGTTAATGTGAAGTCC (SEQ ID NO: 217) |
| CCR5 | 2F | CACCGTCCCCCTCTACATTTAAAGT (SEQ ID NO: 218) |
| CCR5 | 3F | CACCGCATTTAAAGTTGGTTTAAGT (SEQ ID NO: 219) |
| CCR5 | 4F | CACCGTTAGAAAATATAAAGAATAA (SEQ ID NO: 220) |
| CCR5 | 5 | CACCGTAAATGCTTACTGGTTTGAA (SEQ ID NO: 221) |
| CCR5 | 6F | CACCGTCCTGGGTCCAGAAAAAGAT (SEQ ID NO: 222) |
| CCR5 | 7F | CACCGTTGGGTGGTGAGCATCTGTG (SEQ ID NO: 223) |
| CCR5 | 8F | CACCGCGGGGAGAGTGGAGAAAAAG (SEQ ID NO: 224) |
| CCR5 | 9F | CACCGGTTAAAACTCTTTAGACAAC (SEQ ID NO: 225) |
| CCR5 | 10F | CACCGGAAAATCCCCACTAAGATCC (SEQ ID NO: 226) |
| CCR5 | 1R | AAACGGACTTCACATTAACCCTGTC (SEQ ID NO: 227) |
| CCR5 | 2R | AAACACTTTAAATGTAGAGGGGGAC (SEQ ID NO: 228) |
| CCR5 | 3R | AAACACTTAAACCAACTTTAAATGC (SEQ ID NO: 229) |
| CCR5 | 4R | AAACTTATTCTTTATATTTTCTAAC (SEQ ID NO: 230) |
| CCR5 | 5R | AAACTTCAAACCAGTAAGCATTTAC (SEQ ID NO: 231) |
| CCR5 | 6R | AAACATCTTTTTCTGGACCCAGGAC (SEQ ID NO: 232) |
| CCR5 | 7R | AAACCACAGATGCTCACCACCCAAC (SEQ ID NO: 233) |
| CCR5 | 8R | AAACCTTTTTCTCCACTCTCCCCGC (SEQ ID NO: 234) |
| CCR5 | 9R | AAACGTTGTCTAAAGAGTTTTAACC (SEQ ID NO: 235) |
| CCR5 | 10R | AAACGGATCTTAGTGGGGATTTTCC (SEQ ID NO: 236) |
| CCR5 | gCCR5-1 | AGTAGCAGTAATGAAGCTGG (SEQ ID NO: 237) |
| CCR5 | gCCR5-2 | ATACCCAGACGAGAAAGCTG (SEQ ID NO: 238) |
| CCR5 | gCCR5-3 | TACCCAGACGAGAAAGCTGA (SEQ ID NO: 239) |
| CCR5 | gCCR5-4 | GGTGGTGAGCATCTGTGTGG (SEQ ID NO: 240) |
| CCR5 | gCCR5-5 | AAATGAGAAGAAGAGGCACA (SEQ ID NO: 241) |
| CCR5 | gCCR5-6 | CTTGTGGCCTGGGAGAGCTG (SEQ ID NO: 242) |
| CCR5 | gCCR5-7 | GCTGTAGAAGGAGACAGAGC (SEQ ID NO: 243) |
| CCR5 | gCCR5-8 | GAGCTGGTTGGGAAGACATG (SEQ ID NO: 244) |
| CCR5 | gCCR5-9 | CTGGTTGGGAAGACATGGGG (SEQ ID NO: 245) |
| CCR5 | gCCR5-10 | CGTGAGGATGGGAAGGAGGG (SEQ ID NO: 246) |
| CCR5 | gCCR5-11 | ATGCAGAGTCAGCAGAACTG (SEQ ID NO: 247) |
| CCR5 | gCCR5-12 | AAGACATCAAGCACAGAAGG (SEQ ID NO: 248) |
| CCR5 | gCCR5-13 | TCAAGCACAGAAGGAGGAGG (SEQ ID NO: 249) |
| CCR5 | gCCR5-14 | AACCGTCAATAGGCAAAGGG (SEQ ID NO: 250) |
| CCR5 | gCCR5-15 | CCGTATTTCAGACTGAATGG (SEQ ID NO: 251) |
| CCR5 | gCCR5-16 | GAGAGGACAGGTGCTACAGG (SEQ ID NO: 252) |
| CCR5 | gCCR5-17 | AACCAAGGAAGGGCAGGAGG (SEQ ID NO: 253) |
| CCR5 | gCCR5-18 | GACCTCTGGGTGGAGACAGA (SEQ ID NO: 254) |
| CCR5 | gCCR5-19 | CAGATGACCATGACAAGCAG (SEQ ID NO: 255) |
| CCR5 | gCCR5-20 | AACACCAGTGAGTAGAGCGG (SEQ ID NO: 256) |
| CCR5 | gCCR5-21 | AGGACCTTGAAGCACAGAGA (SEQ ID NO: 257) |
| CCR5 | gCCR5-22 | TACAGAGGCAGACTAACCCA (SEQ ID NO: 258) |
| CCR5 | gCCR5-23 | ACAGAGGCAGACTAACCCAG (SEQ ID NO: 259) |
| CCR5 | gCCR5-24 | TAAATGACGTGCTAGACCTG (SEQ ID NO: 260) |
| CCR5 | gCCR5-25 | AGTAACCACTCAGGACAGGG (SEQ ID NO: 261) |
| chr2 | gchr2-1 | ACCACAAAACAGAAACACCA (SEQ ID NO: 262) |
| chr2 | gchr2-2 | GTTTGAAGACAAGCCTGAGG (SEQ ID NO: 263) |
| chr4 | gchr4-1 | GCTGAACCCCAAAAGACAGG (SEQ ID NO: 264) |
| chr4 | gchr4-2 | GCAGCTGAGACACACACCAG (SEQ ID NO: 265) |
| chr4 | gchr4-3 | AGGACACCCCAAAGAAGCTG (SEQ ID NO: 266) |
| chr4 | gchr4-4 | GGACACCCCAAAGAAGCTGA (SEQ ID NO: 267) |
| chr6 | gchr6-1 | CCAGTGCAATGGACAGAAGA (SEQ ID NO: 268) |
| chr6 | gchr6-2 | AGAAGAGGGAGCCTGCAAGT (SEQ ID NO: 269) |
| chr6 | gchr6-3 | GTGTTTGGGCCCTAGAGCGA (SEQ ID NO: 270) |
| chr6 | gchr6-4 | CATGTGCCTGGTGCAATGCA (SEQ ID NO: 271) |
| chr6 | gchr6-5 | TACAAAGAGGAAGATAAGTG (SEQ ID NO: 272) |
| chr6 | gchr6-6 | GTCACAGAATACACCACTAG (SEQ ID NO: 273) |
| chr6 | gchr6-7 | GGGTTACCCTGGACATGGAA (SEQ ID NO: 274) |
| chr6 | gchr6-8 | CATGGAAGGGTATTCACTCG (SEQ ID NO: 275) |
| chr6 | gchr6-9 | AGAGTGGCCTAGACAGGCTG (SEQ ID NO: 276) |
| chr6 | gchr6-10 | CATGCTGGACAGCTCGGCAG (SEQ ID NO: 277) |
| chr6 | gchr6-11 | AGTGAAAGAAGAGAAAATTC (SEQ ID NO: 278) |
| chr6 | gchr6-12 | TGGTAAGTCTAAGAAACCTA (SEQ ID NO: 279) |
| chr6 | gchr6-13 | CCCACAGCCTAACCACCCTA (SEQ ID NO: 280) |
| chr6 | gchr6-14 | AATATTTCAAAGCCCTAGGG (SEQ ID NO: 281) |
| chr6 | gchr6-15 | GCACTCGGAACAGGGTCTGG (SEQ ID NO: 282) |
| chr6 | gchr6-16 | AGATAGGAGCTCCAACAGTG (SEQ ID NO: 283) |
| chr6 | gchr6-17 | AAGTTAGAGCAGCCAGGAAA (SEQ ID NO: 284) |
| chr6 | gchr6-18 | TAGAGCAGCCAGGAAAGGGA (SEQ ID NO: 285) |
| chr6 | gchr6-19 | TGAATACCCTTCCATGTCCA (SEQ ID NO: 286) |
| chr6 | gchr6-20 | CCTGCATTGCACCAGGCACA (SEQ ID NO: 287) |
| chr6 | gchr6-21 | TCTAGGGCCCAAACACACCT (SEQ ID NO: 288) |
| chr6 | gchr6-22 | TCCCTCCATCTATCAAAAGG (SEQ ID NO: 289) |
| chr10 | gchr10-1 | AGCCCTGAGACAGAAGCAGG (SEQ ID NO: 290) |
| chr10 | gchr10-2 | GCCCTGAGACAGAAGCAGGT (SEQ ID NO: 291) |
| chr10 | gchr10-3 | AGGAGATGCAGTGATACGCA (SEQ ID NO: 292) |
| chr10 | gchr10-4 | ACAATACCAAGGGTATCCGG (SEQ ID NO: 293) |
| chr10 | gchr10-5 | TGATAAAGAAAACAAAGTGA (SEQ ID NO: 294) |
| chr10 | gchr10-6 | AAAGAAAACAAAGTGAGGGA (SEQ ID NO: 295) |
| chr10 | gchr10-7 | GTGGCAAGTGGAGAAATTGA (SEQ ID NO: 296) |
| chr10 | gchr10-8 | CAAGTGGAGAAATTGAGGGA (SEQ ID NO: 297) |
| chr10 | gchr10-9 | GTGGTGATGATTGCAGCTGG (SEQ ID NO: 298) |
| chr11 | gchr11-1 | CTATGTGCCTGACACACAGG (SEQ ID NO: 299) |
| chr11 | gchr11-2 | GGGTTGGACCAGGAAAGAGG (SEQ ID NO: 300) |
| chr17 | gchr17-1 | GATGCCTGGAAAAGGAAAGA (SEQ ID NO: 301) |
| chr17 | gchr17-2 | TAGTATGCACCTGCAAGAGG (SEQ ID NO: 302) |
| chr17 | gchr17-3 | TATGCACCTGCAAGAGGCGG (SEQ ID NO: 303) |
| chr17 | gchr17-4 | AGGGGAAGAAGAGAAGCAGA (SEQ ID NO: 304) |
| chr17 | gchr17-5 | GCTGAATCAAGAGACAAGCG (SEQ ID NO: 305) |
| chr17 | gchr17-6 | AAGCAAATAAATCTCCTGGG (SEQ ID NO: 306) |
| chr17 | gchr17-7 | AGATGAGTGCTAGAGACTGG (SEQ ID NO: 307) |
| chr17 | gchr17-8 | CTGATGGTTGAGCACAGCAG (SEQ ID NO: 308) |

In embodiments, gRNAs for targeting human genomic safe harbor sites using any of the gRNA-based targeting elements, e.g., without limitation dCas, to the TTAA site in hROSA26 (e.g., hg38 chr3:9 396,133-9,396,305) are shown in TABLE 3B.

TABLE 3B

| HROSA26 | |
|---|---|
| GUIDE NO. | DNA SEQUENCE |
| GUIDE 44 | AATCGAGAAGCGACTCGACA (SEQ ID NO: |
| | 425) |
| GUIDE 45-C | GTCCCTGGGCGTTGCCCTGC (SEQ ID NO: |
| | 441) |
| GUIDE 46-C | CCCTGGGCGTTGCCCTGCAG (SEQ ID NO: |
| | 442) |
| SPG GUIDE1-C | GAGTGAGCAGCTGTAAGATT (SEQ ID NO: |
| | 443) |
| SPG GUIDE2-C | CAGGGGAGTGAGCAGCTGTA (SEQ ID NO: |
| | 444) |
| SPG GUIDE3-C | CCTGCAGGGGAGTGAGCAGC (SEQ ID NO: |
| | 428) |
| SPG GUIDE4-C | TGCCCTGCAGGGGAGTGAGC (SEQ ID NO: |
| | 426) |
| SPG GUIDE5-C | CGTTGCCCTGCAGGGGAGTG (SEQ ID NO: |
| | 445) |
| SPG GUIDE6-C | TGGGCGTTGCCCTGCAGGGG (SEQ ID NO: |
| | 446) |
| SPG GUIDE7-C | TTGGTCCCTGGGCGTTGCCC (SEQ ID NO: |
| | 447) |
| SPG GUIDE8 | AAGAATCCCGCCCATAATCG (SEQ ID NO: |
| | 448) |
| SPG GUIDE9 | AATCCCGCCCATAATCGAGA (SEQ ID NO: |
| | 449) |
| SPG GUIDE10 | TCCCGCCCATAATCGAGAAG (SEQ ID NO: |
| | 450) |
| SPG GUIDE11 | CCCATAATCGAGAAGCGACT (SEQ ID NO: |
| | 451) |
| SPG GUIDE12 | GAGAAGCGACTCGACATGGA (SEQ ID NO: |
| | 452) |
| SPG GUIDE13 | GAAGCGACTCGACATGGAGG (SEQ ID NO: |
| | 427) |
| SPG GUIDE14 | GCGACTCGACATGGAGGCGA (SEQ ID NO: |
| | 453) |
| GUIDE N1 | CCGTGGGAAGATAAACTAAT (SEQ ID NO: |
| | 454) |
| GUIDE N2 | TCCCCTGCAGGGCAACGCCC (SEQ ID NO: |
| | 455) |
| GUIDE N3-C | GTCGAGTCGCTTCTCGATTA (SEQ ID NO: |
| | 456) |
| GUIDE O12 | CGACACCAACTCTAGTCCGT (SEQ ID NO: |
| | 457) |
| GUIDE O13 | CAGCTGCTCACTCCCCTGCA (SEQ ID NO: |
| | 458) |
| GUIDE O14-C | AGTCGCTTCTCGATTATGGG (SEQ ID NO: |
| | 459) |

In embodiments, gRNAs for targeting human genomic safe harbor sites using any of the gRNA-based targeting elements, e.g., without limitation dCas, to the AAVS1 (e.g., hg38 chr19:55,112,851-55,113,324) are shown in TABLE 3C.

TABLE 3C

| AAVS1 | |
|---|---|
| GUIDE NO. | DNA SEQUENCE |
| AAV GUIDE 12 | ACCCTTGGAAGGACCTGGCTGGG (SEQ ID NO: |
| | 460) |
| AAV GUIDE 13c | TCCGAGCTTGACCCTTGGAA (SEQ ID NO: |
| | 461) |
| AAV GUIDE 14 | GGAGCCACGAAAACAGATCCAGG (SEQ ID NO: |
| | 462) |
| AAV GUIDE 14c | TGGTTTCCGAGCTTGACCCT (SEQ ID NO: |
| | 112) |
| AAV GUIDE 16 | AGATCCAGGGACACGGTGCTAGG (SEQ ID NO: |
| | 463) |
| AAV GUIDE 17 | GACACGGTGCTAGGACAGTGGGG (SEQ ID NO: |
| | 464) |
| AAV GUIDE 18 | GAAAATGACCCAACAGCCTCTGG (SEQ ID NO: |
| | 465) |
| AAV GUIDE 19 | GCCTGGCCGGCCTGACCACTGGG (SEQ ID NO: |
| | 466) |
| AAV GUIDE 20 | CTGAGCACTGAAGGCCTGGCCGG (SEQ ID NO: |
| | 467) |
| AAV GUIDE 21 | TGGTTTCCACTGAGCACTGAAGG (SEQ ID NO: |
| | 468) |
| AAV GUIDE 22 | GGTGCTTTCCTGAGGACCGATAG (SEQ ID NO: |
| | 469) |
| AAV GUIDE 23 | GCGCTTCCAGTGCTCAGACTAGG (SEQ ID NO: |
| | 470) |
| AAV GUIDE 24 | CAGTGCTCAGACTAGGGAAGAGG (SEQ ID NO: |
| | 471) |
| AAV GUIDE 25 | GCCCCTCCTCCTTCAGAGCCAGG (SEQ ID NO: |
| | 472) |
| AAV GUIDE 26 | TCCTTCAGAGCCAGGAGTCCTGG (SEQ ID NO: |
| | 473) |
| AAV GUIDE 27 | CCAAGGGTCAAGCTCGGAAACCA (SEQ ID NO: |
| | 474) |
| AAV GUIDE 28 | CTGCAGAGTATCTGCTGGGGTGG (SEQ ID NO: |
| | 475) |
| AAV GUIDE 29 | CGTTCCTGCAGAGTATCTGCTGG (SEQ ID NO: |
| | 476) |
| AAV GUIDE 30c | GTGGGGAAAATGACCCAACA (SEQ ID NO: |
| | 477) |
| AAV GUIDE 31 | GAAGGCCTGGCCGGCCTGAC (SEQ ID NO: |
| | 478) |
| AAV GUIDE 32c | ACTCCTGGCTCTGAAGGAGG (SEQ ID NO: |
| | 479) |
| AAV GUIDE 33c | GGGCTGGGGGCCAGGACTCC (SEQ ID NO: |
| | 480) |
| AAV GUIDE 34 | GTCCTTCCAAGGGTCAAGCT (SEQ ID NO: |
| | 481) |
| AAV GUIDE 35 | TCAAGCTCGGAAACCACCCC (SEQ ID NO: |
| | 482) |

In embodiments, gRNAs for targeting human genomic safe harbor sites using any of the gRNA-based targeting elements, e.g., without limitation dCas, to Chromosome 4 (e.g., hg38 chr4:30,793,534-30,875,476 or hg38 chr4:30,793,533-30,793,537 (9677); chr4:30,875,472-30,875,476 (8948)) are shown in TABLE 3D.

TABLE 3D

| CHR4 | |
|---|---|
| GUIDE NO. | DNA SEQUENCE |
| Guide C4-1 | ATTGTCTTCACTAAACCCGTTGG (SEQ ID NO: |
| | 483) |
| Guide C4-2 | TAAACCCGTTGGGAATACAATGG (SEQ ID NO: |
| | 484) |
| Guide C4-3 | TTGTCTTCACTAAACCCGTTGGG (SEQ ID NO: |
| | 485) |
| Guide C4-4 | TGATTCATAGGAGTCTATTAAGG (SEQ ID NO: |
| | 486) |
| Guide C4-5 | TTACATATGCTTCGAGTTTGTGG (SEQ ID NO: |
| | 487) |
| Guide C4-6 | ACTCTTAAGGTAGGACTAATTGG (SEQ ID NO: |
| | 488) |
| Guide C4-7 | TATGTGTGCAATAGCGTTAAAGG (SEQ ID NO: |
| | 489) |
| Guide C4-8 | CGTTGGGAATACAATGGCTTAGG (SEQ ID NO: |
| | 490) |
| Guide C4-9 | TCACAATGGAACTCTGCCTTTGG (SEQ ID NO: |
| | 491) |
| Guide C4-10 | GACCACAAATCAATGCCCAAAGG (SEQ ID NO: |
| | 492) |
| Guide C4-11 | CTAAGCCATTGTATTCCCAACGG (SEQ ID NO: |
| | 493) |
| Guide C4-12 | AGCATTCTGGAGTGTCACAATGG (SEQ ID NO: |
| | 494) |
| Guide C4-13 | CAATAGCCCACTTTAATACTAGG (SEQ ID NO: |
| | 495) |
| Guide C4-14 | CTTTATCCAAGTGAATCCTTTGG (SEQ ID NO: |
| | 496) |
| Guide C4-15 | GGCATTGATTTGTGGTCATTTGG (SEQ ID NO: |
| | 497) |
| Guide C4-16 | TAAGCCATTGTATTCCCAACGGG (SEQ ID NO: |
| | 498) |
| Guide C4-17 | AATACAATCACTCTTAAGGTAGG (SEQ ID NO: |
| | 499) |
| Guide C4-18 | GAAGTACCTTTCACTATTTTGGG (SEQ ID NO: |
| | 500) |
| Guide C4-19 | CAAGCAACAAATGACTTCTAAGG (SEQ ID NO: |
| | 501) |
| Guide C4-20 | TTTGAATACAATCACTCTTAAGG (SEQ ID NO: |
| | 502) |
| Guide C4A1 | ACAAACGGACTACGTAAACTTGG (SEQ ID NO: |
| | 503) |
| Guide C4A2 | ACAAGATGTGAACACGACGATGG (SEQ ID NO: |
| | 504) |
| Guide C4A3 | GTTGCACCGTTGATTCCTTCAGG (SEQ ID NO: |
| | 505) |
| Guide C4A4 | AGTAATATTGAATTAGGGCGTGG (SEQ ID NO: |
| | 506) |
| Guide C4A5 | CCTGATGTTGGCTCGACATTAGG (SEQ ID NO: |
| | 507) |
| Guide C4A6 | CTTTGTTGGGTCTTAGCTTAAGG (SEQ ID NO: |
| | 508) |
| Guide C4A7 | TCGGAACAGCTCCTTCCTGAAGG (SEQ ID NO: |
| | 509) |
| Guide C4A8 | AGTAGTTTCTGAGGTCATGTTGG (SEQ ID NO: |
| | 510) |
| Guide C4A9 | CTTGAAAATACGATGATGTGAGG (SEQ ID NO: |
| | 511) |
| Guide C4A10 | GCATTAATCTAGAGAGAGGGAGG (SEQ ID NO: |
| | 512) |
| Guide C4A11 | GGGTCATGTTAGAATTCATGTGG (SEQ ID NO: |
| | 513) |
| Guide C4A12 | TGATGCATTAATCTAGAGAGAGG (SEQ ID NO: |
| | 514) |
| Guide C4A13 | ACATCATCGTATTTTCAAGTTGG (SEQ ID NO: |
| | 515) |
| Guide C4A14 | CTAGCTGACAAACATGTGAGTGG (SEQ ID NO: |
| | 516) |
| Guide C4A15 | AACATGACCCAAGTGAGTCCAGG (SEQ ID NO: |
| | 517) |
| Guide C4A16 | GATTCCGTATTTGCTTTGTTGGG (SEQ ID NO: |
| | 518) |
| Guide C4A17 | TACGATGATGTGAGGAAATAAGG (SEQ ID NO: |
| | 519) |
| Guide C4A18 | GTAATATGTCTAAGTACTGATGG (SEQ ID NO: |
| | 520) |
| Guide C4A19 | GTAAAGTGAGCTGGTTCATTAGG (SEQ ID NO: |
| | 521) |
| Guide C4A20 | ACTAGAGTCCTTAAGAAGGGGGG (SEQ ID NO: |
| | 522) |

In embodiments, gRNAs for targeting human genomic safe harbor sites using any of the gRNA-based targeting elements, e.g., without limitation dCas, to Chromosome 22 (e.g., hg38 chr22:35,370,000-35,380,000 or hg38 chr22:35,373,912-35,373,916 (861); chr22:35,377,843-35,377,847 (1153)) are shown in TABLE 3E.

TABLE 3E

| CHR22 | |
|---|---|
| GUIDE NO. | DNA SEQUENCE |
| Guide C22-1 | ATAACACGTGAGCCGTCCTAAGG (SEQ ID NO: |
| | 523) |
| Guide C22-2 | GGAAGACTTTTCTCTATACGAGG (SEQ ID NO: |
| | 524) |
| Guide C22-3 | GCATTCCTTTCATCCATGGCAGG (SEQ ID NO: |
| | 525) |
| Guide C22-4 | GACATATGGTTATAAAAATCAGG (SEQ ID NO: |
| | 526) |
| Guide C22-5 | GGAGTGCAGTCCCTGACATATGG (SEQ ID NO: |
| | 527) |
| Guide C22-6 | GTGGGTTAGGGTGGTTAACTGGG (SEQ ID NO: |
| | 528) |
| Guide C22-7 | AGGTGCAAAAAGGTTGCTGTGGG (SEQ ID NO: |
| | 529) |
| Guide C22-8 | CGTGACAAGGCAAAGTGGCGTGG (SEQ ID NO: |
| | 530) |
| Guide C22-9 | GAAGGACTGCCCCTGACGTCAGG (SEQ ID NO: |
| | 531) |
| Guide C22-10 | CTGCCCCTGACGTCAGGAGTTGG (SEQ ID NO: |
| | 532) |
| Guide C22-11 | TGTGGGTTAGGGTGGTTAACTGG (SEQ ID NO: |
| | 533) |
| Guide C22-12 | ACCCTTTTAGAGTTTTCTGCTGG (SEQ ID NO: |
| | 534) |
| Guide C22-13 | AACTTCCTGCCATGGATGAAAGG (SEQ ID NO: |
| | 535) |
| Guide C22-14 | GCAAAAAGGTTGCTGTGGGTTGG (SEQ ID NO: |
| | 536) |
| Guide C22-15 | AATTTGGGGGTAGATAGGCATGG (SEQ ID NO: |
| | 537) |
| Guide C22-16 | AGAAAACTCTAAAAGGGTATAGG (SEQ ID NO: |
| | 538) |
| Guide C22-17 | ATTAGCATTCCTTTCATCCATGG (SEQ ID NO: |
| | 539) |
| Guide C22-18 | CCCAGCAGAAAACTCTAAAAGGG (SEQ ID NO: |
| | 540) |
| Guide C22-19 | CAGGTGCAAAAAGGTTGCTGTGG (SEQ ID NO: |
| | 541) |
| Guide C22-20 | GCAAGAGATGAAATTCCATATGG (SEQ ID NO: |
| | 542) |
| Guide C22A1 | GGGCTGTTCTAACGAAGTCTGGG (SEQ ID NO: |
| | 543) |
| Guide C22A2 | TGTCCATTCAGCGACCCTAGAGG (SEQ ID NO: |
| | 544) |
| Guide C22A3 | GGCTGTTCTAACGAAGTCTGGGG (SEQ ID NO: |
| | 545) |
| Guide C22A4 | GTCCATTCAGCGACCCTAGAGGG (SEQ ID NO: |
| | 546) |
| Guide C22A5 | GGGGCTGTTCTAACGAAGTCTGG (SEQ ID NO: |
| | 547) |
| Guide C22A6 | GGCTGAATCAGCATGCGAAAGGG (SEQ ID NO: |
| | 548) |
| Guide C22A7 | TTCCAATGGGGGGCATAGCCTGG (SEQ ID NO: |
| | 549) |
| Guide C22A8 | TACCCTCTAGGGTCGCTGAATGG (SEQ ID NO: |
| | 550) |
| Guide C22A9 | ATCCTCTTGGGCCTTATAAGAGG (SEQ ID NO: |
| | 551) |
| Guide C22A10 | GGCCAGGCTATGCCCCCCATTGG (SEQ ID NO: |
| | 552) |
| Guide C22A11 | CTAGAGGACCAGAACAACTCTGG (SEQ ID NO: |
| | 553) |
| Guide C22A12 | TCCCTCTTATAAGGCCCAAGAGG (SEQ ID NO: |
| | 554) |
| Guide C22A13 | AGGCTGAATCAGCATGCGAAAGG (SEQ ID NO: |
| | 555) |
| Guide C22A14 | GGACCAGAACAACTCTGGCCTGG (SEQ ID NO: |
| | 556) |
| Guide C22A15 | GGGCTTTTATTTGGCCCAGCAGG (SEQ ID NO: |
| | 557) |
| Guide C22A16 | GTCGCTGAATGGACAGACTCTGG (SEQ ID NO: |
| | 558) |
| Guide C22A17 | CTCATGAGTTTTACCCTCTAGGG (SEQ ID NO: |
| | 559) |
| Guide C22A18 | TCCTCTTGGGCCTTATAAGAGGG (SEQ ID NO: |
| | 560) |
| Guide C22A19 | TCTTGGGCCTTATAAGAGGGAGG (SEQ ID NO: |
| | 561) |
| Guide C22A20 | TAGAACAGCCCCCCACACAGTGG (SEQ ID NO: |
| | 562) |

In embodiments, gRNAs for targeting human genomic safe harbor sites using any of the gRNA-based targeting elements, e.g., without limitation dCas, to Chromosome X (e.g., hg38 chrX:134,419,661-134,541,172 or hg38 chrX:134,476,304-134,476,307 (85); chrX:134,476,337-134,476,340 (51)) are shown in TABLE 3F.

TABLE 3F

| CHRX | |
|---|---|
| GUIDE NO. | DNA SEQUENCE |
| Guide CX-1 | GTTACGTTATGACTAATCTTTGG (SEQ ID NO: |
| | 563) |
| Guide CX-2 | TACGTTATGACTAATCTTTGGGG (SEQ ID NO: |
| | 564) |
| Guide CX-3 | GGAAGTAGTGTTATGATGTATGG (SEQ ID NO: |
| | 565) |
| Guide CX-4 | GTTATGATGTATGGGCATAAAGG (SEQ ID NO: |
| | 566) |
| Guide CX-5 | GAAGTAGTGTTATGATGTATGGG (SEQ ID NO: |
| | 567) |
| Guide CX-6 | ATAGCTGCTGGCAGTATAACTGG (SEQ ID NO: |
| | 568) |
| Guide CX-7 | GCATCACAACATTGACACTGTGG (SEQ ID NO: |
| | 569) |
| Guide CX-8 | AAGGCGAGTTTCTACAAAGATGG (SEQ ID NO: |
| | 570) |
| Guide CX-9 | TTACGTTATGACTAATCTTTGGG (SEQ ID NO: |
| | 571) |
| Guide CX-10 | CAAGACTGATTAAGACTGATGGG (SEQ ID NO: |
| | 572) |
| Guide CX-11 | AGCAGCAATGTATTAAAGGCTGG (SEQ ID NO: |
| | 573) |
| Guide CX-12 | CTACAGGATTGATGTAAACATGG (SEQ ID NO: |
| | 574) |
| Guide CX-13 | TGGGCATAAAGGGTTTTAATGGG (SEQ ID NO: |
| | 575) |
| Guide CX-14 | ACATCAATCCTGTAGGTGATTGG (SEQ ID NO: |
| | 576) |
| Guide CX-15 | ATTCTAGTCATTATAGCTGCTGG (SEQ ID NO: |
| | 577) |
| Guide CX-16 | CATCAATCCTGTAGGTGATTGGG (SEQ ID NO: |
| | 578) |
| Guide CX-17 | GTTATAAGATCAATTCTGAGTGG (SEQ ID NO: |
| | 579) |
| Guide CX-18 | GGCAGACTGTGGATCAAAAGTGG (SEQ ID NO: |
| | 580) |
| Guide CX-19 | ATGGCTGCCCAATCACCTACAGG (SEQ ID NO: |
| | 581) |
| Guide CX-20 | TCAAAGCATGTACTTAGAGTTGG (SEQ ID NO: |
| | 582) |

In embodiments, the gRNA comprises one or more of the sequences outlined herein or a variant sequence having at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, a Cas-based targeting element comprises Cas12 or a variant thereof, e.g., without limitation, Cas12a (e.g., dCas12a), or Cas12j (e.g., dCas12j), or Cas12k (e.g., dCas12k). In embodiments, the targeting element comprises a Cas12 enzyme guide RNA complex. In embodiments, comprises a nuclease-deficient dCas12 guide RNA complex, optionally dCas12j guide RNA complex or dCas12a guide RNA complex

In embodiments, the targeting element is selected from a zinc finger (ZF), catalytically inactive Zinc finger, transcription activator-like effector (TALE), meganuclease, and clustered regularly interspaced short palindromic repeat (CRISPR)-associated protein, any of which are, in embodiments, catalytically inactive. In embodiments, the CRISPR-associated protein is selected from Cas9, CasX, CasY, Cas12a (Cpf1), and gRNA complexes thereof. In embodiments, the CRISPR-associated protein is selected from Cas9, xCas9, Cas 6, Cas7, Cas8, Cas12a (Cpf1), Cas13a, Cas14, CasX, CasY, a Class 1 Cas protein, a Class 2 Cas protein, MAD7, MG1 nuclease, MG2 nuclease, MG3 nuclease, or catalytically inactive forms thereof, and gRNA complexes thereof.

In embodiments, the helper enzyme is capable of inserting a donor DNA at a TA dinucleotide site or a TTAA tetranucleotide site in a genomic safe harbor site (GSHS) of a nucleic acid molecule. The helper enzyme is suitable for causing insertion of the donor DNA in a GSHS when contacted with a biological cell.

In embodiments, the targeting element is suitable for directing the helper enzyme to the GSHS sequence.

In embodiments, the targeting element comprises transcription activator-like effector (TALE) DNA binding domain (DBD). The TALE DBD comprises one or more repeat sequences. For example, in embodiments, the TALE DBD comprises about 14, or about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise a repeat variable di-residue (RVD) at residue 12 or 13 of the 33 or 34 amino acids.

In embodiments, the targeting element (e.g., TALE or Cas (e.g., Cas9 or Cas12, or variants thereof) DBDs cause the mammalian helper enzyme to bind specifically to human GSHS. In embodiments, the TALEs or Cas DBDs sequester the helper enzyme to GSHS and promote transposition to nearby TA dinucleotide or a TTAA tetranucleotide sites which can be located in proximity to the repeat variable di-residues (RVD) TALE or gRNA nucleotide sequences. The GSHS regions are located in open chromatin sites that are susceptible to helper enzyme activity. Accordingly, the mammalian helper enzyme does not only operate based on its ability to recognize TA or TTAA sites, but it also directs a donor DNA (having a transgene) to specific locations in proximity to a TALE or Cas DBD. The chimeric helper enzyme in accordance with embodiments of the present disclosure has negligible risk of genotoxicity and exhibits superior features as compared to existing gene therapies.

In embodiments, a chimeric helper enzyme is mutated to be characterized by reduced or inhibited binding of off-target sequences and consequently reliant on a DBD fused thereto, such as a TALE or Cas DBD, for transposition.

The described cells, compositions, and methods allow reducing vector and transgene insertions that increase a mutagenic risk. The described cells and methods make use of a gene transfer system that reduces genotoxicity compared to viral- and nuclease-mediated gene therapies. The dual system is designed to avoid the persistence of an active helper enzyme and efficiently transfect human cell lines without significant cytotoxicity.

In embodiments, TALE or Cas DBDs are customizable, such as a TALE or Cas DBDs is selected for targeting a specific genomic location. In embodiments, the genomic location is in proximity to a TA dinucleotide site or a TTAA (SEQ ID NO: 440) tetranucleotide site.

Embodiments of the present disclosure make use of the ability of TALE or Cas or dCas9/gRNA DBDs to target specific sites in a host genome. The DNA targeting ability of a TALE or Cas DBD or dCas9/gRNA DBD is provided by TALE repeat sequences (e.g., modular arrays) or gRNA which are linked together to recognize flanking DNA sequences. Each TALE or gRNA can recognize certain base pair(s) or residue(s).

TALE nucleases (TALENs) are a known tool for genome editing and introducing targeted double-stranded breaks. TALENs comprise endonucleases, such as FokI nuclease domain, fused to a customizable DBD. This DBD is composed of highly conserved repeats from TALEs, which are proteins secreted by Xanthomonas bacteria to alter transcription of genes in host plant cells. The DBD includes a repeated highly conserved 33-34 amino acid sequence with divergent 12th and 13th amino acids. These two positions, referred to as the RVD, are highly variable and show a strong correlation with specific base pair or nucleotide recognition. This straightforward relationship between amino acid sequence and DNA recognition has allowed for the engineering of specific DBDs by selecting a combination of repeat segments containing the appropriate RVDs. Boch et al. Nature Biotechnology. 2011; 29 (2): 135-6.

Accordingly, TALENs can be readily designed using a “protein-DNA code” that relates modular DNA-binding TALE repeat domains to individual bases in a target-binding site. See Joung et al. Nat Rev Mol Cell Biol. 2013; 14(1):49-55. doi:10.1038/nrm3486. The following table, TABLE 2, for example, shows such code.

TABLE 2

| RVD | Nucleotide | RVD | Nucleotide |
|---|---|---|---|
| HD | C | NI | A |
| NH | G | NN | G, A |
| NK | G | NS | G, C, A |
| NG | T, mC | | |

It has been demonstrated that TALENs can be used to target essentially any DNA sequence of interest in human cell. Miller et al. Nat Biotechnol. 2011; 29:143-148. Guidelines for selection of potential target sites and for use of particular TALE repeat domains (harboring NH residues at the hypervariable positions) for recognition of G bases have been proposed. See Streubel et al. Nat Biotechnol. 2012; 30:593-595.

Accordingly, in embodiments, the TALE DBD comprises one or more repeat sequences. In embodiments, the TALE DBD comprises about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise an RVD at residue 12 or 13 of the 33 or 34 amino acids. The RVD can recognize certain base pair(s) or residue(s). In embodiments, the RVD recognizes one base pair in the nucleic acid molecule. In embodiments, the RVD recognizes a C residue in the nucleic acid molecule and is selected from HD, N(gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(gap), and IG.

In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor; and human Rosa26 locus. In embodiments, the GSHS is located on human chromosome 2, 4, 6, 10, 11, 17, 22, or X.

In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the GSHS comprises one or more of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGCGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD binds to one of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGCGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD comprises one or more of

(SEQ ID NO: 355)
NH NH HD HD NH NH HD HD NG NH NI HD HD NI HD NG NH NH,
(SEQ ID NO: 356)
NH NI NI NH NH HD HD NG NH NH HD HD NH NH HD HD NG NH,
(SEQ ID NO: 357)
NH NI NH HD NI HD NG NH NI NI NH NH HD HD NG NH NH HD,
(SEQ ID NO: 358)
HD HD NI HD NG NH NI NH HD NI HD NG NH NI NI NH NH HD,
(SEQ ID NO: 359)
NH NH NG NG NG HD HD NI HD NG NH NI NH HD NI HD NG NH,
(SEQ ID NO: 360)
NH NH NH NH NI NI NI NI NG NH NI HD HD HD NI NI HD NI,
(SEQ ID NO: 361)
NI NH NH NI HD NI NH NG NH NH NH NH NI NI NI NI NG NH,
(SEQ ID NO: 362)
HD HD NI NH NH NH NI HD NI HD NH NH NG NH HD NG NI NH,
(SEQ ID NO: 363)
HD NI NH NI NH HD HD NI NH NH NI NH NG HD HD NG NH NH,
(SEQ ID NO: 364)
HD HD NG NG HD NI NH NI NH HD HD NI NH NH NI NH NG HD,
(SEQ ID NO: 365)
HD HD NG HD HD NG NG HD NI NH NI NH HD HD NI NH NH NI,
(SEQ ID NO: 366)
HD HD NI NH HD HD HD HD NG HD HD NG HD HD NG NG HD NI,
(SEQ ID NO: 367)
HD HD NH NI NH HD NG NG NH NI HD HD HD NG NG NH NH NI,
(SEQ ID NO: 368)
NH NH NG NG NG HD HD NH NI NH HD NG NG NH NI HD HD HD,
(SEQ ID NO: 369)
NH NH NH NH NG NH NH NG NG NG HD HD NH NI NH HD NG NG,
(SEQ ID NO: 370)
HD NG NH HD NG NH NH NH NH NG NH NH NG NG NG HD HD NH,
(SEQ ID NO: 371)
NH HD NI NH NI NH NG NI NG HD NG NH HD NG NH NH NH NH,
(SEQ ID NO: 372)
HD HD NI NI NG HD HD HD HD NG HD NI NH NG,
(SEQ ID NO: 373)
HD NI NH NG NH HD NG HD NI NH NG NH NH NI NI,
(SEQ ID NO: 374)
NH NI NI NI HD NI NG HD HD NH NH HD NH NI HD NG HD NI,
(SEQ ID NO: 375)
HD NH HD HD HD HD NG HD NI NI NI NG HD NG NG NI HD NI,
(SEQ ID NO: 376)
HD NI NI NI NG HD NG NG NI HD NI NH HD NG NH HD NG HD,
(SEQ ID NO: 377)
HD NG NG NI HD NI NH HD NG NH HD NG HD NI HD NG HD HD,
(SEQ ID NO: 378)
NI HD NI NH HD NG NH HD NG HD NI HD NG HD HD HD HD NG,
(SEQ ID NO: 379)
NH HD NG HD NI HD NG HD HD HD HD NG NH HD NI NH NH NH,
(SEQ ID NO: 380)
HD HD HD HD NG NH HD NI NH NH NH HD NI NI HD NH HD HD,
(SEQ ID NO: 381)
NH HD NI NH NH NH HD NI NI HD NH HD HD HD NI NH NH NH,
(SEQ ID NO: 382)
HD NG HD NH NI NG NG NI NG NH NH NH HD NH NH NH NI NG,
(SEQ ID NO: 383)
HD NH HD NG NG HD NG HD NH NI NG NG NI NG NH NH NH HD,
(SEQ ID NO: 384)
NH NG HD NH NI NH NG HD NH HD NG NG HD NG HD NH NI NG,
(SEQ ID NO: 385)
HD HD NI NG NH NG HD NH NI NH NG HD NH HD NG NG HD NG,
(SEQ ID NO: 386)
HD NH HD HD NG HD HD NI NG NH NG HD NH NI NH NG HD NH,
(SEQ ID NO: 387)
HD NH NG HD NI NG HD NH HD HD NG HD HD NI NG NH NG HD,
(SEQ ID NO: 388)
NH NI NG HD NG HD NH NG HD NI NG HD NH HD HD NG HD HD,
(SEQ ID NO: 389)
NH HD NG NG HD NI NH HD NG NG HD HD NG NI,
(SEQ ID NO: 390)
HD NG NK NG NH NI NG HD NI NG NH HD HD NI,
(SEQ ID NO: 391)
NI HD NI NN NG NN NN NG NI HD NI HD NI HD HD NG,
(SEQ ID NO: 392)
HD HD NI HD HD HD HD HD HD NI HD NG NI NI NN,
(SEQ ID NO: 393)
HD NI NG NG NN NN HD HD NN NN NN HD NI HD,
(SEQ ID NO: 394)
NN HD NG NG NN NI NI HD HD HD NI NN NN NI NN NI,
(SEQ ID NO: 395)
NI HD NI HD HD HD NN NI NG HD HD NI HD NG NN NN NN,
(SEQ ID NO: 396)
NN HD NG NN HD NI NG HD NI NI HD HD HD HD,
(SEQ ID NO: 397)
NN NN HD NI HD NN NI NI NI HD NI HD HD HD NG HD HD,
(SEQ ID NO: 398)
NN NN NG NN NN HD NG HD NI NG NN HD HD NG NN,
(SEQ ID NO: 399)
NN NI NG NG NG NN HD NI HD NI NN HD NG HD NI NG,
(SEQ ID NO: 400)
NI NI NH HD NG HD NG NH NI NH NH NI NH HD,
(SEQ ID NO: 401)
HD HD HD NG NI NK HD NG NH NG HD HD HD HD,
(SEQ ID NO: 402)
NH HD HD NG NI NH HD NI NG NH HD NG NI NH,
(SEQ ID NO: 403)
NI NG NH NH NH HD NG NG HD NI HD NH NH NI NG,
(SEQ ID NO: 404)
NH NI NI NI HD NG NI NG NH HD HD NG NH HD,
(SEQ ID NO: 405)
NH HD NI HD HD NI NG NG NH HD NG HD HD HD,
(SEQ ID NO: 406)
NH NI HD NI NG NH HD NI NI HD NG HD NI NH,
(SEQ ID NO: 407)
NI HD NI HD HD NI HD NG NI NH NH NH NH NG,
(SEQ ID NO: 408)
NH NG HD NG NH HD NG NI NH NI HD NI NH NH,
(SEQ ID NO: 409)
NH NH HD HD NG NI NH NI HD NI NH NH HD NG NH,
(SEQ ID NO: 410)
NH NI NH NH HD NI NG NG HD NG NG NI NG HD NH,
(SEQ ID NO: 411)
NN HD HD NG NN NN NI NI NI HD NN NG NG HD HD,
(SEQ ID NO: 412)
NN NG NN HD NG HD NG NN NI HD NI NI NG NI,
(SEQ ID NO: 413)
NN NG NG NG NG NN HD NI NN HD HD NG HD HD,
(SEQ ID NO: 414)
NI HD NI NN HD NG NN NG NN NN NI NI HD NN NG,
(SEQ ID NO: 415)
HD NI NI NN NI HD HD NN NI NN HD NI HD NG NN HD NG NN,
(SEQ ID NO: 416)
HD NG NI NG HD HD HD NI NI NI NI HD NG HD NG,
(SEQ ID NO: 417)
NH NI NI NI NI NI HD NG NING NH NG NI NG,
(SEQ ID NO: 418)
NI NH NH HD NI NH NH HD NG NH NH NG NG NH NI,
(SEQ ID NO: 419)
HD NI NI NG NI HD NI NI HD HD NI HD NN HD,
(SEQ ID NO: 420)
NI NG NN NI HD NN NN NI HD NG HD NI NI HD NG,
(SEQ ID NO: 421)
HD NI HD NI NI HD NI NG NG NG NN NG NI NI,
and
(SEQ ID NO: 422)
NI NG NG NG HD HD NI NN NG NN HD NI HD NI.

In embodiments, the GSHS is selected from sites listed in FIG. 15A and the TALE DBD comprises a sequence of FIG. 15A.

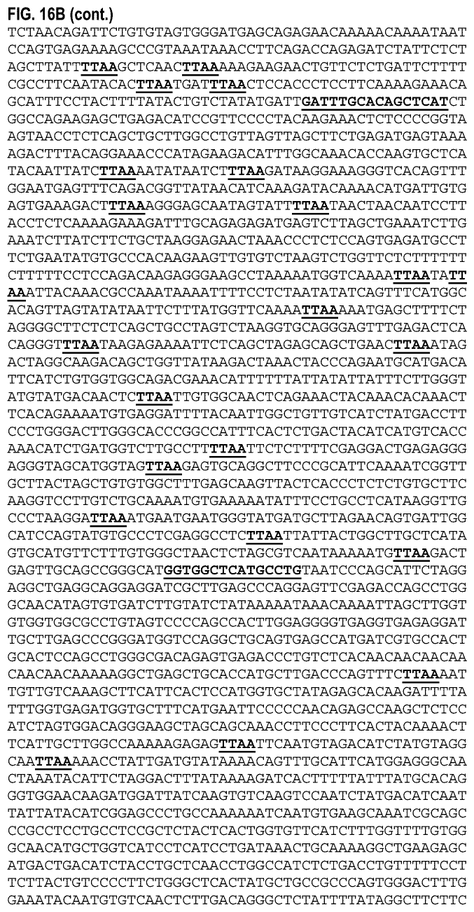

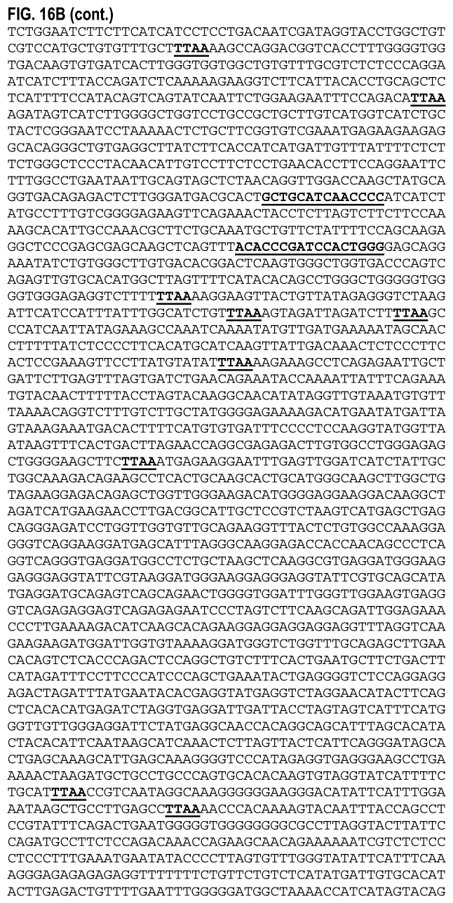

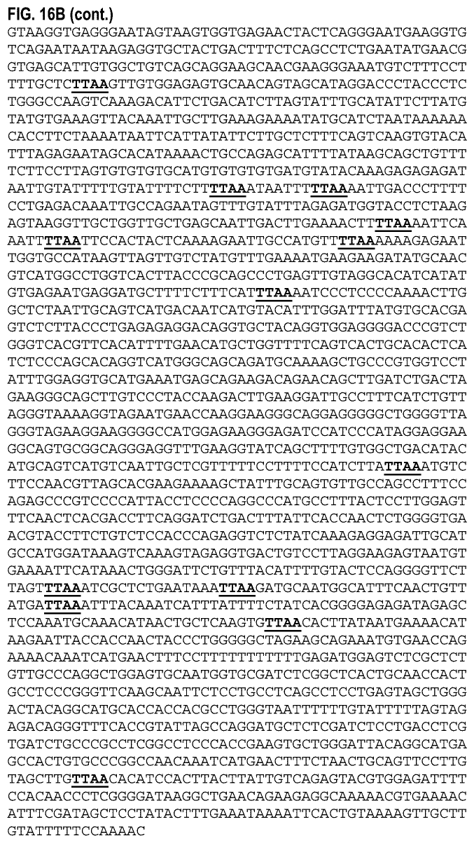

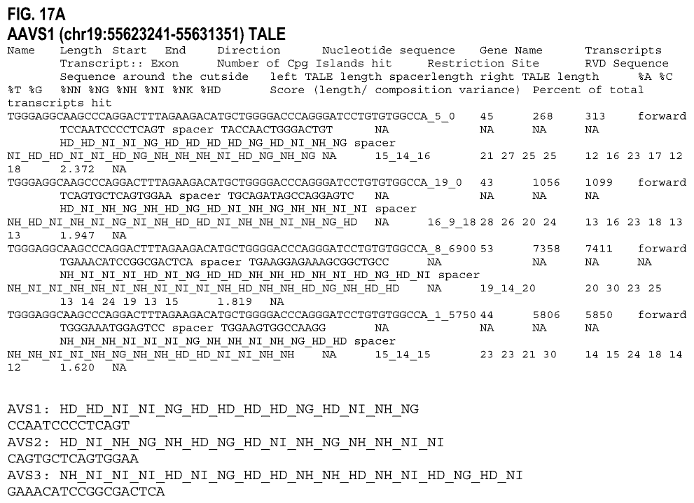

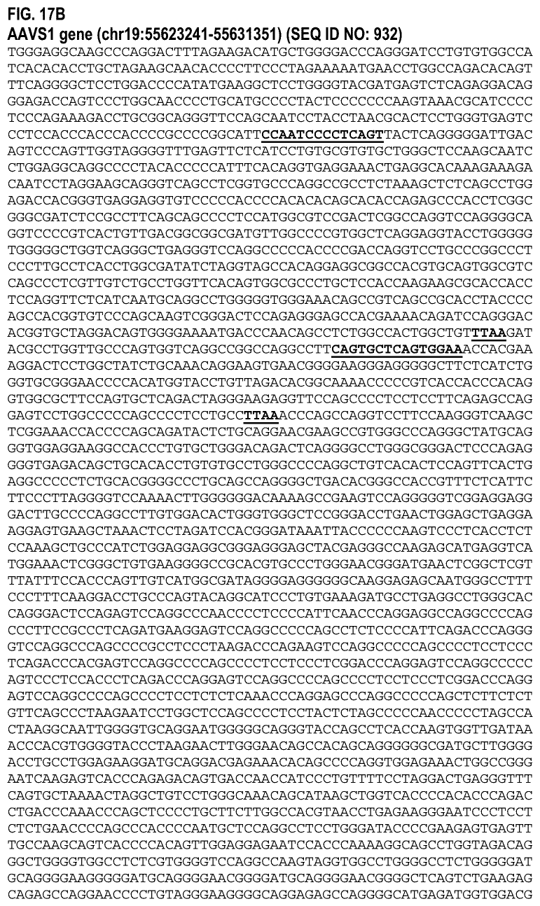

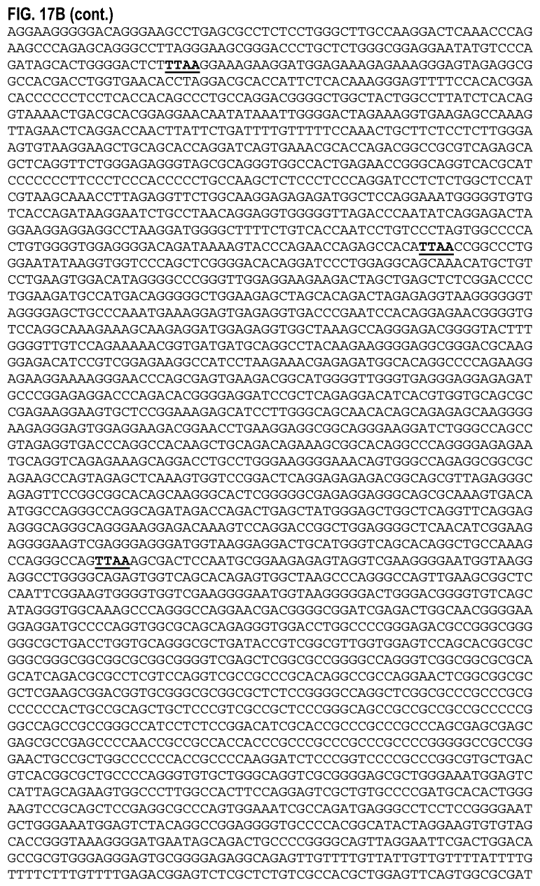

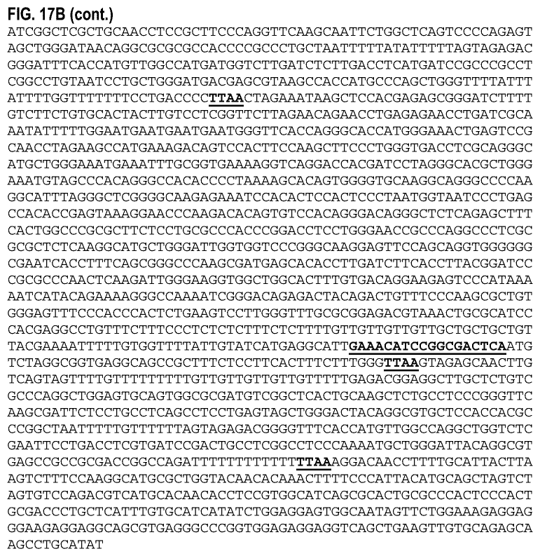

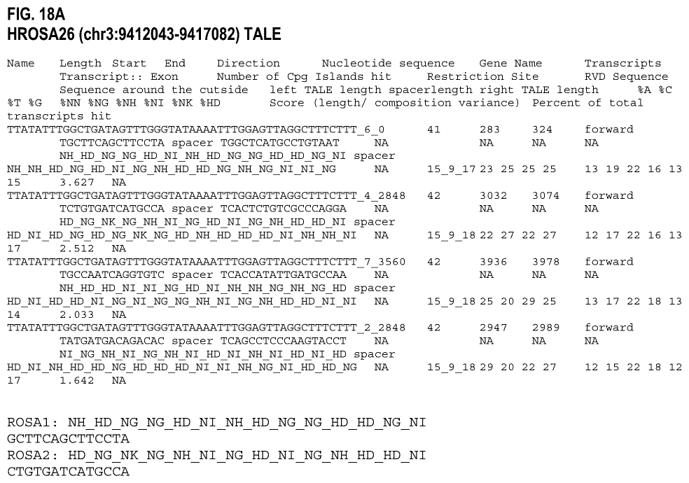

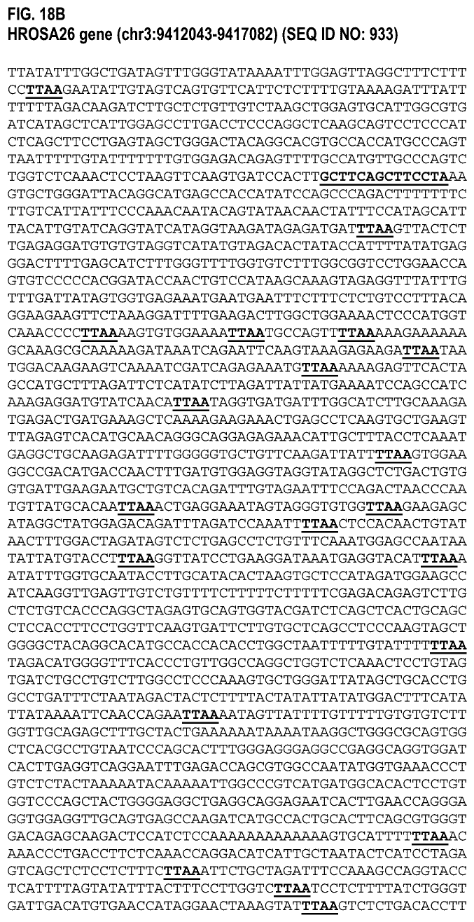

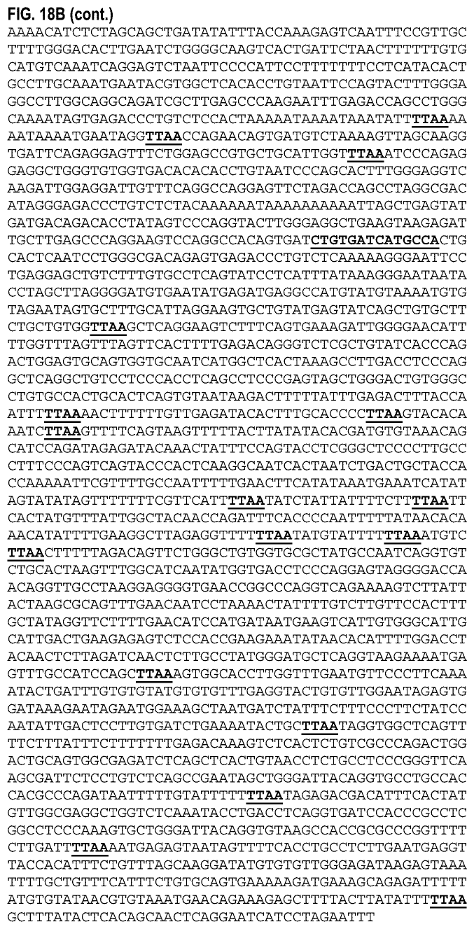

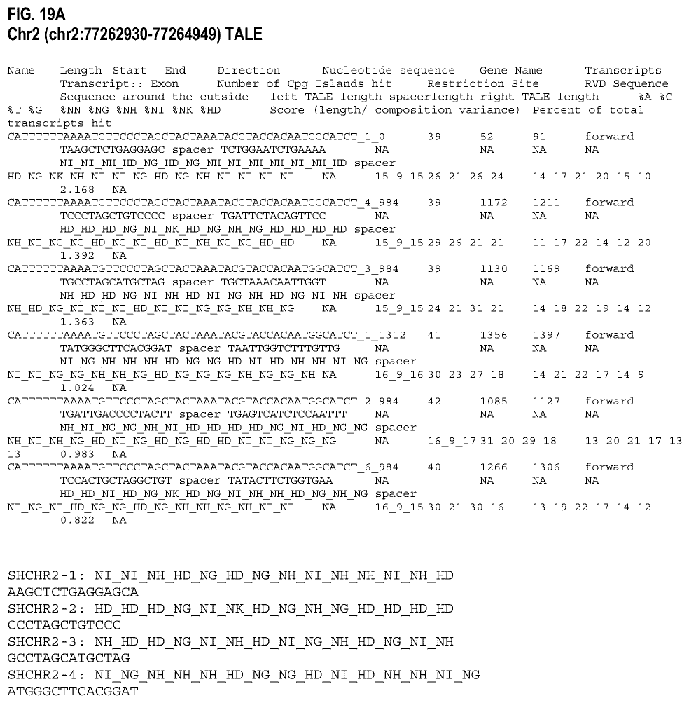

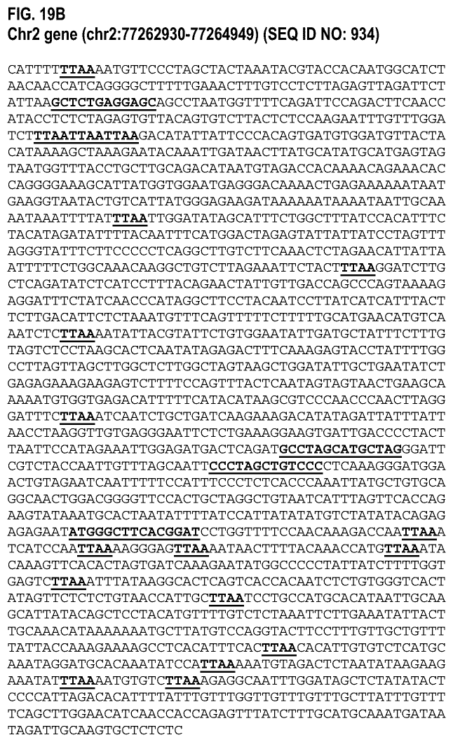

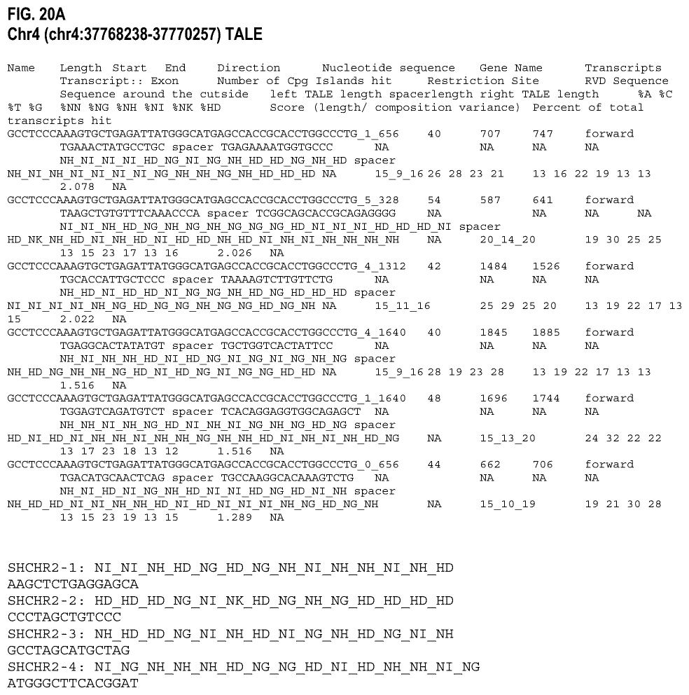

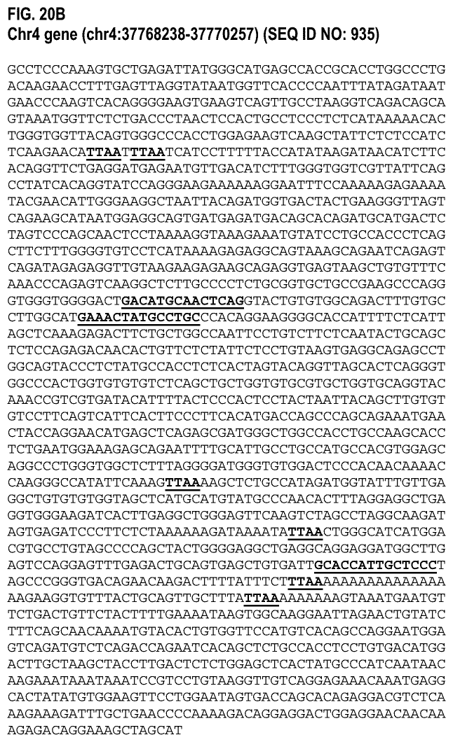

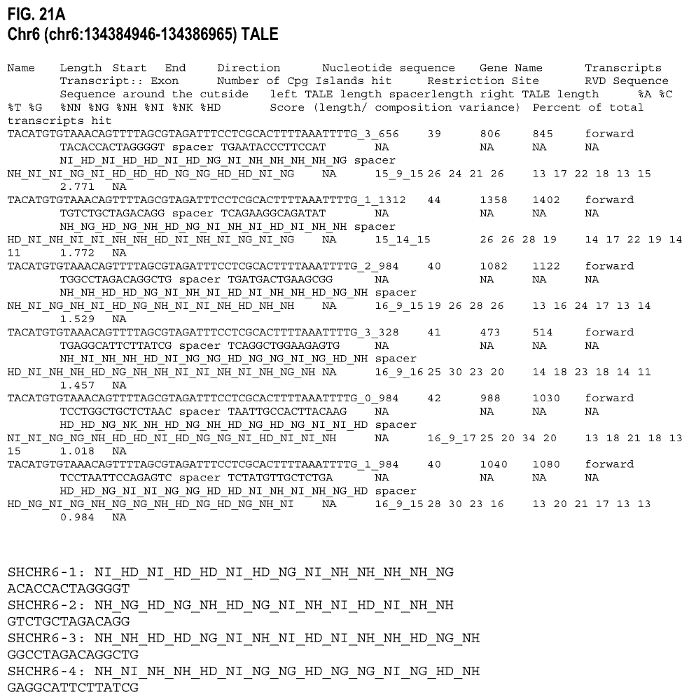

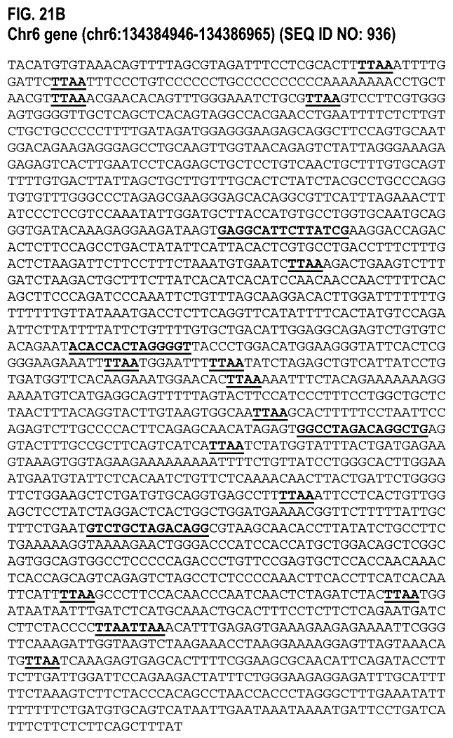

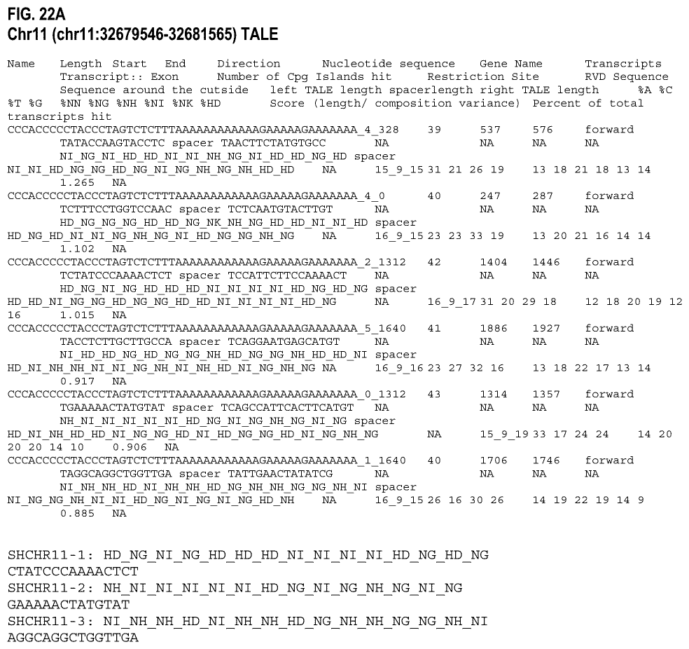

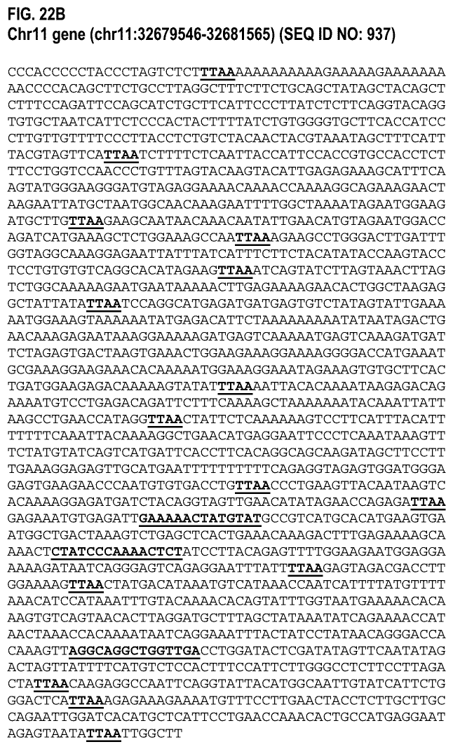

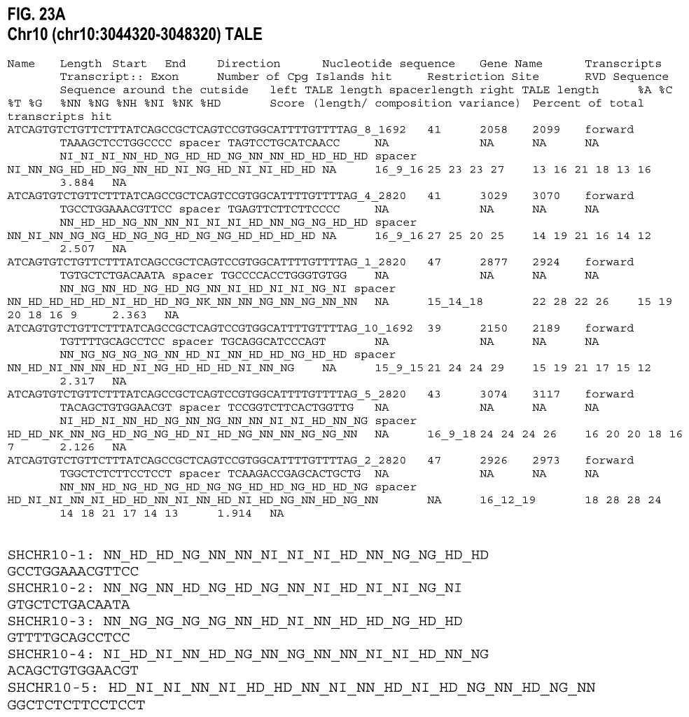

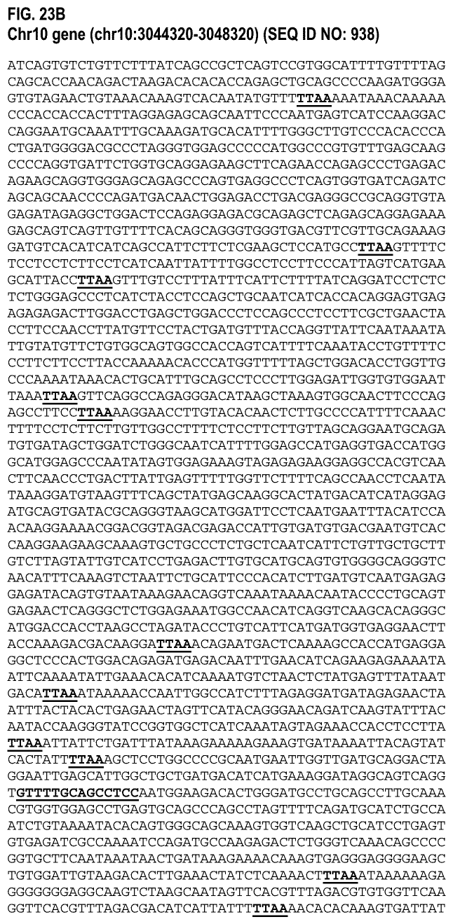

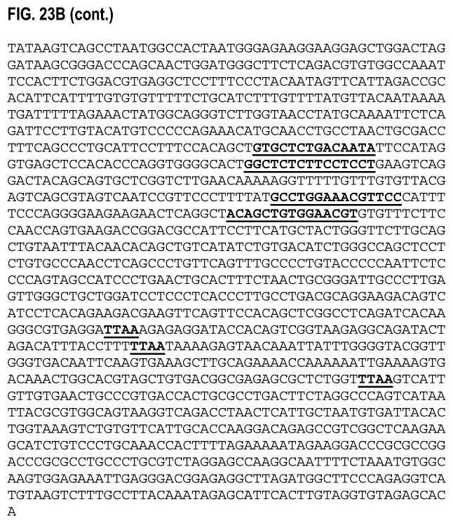

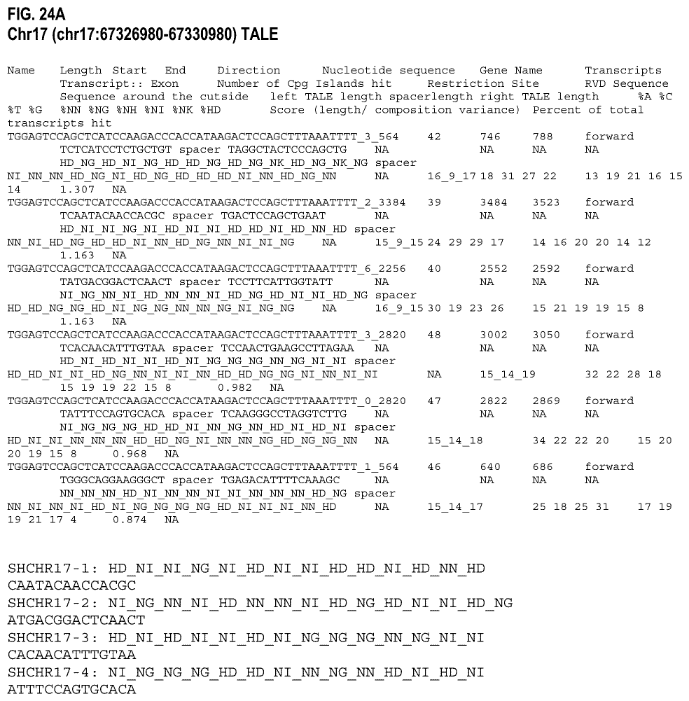

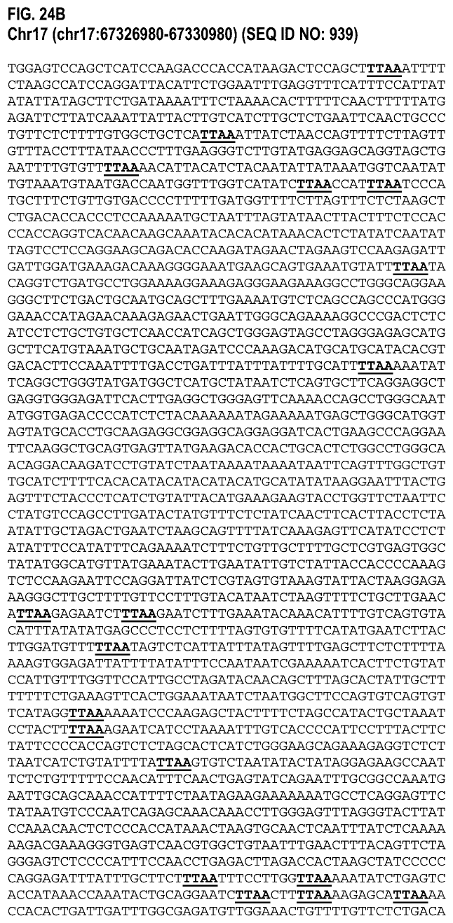

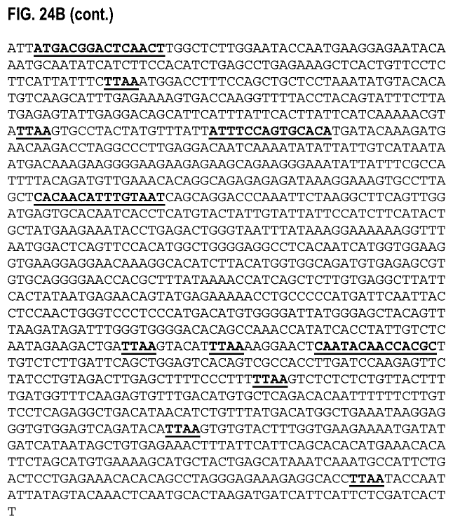

In embodiments, the TALE DBD comprises one or more of the sequences of FIG. 16A, FIG. 17A, FIG. 18A, FIG. 19A, FIG. 20A, FIG. 21A, FIG. 22A, FIG. 23A, or FIG. 24A, or a sequence having at least about 90%, or at least about 93%, or at least about 95%, or at least about 97%, or at least about 98%, or at least about 99% identity thereto.

In embodiments, the TALE DBD comprises one or more of the sequences outlined herein or a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, the GSHS and the TALE DBD sequences are selected from:

(SEQ ID NO: 23)
TGGCCGGCCTGACCACTGG
and
(SEQ ID NO: 355)
NH NH HD HD NH NH HD HD NG NH NI HD HD NI HD NG NH
NH;
(SEQ ID NO: 24)
TGAAGGCCTGGCCGGCCTG
and
(SEQ ID NO: 356)
NH NI NI NH NH HD HD NG NH NH HD HD NH NH HD HD NG
NH;
(SEQ ID NO: 25)
TGAGCACTGAAGGCCTGGC
and
(SEQ ID NO: 357)
NH NI NH HD NI HD NG NH NI NI NH NH HD HD NG NH NH
HD;
(SEQ ID NO: 26)
TCCACTGAGCACTGAAGGC
and
(SEQ ID NO: 358)
HD HD NI HD NG NH NI NH HD NI HD NG NH NI NI NH NH HD;
(SEQ ID NO: 27)
TGGTTTCCACTGAGCACTG
and
(SEQ ID NO: 359)
NH NH NG NG NG HD HD NI HD NG NH NI NH HD NI HD NG
NH;
(SEQ ID NO: 28)
TGGGGAAAATGACCCAACA
and
(SEQ ID NO: 360)
NH NH NH NH NI NI NI NI NG NH NI HD HD HD NI NI HD NI;
(SEQ ID NO: 29)
TAGGACAGTGGGGAAAATG
and
(SEQ ID NO: 361)
NI NH NH NI HD NI NH NG NH NH NH NH NI NI NI NI NG NH;
(SEQ ID NO: 30)
TCCAGGGACACGGTGCTAG
and
(SEQ ID NO: 362)
HD HD NI NH NH NH NI HD NI HD NH NH NG NH HD NG NI
NH;
(SEQ ID NO: 31)
TCAGAGCCAGGAGTCCTGG
and
(SEQ ID NO: 363)
HD NI NH NI NH HD HD NI NH NH NI NH NG HD HD NG NH
NH;
(SEQ ID NO: 32)
TCCTTCAGAGCCAGGAGTC
and
(SEQ ID NO: 364)
HD HD NG NG HD NI NH NI NH HD HD NI NH NH NI NH NG HD;
(SEQ ID NO: 33)
TCCTCCTTCAGAGCCAGGA
and
(SEQ ID NO: 365)
HD HD NG HD HD NG NG HD NI NH NI NH HD HD NI NH NH
NI;
(SEQ ID NO: 34)
TCCAGCCCCTCCTCCTTCA
and
(SEQ ID NO: 366)
HD HD NI NH HD HD HD HD NG HD HD NG HD HD NG NG HD
NI;
(SEQ ID NO: 35)
TCCGAGCTTGACCCTTGGA
and
(SEQ ID NO: 367)
HD HD NH NI NH HD NG NG NH NI HD HD HD NG NG NH NH
NI;
(SEQ ID NO: 36)
TGGTTTCCGAGCTTGACCC
and
(SEQ ID NO: 368)
NH NH NG NG NG HD HD NH NI NH HD NG NG NH NI HD HD
HD;
(SEQ ID NO: 37)
TGGGGTGGTTTCCGAGCTT
and
(SEQ ID NO: 369)
NH NH NH NH NG NH NH NG NG NG HD HD NH NI NH HD NG
NG;
(SEQ ID NO: 38)
TCTGCTGGGGTGGTTTCCG
and
(SEQ ID NO: 370)
HD NG NH HD NG NH NH NH NH NG NH NH NG NG NG HD
HD NH;
(SEQ ID NO: 39)
TGCAGAGTATCTGCTGGGG
and
(SEQ ID NO: 371)
NH HD NI NH NI NH NG NI NG HD NG NH HD NG NH NH NH
NH;
(SEQ ID NO: 40)
CCAATCCCCTCAGT
and
(SEQ ID NO: 372)
HD HD NI NI NG HD HD HD HD NG HD NI NH NG;
(SEQ ID NO: 41)
CAGTGCTCAGTGGAA
and
(SEQ ID NO: 373)
HD NI NH NG NH HD NG HD NI NH NG NH NH NI NI;
(SEQ ID NO: 42)
GAAACATCCGGCGACTCA
and
(SEQ ID NO: 374)
NH NI NI NI HD NI NG HD HD NH NH HD NH NI HD NG HD NI;
(SEQ ID NO: 43)
TCGCCCCTCAAATCTTACA
and
(SEQ ID NO: 375)
HD NH HD HD HD HD NG HD NI NI NI NG HD NG NG NI HD NI;
(SEQ ID NO: 44)
TCAAATCTTACAGCTGCTC
and
(SEQ ID NO: 376)
HD NI NI NI NG HD NG NG NI HD NI NH HD NG NH HD NG HD;
(SEQ ID NO: 45)
TCTTACAGCTGCTCACTCC
and
(SEQ ID NO: 377)
HD NG NG NI HD NI NH HD NG NH HD NG HD NI HD NG HD
HD;
(SEQ ID NO: 46)
TACAGCTGCTCACTCCCCT
and
(SEQ ID NO: 378)
NI HD NI NH HD NG NH HD NG HD NI HD NG HD HD HD HD
NG;
(SEQ ID NO: 47)
TGCTCACTCCCCTGCAGGG
and
(SEQ ID NO: 379)
NH HD NG HD NI HD NG HD HD HD HD NG NH HD NI NH NH
NH;
(SEQ ID NO: 48)
TCCCCTGCAGGGCAACGCC
and
(SEQ ID NO: 380)
HD HD HD HD NG NH HD NI NH NH NH HD NI NI HD NH HD
HD;
(SEQ ID NO: 49)
TGCAGGGCAACGCCCAGGG
and
(SEQ ID NO: 381)
NH HD NI NH NH NH HD NI NI HD NH HD HD HD NI NH NH
NH;
(SEQ ID NO: 50)
TCTCGATTATGGGCGGGAT
and
(SEQ ID NO: 382)
HD NG HD NH NI NG NG NING NH NH NH HD NH NH NH NI
NG;
(SEQ ID NO: 51)
TCGCTTCTCGATTATGGGC
(SEQ ID NO: 383)
and HD NH HD NG NG HD NG HD NH NI NG NG NING NH NH NH
HD;
(SEQ ID NO: 52)
TGTCGAGTCGCTTCTCGAT
and
(SEQ ID NO: 384)
NH NG HD NH NI NH NG HD NH HD NG NG HD NG HD NH NI
NG;
(SEQ ID NO: 53)
TCCATGTCGAGTCGCTTCT
and
(SEQ ID NO: 385)
HD HD NI NG NH NG HD NH NI NH NG HD NH HD NG NG HD
NG;
(SEQ ID NO: 54)
TCGCCTCCATGTCGAGTCG
and
(SEQ ID NO: 386)
HD NH HD HD NG HD HD NI NG NH NG HD NH NI NH NG HD
NH;
(SEQ ID NO: 55)
TCGTCATCGCCTCCATGTC
and
(SEQ ID NO: 387)
HD NH NG HD NI NG HD NH HD HD NG HD HD NI NG NH NG
HD;
(SEQ ID NO: 56)
TGATCTCGTCATCGCCTCC
and
(SEQ ID NO: 388)
NH NI NG HD NG HD NH NG HD NI NG HD NH HD HD NG HD
HD;
(SEQ ID NO: 57)
GCTTCAGCTTCCTA
and
(SEQ ID NO: 389)
NH HD NG NG HD NI NH HD NG NG HD HD NG NI;
(SEQ ID NO: 58)
CTGTGATCATGCCA
and
(SEQ ID NO: 390)
HD NG NK NG NH NI NG HD NI NG NH HD HD NI;
(SEQ ID NO: 59)
ACAGTGGTACACACCT
and
(SEQ ID NO: 391)
NI HD NI NN NG NN NN NG NI HD NI HD NI HD HD NG;
(SEQ ID NO: 60)
CCACCCCCCACTAAG
and
(SEQ ID NO: 392)
HD HD NI HD HD HD HD HD HD NI HD NG NI NI NN;
(SEQ ID NO: 61)
CATTGGCCGGGCAC
and
(SEQ ID NO: 393)
HD NI NG NG NN NN HD HD NN NN NN HD NI HD;
(SEQ ID NO: 62)
GCTTGAACCCAGGAGA
and
(SEQ ID NO: 394)
NN HD NG NG NN NI NI HD HD HD NI NN NN NI NN NI;
(SEQ ID NO: 63)
ACACCCGATCCACTGGG
and
(SEQ ID NO: 395)
NI HD NI HD HD HD NN NI NG HD HD NI HD NG NN NN NN;
(SEQ ID NO: 64)
GCTGCATCAACCCC
and
(SEQ ID NO: 396)
NN HD NG NN HD NI NG HD NI NI HD HD HD HD;
(SEQ ID NO: 65)
GCCACAAACAGAAATA
and
(SEQ ID NO: 397)
NN NN HD NI HD NN NI NI NI HD NI HD HD HD NG HD HD;
(SEQ ID NO: 66)
GGTGGCTCATGCCTG
and
(SEQ ID NO: 398)
NN NN NG NN NN HD NG HD NI NG NN HD HD NG NN;
(SEQ ID NO: 67)
GATTTGCACAGCTCAT
and
(SEQ ID NO: 399)
NN NI NG NG NG NN HD NI HD NI NN HD NG HD NI NG;
(SEQ ID NO: 68)
AAGCTCTGAGGAGCA
and
(SEQ ID NO: 400)
NI NI NH HD NG HD NG NH NI NH NH NI NH HD;
(SEQ ID NO: 69)
CCCTAGCTGTCCC
and
(SEQ ID NO: 401)
HD HD HD NG NI NK HD NG NH NG HD HD HD HD;
(SEQ ID NO: 70)
GCCTAGCATGCTAG
and
(SEQ ID NO: 402)
NH HD HD NG NI NH HD NI NG NH HD NG NI NH;
(SEQ ID NO: 71)
ATGGGCTTCACGGAT
and
(SEQ ID NO: 403)
NI NG NH NH NH HD NG NG HD NI HD NH NH NI NG;
(SEQ ID NO: 72)
GAAACTATGCCTGC
and
(SEQ ID NO: 404)
NH NI NI NI HD NG NING NH HD HD NG NH HD;
(SEQ ID NO: 73)
GCACCATTGCTCCC
and
(SEQ ID NO: 405)
NH HD NI HD HD NING NG NH HD NG HD HD HD;
(SEQ ID NO: 74)
GACATGCAACTCAG
and
(SEQ ID NO: 406)
NH NI HD NI NG NH HD NI NI HD NG HD NI NH;
(SEQ ID NO: 75)
ACACCACTAGGGGT
and
(SEQ ID NO: 407)
NI HD NI HD HD NI HD NG NI NH NH NH NH NG;
(SEQ ID NO: 76)
GTCTGCTAGACAGG
(SEQ ID NO: 408)
and NH NG HD NG NH HD NG NI NH NI HD NI NH NH;
(SEQ ID NO: 77)
GGCCTAGACAGGCTG
and
(SEQ ID NO: 409)
NH NH HD HD NG NI NH NI HD NI NH NH HD NG NH;
(SEQ ID NO: 78)
GAGGCATTCTTATCG
and
(SEQ ID NO: 410)
NH NI NH NH HD NI NG NG HD NG NG NI NG HD NH;
(SEQ ID NO: 79)
GCCTGGAAACGTTCC
and
(SEQ ID NO: 411)
NN HD HD NG NN NN NI NI NI HD NN NG NG HD HD;
(SEQ ID NO: 80)
GTGCTCTGACAATA
and
(SEQ ID NO: 412)
NN NG NN HD NG HD NG NN NI HD NI NI NG NI;
(SEQ ID NO: 81)
GTTTTGCAGCCTCC
and
(SEQ ID NO: 413)
NN NG NG NG NG NN HD NI NN HD HD NG HD HD;
(SEQ ID NO: 82)
ACAGCTGTGGAACGT
and
(SEQ ID NO: 414)
NI HD NI NN HD NG NN NG NN NN NI NI HD NN NG;
(SEQ ID NO: 83)
GGCTCTCTTCCTCCT
and
(SEQ ID NO: 415)
HD NI NI NN NI HD HD NN NI NN HD NI HD NG NN HD NG NN;
(SEQ ID NO: 84)
CTATCCCAAAACTCT
and
(SEQ ID NO: 416)
HD NG NI NG HD HD HD NI NI NI NI HD NG HD NG;
(SEQ ID NO: 85)
GAAAAACTATGTAT
and
(SEQ ID NO: 417)
NH NI NI NI NI NI HD NG NI NG NH NG NI NG;
(SEQ ID NO: 86)
AGGCAGGCTGGTTGA
and
(SEQ ID NO: 418)
NI NH NH HD NI NH NH HD NG NH NH NG NG NH NI;
(SEQ ID NO: 87)
CAATACAACCACGC
and
(SEQ ID NO: 419)
HD NI NI NG NI HD NI NI HD HD NI HD NN HD;
(SEQ ID NO: 88)
ATGACGGACTCAACT
and
(SEQ ID NO: 420)
NI NG NN NI HD NN NN NI HD NG HD NI NI HD NG;
and
(SEQ ID NO: 89)
CACAACATTTGTAA
and
(SEQ ID NO: 421)
HD NI HD NI NI HD NI NG NG NG NN NG NI NI.

In embodiments, the GSHS is within about 25, or about 50, or about 100, or about 150, or about 200, or about 300, or about 500 nucleotides of the TA dinucleotide site or TTAA (SEQ ID NO: 440) tetranucleotide site.

In embodiments, the positions of the GSHS and TTAA tetranucleotide site are as depicted in FIG. 16B, FIG. 18B, FIG. 19B, FIG. 20B, FIG. 21B, FIG. 22B, FIG. 23B, or FIG. 24B.

In embodiments, guide RNAs (gRNAs) for dCas9 to target human genomic safe harbor sites in areas of open chromatin are as shown in the example of FIG. 15B.

Illustrative DNA binding codes for human genomic safe harbor in areas of open chromatin via TALEs, encompassed by various embodiments are provided in TABLE 4A-4F. In embodiments, there is provided a variant of the TALEs, encompassed by various embodiments are provided in TABLE 4A-4F, e.g., having a sequence having at least about 90%, or at least about 93%, or at least about 95%, or at least about 97%, or at least about 98%, or at least about 99% identity to any of the sequences in TABLE 4A-4F.

Illustrative DNA binding codes for human genomic safe harbor in areas of open chromatin via TALEs, encompassed by various embodiments are provided in TABLE 4A.

TABLE 4A

| GSHS | ID | Sequence | TALE (DNA binding code) |
|---|---|---|---|
| AAVS1 | 1 | TGGCCGGCCTGACCACTGG | NH NH HD HD NH NH HD HD NG |
| | | (SEQ ID NO: 23) | NH NI HD HD NI HD NG NH NH |
| | | | (SEQ ID NO: 355) |
| AAVS1 | 2 | TGAAGGCCTGGCCGGCCTG | NH NI NI NH NH HD HD NG NH NH |
| | | (SEQ ID NO: 24) | HD HD NH NH HD HD NG NH |
| | | | (SEQ ID NO: 356) |
| AAVS1 | 3 | TGAGCACTGAAGGCCTGGC | NH NI NH HD NI HD NG NH NI NI |
| | | (SEQ ID NO: 25) | NH NH HD HD NG NH NH HD |
| | | | (SEQ ID NO: 357) |
| AAVS1 | 4 | TCCACTGAGCACTGAAGGC | HD HD NI HD NG NH NI NH HD NI |
| | | (SEQ ID NO: 26) | HD NG NH NI NI NH NH HD (SEQ |
| | | | ID NO: 358) |
| AAVS1 | 5 | TGGTTTCCACTGAGCACTG | NH NH NG NG NG HD HD NI HD |
| | | (SEQ ID NO: 27) | NG NH NI NH HD NI HD NG NH |
| | | | (SEQ ID NO: 359) |
| AAVS1 | 6 | TGGGGAAAATGACCCAACA | NH NH NH NH NI NI NI NI NG NH |
| | | (SEQ ID NO: 28) | NI HD HD HD NI NI HD NI (SEQ ID |
| | | | NO: 360) |
| AAVS1 | 7 | TAGGACAGTGGGGAAAATG | NI NH NH NI HD NI NH NG NH NH |
| | | (SEQ ID NO: 29) | NH NH NI NI NI NING NH (SEQ ID |
| | | | NO: 361) |
| AAVS1 | 8 | TCCAGGGACACGGTGCTAG | HD HD NI NH NH NH NI HD NI HD |
| | | (SEQ ID NO: 30) | NH NH NG NH HD NG NI NH (SEQ |
| | | | ID NO: 362) |
| AAVS1 | 9 | TCAGAGCCAGGAGTCCTGG | HD NI NH NI NH HD HD NI NH NH |
| | | (SEQ ID NO: 31) | NI NH NG HD HD NG NH NH (SEQ |
| | | | ID NO: 363) |
| AAVS1 | 10 | TCCTTCAGAGCCAGGAGTC | HD HD NG NG HD NI NH NI NH HD |
| | | (SEQ ID NO: 32) | HD NI NH NH NI NH NG HD (SEQ |
| | | | ID NO: 364) |
| AAVS1 | 11 | TCCTCCTTCAGAGCCAGGA | HD HD NG HD HD NG NG HD NI |
| | | (SEQ ID NO: 33) | NH NI NH HD HD NI NH NH NI |
| | | | (SEQ ID NO: 365) |
| AAVS1 | 12 | TCCAGCCCCTCCTCCTTCA | HD HD NI NH HD HD HD HD NG |
| | | (SEQ ID NO: 34) | HD HD NG HD HD NG NG HD NI |
| | | | (SEQ ID NO: 366) |
| AAVS1 | 13 | TCCGAGCTTGACCCTTGGA | HD HD NH NI NH HD NG NG NH NI |
| | | (SEQ ID NO: 35) | HD HD HD NG NG NH NH NI (SEQ |
| | | | ID NO: 367) |
| AAVS1 | 14 | TGGTTTCCGAGCTTGACCC | NH NH NG NG NG HD HD NH NI |
| | | (SEQ ID NO: 36) | NH HD NG NG NH NI HD HD HD |
| | | | (SEQ ID NO: 368) |
| AAVS1 | 15 | TGGGGTGGTTTCCGAGCTT | NH NH NH NH NG NH NH NG NG |
| | | (SEQ ID NO: 37) | NG HD HD NH NI NH HD NG NG |
| | | | (SEQ ID NO: 369) |
| AAVS1 | 16 | TCTGCTGGGGTGGTTTCCG | HD NG NH HD NG NH NH NH NH |
| | | (SEQ ID NO: 38) | NG NH NH NG NG NG HD HD NH |
| | | | (SEQ ID NO: 370) |
| AAVS1 | 17 | TGCAGAGTATCTGCTGGGG | NH HD NI NH NI NH NG NI NG HD |
| | | (SEQ ID NO: 39) | NG NH HD NG NH NH NH NH |
| | | | (SEQ ID NO: 371) |
| AAVS1 | AVS1 | CCAATCCCCTCAGT (SEQ | HD HD NI NI NG HD HD HD HD NG |
| | | ID NO: 40) | HD NI NH NG (SEQ ID NO: 372) |
| AAVS1 | AVS2 | CAGTGCTCAGTGGAA (SEQ | HD NI NH NG NH HD NG HD NI NH |
| | | ID NO: 41) | NG NH NH NI NI (SEQ ID NO: 373) |
| AAVS1 | AVS3 | GAAACATCCGGCGACTCA | NH NI NI NI HD NI NG HD HD NH |
| | | (SEQ ID NO: 42) | NH HD NH NI HD NG HD NI (SEQ |
| | | | ID NO: 374) |
| hROSA26 | 1F | TCGCCCCTCAAATCTTACA | HD NH HD HD HD HD NG HD NI NI |
| | | (SEQ ID NO: 43) | NING HD NG NG NI HD NI (SEQ |
| | | | ID NO: 375) |
| hROSA26 | 2F | TCAAATCTTACAGCTGCTC | HD NI NI NI NG HD NG NG NI HD |
| | | (SEQ ID NO: 44) | NI NH HD NG NH HD NG HD (SEQ |
| | | | ID NO: 376) |
| hROSA26 | 3F | TCTTACAGCTGCTCACTCC | HD NG NG NI HD NI NH HD NG NH |
| | | (SEQ ID NO: 45) | HD NG HD NI HD NG HD HD (SEQ |
| | | | ID NO: 377) |
| hROSA26 | 4F | TACAGCTGCTCACTCCCCT | NI HD NI NH HD NG NH HD NG HD |
| | | (SEQ ID NO: 46) | NI HD NG HD HD HD HD NG (SEQ |
| | | | ID NO: 378) |
| hROSA26 | 5F | TGCTCACTCCCCTGCAGGG | NH HD NG HD NI HD NG HD HD |
| | | (SEQ ID NO: 47) | HD HD NG NH HD NI NH NH NH |
| | | | (SEQ ID NO: 379) |
| hROSA26 | 6F | TCCCCTGCAGGGCAACGCC | HD HD HD HD NG NH HD NI NH |
| | | (SEQ ID NO: 48) | NH NH HD NI NI HD NH HD HD |
| | | | (SEQ ID NO: 380) |
| hROSA26 | 7F | TGCAGGGCAACGCCCAGGG | NH HD NI NH NH NH HD NI NI HD |
| | | (SEQ ID NO: 49) | NH HD HD HD NI NH NH NH (SEQ |
| | | | ID NO: 381) |
| hROSA26 | 8R | TCTCGATTATGGGGGGGAT | HD NG HD NH NI NG NG NING |
| | | (SEQ ID NO: 50) | NH NH NH HD NH NH NH NI NG |
| | | | (SEQ ID NO: 382) |
| hROSA26 | 9R | TCGCTTCTCGATTATGGGC | HD NH HD NG NG HD NG HD NH |
| | | (SEQ ID NO: 51) | NI NG NG NING NH NH NH HD |
| | | | (SEQ ID NO: 383) |
| hROSA26 | 10R | TGTCGAGTCGCTTCTCGAT | NH NG HD NH NI NH NG HD NH |
| | | (SEQ ID NO: 52) | HD NG NG HD NG HD NH NI NG |
| | | | (SEQ ID NO: 384) |
| hROSA26 | 11R | TCCATGTCGAGTCGCTTCT | HD HD NI NG NH NG HD NH NI NH |
| | | (SEQ ID NO: 53) | NG HD NH HD NG NG HD NG |
| | | | (SEQ ID NO: 385) |
| hROSA26 | 12R | TCGCCTCCATGTCGAGTCG | HD NH HD HD NG HD HD NI NG |
| | | (SEQ ID NO: 54) | NH NG HD NH NI NH NG HD NH |
| | | | (SEQ ID NO: 386) |
| hROSA26 | 13R | TCGTCATCGCCTCCATGTC | HD NH NG HD NI NG HD NH HD |
| | | (SEQ ID NO: 55) | HD NG HD HD NI NG NH NG HD |
| | | | (SEQ ID NO: 387) |
| hROSA26 | 14R | TGATCTCGTCATCGCCTCC | NH NI NG HD NG HD NH NG HD NI |
| | | (SEQ ID NO: 56) | NG HD NH HD HD NG HD HD |
| | | | (SEQ ID NO: 388) |
| hROSA26 | ROSA1 | GCTTCAGCTTCCTA (SEQ | NH HD NG NG HD NI NH HD NG |
| | | ID NO: 57) | NG HD HD NG NI (SEQ ID NO: |
| | | | 389) |
| hROSA26 | ROSA2 | CTGTGATCATGCCA (SEQ | HD NG NK NG NH NI NG HD NI NG |
| | | ID NO: 58) | NH HD HD NI (SEQ ID NO: 390) |
| hROSA26 | TALER2 | ACAGTGGTACACACCT | NI HD NI NN NG NN NN NG NI HD |
| | | (SEQ ID NO: 59) | NI HD NI HD HD NG (SEQ ID NO: |
| | | | 391) |
| hROSA26 | TALER3 | CCACCCCCCACTAAG (SEQ | HD HD NI HD HD HD HD HD HD NI |
| | | ID NO: 60) | HD NG NI NI NN (SEQ ID NO: 392) |
| hROSA26 | TALER4 | CATTGGCCGGGCAC (SEQ | HD NI NG NG NN NN HD HD NN |
| | | ID NO: 61) | NN NN HD NI HD (SEQ ID NO: 393) |
| hROSA26 | TALER5 | GCTTGAACCCAGGAGA | NN HD NG NG NN NI NI HD HD HD |
| | | (SEQ ID NO: 62) | NI NN NN NI NN NI (SEQ ID NO: |
| | | | 394) |
| CCR5 | TALC3 | ACACCCGATCCACTGGG | NI HD NI HD HD HD NN NI NG HD |
| | | (SEQ ID NO: 63) | HD NI HD NG NN NN NN (SEQ ID |
| | | | NO: 395) |
| CCR5 | TALC4 | GCTGCATCAACCCC (SEQ | NN HD NG NN HD NI NG HD NI NI |
| | | ID NO: 64) | HD HD HD HD (SEQ ID NO: 396) |
| CCR5 | TALC5 | GCCACAAACAGAAATA | NN NN HD NI HD NN NI NI NI HD |
| | | (SEQ ID NO: 65) | NI HD HD HD NG HD HD (SEQ ID |
| | | | NO: 397) |
| CCR5 | TALC7 | GGTGGCTCATGCCTG (SEQ | NN NN NG NN NN HD NG HD NI |
| | | ID NO: 66) | NG NN HD HD NG NN (SEQ ID NO: |
| | | | 398) |
| CCR5 | TALC8 | GATTTGCACAGCTCAT | NN NI NG NG NG NN HD NI HD NI |
| | | (SEQ ID NO: 67) | NN HD NG HD NI NG (SEQ ID NO: |
| | | | 399) |
| Chr 2 | SHCHR2-1 | AAGCTCTGAGGAGCA (SEQ | NI NI NH HD NG HD NG NH NI NH |
| | | ID NO: 68) | NH NI NH HD (SEQ ID NO: 400) |
| Chr 2 | SHCHR2-2 | CCCTAGCTGTCCC (SEQ | HD HD HD NG NI NK HD NG NH |
| | | ID NO: 69) | NG HD HD HD HD (SEQ ID NO: |
| | | | 401) |
| Chr 2 | SHCHR2-3 | GCCTAGCATGCTAG (SEQ | NH HD HD NG NI NH HD NI NG NH |
| | | ID NO: 70) | HD NG NI NH (SEQ ID NO: 402) |
| Chr 2 | SHCHR2-4 | ATGGGCTTCACGGAT (SEQ | NI NG NH NH NH HD NG NG HD NI |
| | | ID NO: 71) | HD NH NH NI NG (SEQ ID NO: |
| | | | 403 |
| Chr 4 | SHCHR4-1 | GAAACTATGCCTGC (SEQ | NH NI NI NI HD NG NING NH HD |
| | | ID NO: 72) | HD NG NH HD (SEQ ID NO: 404) |
| Chr 4 | SHCHR4-2 | GCACCATTGCTCCC (SEQ | NH HD NI HD HD NI NG NG NH HD |
| | | ID NO: 73) | NG HD HD HD (SEQ ID NO: 405) |
| Chr 4 | SHCHR4-3 | GACATGCAACTCAG (SEQ | NH NI HD NI NG NH HD NI NI HD |
| | | ID NO: 74) | NG HD NI NH (SEQ ID NO: 406) |
| Chr 6 | SHCHR6-1 | ACACCACTAGGGGT (SEQ | NI HD NI HD HD NI HD NG NI NH |
| | | ID NO: 75) | NH NH NH NG (SEQ ID NO: 407) |
| Chr 6 | SHCHR6-2 | GTCTGCTAGACAGG (SEQ | NH NG HD NG NH HD NG NI NH NI |
| | | ID NO: 76) | HD NI NH NH (SEQ ID NO: 408) |
| Chr 6 | SHCHR6-3 | GGCCTAGACAGGCTG (SEQ | NH NH HD HD NG NI NH NI HD NI |
| | | ID NO: 77) | NH NH HD NG NH (SEQ ID NO: |
| | | | 409) |
| Chr 6 | SHCHR6-4 | GAGGCATTCTTATCG (SEQ | NH NI NH NH HD NI NG NG HD NG |
| | | ID NO: 78) | NG NI NG HD NH (SEQ ID NO: |
| | | | 410) |
| Chr 10 | SHCHR10- | GCCTGGAAACGTTCC (SEQ | NN HD HD NG NN NN NI NI NI HD |
| | 1 | ID NO: 79) | NN NG NG HD HD (SEQ ID NO: |
| | | | 411) |
| Chr 10 | SHCHR10- | GTGCTCTGACAATA (SEQ | NN NG NN HD NG HD NG NN NI |
| | 2 | ID NO: 80) | HD NI NI NG NI (SEQ ID NO: 412) |
| Chr 10 | SHCHR10- | GTTTTGCAGCCTCC (SEQ | NN NG NG NG NG NN HD NI NN |
| | 3 | ID NO: 81) | HD HD NG HD HD (SEQ ID NO: |
| | | | 413) |
| Chr 10 | SHCHR10- | ACAGCTGTGGAACGT (SEQ | NI HD NI NN HD NG NN NG NN NN |
| | 4 | ID NO: 82) | NI NI HD NN NG (SEQ ID NO: 414) |
| Chr 10 | SHCHR10- | GGCTCTCTTCCTCCT (SEQ | HD NI NI NN NI HD HD NN NI NN |
| | 5 | ID NO: 83) | HD NI HD NG NN HD NG NN (SEQ |
| | | | ID NO: 415) |
| Chr 11 | SHCHR11- | CTATCCCAAAACTCT (SEQ | HD NG NI NG HD HD HD NI NI NI |
| | 1 | ID NO: 84) | NI HD NG HD NG (SEQ ID NO: |
| | | | 416) |
| Chr 11 | SHCHR11- | GAAAAACTATGTAT (SEQ | NH NI NI NI NI NI HD NG NING NH |
| | 2 | ID NO: 85) | NG NI NG (SEQ ID NO: 417) |
| Chr 11 | SHCHR11- | AGGCAGGCTGGTTGA (SEQ | NI NH NH HD NI NH NH HD NG NH |
| | 3 | ID NO: 86) | NH NG NG NH NI (SEQ ID NO: |
| | | | 418) |
| Chr 17 | SHCHR17- | CAATACAACCACGC (SEQ | HD NI NI NG NI HD NI NI HD HD NI |
| | 1 | ID NO: 87) | HD NN HD (SEQ ID NO: 419) |
| Chr 17 | SHCHR17- | ATGACGGACTCAACT (SEQ | NI NG NN NI HD NN NN NI HD NG |
| | 2 | ID NO: 88) | HD NI NI HD NG (SEQ ID NO: 420) |
| Chr 17 | SHCHR17- | CACAACATTTGTAA (SEQ | HD NI HD NI NI HD NI NG NG NG |
| | 3 | ID NO: 89) | NN NG NI NI (SEQ ID NO: 421) |
| Chr 17 | SHCHR17- | ATTTCCAGTGCACA (SEQ | NI NG NG NG HD HD NI NN NG |
| | 4 | ID NO: 90) | NN HD NI HD NI (SEQ ID NO: 422) |

In embodiments, TALEs for targeting human genomic safe harbor sites using any of the TALE-based targeting elements to the TTAA site in hROSA26 (e.g., hg38 chr3:9,396,133-9,396,305) are shown in TABLE 4B.

TABLE 4B

| NAME | DNA SEQUENCE | RVD AMINO ACID CODE |
|---|---|---|
| R1 | TCGCCCCTCAAATCTTACAG | HD NH HD HD HD HD NG HD NI NI NI NG HD NG NG NI HD NI NH |
| | (SEQ ID NO: 583) | (SEQ ID NO: 596) |
| R2 | TCAAATCTTACAGCTGCTCA | HD NI NI NI NG HD NG NG NI HD NI NH HD NG NH HD NG HD NI |
| | (SEQ ID NO: 584) | (SEQ ID NO: 597) |
| R3 | TCTTACAGCTGCTCACTCCC | HD NG NG NI HD NI NH HD NG NH HD NG HD NI HD NG HD HD HD |
| | (SEQ ID NO: 585) | (SEQ ID NO: 598) |
| R4 | TACAGCTGCTCACTCCCCTG | NI HD NI NH HD NG NH HD NG HD NI HD NG HD HD HD HD NG NH |
| | (SEQ ID NO: 586) | (SEQ ID NO: 599) |
| R5 | TGCTCACTCCCCTGCAGGGC | NH HD NG HD NI HD NG HD HD HD HD NG NH HD NI NH NH NH HD |
| | (SEQ ID NO: 587) | (SEQ ID NO: 600) |
| R6 | TCCCCTGCAGGGCAACGCCC | HD HD HD HD NG NH HD NI NH NH NH HD NI NI HD NH HD HD HD |
| | (SEQ ID NO: 455) | (SEQ ID NO: 601) |
| R7 | TGCAGGGCAACGCCCAGGGA | NH HD NI NH NH NH HD NI NI HD NH HD HD HD NI NH NH NH NI |
| | (SEQ ID NO: 588) | (SEQ ID NO: 602) |
| R8 | TCTCGATTATGGGCGGGATT | HD NG HD NH NI NG NG NI NG NH NH NH HD NH NH NH NI NG NG |
| | (SEQ ID NO: 589) | (SEQ ID NO: 603) |
| R9 | TCGCTTCTCGATTATGGGCG | HD NH HD NG NG HD NG HD NH NI NG NG NI NG NH NH NH HD NH |
| | (SEQ ID NO: 590) | (SEQ ID NO: 604) |
| R10 | TGTCGAGTCGCTTCTCGATT | NH NG HD NH NI NH NG HD NH HD NG NG HD NG HD NH NI NG NG |
| | (SEQ ID NO: 591) | (SEQ ID NO: 605) |
| R11 | TCCATGTCGAGTCGCTTCTC | HD HD NI NG NH NG HD NH NI NH NG HD HD NH HD NG NG HD NG |
| | (SEQ ID NO: 592) | HD (SEQ ID NO: 606) |
| R12 | TCGCCTCCATGTCGAGTCGC | HD NH HD HD NG HD HD NI NG NH NG HD NH NI NH NG HD NH HD |
| | (SEQ ID NO: 593) | (SEQ ID NO: 607) |
| R13 | TCGTCATCGCCTCCATGTCG | HD NH NG HD NI NG HD NH HD HD NG HD HD NI NG NH NG HD NH |
| | (SEQ ID NO: 594) | (SEQ ID NO: 608) |
| R14 | TGATCTCGTCATCGCCTCCA | NH NI NG HD NG HD NH NG HD NI NG HD NH HD HD NG HD HD NI |
| | (SEQ ID NO: 595) | (SEQ ID NO: 609) |

In embodiments, TALEs for targeting human genomic safe harbor sites using any of the TALE-based targeting elements to the AAVS1 (e.g., hg38 chr19:55,112,851-55,113,324) are shown in TABLE 4C.

TABLE 4C
RVD AMINO ACID CODE

| NAME | DNA SEQUENCE | RVD AMINO ACID CODE |
|---|---|---|
| AAV1c | TGGCCGGCCTGACCACTGGG (SEQ ID NO: 610) | NH NH HD HD NH NH HD HD NG NH NI HD HD NI |
| | | HD NG NH NH NH (SEQ ID NO: 625) |
| AAV2c | TGAAGGCCTGGCCGGCCTGA (SEQ ID NO: 611) | NH NI NI NH NH HD HD NG NH NH HD HD NH NH |
| | | HD HD NG NH NI (SEQ ID NO: 626) |
| AAV3c | TGAGCACTGAAGGCCTGGCC (SEQ ID NO: 612) | NH NI NH HD NI HD NG NH NI NI NH NH HD HD |
| | | NG NH NH HD HD (SEQ ID NO: 627) |
| AAV4c | TCCACTGAGCACTGAAGGCC (SEQ ID NO: 613) | HD HD NI HD NG NH NI NH HD NI HD NG NH NI |
| | | NI NH NH HD HD (SEQ ID NO: 628) |
| AAV5c | TGGTTTCCACTGAGCACTGA (SEQ ID NO: 614) | NH NH NG NG NG HD HD NI HD NG NH NI NH HD |
| | | NI HD NG NH NI (SEQ ID NO: 629) |
| AAV6 | TGGGGAAAATGACCCAACAG (SEQ ID NO: 615) | NH NH NH NH NI NI NI NI NG NH NI HD HD HD |
| | | NI NI HD NI NH (SEQ ID NO: 630) |
| AAV7 | TAGGACAGTGGGGAAAATGA (SEQ ID NO: 616) | NI NH NH NI HD NI NH NG NH NH NH NH NI NI |
| | | NI NI NG NH NI (SEQ ID NO: 631) |
| AAV8 | TCCAGGGACACGGTGCTAGG (SEQ ID NO: 617) | HD HD NI NH NH NH NI HD NI HD NH NH NG NH |
| | | HD NG NI NH NH (SEQ ID NO: 632) |
| AAV9 | TCAGAGCCAGGAGTCCTGGC (SEQ ID NO: 618) | HD NI NH NI NH HD HD NI NH NH NI NH NG HD |
| | | HD NG NH NH HD (SEQ ID NO: 633) |
| AAV10 | TCCTTCAGAGCCAGGAGTCC (SEQ ID NO: 619) | HD HD NG NG HD NI NH NI NH HD HD NI NH NH |
| | | NI NH NG HD HD (SEQ ID NO: 634) |
| AAV11 | TCCTCCTTCAGAGCCAGGAG (SEQ ID NO: 620) | HD HD NG HD HD NG NG HD NI NH NI NH HD HD |
| | | NI NH NH NI NH (SEQ ID NO: 635) |
| AAV12 | TCCAGCCCCTCCTCCTTCAG (SEQ ID NO: 621) | HD HD NI NH HD HD HD HD NG HD HD NG HD HD |
| | | NG NG HD NI NH (SEQ ID NO: 636) |
| AAV13c | TCCGAGCTTGACCCTTGGAA (SEQ ID NO: 461) | HD HD NH NI NH HD NG NG NH NI HD HD HD NG |
| | | NG NH NH NI NI (SEQ ID NO: 637) |
| AAV14c | TGGTTTCCGAGCTTGACCCT (SEQ ID NO: 112) | NH NH NG NG NG HD HD NH NI NH HD NG NG NH |
| | | NI HD HD HD NG (SEQ ID NO: 638) |
| AAV15c | TGGGGTGGTTTCCGAGCTTG (SEQ ID NO: 622) | NH NH NH NH NG NH NH NG NG NG HD HD NH NI |
| | | NH HD NG NG NH (SEQ ID NO: 639) |
| AAV16c | TCTGCTGGGGTGGTTTCCGA (SEQ ID NO: 623) | HD NG NH HD NG NH NH NH NH NG NH NH NG NG |
| | | NG HD HD NH NI (SEQ ID NO: 640) |
| AAV17c | TGCAGAGTATCTGCTGGGGT (SEQ ID NO: 624) | NH HD NI NH NI NH NG NI NG HD NG NH HD NG |
| | | NH NH NH NH NG (SEQ ID NO: 641) |

In embodiments, TALEs for targeting human genomic safe harbor sites using any of the TALE-based targeting elements to Chromosome 4 (e.g., hg38 chr4:30,793,534-30,875,476 or hg38 chr4:30,793,533-30,793,537 (9677); chr4:30,875,472-30,875,476 (8948)) are shown in TABLE 4D.

TABLE 4D

| NAME | DNA SEQUENCE | RVD AMINO ACID CODE |
|---|---|---|
| TALE4-R001 | TCTTCCTAGTATTAAAGT (SEQ ID | HD NG NG HD HD NG NI NH NG NI NG NG NI NI NI |
| | NO: 642) | NH NG (SEQ ID NO: 662) |
| TALE4-R002 | TCCTTAATATTACCAGT (SEQ ID NO: | HD HD NG NG NI NI NG NI NG NG NI HD HD NI NH |
| | 643) | NG (SEQ ID NO: 663) |
| TALE4-F003 | TACCAAGCTGAAATGACACAAAAGT | NI HD HD NI NI NH HD NG NH NI NI NI NG NH NI |
| | (SEQ ID NO: 644) | HD NI HD NI NI NI NI NH NG (SEQ ID NO: 664) |
| TALE4-F004 | TGGCTGTGTCACATACCAGCAGAAT | NH NH HD NG NH NG NH NG HD NI HD NI NG NI HD |
| | (SEQ ID NO: 645) | HD NI NH HD NI NH NI NI NG (SEQ ID NO: 665) |
| TALE4-F005 | TGTTAATTTGAATACAATCACT (SEQ | NH NG NG NI NI NG NG NG NH NI NI NG NI HD NI |
| | ID NO: 646) | NI NG HD NI HD NG (SEQ ID NO: 666) |
| TALE4-F006 | TGTGTCACATACCAGCAGAAT (SEQ ID | NH NG NH NG HD NI HD NI NG NI HD HD NI NH HD |
| | NO: 647) | NI NH NI NI NG (SEQ ID NO: 667) |
| TALE4-R007 | TGGTAACTACTAATTT (SEQ ID NO: | NH NH NG NI NI HD NG NI HD NG NI NI NG NG NG |
| | 648) | (SEQ ID NO: 668) |
| TALE4-F008 | TGTCACATACCAGCAGAAT (SEQ ID | NH NG HD NI HD NI NG NI HD HD NI NH HD NI NH |
| | NO: 649) | NI NI NG (SEQ ID NO: 669) |
| TALE4-R009 | TGTGACACAGCCATCAACAAT (SEQ ID | NH NG NH NI HD NI HD NI NH HD HD NI NG HD NI |
| | NO: 650) | NI HD NI NI NG (SEQ ID NO: 670) |
| TALE4-F010 | TCCTTTGATGAACAGT (SEQ ID NO: | HD HD NG NG NG NH NI NG NH NI NI HD NI NH NG |
| | 651) | (SEQ ID NO: 671) |
| TALE4-F011 | TGTGTGCAATAGCGTTAAAGGAACTACAT | NH NG NH NG NH HD NI NI NG NI NH HD NH NG NG |
| | (SEQ ID NO: 652) | NI NI NI NH NH NI NI HD NG NI HD NI NG (SEQ ID |
| | | NO: 672) |
| TALE4-F012 | TCTTTCAATAGCCCACT (SEQ ID NO: | HD NG NG NG HD NI NI NG NI NH HD HD HD NI HD |
| | 653) | NG (SEQ ID NO: 673) |
| TALE4-R013 | TCTCAAATGACAAGAGCACAGT (SEQ | HD NG HD NI NI NI NG NH NI HD NI NI NH NI NH |
| | ID NO: 654) | HD NI HD NI NH NG (SEQ ID NO: 674) |
| TALE4-F014 | TACCAGTTAATTAGCACT (SEQ ID | NI HD HD NI NH NG NG NI NI NG NG NI NH HD NI |
| | NO: 655) | HD NG (SEQ ID NO: 675) |
| TALE4-F015 | TGTTGTGACCTAAGCCAT (SEQ ID | NH NG NG NH NG NH NI HD HD NG NI NI NH HD HD |
| | NO: 656) | NI NG (SEQ ID NO: 676) |
| TALE4-R016 | TCTCATGTTTTAAAGTCAAGAAT (SEQ | HD NG HD NI NG NH NG NG NG NG NI NI NI NH NG |
| | ID NO: 657) | HD NI NI NH NI NI NG (SEQ ID NO: 677) |
| TALE4-F017 | TCCTGAATTCAGAACAGAT (SEQ ID | HD HD NG NH NI NI NG NG HD NI NH NI NI HD NI |
| | NO: 658) | NH NI NG (SEQ ID NO: 678) |
| TALE4-F018 | TAGCATGATGTTTCATGTTGTGACCT | NI NH HD NI NG NH NI NG NH NG NG NG HD NI NG |
| | (SEQ ID NO: 659) | NH NG NG NH NG NH NI HD HD NG (SEQ ID NO: 679) |
| TALE4-F019 | TGTTTCATGTTGTGACCTAAGCCAT | NH NG NG NG HD NI NG NH NG NG NH NG NH NI HD |
| | (SEQ ID NO: 660) | HD NG NI NI NH HD HD NI NG (SEQ ID NO: 680) |
| TALE4-F020 | TACAACAGTCTATTTCAT (SEQ ID | NI HD NI NI HD NI NH NG HD NG NI NG NG NG HD |
| | NO: 661) | NI NG (SEQ ID NO: 681) |

In embodiments, TALEs for targeting human genomic safe harbor sites using any of the TALE-based targeting elements to Chromosome 22 (e.g., hg38 chr22:35,370,000-35,380,000 or hg38 chr22:35,373,912-35,373,916 (861); chr22:35,377,843-35,377,847 (1153)) are shown in TABLE 4E.

TABLE 4E

| NAME | DNA SEQUENCE | RVD AMINO ACID CODE |
|---|---|---|
| TALE22F - | TCTTCCTAGTCTCTTCTCTACCCAGT (SEQ ID | HD NG NG HD HD NG NI NH NG HD NG HD NG NG |
| R001 | NO: 682) | HD NG HD NG NI HD HD HD NI NH NG (SEQ ID |
| | | NO: 702) |
| TALE22 - | TACACTCCAGCCTGGGAAACAGAGT (SEQ ID | NI HD NI HD NG HD HD NI NH HD HD NG NH NH |
| F002 | NO: 683) | NH NI NI NI HD NI NH NI NH NG (SEQ ID NO: |
| | | 703) |
| TALE22 - | TCTTTTCCTTAGGACGGCT (SEQ ID NO: | HD NG NG NG NG HD HD NG NG NI NH NH NI HD |
| F003 | 684) | NH NH HD NG (SEQ ID NO: 704) |
| TALE22 - | TCGCTCAGGCCTGTCAT (SEQ ID NO: 685) | HD NH HD NG HD NI NH NH HD HD NG NH NG HD |
| F004 | | NI NG (SEQ ID NO: 705) |
| TALE22 - | TCCATATGGAAGACTT (SEQ ID NO: 686) | HD HD NI NG NI NG NH NH NI NI NH NI HD NG |
| F005 | | NG (SEQ ID NO: 706) |
| TALE22 - | TACCCAGTTAACCACCCT (SEQ ID NO: 687) | NI HD HD HD NI NH NG NG NI NI HD HD NI HD |
| F006 | | HD HD NG (SEQ ID NO: 707) |
| TALE22 - | TGGCGCATGCCTGTAATCCCAGCTACT (SEQ ID | NH NH HD NH HD NI NG NH HD HD NG NH NG NI |
| F007 | NO: 688) | NI NG HD HD HD NI NH HD NG NI HD NG (SEQ |
| | | ID NO: 708) |
| TALE22 - | TATACGAGGAGAAAATTAGCATTCCT (SEQ ID | NI NG NI HD NH NI NH NH NI NH NI NI NI NI |
| F008 | NO: 689) | NG NG NI NH HD NI NG NG HD HD NG (SEQ ID |
| | | NO: 709) |
| TALE22 - | TCTGCCTCCCAGGTTCACGCAAT (SEQ ID NO: | HD NG NH HD HD NG HD HD HD NI NH NH NG NG |
| R009 | 690) | HD NI HD NH HD NI NI NG (SEQ ID NO: 710) |
| TALE22 - | TGCCTTGTCACGTTTTCACAGT (SEQ ID NO: | NH HD HD NG NG NH NG HD NI HD NH NG NG NG |
| F010 | 691) | NG HD NI HD NI NH NG (SEQ ID NO: 711) |
| TALE22 - | TGTCACCTTCTGTATGTGCAACCAT (SEQ ID | NH NG HD NI HD HD NG NG HD NG NH NG NI NG |
| F001A | NO: 692) | NH NG NH HD NI NI HD HD NI NG (SEQ ID NO: |
| | | 712) |
| TALE22 - | TCTGTATGTGCAACCAT (SEQ ID NO: 693) | HD NG NH NG NI NG NH NG NH HD NI NI HD HD |
| F002A | | NI NG (SEQ ID NO: 713) |
| TALE22 - | TAGTCAAGCAACAGGAT (SEQ ID NO: 694) | NI NH NG HD NI NI NH HD NI NI HD NI NH NH |
| R03A | | NI NG (SEQ ID NO: 714) |
| TALE22 - | TCCAAGATAATTCCCCAT (SEQ ID NO: 695) | HD HD NI NI NH NI NG NI NI NG NG HD HD HD |
| F004A | | HD NI NG (SEQ ID NO: 715) |
| TALE22 - | TCTGCAAGATCCTTTT (SEQ ID NO: 696) | HD NG NH HD NI NI NH NI NG HD HD NG NG NG |
| F005A | | NG (SEQ ID NO: 716) |
| TALE22 - | TGCTATGTAAGGTAGCAAAAAGGTAACCT (SEQ | NH HD NG NI NG NH NG NI NI NH NH NG NI NH |
| F006A | ID NO: 697) | HD NI NI NI NI NI NH NH NG NI NI HD HD NG |
| | | (SEQ ID NO: 717) |
| TALE22 - | TCTCTCTCCTCCTGCT (SEQ ID NO: 698) | HD NG HD NG HD NG HD HD NG HD HD NG NH HD |
| R007A | | NG (SEQ ID NO: 718) |
| TALE22 - | TCCAAATGCTATTCTCTCT (SEQ ID NO: | HD HD NI NI NI NG NH HD NG NI NG NG HD NG |
| R008A | 699) | HD NG HD NG (SEQ ID NO: 719) |
| TALE22 - | TGCTGATTCAGCCTCCT (SEQ ID NO: 700) | NH HD NG NH NI NG NG HD NI NH HD HD NG HD |
| R009A | | HD NG (SEQ ID NO: 720) |
| TALE22 - | TAGAACAGCCCCCCACACAGT (SEQ ID NO: | NI NH NI NI HD NI NH HD HD HD HD HD HD NI |
| F010A | 701) | HD NI HD NI NH NG (SEQ ID NO: 721) |

In embodiments, TALEs for targeting human genomic safe harbor sites using any of the TALE-based targeting elements to Chromosome X (e.g., hg38 chrX:134,419,661-134,541,172 or hg38 chrX:134,476,304-134,476,307 (85); chrX:134,476,337-134,476,340 (51)) are shown in TABLE 4F.

TABLE 4F

| NAME | DNA SEQUENCE | RVD AMINO ACID CODE |
|---|---|---|
| TALE F002 | TTTAGCAGATGCATCAGC (SEQ ID | NG NG NI NH HD NI NH NI NG NH HD NI NG HD NI NH HD |
| | NO: 722) | (SEQ ID NO: 746) |
| TALE F003 | TGACCAGGGGCATGTCCTGG (SEQ ID | NH NI HD HD NI NH NH NH NH HD NI NG NH NG HD HD NG |
| | NO: 723) | NH NH (SEQ ID NO: 747) |
| TALE F004 | TGGTCCACCTACCTGAAAATG (SEQ ID | HD NI NI NH NH NI NH NG NG HD NG NH NH HD NG NH NH |
| | NO: 724) | NH NG HD (SEQ ID NO: 748) |
| TALE F007 | TGTCCCACAGGTATTACGGGC (SEQ ID | NH NG HD HD HD NI HD NI NH NH NG NI NG NG NI HD NH |
| | NO: 725) | NH NH HD (SEQ ID NO: 749) |
| TALE F008 | TACGGGCCAACCTGACAATAC (SEQ ID | NI HD NH NH NH HD HD NI NI HD HD NG NH NI HD NI NI |
| | NO: 726) | NG NI HD (SEQ ID NO: 750) |
| TALE F009 | TGAGCTTTGGGGACTGAAAGA (SEQ ID | NH NI NH HD NG NG NG NH NH NH NH NI HD NG NH NI NI |
| | NO: 727) | NI NH NI (SEQ ID NO: 751) |
| TALE R002 | CTGGCATAATCTTTTCCCCCA (SEQ ID | NH NH NH NH NH NI NI NI NI NH NI NG NG NI NG NH HD |
| | NO: 728) | HD NI NH (SEQ ID NO: 752) |
| TALE R003 | CCAGCCTCCTGGCCATGTGCA (SEQ ID | NH HD NI HD NI NG NH NH HD HD NI NH NH NI NH NH HD |
| | NO: 729) | NG NH NH (SEQ ID NO: 753) |
| TALE R004 | GGCCATGTGCACAGGGGCTGA (SEQ ID | HD NI NH HD HD HD HD NG NH NG NH HD NI HD NI NG NH |
| | NO: 730) | NH HD HD (SEQ ID NO: 754) |
| TALE R005 | CTGATATGTGAAGGTTTAGCA (SEQ ID | NH HD NG NI NI NI HD HD NG NG HD NI HD NI NG NI NG |
| | NO: 731) | HD NI NH (SEQ ID NO: 755) |
| TALE R007 | TGACCAGGCGTGGTGGCTCAC (SEQ ID | NH NI HD HD NI NH NH HD NH NG NH NH NG NH NH HD NG |
| | NO: 732) | HD NI HD (SEQ ID NO: 756) |
| TALE F020* | TATAGACATTTTCACT (SEQ ID NO: | NI NG NI NH NI HD NI NG NG NG NG HD NI HD NG (SEQ |
| | 733) | ID NO: 757) |
| TALE F021* | TCTACATTTAACTATCAACCT (SEQ ID | HD NG NI HD NI NG NG NG NI NI HD NG NI NG HD NI NI |
| | NO: 734) | HD HD NG (SEQ ID NO: 758) |
| TALE F030* | TCGTGCAAACGTTTGAT (SEQ ID NO: | HD NH NG NH HD NI NI NI HD NH NG NG NG NH NI NG |
| | 735) | (SEQ ID NO: 759) |
| TALE F031* | TACATCAATCCTGTAGGT* (SEQ ID | NI HD NI NG HD NI NI NG HD HD NG NH NG NI NH NH NG |
| | NO: 736) | (SEQ ID NO: 760) |
| TALE F034* | TCTATTTTAGTGACCCAAGT (SEQ ID | HD NG NI NG NG NG NG NI NH NG NH NI HD HD HD NI NI |
| | NO: 737) | NH NG (SEQ ID NO: 761) |
| TALE F036* | TAGAGTCAAAGCATGTACT (SEQ ID | NI NH NI NH NG HD NI NI NI NH HD NI NG NH NG NI HD |
| | NO: 738) | NG (SEQ ID NO: 762) |
| TALE F037* | TCCTACCCATAAGCTCCT (SEQ ID | HD HD NG NI HD HD HD NI NG NI NI NH HD NG HD HD NG |
| | NO: 739) | (SEQ ID NO: 763) |
| TALE F040* | TCCCCATCCCCATCAGT (SEQ ID NO: | HD HD HD HD NI NG HD HD HD HD NI NG HD NI NH NG |
| | 740) | (SEQ ID NO: 764) |
| TALE R022* | TCTTTAATTCAAGCAAGACTTTAACAAGT | HD NG NG NG NI NI NG NG HD NI NI NH HD NI NI NH NI |
| | (SEQ ID NO: 741) | HD NG NG NG NI NI HD NI NI NH NG (SEQ ID NO: 765) |
| TALE R033* | TGCAGTCCCCTTTCTT (SEQ ID NO: | NH HD NI NH NG HD HD HD HD NG NG NG HD NG NG (SEQ |
| | 742) | ID NO: 766) |
| TALE R035* | TCTGCACAAATCCCCAAAGAT (SEQ ID | NH HD NI NH NG HD HD HD HD NG NG NG HD NG NG (SEQ |
| | NO: 743) | ID NO: 767) |
| TALE R038* | TACATGCTTTGACTCT (SEQ ID NO: | NH HD NI NH NG HD HD HD HD NG NG NG HD NG NG (SEQ |
| | 744) | ID NO: 768) |
| TALE R039* | TGGCCAGTTATACTGCCAGCAGCTATAAT | NH NH HD HD NI NH NG NG NI NG NI HD NG NH HD HD NI |
| | (SEQ ID NO: 745) | NH HD NI NH HD NG NI NG NI NI NG (SEQ ID NO: 769) |

In embodiments, the helper enzyme is capable of inserting a donor DNA at a TA dinucleotide site. In embodiments, the helper enzyme is capable of inserting a donor DNA at a TTAA (SEQ ID NO: 440) tetranucleotide site.

Illustrative DNA binding codes for human genomic safe harbor in areas of open chromatin via ZNFs, encompassed by various embodiments are provided in TABLE 5A-5E. In embodiments, there is provided a variant of the ZNFs, encompassed by various embodiments are provided in TABLE 5A-5E, e.g., having a sequence having at least about 90%, or at least about 93%, or at least about 95%, or at least about 97%, or at least about 98%, or at least about 99% identity to any of the sequences in TABLE 5A-5E.

In embodiments, ZNFs for targeting human genomic safe harbor sites using any of the ZNF-based targeting elements to the TTAA site in hROSA26 (e.g., hg38 chr3:9,396,133-9,396,305) are shown in TABLE 5A.

TABLE 5A

| hROSA26 | | | | |
|---|---|---|---|---|
| TTAA | NAME | TARGET | SCORE | ZFP AMINO ACID CODE |
| 5′ | ZnF3a | TGG GAA GAT | 58.64 | LEPGEKPYKCPECGKSFSONSTLTEHQRTHTGEKPYKCPECGKSFSQRANLRAHQ |
| | | AAA CTA (SEQ | | RTHTGEKPYKCPECGKSFSTSGNLVRHQRTHTGEKPYKCPECGKSFSQSSNLVRH |
| | | ID NO: 770) | | QRTHTGEKPYKCPECGKSFSRSDHLTTHQRTHTGKKTS (SEQ ID NO: 783) |
| 5′ | ZnF5a | ACT CCC CTG | 56.25 | LEPGEKPYKCPECGKSFSDSGNLRVHQRTHTGEKPYKCPECGKSFSDPGHLVRHQ |
| | | CAG GGC AAC | | RTHTGEKPYKCPECGKSFSRADNLTEHQRTHTGEKPYKCPECGKSFSRNDALTEH |
| | | (SEQ ID NO: | | QRTHTGEKPYKCPECGKSFSSKKHLAEHQRTHTGEKPYKCPECGKSFSTHLDLIR |
| | | 771) | | HQRTHTGKKTS (SEQ ID NO: 784) |
| 5′ | ZnF5b | CCC CTG CAG | 56.25 | LEPGEKPYKCPECGKSFSDCRDLARHQRTHTGEKPYKCPECGKSFSDSGNLRVHQ |
| | | GGC AAC GCC | | RTHTGEKPYKCPECGKSFSDPGHLVRHQRTHTGEKPYKCPECGKSFSRADNLTEH |
| | | (SEQ ID NO: | | QRTHTGEKPYKCPECGKSFSRNDALTEHQRTHTGEKPYKCPECGKSFSSKKHLAE |
| | | 772) | | HQRTHTGKKTS (SEQ ID NO: 785) |
| 5′ | ZnF5c | CTG CAG GGC | 60.58 | LEPGEKPYKCPECGKSFSRADNLTEHQRTHTGEKPYKCPECGKSFSDCRDLARHQ |
| | | AAC GCC CAG | | RTHTGEKPYKCPECGKSFSDSGNLRVHQRTHTGEKPYKCPECGKSFSDPGHLVRH |
| | | (SEQ ID NO: | | QRTHTGEKPYKCPECGKSFSRADNLTEHQRTHTGEKPYKCPECGKSFSRNDALTE |
| | | 773) | | HQRTHTGKKTS (SEQ ID NO: 786) |
| 5′ | ZnF5d | CAG GGC AAC | 58.08 | LEPGEKPYKCPECGKSFSQRAHLERHQRTHTGEKPYKCPECGKSFSRADNLTEHQ |
| | | GCC CAG GGA | | RTHTGEKPYKCPECGKSFSDCRDLARHQRTHTGEKPYKCPECGKSFSDSGNLRVH |
| | | (SEQ ID NO: | | QRTHTGEKPYKCPECGKSFSDPGHLVRHQRTHTGEKPYKCPECGKSFSRADNLTE |
| | | 774) | | HQRTHTGKKTS (SEQ ID NO: 787) |
| 5′ | ZnF5e | GGC AAC GCC | 57.32 | LEPGEKPYKCPECGKSFSTSHSLTEHQRTHTGEKPYKCPECGKSFSQRAHLERHQ |
| | | CAG GGA CCA | | RTHTGEKPYKCPECGKSFSRADNLTEHQRTHTGEKPYKCPECGKSFSDCRDLARH |
| | | (SEQ ID NO: | | QRTHTGEKPYKCPECGKSFSDSGNLRVHQRTHTGEKPYKCPECGKSFSDPGHLVR |
| | | 775) | | HQRTHTGKKTS (SEQ ID NO: 788) |
| 5′ | ZnF5f | AAC GCC CAG | 54.99 | LEPGEKPYKCPECGKSFSHRTTLTNHQRTHTGEKPYKCPECGKSFSTSHSLTEHQ |
| | | GGA CCA AGT | | RTHTGEKPYKCPECGKSFSQRAHLERHQRTHTGEKPYKCPECGKSFSRADNLTEH |
| | | (SEQ ID NO: | | QRTHTGEKPYKCPECGKSFSDCRDLARHQRTHTGEKPYKCPECGKSFSDSGNLRV |
| | | 776) | | HQRTHTGKKTS (SEQ ID NO: 789) |
| 5′ | ZnF5g | GCC CAG GGA | 55.31 | LEPGEKPYKCPECGKSFSREDNLHTHQRTHTGEKPYKCPECGKSFSHRTTLTNHQ |
| | | CCA AGT TAG | | RTHTGEKPYKCPECGKSFSTSHSLTEHQRTHTGEKPYKCPECGKSFSQRAHLERH |
| | | (SEQ ID NO: | | QRTHTGEKPYKCPECGKSFSRADNLTEHQRTHTGEKPYKCPECGKSFSDCRDLAR |
| | | 777) | | HQRTHTGKKTS (SEQ ID NO: 790) |
| 5′ | ZnF5h | CAG GGA CCA | 50.76 | LEPGEKPYKCPECGKSFSSKKHLAEHQRTHTGEKPYKCPECGKSFSREDNLHTHQ |
| | | AGT TAG CCC | | RTHTGEKPYKCPECGKSFSHRTTLTNHQRTHTGEKPYKCPECGKSFSTSHSLTEH |
| | | (SEQ ID NO: | | QRTHTGEKPYKCPECGKSFSQRAHLERHQRTHTGEKPYKCPECGKSFSRADNLTE |
| | | 778) | | HQRTHTGKKTS (SEQ ID NO: 791) |
| 3′ | ZnF12a | GCC TAG GCA | 59.09 | LEPGEKPYKCPECGKSFSQSSNLVRHQRTHTGEKPYKCPECGKSFSQRANLRAHQ |
| | | AAA GAA (SEQ | | RTHTGEKPYKCPECGKSFSQSGDLRRHQRTHTGEKPYKCPECGKSFSREDNLHTH |
| | | ID NO: 779) | | QRTHTGEKPYKCPECGKSFSDCRDLARHQRTHTGKKTS (SEQ ID NO: 792) |
| 3′ | ZnF13a | CGC GAG GAG | 57.19 | LEPGEKPYKCPECGKSFSRSDHLTNHQRTHTGEKPYKCPECGKSFSRSDHLTNHQ |
| | | GAA AGG AGG | | RTHTGEKPYKCPECGKSFSQSSNLVRHQRTHTGEKPYKCPECGKSFSRSDNLVRH |
| | | (SEQ ID NO: | | QRTHTGEKPYKCPECGKSFSRSDNLVRHQRTHTGEKPYKCPECGKSFSHTGHLLE |
| | | 780) | | HQRTHTGKKTS (SEQ ID NO: 793) |
| 3′ | ZnF13b | GAG GAG GAA | 57.80 | LEPGEKPYKCPECGKSFSRSDNLVRHQRTHTGEKPYKCPECGKSFSRSDHLTNHQ |
| | | AGG AGG GAG | | RTHTGEKPYKCPECGKSFSRSDHLTNHQRTHTGEKPYKCPECGKSFSQSSNLVRH |
| | | (SEQ ID NO: | | QRTHTGEKPYKCPECGKSFSRSDNLVRHQRTHTGEKPYKCPECGKSFSRSDNLVR |
| | | 781) | | HQRTHTGKKTS (SEQ ID NO: 794) |
| 3′ | ZnF13c | GAG GAA AGG | 57.61 | LEPGEKPYKCPECGKSFSDPGHLVRHQRTHTGEKPYKCPECGKSFSRSDNLVRHQ |
| | | AGG GAG GGC | | RTHTGEKPYKCPECGKSFSRSDHLTNHQRTHTGEKPYKCPECGKSFSRSDHLTNH |
| | | (SEQ ID NO: | | QRTHTGEKPYKCPECGKSFSQSSNLVRHQRTHTGEKPYKCPECGKSFSRSDNLVR |
| | | 782) | | HQRTHTGKKTS (SEQ ID NO: 795) |

In embodiments, ZNFs for targeting human genomic safe harbor sites using any of the ZNF-based targeting elements to the AAVS1 (e.g., hg38 chr19:55,112,851-55,113,324) are shown in TABLE 5B.

TABLE 5B

| AAVS1 | | | | |
|---|---|---|---|---|
| TTAA | NAME | TARGET | SCORE | ZFP AMINO ACID CODE |
| 5′ | ZnF11a | TAG GAC AGT GGG GAA AAT GAC | 57.08 | LEPGEKPYKCPECGKSFSDCRDLARHQRTHTGEKPYKCPECG |
| | | CCA ACA GCC (SEQ ID NO: | | KSFSSPADLTRHQRTHTGEKPYKCPECGKSFSTSHSLTEHQR |
| | | 796) | | THTGEKPYKCPECGKSFSDPGNLVRHQRTHTGEKPYKCPECG |
| | | | | KSFSTTGNLTVHQRTHTGEKPYKCPECGKSFSQSSNLVRHQR |
| | | | | THTGEKPYKCPECGKSFSRSDKLVRHQRTHTGEKPYKCPECG |
| | | | | KSFSHRTTLTNHQRTHTGEKPYKCPECGKSFSDPGNLVRHQR |
| | | | | THTGEKPYKCPECGKSFSREDNLHTHQRTHTGKKTS (SEQ |
| | | | | ID NO: 811) |
| 5′ | ZnF10a | AGA GGG AGC CAC GAA AAC AGA | 56.91 | LEPGEKPYKCPECGKSFSQLAHLRAHQRTHTGEKPYKCPECG |
| | | (SEQ ID NO: 797) | | KSFSDSGNLRVHQRTHTGEKPYKCPECGKSFSQSSNLVRHQR |
| | | | | THTGEKPYKCPECGKSFSSKKALTEHQRTHTGEKPYKCPECG |
| | | | | KSFSERSHLREHQRTHTGEKPYKCPECGKSFSRSDKLVRHQR |
| | | | | THTGEKPYKCPECGKSFSQLAHLRAHQRTHTGKKTS (SEQ |
| | | | | ID NO: 812) |
| 3′ | ZnF12b | GCA GAT AGC CAG GAG (SEQ ID | 59.97 | LEPGEKPYKCPECGKSFSRSDNLVRHQRTHTGEKPYKCPECG |
| | | NO: 798) | | KSFSRADNLTEHQRTHTGEKPYKCPECGKSFSERSHLREHQR |
| | | | | THTGEKPYKCPECGKSFSTSGNLVRHQRTHTGEKPYKCPECG |
| | | | | KSFSQSGDLRRHQRTHTGKKTS (SEQ ID NO: 813) |
| 3′ | ZnF13b | AGA TAG CCA GGA GTC CTT | 56.80 | LEPGEKPYKCPECGKSFSTTGALTEHQRTHTGEKPYKCPECG |
| | | (SEQ ID NO: 799) | | KSFSDPGALVRHQRTHTGEKPYKCPECGKSFSQRAHLERHQR |
| | | | | THTGEKPYKCPECGKSFSTSHSLTEHQRTHTGEKPYKCPECG |
| | | | | KSFSREDNLHTHQRTHTGEKPYKCPECGKSFSQLAHLRAHQR |
| | | | | THTGKKTS (SEQ ID NO: 814) |
| 5′ | ZnF14a | CCC AGT GGT CAG GCC GGC CAG | 61.78 | LEPGEKPYKCPECGKSFSDCRDLARHQRTHTGEKPYKCPECG |
| | | GCC (SEQ ID NO: 800) | | KSFSRADNLTEHQRTHTGEKPYKCPECGKSFSDPGHLVRHQR |
| | | | | THTGEKPYKCPECGKSFSDCRDLARHQRTHTGEKPYKCPECG |
| | | | | KSFSRADNLTEHQRTHTGEKPYKCPECGKSFSTSGHLVRHQR |
| | | | | THTGEKPYKCPECGKSFSHRTTLTNHQRTHTGEKPYKCPECG |
| | | | | KSFSSKKHLAEHQRTHTGKKTS (SEQ ID NO: 815) |
| 5′ | ZnF15a | GGC CGG CCA GGC CTT CAG | 58.15 | LEPGEKPYKCPECGKSFSRADNLTEHQRTHTGEKPYKCPECG |
| | | (SEQ ID NO: 801) | | KSFSTTGALTEHQRTHTGEKPYKCPECGKSFSDPGHLVRHQR |
| | | | | THTGEKPYKCPECGKSFSTSHSLTEHQRTHTGEKPYKCPECG |
| | | | | KSFSRSDKLTEHQRTHTGEKPYKCPECGKSFSDPGHLVRHQR |
| | | | | THTGKKTS (SEQ ID NO: 816) |
| 5′ | ZnF16a | AGT GCT CAG TGG AAA CCA CGA | 58.65 | LEPGEKPYKCPECGKSFSDPGNLVRHQRTHTGEKPYKCPECG |
| | | AAG GAC (SEQ ID NO: 802) | | KSFSRKDNLKNHQRTHTGEKPYKCPECGKSFSQSGHLTEHQR |
| | | | | THTGEKPYKCPECGKSFSTSHSLTEHQRTHTGEKPYKCPECG |
| | | | | KSFSQRANLRAHQRTHTGEKPYKCPECGKSFSRSDHLTTHQR |
| | | | | THTGEKPYKCPECGKSFSRADNLTEHQRTHTGEKPYKCPECG |
| | | | | KSFSTSGELVRHQRTHTGEKPYKCPECGKSFSHRTTLTNHQR |
| | | | | THTGKKTS (SEQ ID NO: 817) |
| 5′ | ZnF17a | TGG CCC CCA GCC CCT CCT GCC | 60.89 | LEPGEKPYKCPECGKSFSDCRDLARHQRTHTGEKPYKCPECG |
| | | (SEQ ID NO: 803) | | KSFSTKNSLTEHQRTHTGEKPYKCPECGKSFSTKNSLTEHQR |
| | | | | THTGEKPYKCPECGKSFSDCRDLARHQRTHTGEKPYKCPECG |
| | | | | KSFSTSHSLTEHQRTHTGEKPYKCPECGKSFSSKKHLAEHQR |
| | | | | THTGEKPYKCPECGKSFSRSDHLTTHQRTHTGKKTS (SEQ |
| | | | | ID NO: 818) |
| 5′ | ZnF18a | AGA GCC AGG AGT CCT GGC CCC | 57.23 | LEPGEKPYKCPECGKSFSSKKHLAEHQRTHTGEKPYKCPECG |
| | | CAG CCC (SEQ ID NO: 804) | | KSFSRADNLTEHQRTHTGEKPYKCPECGKSFSSKKHLAEHQR |
| | | | | THTGEKPYKCPECGKSFSDPGHLVRHQRTHTGEKPYKCPECG |
| | | | | KSFSTKNSLTEHQRTHTGEKPYKCPECGKSFSHRTTLTNHQR |
| | | | | THTGEKPYKCPECGKSFSRSDHLTNHQRTHTGEKPYKCPECG |
| | | | | KSFSDCRDLARHQRTHTGEKPYKCPECGKSFSQLAHLRAHQR |
| | | | | THTGKKTS (SEQ ID NO: 819) |
| 3′ | ZnF19a | GCA GGA GGG GCT GGG GGC CAG | 59.93 | LEPGEKPYKCPECGKSFSDPGNLVRHQRTHTGEKPYKCPECG |
| | | GAC (SEQ ID NO: 805) | | KSFSRADNLTEHQRTHTGEKPYKCPECGKSFSDPGHLVRHQR |
| | | | | THTGEKPYKCPECGKSFSRSDKLVRHQRTHTGEKPYKCPECG |
| | | | | KSFSTSGELVRHQRTHTGEKPYKCPECGKSFSRSDKLVRHQR |
| | | | | THTGEKPYKCPECGKSFSQRAHLERHQRTHTGEKPYKCPECG |
| | | | | KSFSQSGDLRRHQRTHTGKKTS (SEQ ID NO: 820) |
| 3′ | ZnF20b | ATA GCC CTG GGC CCA CGG CTT | 59.53 | LEPGEKPYKCPECGKSFSSRRTCRAHQRTHTGEKPYKCPECG |
| | | CGT (SEQ ID NO: 806) | | KSFSTTGALTEHQRTHTGEKPYKCPECGKSFSRSDKLTEHQR |
| | | | | THTGEKPYKCPECGKSFSTSHSLTEHQRTHTGEKPYKCPECG |
| | | | | KSFSDPGHLVRHQRTHTGEKPYKCPECGKSFSRNDALTEHQR |
| | | | | THTGEKPYKCPECGKSFSDCRDLARHQRTHTGEKPYKCPECG |
| | | | | KSFSQKSSLIAHQRTHTGKKT (SEQ ID NO: 821) |
| 3′ | ZnF21b | GAA GGA CCT GGC TGG (SEQ ID | 55.22 | LEPGEKPYKCPECGKSFSRSDHLTTHQRTHTGEKPYKCPECG |
| | | NO: 807) | | KSFSDPGHLVRHQRTHTGEKPYKCPECGKSFSTKNSLTEHQR |
| | | | | THTGEKPYKCPECGKSFSQRAHLERHQRTHTGEKPYKCPECG |
| | | | | KSFSQSSNLVRHQRTHTGKKTS (SEQ ID NO: 822) |
| 5′ | ZnF22a | GCA GGA ACG AAG CCG TGG GCC | 56.47 | LEPGEKPYKCPECGKSFSDPGHLVRHQRTHTGEKPYKCPECG |
| | | CAG GGC (SEQ ID NO: 808) | | KSFSRADNLTEHQRTHTGEKPYKCPECGKSFSDCRDLARHQR |
| | | | | THTGEKPYKCPECGKSFSRSDHLTTHQRTHTGEKPYKCPECG |
| | | | | KSFSRNDTLTEHQRTHTGEKPYKCPECGKSFSRKDNLKNHQR |
| | | | | THTGEKPYKCPECGKSFSRTDTLRDHQRTHTGEKPYKCPECG |
| | | | | KSFSQRAHLERHQRTHTGEKPYKCPECGKSFSQSGDLRRHQR |
| | | | | THTGKKTS (SEQ ID NO: 823) |
| 5′ | ZnF23a | GGA AAC CAC CCC AGC AGA | 52.63 | LEPGEKPYKCPECGKSFSQLAHLRAHQRTHTGEKPYKCPECG |
| | | (SEQ ID NO: 809) | | KSFSERSHLREHQRTHTGEKPYKCPECGKSFSSKKHLAEHQR |
| | | | | THTGEKPYKCPECGKSFSSKKALTEHQRTHTGEKPYKCPECG |
| | | | | KSFSDSGNLRVHQRTHTGEKPYKCPECGKSFSQRAHLERHQR |
| | | | | THTGKKTS (SEQ ID NO: 824) |
| 5′ | ZnF24a | AAG GGT CAA GCT CGG AAA CCA | 55.09 | LEPGEKPYKCPECGKSFSQKSSLIAHQRTHTGEKPYKCPECG |
| | | CCC CAG CAG ATA (SEQ ID NO: | | KSFSRADNLTEHQRTHTGEKPYKCPECGKSFSRADNLTEHQR |
| | | 810) | | THTGEKPYKCPECGKSFSSKKHLAEHQRTHTGEKPYKCPECG |
| | | | | KSFSTSHSLTEHQRTHTGEKPYKCPECGKSFSQRANLRAHQR |
| | | | | THTGEKPYKCPECGKSFSRSDKLTEHQRTHTGEKPYKCPECG |
| | | | | KSFSTSGELVRHQRTHTGEKPYKCPECGKSFSQSGNLTEHQR |
| | | | | THTGEKPYKCPECGKSFSTSGHLVRHQRTHTGEKPYKCPECG |
| | | | | KSFSRKDNLKNHQRTHTGKKTS (SEQ ID NO: 825) |

In embodiments, ZNFs for targeting human genomic safe harbor sites using any of the ZNF-based targeting elements to Chromosome 4 (a g., hg38 chr4:30,793,534-30,875,476 or hg38 chr4:30,793,533-30,793,537 (9677); chr4:30,875,472-30,875,476 (8948)) are shown in TABLE 5C.

TABLE 5C

| Chr4 | | | | |
|---|---|---|---|---|
| TTAA | NAME | TARGET | SCORE | ZFP AMINO ACID CODE |
| 5′ | ZnF31F | CTTTGATGAACAGTCACA (SEQ ID | 58.41 | LEPGEKPYKCPECGKSFSSPADLTRHQRTHTGEKPYKCPECG |
| | | NO: 826) | | KSFSDPGALVRHQRTHTGEKPYKCPECGKSFSSPADLTRHQR |
| | | | | THTGEKPYKCPECGKSFSQAGHLASHQRTHTGEKPYKCPECG |
| | | | | KSFSQAGHLASHQRTHTGEKPYKCPECGKSFSTTGALTEHQR |
| | | | | THTGKKTS (SEQ ID NO: 835) |
| 5′ | ZnF32F | CTTCCAATTAGTCCTACC (SEQ ID | 55.84 | LEPGEKPYKCPECGKSFSDKKDLTRHQRTHTGEKPYKCPECG |
| | | NO: 827) | | KSFSTKNSLTEHQRTHTGEKPYKCPECGKSFSHRTTLTNHQR |
| | | | | THTGEKPYKCPECGKSFSHKNALQNHQRTHTGEKPYKCPECG |
| | | | | KSFSTSHSLTEHQRTHTGEKPYKCPECGKSFSTTGALTEHQR |
| | | | | THTGKKTS (SEQ ID NO: 836) |
| 5′ | ZnF33F | ATACTAGGAAGAAATACAATA (SEQ | 57.27 | LEPGEKPYKCPECGKSFSQKSSLIAHQRTHTGEKPYKCPECG |
| | | ID NO: 828) | | KSFSSPADLTRHQRTHTGEKPYKCPECGKSFSTTGNLTVHQR |
| | | | | THTGEKPYKCPECGKSFSQLAHLRAHQRTHTGEKPYKCPECG |
| | | | | KSFSQRAHLERHQRTHTGEKPYKCPECGKSFSONSTLTEHQR |
| | | | | THTGEKPYKCPECGKSFSQKSSLIAHQRTHTGKKTS (SEQ |
| | | | | ID NO: 837) |
| 5′ | ZnF34F | GCTCTTGTCATTTGAGAT (SEQ ID | 57.38 | LEPGEKPYKCPECGKSFSTSGNLVRHQRTHTGEKPYKCPECG |
| | | NO: 829) | | KSFSQAGHLASHQRTHTGEKPYKCPECGKSFSHKNALQNHQR |
| | | | | THTGEKPYKCPECGKSFSDPGALVRHQRTHTGEKPYKCPECG |
| | | | | KSFSTTGALTEHQRTHTGEKPYKCPECGKSFSTSGELVRHQR |
| | | | | THTGKKTS (SEQ ID NO: 838) |
| 5′ | ZnF35F | CCAAGCTGAAATGACACAAAAGTTAA | 58.23 | LEPGEKPYKCPECGKSFSRKDNLKNHQRTHTGEKPYKCPECG |
| | | AACAAAG (SEQ ID NO: 830) | | KSFSSPADLTRHQRTHTGEKPYKCPECGKSFSQRANLRAHQR |
| | | | | THTGEKPYKCPECGKSFSTSGSLVRHQRTHTGEKPYKCPECG |
| | | | | KSFSQRANLRAHQRTHTGEKPYKCPECGKSFSSPADLTRHQR |
| | | | | THTGEKPYKCPECGKSFSDPGNLVRHQRTHTGEKPYKCPECG |
| | | | | KSFSTTGNLTVHQRTHTGEKPYKCPECGKSFSQAGHLASHQR |
| | | | | THTGEKPYKCPECGKSFSERSHLREHQRTHTGEKPYKCPECG |
| | | | | KSFSTSHSLTEHQRTHTGKKTS (SEQ ID NO: 839) |
| 5′ | ZnF36F | CTTATACCAGTTAATTAGCAC (SEQ | 49.93 | LEPGEKPYKCPECGKSFSSKKALTEHQRTHTGEKPYKCPECG |
| | | ID NO: 831) | | KSFSREDNLHTHQRTHTGEKPYKCPECGKSFSTTGNLTVHQR |
| | | | | THTGEKPYKCPECGKSFSTSGSLVRHQRTHTGEKPYKCPECG |
| | | | | KSFSTSHSLTEHQRTHTGEKPYKCPECGKSFSQKSSLIAHQR |
| | | | | THTGEKPYKCPECGKSFSTTGALTEHQRTHTGKKTS (SEQ |
| | | | | ID NO: 840) |
| 3′ | ZnF37R | AACGCTATTGCACACATAGTTACA | 57.67 | LEPGEKPYKCPECGKSFSSPADLTRHQRTHTGEKPYKCPECG |
| | | (SEQ ID NO: 832) | | KSFSTSGSLVRHQRTHTGEKPYKCPECGKSFSQKSSLIAHQR |
| | | | | THTGEKPYKCPECGKSFSSKKALTEHQRTHTGEKPYKCPECG |
| | | | | KSFSQSGDLRRHQRTHTGEKPYKCPECGKSFSHKNALQNHQR |
| | | | | THTGEKPYKCPECGKSFSTSGELVRHQRTHTGEKPYKCPECG |
| | | | | KSFSDSGNLRVHQRTHTGKKTS (SEQ ID NO: 841) |
| 3′ | ZnF38R | TGAATTCAGGAACAAAGTATA (SEQ | 53.21 | LEPGEKPYKCPECGKSFSQKSSLIAHQRTHTGEKPYKCPECG |
| | | ID NO: 833) | | KSFSHRTTLTNHQRTHTGEKPYKCPECGKSFSQSGNLTEHQR |
| | | | | THTGEKPYKCPECGKSFSQSSNLVRHQRTHTGEKPYKCPECG |
| | | | | KSFSRADNLTEHQRTHTGEKPYKCPECGKSFSHKNALQNHQR |
| | | | | THTGEKPYKCPECGKSFSQAGHLASHQRTHTGKKTS (SEQ |
| | | | | ID NO: 842) |
| 3′ | ZnF39R | GCTGGTATGTGACACAGCCATCAACA | 50.63 | LEPGEKPYKCPECGKSFSQSGNLTEHQRTHTGEKPYKCPECG |
| | | A (SEQ ID NO: 834) | | KSFSQSGNLTEHQRTHTGEKPYKCPECGKSFSTSGNLTEHQR |
| | | | | THTGEKPYKCPECGKSFSERSHLREHQRTHTGEKPYKCPECG |
| | | | | KSFSSKKALTEHQRTHTGEKPYKCPECGKSFSQAGHLASHQR |
| | | | | THTGEKPYKCPECGKSFSRRDELNVHQRTHTGEKPYKCPECG |
| | | | | KSFSTSGHLVRHQRTHTGEKPYKCPECGKSFSTSGELVRHQR |
| | | | | THTGKKTS (SEQ ID NO: 843) |

In embodiments, ZNFs for targeting human genomic safe harbor sites using any of the ZNF-based targeting elements to Chromosome 22 (e.g., hg38 chr22:35,370,000-35,380,000 or hg38 chr22:35,373,912-35,373,916 (861); chr22:35,377,843-35,377,847 (1153)) are shown in TABLE 5D.

TABLE 5D

| Chr22 | | | | |
|---|---|---|---|---|
| TTAA | NAME | TARGET | SCORE | ZFP |
| 5′ | ZnF1a | CTTCCTGAAAGCAAGAGAT | 57.34 | LEPGEKPYKCPECGKSFSTTGNLTVHQRTHTGEKPYKCPECGKSFSQAGHLASH |
| | | GAAAT (SEQ ID NO: | | QRTHTGEKPYKCPECGKSFSQLAHLRAHQRTHTGEKPYKCPECGKSFSRKDNLK |
| | | 844) | | NHQRTHTGEKPYKCPECGKSFSERSHLREHQRTHTGEKPYKCPECGKSFSQSSN |
| | | | | LVRHQRTHTGEKPYKCPECGKSFSTKNSLTEHQRTHTGEKPYKCPECGKSFSTT |
| | | | | GALTEHQRTHTGKKTS (SEQ ID NO: 861) |
| 5′ | ZnF1b | CTGAAAGCAAGAGATGAAA | 58.92 | LEPGEKPYKCPECGKSFSTSHSLTEHQRTHTGEKPYKCPECGKSFSHKNALQNH |
| | | TTCCA (SEQ ID NO: | | QRTHTGEKPYKCPECGKSFSQSSNLVRHQRTHTGEKPYKCPECGKSFSTSGNLV |
| | | 845) | | RHQRTHTGEKPYKCPECGKSFSQLAHLRAHQRTHTGEKPYKCPECGKSFSQSGD |
| | | | | LRRHQRTHTGEKPYKCPECGKSFSQRANLRAHQRTHTGEKPYKCPECGKSFSRN |
| | | | | DALTEHQRTHTGKKTS (SEQ ID NO: 862) |
| 5′ | ZnF2a | ATACGAGGAGAAAATTAGC | 51.25 | LEPGEKPYKCPECGKSFSTSGNLTEHQRTHTGEKPYKCPECGKSFSREDNLHTH |
| | | AT (SEQ ID NO: 846) | | QRTHTGEKPYKCPECGKSFSTTGNLTVHQRTHTGEKPYKCPECGKSFSQSSNLV |
| | | | | RHQRTHTGEKPYKCPECGKSFSQRAHLERHQRTHTGEKPYKCPECGKSFSQSGH |
| | | | | LTEHQRTHTGEKPYKCPECGKSFSQKSSLIAHQRTHTGKKTS (SEQ ID NO: |
| | | | | 863) |
| 5′ | ZnF3a | CATCCATGGCAGGAAGTTG | 58.67 | LEPGEKPYKCPECGKSFSRNDALTEHQRTHTGEKPYKCPECGKSFSTTGNLTVH |
| | | AAGCCAAAATAAATCTG | | QRTHTGEKPYKCPECGKSFSQKSSLIAHQRTHTGEKPYKCPECGKSFSQRANLR |
| | | (SEQ ID NO: 847) | | AHQRTHTGEKPYKCPECGKSFSDCRDLARHQRTHTGEKPYKCPECGKSFSQSSN |
| | | | | LVRHQRTHTGEKPYKCPECGKSFSTSGSLVRHQRTHTGEKPYKCPECGKSFSQS |
| | | | | SNLVRHQRTHTGEKPYKCPECGKSFSRADNLTEHQRTHTGEKPYKCPECGKSFS |
| | | | | RSDHLTTHQRTHTGEKPYKCPECGKSFSTSHSLTEHQRTHTGEKPYKCPECGKS |
| | | | | FSTSGNLTEHQRTHTGKKTS (SEQ ID NO: 864) |
| 5′ | ZnF3b | ATGGCAGGAAGTTGAAGCC | 54.14 | LEPGEKPYKCPECGKSFSQRANLRAHQRTHTGEKPYKCPECGKSFSTTGNLTVH |
| | | AAAATAAA (SEQ ID | | QRTHTGEKPYKCPECGKSFSQSGNLTEHQRTHTGEKPYKCPECGKSFSERSHLR |
| | | NO: 848) | | EHQRTHTGEKPYKCPECGKSFSQAGHLASHQRTHTGEKPYKCPECGKSFSHRTT |
| | | | | LTNHQRTHTGEKPYKCPECGKSFSQRAHLERHQRTHTGEKPYKCPECGKSFSQS |
| | | | | GDLRRHQRTHTGEKPYKCPECGKSFSRRDELNVHQRTHTGKKTS (SEQ ID |
| | | | | NO: 865) |
| 3′ | ZnF5aR | GAAAAGAAGACTCAAGGAA | 55.40 | LEPGEKPYKCPECGKSFSSKKALTEHQRTHTGEKPYKCPECGKSFSQRANLRAH |
| | | ACAGAGCCAAACAC (SEQ | | QRTHTGEKPYKCPECGKSFSDCRDLARHQRTHTGEKPYKCPECGKSFSQLAHLR |
| | | ID NO: 849) | | AHQRTHTGEKPYKCPECGKSFSDSGNLRVHQRTHTGEKPYKCPECGKSFSQRAH |
| | | | | LERHQRTHTGEKPYKCPECGKSFSQSGNLTEHQRTHTGEKPYKCPECGKSFSTH |
| | | | | LDLIRHQRTHTGEKPYKCPECGKSFSRKDNLKNHQRTHTGEKPYKCPECGKSFS |
| | | | | RKDNLKNHQRTHTGEKPYKCPECGKSFSQSSNLVRHQRTHTGKKTS (SEQ ID |
| | | | | NO: 866) |
| 3′ | ZnF5bR | AGGAAACAGAGCCAAACAC | 54.66 | LEPGEKPYKCPECGKSFSSPADLTRHQRTHTGEKPYKCPECGKSFSTTGALTEH |
| | | TTACA (SEQ ID NO: | | QRTHTGEKPYKCPECGKSFSSPADLTRHQRTHTGEKPYKCPECGKSFSQSGNLT |
| | | 850) | | EHQRTHTGEKPYKCPECGKSFSERSHLREHQRTHTGEKPYKCPECGKSFSRADN |
| | | | | LTEHQRTHTGEKPYKCPECGKSFSQRANLRAHQRTHTGEKPYKCPECGKSFSRS |
| | | | | DHLTNHQRTHTGKKTS (SEQ ID NO: 867) |
| 3′ | ZnF6aR | ATGCAGATTTGGACACAGA | 58.57 | LEPGEKPYKCPECGKSFSRSDKLVRHQRTHTGEKPYKCPECGKSFSSRRTCRAH |
| | | GTAGTAAACTGTGAAAACG | | QRTHTGEKPYKCPECGKSFSRSDHLTTHQRTHTGEKPYKCPECGKSFSRKDNLK |
| | | TGACAAGGCAAAGTGGCGT | | NHQRTHTGEKPYKCPECGKSFSQSGDLRRHQRTHTGEKPYKCPECGKSFSRKDN |
| | | GGG (SEQ ID NO: | | LKNHQRTHTGEKPYKCPECGKSFSDPGNLVRHQRTHTGEKPYKCPECGKSFSSR |
| | | 851) | | RTCRAHQRTHTGEKPYKCPECGKSFSQRANLRAHQRTHTGEKPYKCPECGKSFS |
| | | | | QAGHLASHQRTHTGEKPYKCPECGKSFSRNDALTEHQRTHTGEKPYKCPECGKS |
| | | | | FSQRANLRAHQRTHTGEKPYKCPECGKSFSHRTTLTNHQRTHTGEKPYKCPECG |
| | | | | KSFSHRTTLTNHQRTHTGEKPYKCPECGKSFSRADNLTEHQRTHTGEKPYKCPE |
| | | | | CGKSFSSPADLTRHQRTHTGEKPYKCPECGKSFSRSDHLTTHQRTHTGEKPYKC |
| | | | | PECGKSFSHKNALQNHQRTHTGEKPYKCPECGKSFSRADNLTEHQRTHTGEKPY |
| | | | | KCPECGKSFSRRDELNVHQRTHTGKKTS (SEQ ID NO: 868) |
| 3′ | ZnF6bR | GGACACAGAGTAGTAAAC | 55.80 | LEPGEKPYKCPECGKSFSDSGNLRVHQRTHTGEKPYKCPECGKSFSQSSSLVRH |
| | | (SEQ ID NO: 852) | | QRTHTGEKPYKCPECGKSFSQSSSLVRHQRTHTGEKPYKCPECGKSFSQLAHLR |
| | | | | AHQRTHTGEKPYKCPECGKSFSSKKALTEHQRTHTGEKPYKCPECGKSFSQRAH |
| | | | | LERHQRTHTGKKTS (SEQ ID NO: 869) |
| 5′ | ZnF10F | AAAGCTAGCAGCATGGCA | 57.55 | LEPGEKPYKCPECGKSFSDSGNLRVHQRTHTGEKPYKCPECGKSFSQSSSLVRH |
| | | (SEQ ID NO: 853) | | QRTHTGEKPYKCPECGKSFSQSSSLVRHQRTHTGEKPYKCPECGKSFSQLAHLR |
| | | | | AHQRTHTGEKPYKCPECGKSFSSKKALTEHQRTHTGEKPYKCPECGKSFSQRAH |
| | | | | LERHQRTHTGKKTS (SEQ ID NO: 870) |
| 5′ | ZnF11F | CCTCTTATAAGGCCCAAGA | 52.55 | LEPGEKPYKCPECGKSFSQKSSLIAHQRTHTGEKPYKCPECGKSFSRSDHLTNH |
| | | GGATA (SEQ ID NO: | | QRTHTGEKPYKCPECGKSFSRKDNLKNHQRTHTGEKPYKCPECGKSFSSKKHLA |
| | | 854) | | EHQRTHTGEKPYKCPECGKSFSRSDHLTNHQRTHTGEKPYKCPECGKSFSQKSS |
| | | | | LIAHQRTHTGEKPYKCPECGKSFSTTGALTEHQRTHTGEKPYKCPECGKSFSTK |
| | | | | NSLTEHQRTHTGKKTS (SEQ ID NO: 871) |
| 5′ | ZnF12F | CAACATCCTTGACTTAATC | 55.00 | LEPGEKPYKCPECGKSFSSKKALTEHQRTHTGEKPYKCPECGKSFSTTGNLTVH |
| | | AC (SEQ ID NO: 855) | | QRTHTGEKPYKCPECGKSFSTTGALTEHQRTHTGEKPYKCPECGKSFSQAGHLA |
| | | | | SHQRTHTGEKPYKCPECGKSFSTKNSLTEHQRTHTGEKPYKCPECGKSFSTSGN |
| | | | | LTEHQRTHTGEKPYKCPECGKSFSQSGNLTEHQRTHTGKKTS (SEQ ID NO: |
| | | | | 872) |
| 5′ | ZnF13F | GGTAGCAAAAAGGTAACC | 46.33 | LEPGEKPYKCPECGKSFSDKKDLTRHQRTHTGEKPYKCPECGKSFSQSSSLVRH |
| | | (SEQ ID NO: 856) | | QRTHTGEKPYKCPECGKSFSRKDNLKNHQRTHTGEKPYKCPECGKSFSQRANLR |
| | | | | AHQRTHTGEKPYKCPECGKSFSERSHLREHQRTHTGEKPYKCPECGKSFSTSGH |
| | | | | LVRHQRTHTGKKTS (SEQ ID NO: 873) |
| 3′ | ZnF14R | TGGGGTGCAAGAGGCCAGG | 61.28 | LEPGEKPYKCPECGKSFSDPGALVRHQRTHTGEKPYKCPECGKSFSRNDALTEH |
| | | CCAGAGTTGTTCTGGTC | | QRTHTGEKPYKCPECGKSFSTSGSLVRHQRTHTGEKPYKCPECGKSFSTSGSLV |
| | | (SEQ ID NO: 857) | | RHQRTHTGEKPYKCPECGKSFSQLAHLRAHQRTHTGEKPYKCPECGKSFSDCRD |
| | | | | LARHQRTHTGEKPYKCPECGKSFSRADNLTEHQRTHTGEKPYKCPECGKSFSDP |
| | | | | GHLVRHQRTHTGEKPYKCPECGKSFSQLAHLRAHQRTHTGEKPYKCPECGKSFS |
| | | | | QSGDLRRHQRTHTGEKPYKCPECGKSFSTSGHLVRHQRTHTGEKPYKCPECGKS |
| | | | | FSRSDHLTTHQRTHTGKKTS (SEQ ID NO: 874) |
| 3′ | ZnF15R | CGCATGCTGATTCAGCCTC | 58.41 | LEPGEKPYKCPECGKSFSDPGNLVRHQRTHTGEKPYKCPECGKSFSTKNSLTEH |
| | | CTGAC (SEQ ID NO: | | QRTHTGEKPYKCPECGKSFSTKNSLTEHQRTHTGEKPYKCPECGKSFSRADNLT |
| | | 858) | | EHQRTHTGEKPYKCPECGKSFSHKNALQNHQRTHTGEKPYKCPECGKSFSRNDA |
| | | | | LTEHQRTHTGEKPYKCPECGKSFSRRDELNVHQRTHTGEKPYKCPECGKSFSHT |
| | | | | GHLLEHQRTHTGKKTS (SEQ ID NO: 875) |
| 3′ | ZnF14R | AGTCAAGCAACAGGATGA | 50.89 | LEPGEKPYKCPECGKSFSQAGHLASHQRTHTGEKPYKCPECGKSFSQRAHLERH |
| | | (SEQ ID NO: 859) | | QRTHTGEKPYKCPECGKSFSSPADLTRHQRTHTGEKPYKCPECGKSFSQSGDLR |
| | | | | RHQRTHTGEKPYKCPECGKSFSQSGNLTEHQRTHTGEKPYKCPECGKSFSHRTT |
| | | | | LTNHQRTHTGKKTS (SEQ ID NO: 876) |
| 3′ | ZnF15R | GTCAAGCAACAGGATGATC | 59.22 | LEPGEKPYKCPECGKSFSHKNALQNHQRTHTGEKPYKCPECGKSFSTSGELVRH |
| | | CAAATGCTATT (SEQ ID | | QRTHTGEKPYKCPECGKSFSTTGNLTVHQRTHTGEKPYKCPECGKSFSTSHSLT |
| | | NO: 860) | | EHQRTHTGEKPYKCPECGKSFSTSGNLVRHQRTHTGEKPYKCPECGKSFSTSGN |
| | | | | LVRHQRTHTGEKPYKCPECGKSFSRADNLTEHQRTHTGEKPYKCPECGKSFSQS |
| | | | | GNLTEHQRTHTGEKPYKCPECGKSFSRKDNLKNHQRTHTGEKPYKCPECGKSFS |
| | | | | DPGALVRHQRTHTGKKTS (SEQ ID NO: 877) |

In embodiments, ZNFs for targeting human genomic safe harbor sites using any of the ZNF-based targeting elements to Chromosome X (e.g., hg38 chrX:134,419,661-134,541,172 or hg38 chrX:134,476,304-134,476,307 (85); chrX:134,476,337-134,476,340 (51)) are shown in TABLE 5E.

TABLE 5E

| ChrX | | | | |
|---|---|---|---|---|
| TTAA | NAME | TARGET | SCORE | ZFP AMINO ACID CODE |
| 5′ | ZnF41F | GTAGAAACTCGCCTTATG (SEQ ID | 54.04 | LEPGEKPYKCPECGKSFSRRDELNVHQRTHTGEKPYKCPECG |
| | | NO: 878) | | KSFSTTGALTEHQRTHTGEKPYKCPECGKSFSHTGHLLEHQR |
| | | | | THTGEKPYKCPECGKSFSTHLDLIRHQRTHTGEKPYKCPECG |
| | | | | KSFSQSSNLVRHQRTHTGEKPYKCPECGKSFSQSSSLVRHQR |
| | | | | THTGKKTS (SEQ ID NO: 886) |
| 5′ | ZnF42F | TGAATGAGTCCTGTCCATCTT (SEQ | 55.08 | LEPGEKPYKCPECGKSFSTTGALTEHQRTHTGEKPYKCPECG |
| | | ID NO: 879) | | KSFSTSGNLTEHQRTHTGEKPYKCPECGKSFSDPGALVRHQR |
| | | | | THTGEKPYKCPECGKSFSTKNSLTEHQRTHTGEKPYKCPECG |
| | | | | KSFSHRTTLTNHQRTHTGEKPYKCPECGKSFSRRDELNVHQR |
| | | | | THTGEKPYKCPECGKSFSQAGHLASHQRTHTGKKTS (SEQ |
| | | | | ID NO: 887) |
| 5′ | ZnF43F | AAGATTAGAACAAATGTCCAG (SEQ | 60.20 | LEPGEKPYKCPECGKSFSRADNLTEHQRTHTGEKPYKCPECG |
| | | ID NO: 880) | | KSFSDPGALVRHQRTHTGEKPYKCPECGKSFSTTGNLTVHQR |
| | | | | THTGEKPYKCPECGKSFSSPADLTRHQRTHTGEKPYKCPECG |
| | | | | KSFSQLAHLRAHQRTHTGEKPYKCPECGKSFSHKNALQNHQR |
| | | | | THTGEKPYKCPECGKSFSRKDNLKNHQRTHTGKKTS (SEQ |
| | | | | ID NO: 888) |
| 3′ | ZnF44R | ACTCTAAGCAGCAATGTA (SEQ ID | 59.94 | LEPGEKPYKCPECGKSFSQSSSLVRHQRTHTGEKPYKCPECG |
| | | NO: 881) | | KSFSTTGNLTVHQRTHTGEKPYKCPECGKSFSERSHLREHQR |
| | | | | THTGEKPYKCPECGKSFSERSHLREHQRTHTGEKPYKCPECG |
| | | | | KSFSQNSTLTEHQRTHTGEKPYKCPECGKSFSTHLDLIRHQR |
| | | | | THTGKKTS (SEQ ID NO: 889) |
| 5′ | ZnF45R | TGGGATAGTGAAAATGTC (SEQ ID | 57.10 | LEPGEKPYKCPECGKSFSDPGALVRHQRTHTGEKPYKCPECG |
| | | NO: 882) | | KSFSTTGNLTVHQRTHTGEKPYKCPECGKSFSQSSNLVRHQR |
| | | | | THTGEKPYKCPECGKSFSHRTTLTNHQRTHTGEKPYKCPECG |
| | | | | KSFSTSGNLVRHQRTHTGEKPYKCPECGKSFSRSDHLTTHQR |
| | | | | THTGKKTS (SEQ ID NO: 890) |
| 5′ | ZnF46R | AAAACTTGGGTCACTAAAATAGATGA | 61.20 | LEPGEKPYKCPECGKSFSTSGNLVRHQRTHTGEKPYKCPECG |
| | | T (SEQ ID NO: 883) | | KSFSTSGNLVRHQRTHTGEKPYKCPECGKSFSQKSSLIAHQR |
| | | | | THTGEKPYKCPECGKSFSQRANLRAHQRTHTGEKPYKCPECG |
| | | | | KSFSTHLDLIRHQRTHTGEKPYKCPECGKSFSDPGALVRHQR |
| | | | | THTGEKPYKCPECGKSFSRSDHLTTHQRTHTGEKPYKCPECG |
| | | | | KSFSTHLDLIRHQRTHTGEKPYKCPECGKSFSQRANLRAHQR |
| | | | | THTGKKTS (SEQ ID NO: 891) |
| 5′ | ZnF47R | AAACATGGAAAAGGTCAAAAACTTGG | 43.59 | LEPGEKPYKCPECGKSFSRSDKLVRHQRTHTGEKPYKCPECG |
| | | G (SEQ ID NO: 884) | | KSFSTTGALTEHQRTHTGEKPYKCPECGKSFSQRANLRAHQR |
| | | | | THTGEKPYKCPECGKSFSQSGNLTEHQRTHTGEKPYKCPECG |
| | | | | KSFSTSGHLVRHQRTHTGEKPYKCPECGKSFSQRANLRAHQR |
| | | | | THTGEKPYKCPECGKSFSQRAHLERHQRTHTGEKPYKCPECG |
| | | | | KSFSTSGNLTEHQRTHTGEKPYKCPECGKSFSQRANLRAHQR |
| | | | | THTGKKTS (SEQ ID NO: 892) |
| 3′ | ZnF48R | AATGACTAGAATGAAGTCCTACTG | 59.44 | LEPGEKPYKCPECGKSFSRNDALTEHQRTHTGEKPYKCPECG |
| | | (SEQ ID NO: 885) | | KSFSQNSTLTEHQRTHTGEKPYKCPECGKSFSDPGALVRHQR |
| | | | | THTGEKPYKCPECGKSFSQSSNLVRHQRTHTGEKPYKCPECG |
| | | | | KSFSTTGNLTVHQRTHTGEKPYKCPECGKSFSREDNLHTHQR |
| | | | | THTGEKPYKCPECGKSFSDPGNLVRHQRTHTGEKPYKCPECG |
| | | | | KSFSTTGNLTVHQRTHTGKKTS (SEQ ID NO: 893) |

In embodiments, the helper enzyme is capable of inserting a donor DNA at a TA dinucleotide site. In embodiments, the helper enzyme is capable of inserting a donor DNA at a TTAA (SEQ ID NO: 440) tetranucleotide site.

In embodiments, the present disclosure relates to a system having nucleic acids encoding the enzyme, e.g., chimeric enzyme, and the donor DNA, respectively.

In embodiments, the targeting element comprises: a gRNA of or comprising a sequence of TABLE 3A-3F, or a variant thereof; or a TALE DBD of or comprising a sequence of TABLE 4A-4F, or a variant thereof; or a ZNF of or comprising a sequence of TABLE 5A-5E, or a variant thereof.

Linkers

In embodiments, the targeting element is or comprises a nucleic acid binding component of the gene-editing system. In embodiments, the enzyme capable of performing targeted genomic integration (e.g., without limitation, a chimeric helper enzyme) and the targeting element, e.g., nucleic acid binding component of the gene-editing system are fused or linked to one another. For example, in embodiments, the helper enzyme and the targeting element, e.g., nucleic acid binding component of the gene-editing system are fused or linked to one another. In embodiments, the helper enzyme and the targeting element, e.g., nucleic acid binding component of the gene-editing system are connected via a linker.

In embodiments, the linker is a flexible linker. In embodiments, the flexible linker is substantially comprised of glycine and serine residues, optionally wherein the flexible linker comprises (Gly₄Ser)ₙ, where n is an integer from 1-12. In embodiments, the flexible linker is of about 20, or about 30, or about 40, or about 50, or about 60 amino acid residues. In embodiments, the flexible linker is about 50, or about 100, or about 150, or about 200 amino acid residues in length. In embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In embodiments, the flexible linker comprises from about 450 nt to about 500 nt.

In embodiments, the enzyme is directly fused to the N-terminus of the targeting element, e.g., without limitation, a dCas9 enzyme.

In embodiments, the enzyme or variant thereof is able to directly or indirectly cause transposition of a target gene. In embodiments, the enzyme or variant thereof is able to directly or indirectly interact and/or form a complex with one or more proteins or nucleic acids.

Nucleic Acids

In embodiments, the composition further comprising a nucleic acid encoding a donor comprising a transgene to be integrated. In embodiments, the transgene comprises a cargo nucleic acid sequence and a first and a second donor end sequences. In embodiments, the cargo nucleic acid sequence is flanked by the first and the second donor end sequences.

In embodiments, the enzyme or variant thereof is incorporated into a vector or a vector-like particle. In embodiments, the vector or a vector-like particle comprises one or more expression cassettes. In embodiments, the vector or a vector-like particle comprises one expression cassette. In embodiments, the expression cassette further comprises the enzyme or variant thereof, the transgene, the donor end sequences, or a combination thereof. In embodiments, the enzyme or variant thereof, the transgene, the donor end sequences, or a combination thereof are incorporated into one or more vectors or vector-like particles. In embodiments, the enzyme or variant thereof, the transgene, the donor end sequences, or combination thereof are incorporated into a same vector or vector-like particle. In embodiments, the enzyme or variant thereof, the transgene, the donor end sequences, or combination thereof is incorporated into different vectors vector-like particles.

In embodiments, the vector or vector-like particle is nonviral.

In embodiments, the composition comprises DNA, RNA, or both. In embodiments, the enzyme or variant thereof is in the form of RNA. In embodiments, a nucleic acid encoding the enzyme is RNA. In embodiments, a nucleic acid encoding the transgene is DNA.

In embodiments, the enzyme (e.g., without limitation, the helper enzyme) is encoded by a recombinant or synthetic nucleic acid. In embodiments, the nucleic acid is RNA, optionally a helper RNA. In embodiments, the nucleic acid is RNA that has a 5′-m7G cap (cap0, or cap1, or cap2), optionally with pseudouridine substitution (e.g., without limitation n-methyl-pseudouridine), and optionally a poly-A tail of about 30, or about 50, or about 100, of about 150 nucleotides in length. In embodiments, the poly-A tail is of about 30 nucleotides in length, optionally 34 nucleotides in length. In embodiments, a nuclear localization signal is placed before the enzyme start codon at the N-terminus, optionally at the C-terminus.

In embodiments, the nucleic acid that is RNA has a 5′-m7G cap (cap 0, or cap 1, or cap 2).

In embodiments, the nucleic acid comprises a 5′ cap structure, a 5′-UTR comprising a Kozak consensus sequence, a comprising a sequence that increases RNA stability in vivo, a 3′-UTR comprising a sequence that increases RNA stability in vivo, and/or a 3′ poly(A) tail.

In embodiments, the enzyme (e.g., without limitation, a helper enzyme) is incorporated into a vector or a vector-like particle. In embodiments, the vector is a non-viral vector.

In embodiments, a nucleic acid encoding the enzyme in accordance with embodiments of the present disclosure, is DNA.

In embodiments, a construct comprising a donor DNA is any suitable genetic construct, such as a nucleic acid construct, a plasmid, or a vector. In embodiments, the construct is DNA, which is referred to herein as a donor DNA. In embodiments, sequences of a nucleic acid encoding the donor DNA is codon optimized to provide improved mRNA stability and protein expression in mammalian systems.

In embodiments, the enzyme and the donor DNA are included in different vectors. In embodiments, the enzyme and the donor DNA are included in the same vector.

In embodiments, a nucleic acid encoding the enzyme capable of performing targeted genomic integration (e.g., without limitation, a helper enzyme which is a chimeric helper enzyme) is RNA (e.g., helper RNA), and a nucleic acid encoding a donor DNA is DNA.

As would be appreciated in the art, a donor DNA often includes an open reading frame that encodes a transgene at the middle of donor DNA and terminal repeat sequences at the 5′ and 3′ end of the donor DNA. The translated helper enzyme binds to the 5′ and 3′ sequence of the donor DNA and carries out the transposition function.

In embodiments, a mobile element, is used to refer to polynucleotides capable of inserting copies of themselves into other polynucleotides. The term mobile element is well known to those skilled in the art and includes classes of mobile elements that can be distinguished on the basis of sequence organization, for example inverted terminal sequences at each end, and/or directly repeated long terminal repeats (LTRs) at the ends. In embodiments, the mobile element as described herein may be described as a piggyBac like element, e.g., a mobile element that is characterized by its traceless excision, which recognizes TTAA (SEQ ID NO: 440) sequence and restores the sequence at the insert site back to the original TTAA (SEQ ID NO: 440) sequence.

In embodiments, donor DNA or transgene are used interchangeably with mobile elements.

In embodiments, the donor DNA is flanked by one or more end sequences or terminal ends. In embodiments, the donor DNA is or comprises a gene encoding a complete polypeptide. In embodiments, the donor DNA is or comprises a gene which is defective or substantially absent in a disease state.

In embodiments, a transgene is associated with various regulatory elements that are selected to ensure stable expression of a construct with the transgene. Thus, in embodiments, a transgene is encoded by a non-viral vector (e.g., without limitation, a DNA plasmid) that can comprise one or more insulator sequences that prevent or mitigate activation or inactivation of nearby genes. The insulators flank the donor DNA (transgene cassette) to reduce transcriptional silencing and position effects imparted by chromosomal sequences. As an additional effect, the insulators can eliminate functional interactions of the transgene enhancer and promoter sequences with neighboring chromosomal sequences.

In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21(8):1536-50, which is incorporated herein by reference in its entirety.

In embodiments, the transgene is inserted into a GSHS location in a host genome. GSHSs is defined as loci well-suited for gene transfer, as integrations within these sites are not associated with adverse effects such as proto-oncogene activation, tumor suppressor inactivation, or insertional mutagenesis. GSHSs can defined by the following criteria: 1) distance of at least 50 kb from the 5′ end of any gene, (2) distance of at least 300 kb from any cancer-related gene, (3) distance of at least 300 kb from any microRNA (miRNA), (4) location outside a transcription unit, and (5) location outside ultra-conserved regions (UCRs) of the human genome. See Papapetrou et al. Nat Biotechnol 2011; 29:73-8; Bejerano et al. Science 2004; 304:1321-5.

Furthermore, the use of GSHS locations can allow stable transgene expression across multiple cell types. One such site, chemokine C—C motif receptor 5 (CCR5) has been identified and used for integrative gene transfer. CCR5 is a member of the beta chemokine receptor family and is required for the entry of R5 tropic viral strains involved in primary infections. A homozygous 32 bp deletion in the CCR5 gene confers resistance to HIV-1 virus infections in humans. Disrupted CCR5 expression, naturally occurring in about 1% of the Caucasian population, does not appear to result in any reduction in immunity. Lobritz at al., Viruses 2010; 2:1069-105. A clinical trial has demonstrated safety and efficacy of disrupting CCR5 via targetable nucleases. Tebas at al., HIV. N Engl J Med 2014; 370:901-10.

In embodiments, the donor DNA is under control of a tissue-specific promoter. The tissue-specific promoter is, e.g., without limitation, a liver-specific promoter. In embodiments, the liver-specific promoter is an LP1 promoter that, in embodiments, is a human LP1 promoter. The LP1 promoter is described, e.g., in Nathwani et al. Blood vol. 2006; 107(7):2653-61, and it is constructed, without limitation, as described in Nathawani et al.

It should be appreciated however that a variety of promoters can be used, including other tissue-specific promoters, inducible promoters, constitutive promoters, etc.

In embodiments, the present nucleic acids include polymeric form of nucleotides of any length, either ribonucleotides or deoxyribonucleotides, or analogs or derivatives thereof. In embodiments, there is provided double- and single-stranded DNA, as well as double- and single-stranded RNA, and RNA-DNA hybrids. In embodiments, transcriptionally-activated polynucleotides such as methylated or capped polynucleotides are provided. In embodiments, the present compositions are mRNA or DNA.

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. Such vectors may include, among others, chromosomal and episomal vectors, e.g., vectors bacterial plasmids, from donor DNAs, from yeast episomes, from insertion elements, from yeast chromosomal elements, and vectors from combinations thereof. The present constructs may contain control regions that regulate as well as engender expression.

In embodiments, the construct comprising the enzyme and/or transgene is codon optimized. Transgene codon optimization is used to optimize therapeutic potential of the transgene and its expression in the host organism. Codon optimization is performed to match the codon usage in the transgene with the abundance of transfer RNA (tRNA) for each codon in a host organism or cell. Codon optimization methods are known in the art and described in, for example, WO 2007/142954, which is incorporated by reference herein in its entirety. Optimization strategies can include, for example, the modification of translation initiation regions, alteration of mRNA structural elements, and the use of different codon biases.

In embodiments, the construct comprising the enzyme and/or transgene includes several other regulatory elements that are selected to ensure stable expression of the construct. Thus, in embodiments, the non-viral vector is a DNA plasmid that can comprise one or more insulator sequences that prevent or mitigate activation or inactivation of nearby genes. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21(8):1536-50, which is incorporated herein by reference in its entirety. In embodiments, the gene of the construct comprising the enzyme and/or transgene is capable of transposition in the presence of a helper enzyme. In embodiments, the non-viral vector in accordance with embodiments of the present disclosure comprises a nucleic acid construct encoding a helper enzyme. The helper enzyme is an RNA helper enzyme plasmid. In embodiments, the non-viral vector further comprises a nucleic acid construct encoding a DNA helper enzyme plasmid. In embodiments, the helper enzyme is an in vitro-transcribed mRNA helper enzyme. The helper enzyme is capable of excising and/or transposing the gene from the construct comprising the enzyme and/or transgene to site- or locus-specific genomic regions.

In embodiments, the enzyme and the donor DNA are included in the same vector.

In embodiments, the enzyme is disposed on the same (cis) or different vector (trans) than a donor DNA with a transgene. Accordingly, in embodiments, the enzyme and the donor DNA encompassing a transgene are in cis configuration such that they are included in the same vector. In embodiments, the enzyme and the donor DNA encompassing a transgene are in trans configuration such that they are included in different vectors. The vector is any non-viral vector in accordance with the present disclosure.

In aspects, a nucleic acid encoding the enzyme capable of performing targeted genomic integration (e.g., a helper enzyme or a chimeric helper enzyme) in accordance with embodiments of the present disclosure is provided. The nucleic acid is or comprises DNA or RNA. In embodiments, the nucleic acid encoding the enzyme is DNA. In embodiments, the nucleic acid encoding the enzyme capable of performing targeted genomic integration (e.g., a chimeric helper enzyme) is RNA such as, e.g., helper RNA. In embodiments, the chimeric helper enzyme is incorporated into a vector. In embodiments, the vector is a non-viral vector.

In embodiments, a nucleic acid encoding the transgene in accordance with embodiments of the present disclosure is provided. The nucleic acid is or comprises DNA or RNA. In embodiments, the nucleic acid encoding the transgene is DNA. In embodiments, the nucleic acid encoding the e transgene is RNA such as, e.g., helper RNA. In embodiments, the transgene is incorporated into a vector. In embodiments, the vector is a non-viral vector.

In embodiments, the present enzyme can be in the form or an RNA or DNA and have one or two N-terminus nuclear localization signal (NLS) to shuttle the protein more efficiently into the nucleus. For example, in embodiments, the present enzyme further comprises one, two, three, four, five, or more NLSs. Examples of NLS are provided in Kosugi et al. (J. Biol. Chem. (2009) 284:478-485; incorporated by reference herein). In a particular embodiment, the NLS comprises the consensus sequence K(K/R)X(K/R) (SEQ ID NO: 348). In an embodiment, the NLS comprises the consensus sequence (K/R)(K/R)X₁₀₋₁₂(K/R)_{3/5} (SEQ ID NO: 349), where (K/R)_{3/5} represents at least three of the five amino acids is either lysine or arginine. In an embodiment, the NLS comprises the c-myc NLS. In a particular embodiment, the c-myc NLS comprises the sequence PAAKRVKLD (SEQ ID NO: 350). In a particular embodiment, the NLS is the nucleoplasmin NLS. In embodiments, the nucleoplasmin NLS comprises the sequence KRPAATKKAGQAKKKK (SEQ ID NO: 351). In embodiments, the NLS comprises the SV40 Large T-antigen NLS. In embodiments, the SV40 Large T-antigen NLS comprises the sequence PKKKRKV (SEQ ID NO: 352). In a particular embodiment, the NLS comprises three SV40 Large T-antigen NLSs (e.g., DPKKKRKVDPKKKRKVDPKKKRKV (SEQ ID NO: 353). In embodiments, the NLS may comprise mutations/variations in the above sequences such that they contain 1 or more substitutions, additions or deletions (e.g., about 1, or about 2, or about 3, or about 4, or about 5, or about 10 substitutions, additions, or deletions).

In aspects, a host cell comprising the nucleic acid in accordance with embodiments of the present disclosure is provided.

In aspects, there is provided a transgenic animal comprising a host cell comprising the nucleic acid in accordance with embodiments of the present disclosure is provided.

Host Cell

In aspects, the present disclosure further provides a host cell comprising the composition in accordance with embodiments of the present disclosure.

Lipids and LNP Delivery

In embodiments, at least one of the first nucleic acid and the second nucleic acid is in the form of a lipid nanoparticle (LNP). In embodiments, a composition comprising the first and second nucleic acids is in the form of an LNP.

In embodiments, a nucleic acid encoding the enzyme and a nucleic acid encoding the transgene are contained within the same lipid nanoparticle (LNP). In embodiments, the nucleic acid encoding the enzyme and the nucleic acid encoding the donor DNA are a mixture incorporated into or associated with the same LNP. In embodiments, the nucleic acid encoding the enzyme and the nucleic acid encoding the donor DNA are in the form of a co-formulation incorporated into or associated with the same LNP.

In embodiments, the LNP is selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GaINAc).

In embodiments, an LNP is as described, e.g., in Patel et al., J Control Release 2019; 303:91-100. The LNP can comprise one or more of a structural lipid (e.g., DSPC), a PEG-conjugated lipid (CDM-PEG), a cationic lipid (MC3), cholesterol, and a targeting ligand (e.g., GaINAc).

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In aspects, the cell in accordance with the present disclosure is prepared via an in vivo genetic modification method. In embodiments, a genetic modification in accordance with the present disclosure is performed via an ex vivo method.

In aspects, the cell in accordance with the present disclosure is prepared by contacting a cell with an enzyme capable of performing targeted genomic integration (e.g., without limitation, a mammalian helper enzyme) in vivo. In embodiments, the cell is contacted with the enzyme ex vivo.

In embodiments, the present method provides reduced insertional mutagenesis or oncogenesis as compared to a method with a non-chimeric helper enzyme.

Methods

In embodiments, a method for inserting a gene into the genome of a cell, comprising contacting a cell with the composition of the present disclosure or host cell of the present disclosure. In embodiments, the method further comprising contacting the cell with a polynucleotide encoding a donor. In embodiments, the donor comprises a gene encoding a complete polypeptide. In embodiments, the donor comprises a gene which is defective or substantially absent in a disease state. In embodiments, the method for treating a disease or disorder ex vivo of the present disclosure comprises contacting a cell with the composition of the present disclosure or host cell of the present disclosure and administering the cell to a subject in need thereof.

In embodiments, a method for treating a disease or disorder in vivo, comprising administering the composition of the present disclosure or host cell of the present disclosure to a subject in need thereof.

Therapeutic Applications

In embodiments, the transgene of interest in accordance with embodiments of the present disclosure can encode various genes.

In embodiments, the helper enzyme and the donor polynucleotide are included in the same pharmaceutical composition.

In embodiments, the helper enzyme and the donor polynucleotide are included in different pharmaceutical compositions.

In embodiments, the helper enzyme and the donor polynucleotide are co-transfected.

In embodiments, the helper enzyme and the donor polynucleotide are transfected separately.

In embodiments, a transfected cell for gene therapy is provided, wherein the transfected cell is generated using the helper enzymes in accordance with embodiments of the present disclosure.

In embodiments, a method of delivering a cell therapy is provided, comprising administering to a patient in need thereof the transfected cell generated using the helper enzymes in accordance with embodiments of the present disclosure.

In embodiments, a method of treating a disease or condition using a cell therapy, comprising administering to a patient in need thereof the transfected cell generated using the helper enzymes in accordance with embodiments of the present disclosure.

In embodiments, the disease or condition may comprise cancer. In embodiments, the cancer is or comprises an adrenal cancer, a biliary track cancer, a bladder cancer, a bone/bone marrow cancer, a brain cancer, a breast cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

In embodiments, the cancer is selected from one or more of the basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; glioblastoma; hepatic carcinoma; hepatoma; intra-epithelial neoplasm; kidney or renal cancer; larynx cancer; leukemia; liver cancer; lung cancer; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma;

sarcoma; skin cancer; squamous cell cancer; stomach cancer; testicular cancer; thyroid cancer; uterine or endometrial cancer; cancer of the urinary system; vulval cancer; Hodgkin's lymphoma; non-Hodgkin's lymphoma; B-cell lymphoma; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's Macroglobulinemia; chronic lymphocytic leukemia (CLL); acute lymphoblastic leukemia (ALL); and Hairy cell leukemia.

In embodiments, the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs syndrome.

In embodiments, the disease or condition is or comprises an infectious disease. In embodiments, the infectious disease is a coronavirus infection, optionally selected from infection with SAR-CoV, MERS-CoV, and SARS-CoV-2, or variants thereof.

In embodiments, the infectious disease is or comprises a disease comprising a viral infection, a parasitic infection, or a bacterial infection. In embodiments, the viral infection is caused by a virus of family Flaviviridae, a virus of family Picornaviridae, a virus of family Orthomyxoviridae, a virus of family Coronaviridae, a virus of family Retroviridae, a virus of family Paramyxoviridae, a virus of family Bunyaviridae, or a virus of family Reoviridae.

In embodiments, the virus of family Coronaviridae comprises a betacoronavirus or an alphacoronavirus, optionally wherein the betacoronavirus is selected from SARS-CoV-2, SARS-CoV, MERS-CoV, HCoV-HKU1, and HCoV-0043, or the alphacoronavirus is selected from a HCoV-NL63 and HCoV-229E. In embodiments, the infectious disease comprises a coronavirus infection 2019 (COVID-19).

In embodiments, the method is used to treat an inherited or acquired disease in a patient in need thereof. For example, in embodiments, the method is used for treating and/or mitigating a class of Inherited Macular Degeneration (I MDs) (also referred to as Macular dystrophies (MDs), including Stargardt disease (STGD), Best disease, X-linked retinoschisis, pattern dystrophy, Sorsby fundus dystrophy and autosomal dominant drusen. The STGD can be STGD Type 1 (STGD1). In embodiments, the STGD can be STGD Type 3 (STGD3) or STGD Type 4 (STGD4) disease. The IMD can be characterized by one or more mutations in one or more of ABCA4, ELOVL4, PROM1, BEST1, and PRPH2. The gene therapy can be performed using mobile element-based vector systems, with the assistance by chimeric helpers in accordance with the present disclosure, which are provided on the same vector as the gene to be transferred (cis) or on a different vector (trans) or as RNA. The donor DNA can comprise an ATP binding cassette subfamily A member 4 (ABCA4), or functional fragment thereof, and the mobile element-based vector systems can operate under the control of a retina-specific promoter.

In embodiments, the method is used for treating and/or mitigating familial hypercholesterolemia (FH), such as homozygous FH (HoFH) or heterozygous FH (HeFH) or disorders associated with elevated levels of low-density lipoprotein cholesterol (LDL-C). The gene therapy can be performed using mobile element-based vector systems, with the assistance by chimeric helpers in accordance with the present disclosure, which are provided on the same vector (cis) as the gene to be transferred or on a different vector (trans). The donor DNA can comprise a very low-density lipoprotein receptor gene (VLDLR) or a low-density lipoprotein receptor gene (LDLR), or a functional fragment thereof. The donor DNA-based vector systems can operate under control of a liver-specific promoter. In embodiments, the liver-specific promoter is an LP1 promoter. The LP1 promoter can be a human LP1 promoter, which can be constructed as described, e.g., in Nathwani et al. Blood vol. 107(7) (2006):2653-61.

In embodiments, the promoter is a cytomegalovirus (CMV) or cytomegalovirus (CMV) enhancer fused to the chicken β-actin (CAG) promoter. See Alexopoulou et al., BMC Cell Biol. 2008; 9:2. Published 2008 January 11.

It should be appreciated that any other inherited or acquired diseases can be treated and/or mitigated using the method in accordance with the present disclosure.

In embodiments, the method requires a single administration. In embodiments, the method requires a plurality of administrations.

Isolated Cell

In aspects of the present disclosure, an isolated cell is provided that comprises the transfected cell in accordance with embodiments of the present disclosure.

In aspects, the present disclosure provides an ex vivo gene therapy approach. Accordingly, in embodiments, the method that is used to treat an inherited or acquired disease in a patient in need thereof comprises (a) contacting a cell obtained from a patient (autologous) or another individual (allogeneic) with a transfected cell in accordance with embodiments of the present disclosure; and (b) administering the cell to a patient in need thereof.

One of the advantages of ex vivo gene therapy is the ability to “sample” the transduced cells before patient administration. This facilitates efficacy and allows performing safety checks before introducing the cell (s) to the patient. For example, the transduction efficiency and/or the clonality of integration can be assessed before infusion of the product. The present disclosure provides transfected cells and methods that can be effectively used for ex vivo gene modification.

In embodiments, a composition comprising transfected cells in accordance with the present disclosure comprises a pharmaceutically acceptable carrier, excipient, or diluent.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile, and the fluid should be easy to draw up by a syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006; 79(3-4): 141-152.

In embodiments, there is provided a method of transforming a cell using the construct comprising the enzyme and/or transgene described herein in the presence of a helper enzyme (e.g., without limitation, the transposase enzyme) to produce a stably transfected cell which results from the stable integration of a gene of interest into the cell. In embodiments, the stable integration comprises an introduction of a polynucleotide into a chromosome or mini-chromosome of the cell and, therefore, becomes a relatively permanent part of the cellular genome.

In embodiments, there is provided a transgenic organism that may comprise cells which have been transformed by the methods of the present disclosure. In embodiments, the organism may be a mammal or an insect. When the organism is a mammal, the organism may include, but is not limited to, a mouse, a rat, a monkey, a brown bear, a dog, a rabbit, and the like. When the organism is an insect, the organism may include, but is not limited to, a fruit fly, a ladybug, a mosquito, a bollworm, and the like.

Kits

In embodiments, a kit is provided that comprises a recombinant mammalian helper enzyme and/or or a nucleic acid according to any embodiments, or combination thereof, of the present disclosure, and instructions for introducing a polynucleotide into a cell using the recombinant mammalian helper.

### Definitions

The following definitions are used in connection with the invention disclosed herein. Unless defined otherwise, all technical and scientific terms used herein have the same meaning as commonly understood to one of skill in the art to which this invention belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid or the like, which is nontoxic and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD₅₀ (the dose lethal to about 50% of the population) and the ED₅₀ (the dose therapeutically effective in about 50% of the population). The dosage can vary depending upon the dosage form employed and the route of administration utilized. The dose ratio between toxic and therapeutic effects is the therapeutic index and can be expressed as the ratio LD₅₀/ED₅₀. In embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC₅₀ as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

As used herein, “methods of treatment” are equally applicable to use of a composition for treating the diseases or disorders described herein and/or compositions for use and/or uses in the manufacture of a medicaments for treating the diseases or disorders described herein.

### Sequences

In embodiments, the present disclosure provides for any of the sequence provided herein, including without limitation SEQ ID Nos: 1-22, and a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

This invention is further illustrated by the following non-limiting examples.

### EXAMPLES

Hereinafter, the present disclosure will be described in further detail with reference to examples. These examples are illustrative purposes only and are not to be construed to limit the scope of the present invention. In addition, various modifications and variations can be made without departing from the technical scope of the present invention.

### Example 1—Identifying and Reviving a Recombinant Mammalian Helper and its Hyperactive Forms

In this study, a sequence of a recombinant mammalian helper enzyme was identified from disparate parts of the sequence in a mammalian genome. In this way, the recombinant mammalian helper was reconstructed, or “revived,” from its inactive parts.

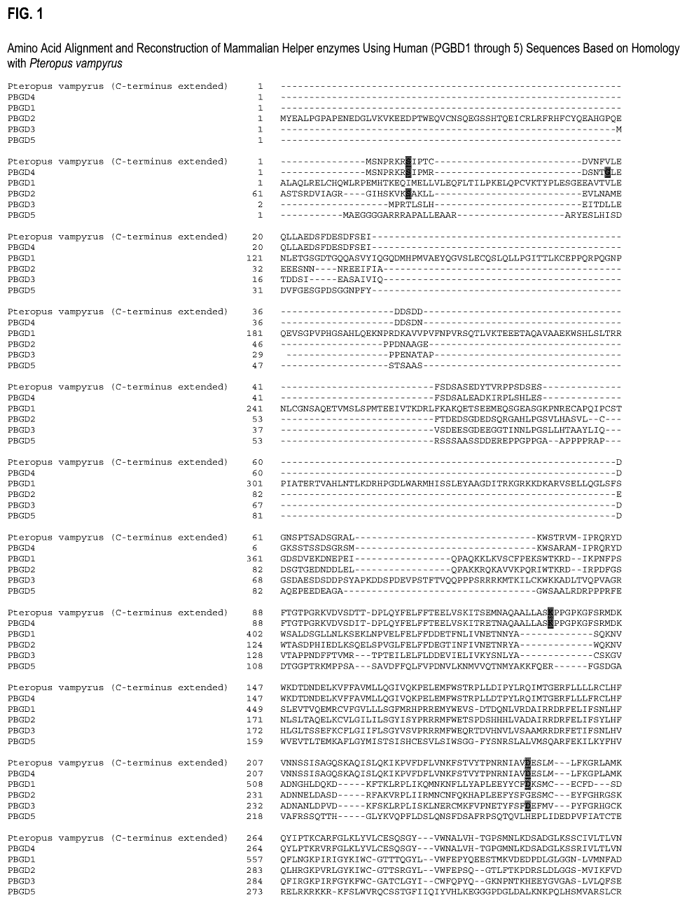

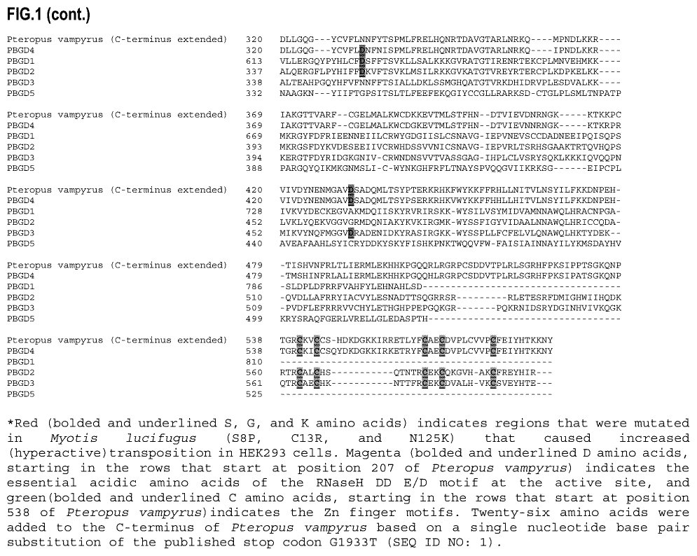

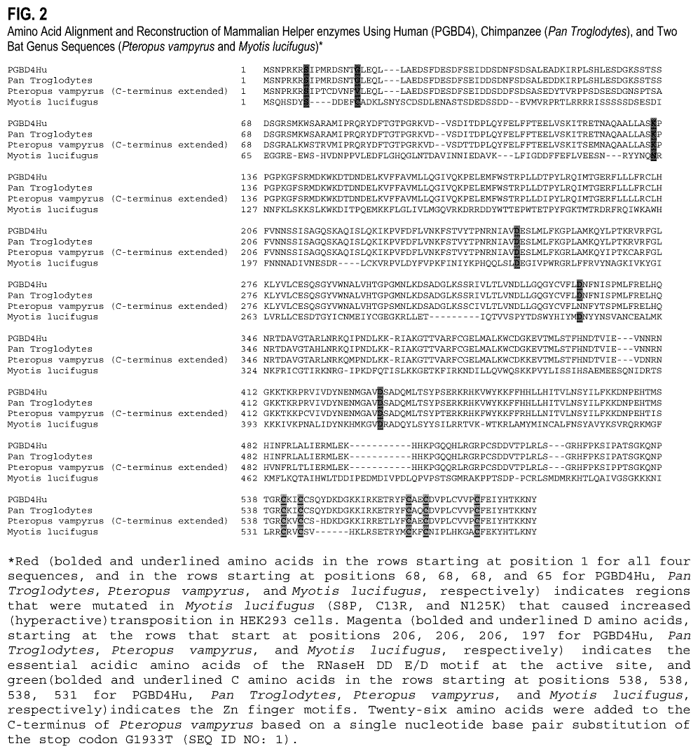

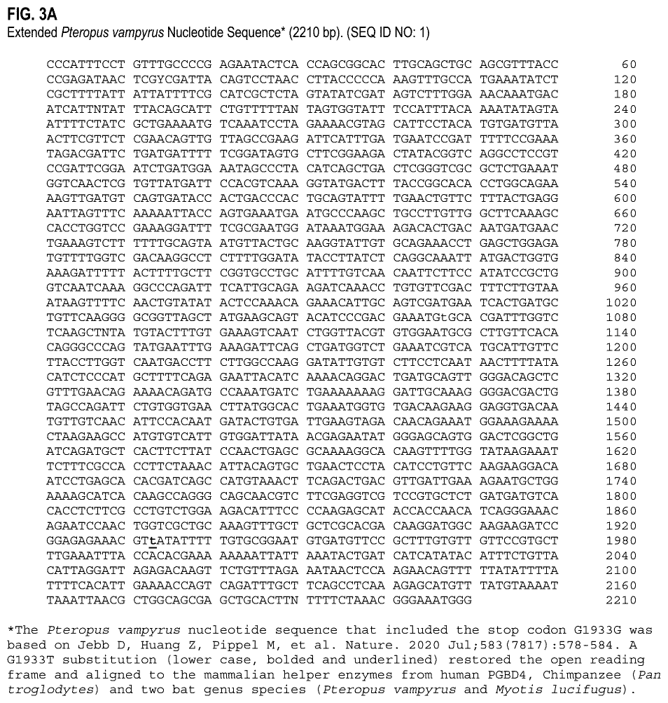

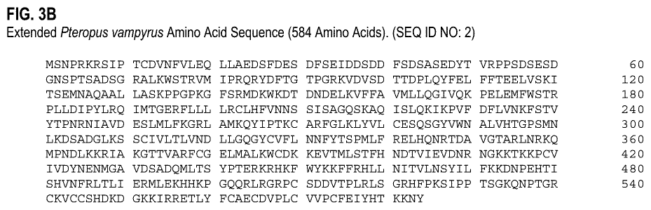

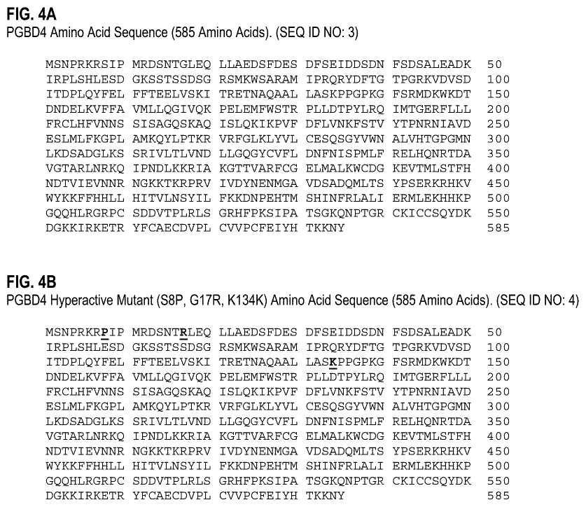

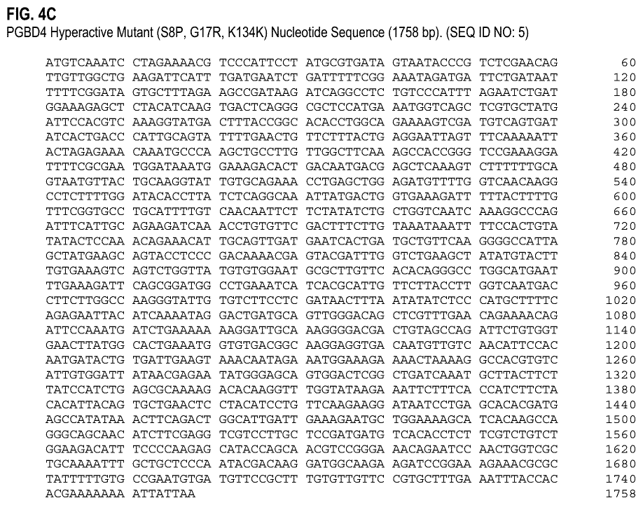

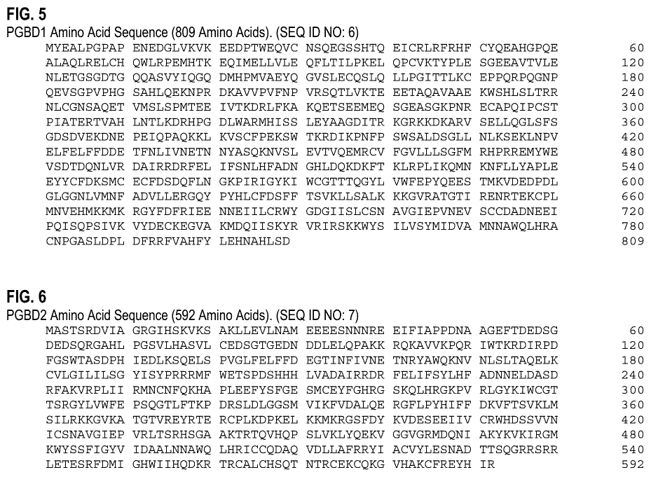

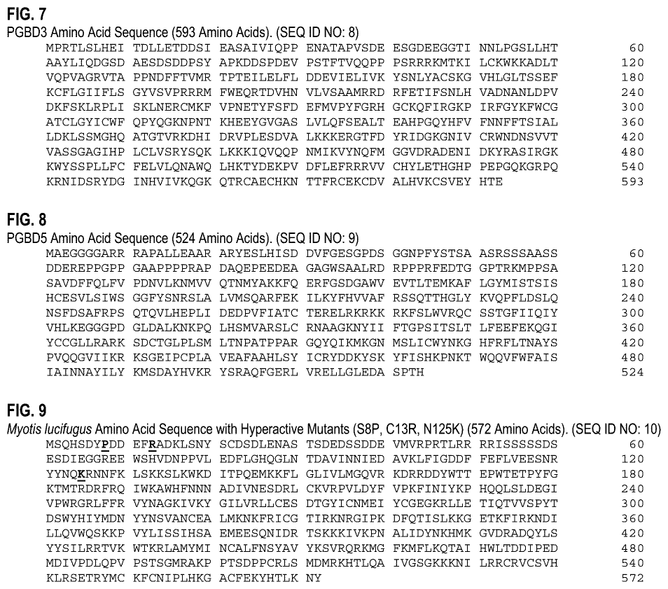

A recombinant mammalian helper enzyme was identified using known PGBD1 (SEQ ID NO: 6), PGBD2 (SEQ ID NO: 7), PGBD3 (SEQ ID NO: 8), PGBD4 (SEQ ID NO: 3), and PGBD5 (SEQ ID NO: 9) sequences from a Homo sapiens genome. As shown in FIG. 1, the amino acid sequences of these sequences were aligned with the amino acid sequence of Pteropus vampyrus. The alignment shown in FIG. 1 was used to reconstruct the recombinant human helpers based on its homology to the active Myotis lucifugus helper in FIG. 2. It was observed that when a stop codon in the nucleotide sequence of Pteropus vampyrus (SEQ ID NO: 1) was corrected with a G1933T substitution, the human and mammalian helper amino acid sequences aligned as in FIG. 1 and FIG. 2 to form active helpers. In FIG. 1, red (bolded and underlined S, G, and K amino acids) indicates regions that were mutated in Myotis lucifugus (S8P, C13R, and N125K) that caused increased (hyperactive) transposition in HEK293 cells. Magenta (bolded and underlined D amino acids, starting in the rows that start at position 207 of Pteropus vampyrus) indicates the essential acidic amino acids of the RNaseH DD E/D motif at the active site, and green (bolded and underlined C amino acids, starting in the rows that start at position 538 of Pteropus vampyrus) indicates the Zn finger motifs. Twenty-six amino acids were added to the C-terminus of Pteropus vampyrus based on a single nucleotide base pair substitution of the published stop codon G1933T. FIG. 3A depicts a nucleotide sequence of Pteropus vampyrus (SEQ ID NO: 1). The amino acid sequence of human helper (PGBD4) (SEQ ID NO: 3) is shown in FIG. 4A.

FIG. 2 depicts an amino acid alignment and reconstruction of mammalian helpers including human helpers (PGBD4, (SEQ ID NO: 3), Pan troglodytes, Pteropus vampyrus, and Myotis lucifugus). Red (bolded and underlined amino acids in the rows starting at position 1 for all four sequences, and in the rows starting at positions 68, 68, 68, and 65 for PGBD4Hu, Pan Troglodytes, Pteropus vampyrus, and Myotis lucifugus, respectively) indicates regions that were mutated in Myotis lucifugus (S8P, C13R, and N125K) that caused increased (hyperactive or Exc+) transposition in HEK293 cells. Magenta (bolded and underlined D amino acids, starting at the rows that start at positions 206, 206, 206, 197 for PGBD4Hu, Pan Troglodytes, Pteropus vampyrus, and Myotis lucifugus, respectively) indicates the essential acidic amino acids of the RNaseH DD E/D motif at the active site, and green (bolded and underlined C amino acids in the rows starting at positions 538, 538, 538, 531 for PGBD4Hu, Pan Troglodytes, Pteropus vampyrus, and Myotis lucifugus, respectively) indicates the Zn finger motifs. Twenty-six amino acids were added to the C-terminus of Pteropus vampyrus based on a single nucleotide base pair substitution of the stop codon G1933T (SEQ ID NO: 1).

A construct in accordance with the present disclosure can include end sequences such as end sequences from Pteropus vampyrus, PGBD4, MER75, MER75B, or MER75A. The end sequences for human helpers were reconstructed from the human genome by alignment with Pteropus vampyrus and sequences in the Dfam Database (on the world wide web at dfam.org/home).

FIG. 4B depicts a hyperactive mutant form of an amino acid sequence of human (PGBD4) helper (SEQ ID NO: 4), and FIG. 4C depicts a hyperactive mutant form of a nucleotide sequence of human (PGBD4) helper (SEQ ID NO: 5).

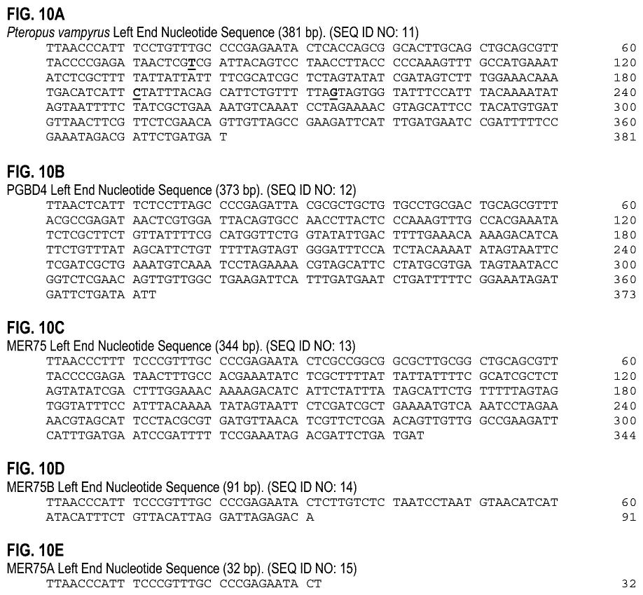

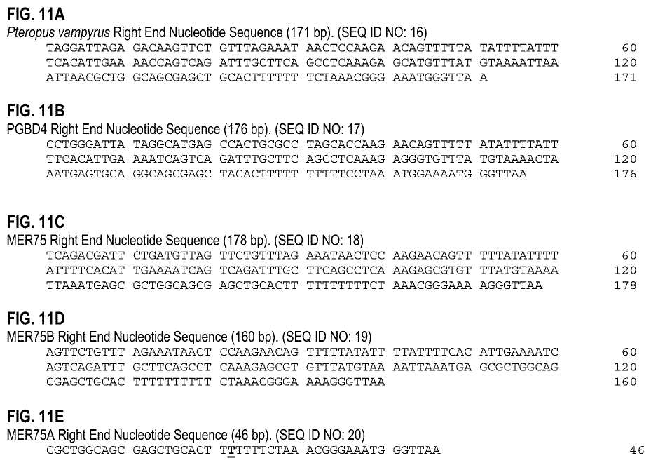

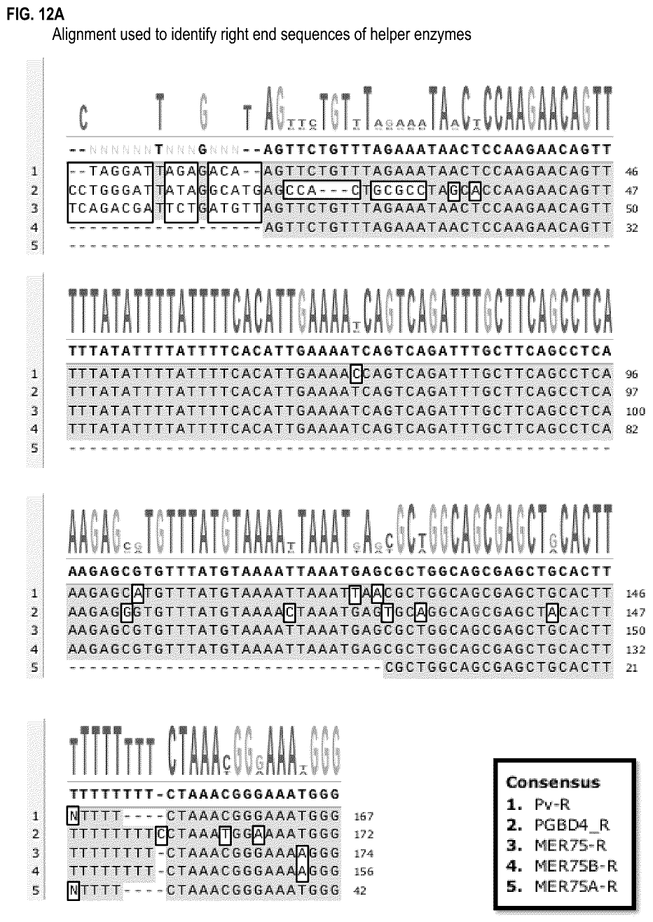

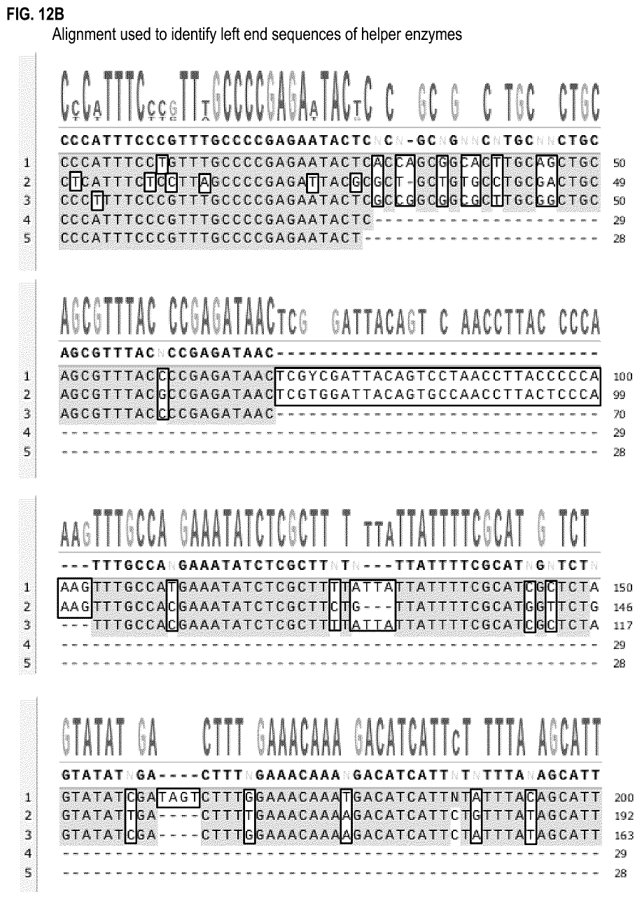

FIG. 10A depicts a left end nucleotide sequence from Pteropus vampyrus (SEQ ID NO: 11). FIG. 11A depicts a right end nucleotide sequence from Pteropus vampyrus (SEQ ID NO: 16). The left end and right end sequences begin with TTAA, the nucleotides that are required for transposition.

FIG. 10B depicts a left end nucleotide sequence from PGBD4 (SEQ ID NO: 12). FIG. 11B depicts a right end nucleotide sequence from PGBD4 (SEQ ID NO: 17). The left end and right end sequence begins with TTAA, the nucleotides that are required for transposition are bolded.

FIG. 10C depicts a left end nucleotide sequence from MER75 (SEQ ID NO: 13). FIG. 11C depicts a right end nucleotide sequence from MER75 (SEQ ID NO: 18). The left end and right end sequences begin with TTAA, the nucleotides that are required for transposition.

FIG. 10D depicts a left end nucleotide sequence from MER75B (SEQ ID NO: 14). FIG. 11D depicts a right end nucleotide sequence from MER75B (SEQ ID NO: 19). The left end and right end sequences begin with TTAA, the nucleotides that are required for transposition.

FIG. 10E depicts a left end nucleotide sequence from MER75A (SEQ ID NO: 15). FIG. 11E depicts a right end nucleotide sequence from MER75A (SEQ ID NO: 20). The left end and right end sequences begin with TTAA, the nucleotides that are required for transposition.

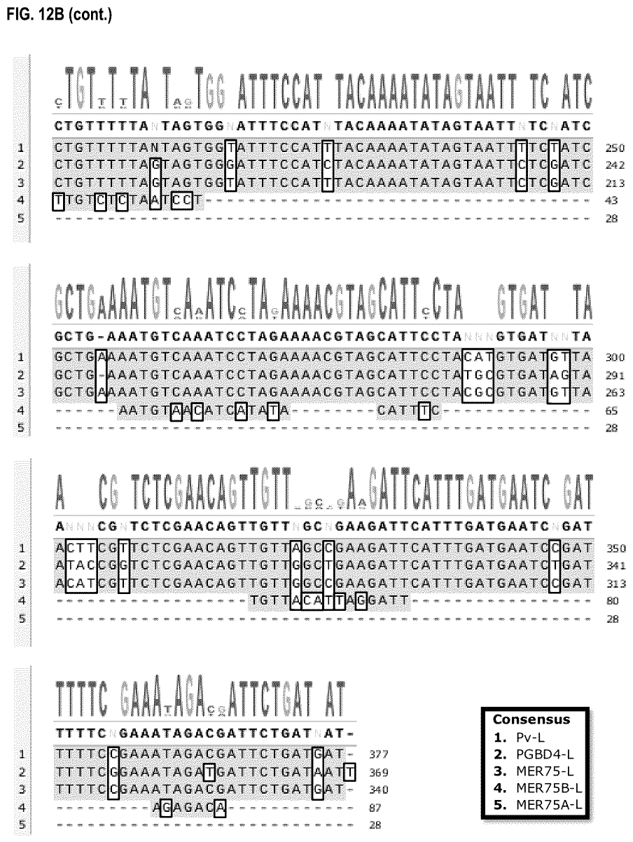

FIGS. 12A and 12B illustrate an alignment that was used in the design and identification of the right and left end sequences, along with a respective consensus sequence. In FIGS. 12A and 12B, sequence logo has 50% CG base composition (see Schneider et al., (1990). Sequence Logos: A New Way to Display Consensus Sequences. Nucleic Acids Res. 18 (20): 6097-6100), consensus threshold is greater than 50%, and bases that do not match the consensus are boxed. FIG. 12A shows the alignment used in identifying the right end sequences, and the following sequences are shown: (1) Pteropus vampyrus (“Pv-R”), (2) PGBD4 (“PGBD4-R”), (3) MER75 (“MER75-R”), (4) MER75B (“MER75B-R”), and (5) MER75A (“MER75A-R”). FIG. 12B shows the alignment used in identifying the left end sequences, and the following sequences are shown: (1) Pteropus vampyrus (“Pv-L”), (2) PGBD4 (“PGBD4-L”), (3) MER75 (“MER75-L”), (4) MER75B (“MER75B-L”), and (5) MER75A (“MER75A-L”). The right and left end sequences were identified by querying the bat and human genomes for sequences that flanked the putative helpers by up to 2-5 kb 5′ and 3, to the alignments shown in FIGS. 12A and 12B. The sequences were analyzed using Dfam database which identifies mobile element sequences. Hubley et al., Nucleic Acids Research (2016) Database Issue 44:D81-89. doi: 10.1093/nar/gkv1272. These sequences were aligned as shown in FIGS. 12A and 12B. The consensus sequence is obtained from the alignment, using the greater than 50%, consensus threshold. Further end sequences can be identified by comparing them to the consensus sequence. Thus, synthetic biology was used by combining the chemical synthesis of DNA with the knowledge of genomics to identify the end DNA sequences and reconstruct, or revive, the helpers by identifying and putting together their disparate, inactive parts that together form a functional helper. These sequences, including mutants, can be assembled into an artificial helper-donor system to insert donor DNA into the human genome.

### Example 2—Design of Recombinant Mammalian Helpers that Target Human Genomic Safe Harbor Sites (GSHS)

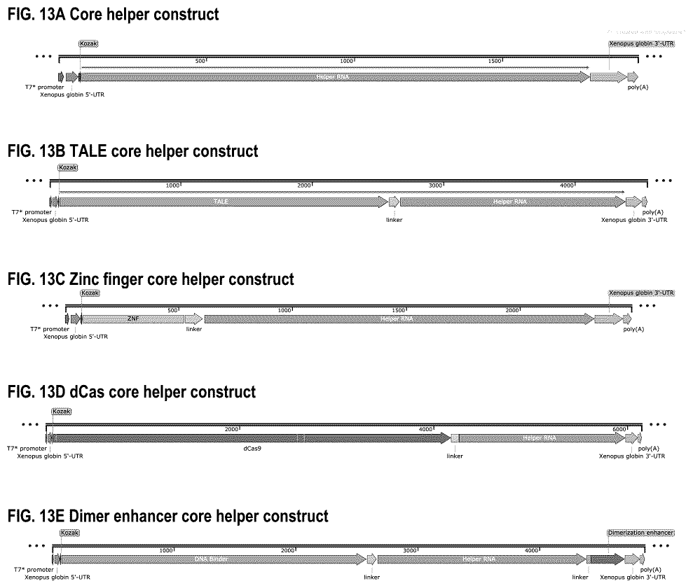

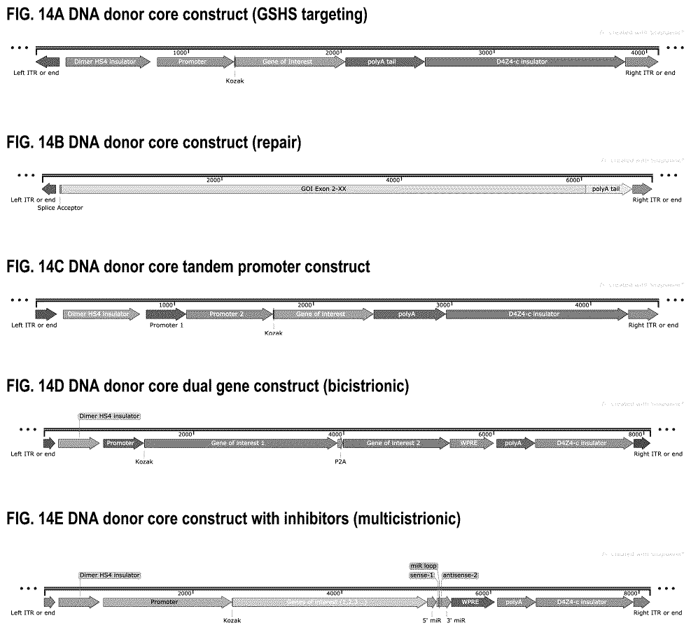

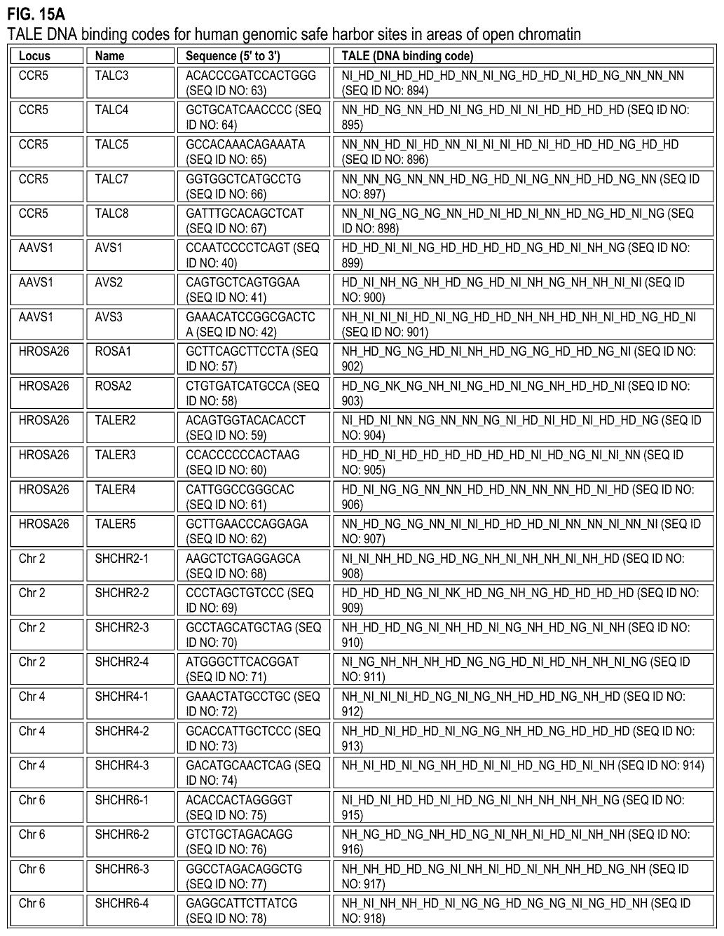

In this example, chimeric helpers are designed using human GSHS TALE, ZnF, Cas9/gRNA DBD, or Cas12/gRNA DBD such as, for example Cas12j or Cas12a. FIGS. 13A-E depict representations of RNA or DNA helper enzymes that are designed to target human GSHS or endogeneous genes using TALE, ZnF, Cas9/guide RNA DNA binders, and enhanced dimerization. In FIG. 13A, the core RNA construct shows the helper ezyme flanked by a glbin 5′- and 3′-UTR, and a short polyA tail. In FIGS. 13B, 13C, and 13D, a TALE, ZnF, or dCas DNA binder is linked to the helper enzyme by a linker that is greater than 23 amino acids in length. See Hew et al., Synth Biol (Oxf) 2019; 4:ysz018. In FIG. 13E, the TALE, ZnF, or dCas is linked to the helper enzyme that is bound to a dimerization enhancer to form an active dimer that pastes the donor DNA (FIG. 14A, 14B, 14C, 14D, or 14E) at TTAA sites within GSHS (See underlined and bolded TTAA regions in FIG. 16B, FIG. 17B, FIG. 18B, FIG. 19B, FIG. 20B, FIG. 21B, FIG. 22B, FIG. 23B, or FIG. 24B near repeat variable di-residues (RVD) nucleotide sequences).

FIGS. 14A-E depict representations of DNA donor comprising DNA with recognition sites called ends or ITRs fused or linked via to insulators, promoters, genes of interest, or miRNA (sense, loop, antisense). The inverted terminal repeat (ITR) recognition sequences are included at the 5′- and 3′-ends and are illustrated in each figure. FIG. 14A depicts an illustrative core donor construct that is contained in a replication backbone (e.g., plasmid or miniplasmid) with a promoter driving a gene of interest (GOI) with a polyA tail flanked by two insulators and ITRs. This construct is used for targeting genomic safe harbor sites (GSHS) or other loci. FIG. 14B depicts an illustrative core donor construct that is contained in a replication backbone (e.g., plasmid or miniplasmid) with a splice acceptor site for exon 2 and other exons of a gene of interest (GOI) followed by a polyA tail and flanked by ITRs. This construct is used for targeting endogenous genes in the first intron (or other introns) to repair downstream mutations. FIG. 14C depicts an illustrative core donor construct that is contained in a replication backbone (e.g., plasmid or miniplasmid) with tandem promoters to affect expression in different tissues (e.g., without limitation, liver specific promoter, cardiac specific promoter) and a gene (s) of interest (GOI) followed by a polyA tail and flanked by ITRs. This construct is used to differentially promote expression of genes in different organs, tissues or cell types. FIG. 14D depicts an illustrative core donor construct that is contained in a replication backbone (e.g., plasmid or miniplasmid) with two or more genes of interest (GOI) linked by P2A “self-cleaving” peptides and followed by WPRE and a polyA tail. The construct is flanked by ITRs. This construct is used for delivering multiple genes or genetic factors. FIG. 14E depicts an illustrative core donor construct that is contained in a replication backbone (e.g., plasmid or miniplasmid) with a promoter(s) driving the expression of two or more genes as in FIG. 14D and linked to a sequence consisting of a 5′-miRNA, a sense and antisense miRNA pair, and completed with the 3′-miRNA. The construct is followed by WPRE and flanked by ITRs. This construct combines protein replacement and miRNA to inhibit the expression of other related proteins.

All RVD are preceded by a thymine (T) to bind to the NTR shown in FIG. 15A. All of these GSHS regions are in open chromatin and are susceptible to helper activity.

### Example 3—Integration Efficiency of Pteropus Vampyrus Helper Enzyme

The goal of this study is to test DNA integration efficiency of the novel Pteropus vampyrus helper enzyme.

HEK293 is seeded at a density of about 1.25×10⁶ cells in duplicate T25 flasks. Lipofectamine LTX (Invitrogen) or an equivalent is used to transfect DNA donor (CMV-GFP):RNA Helper (3.0 ug:1.5 ug). This experiment uses Pteropus vampyrus helper RNA (SEQ ID NO: 2) and donor DNA ends from Pteropus vampyrus left end sequence (SEQ ID NO: 11) and Pteropus vampyrus right end sequence (SEQ ID NO: 16). Cells is split twice a week and % GFP is measured by FACs at 48 hours and three weeks. Percent integration efficiency is calculated from % GFP positive cells at 3 weeks minus % GFP positive cells at 48 hours. The percent integration efficiency is expected to be high relative to the controls. Negative controls of the experiment, which may include mock, RNA alone, and untreated cells, are expected to show little to no GFP fluorescence. Overall cell viability is expected to be high.

### Example 4—Integration Efficiency of Mammalian Helper Enzymes

DNA integration efficiency of PBGD4 helper enzyme with various donor DNA ends were tested. PBGD4 helper RNA (SEQ ID NO: 3) was tested in combination with left end sequence and right end sequence from Pteropus vampyrus (SEQ ID NO: 11 and SEQ ID NO: 16), MER75 (SEQ ID NO: 13 and SEQ ID NO: 18), MER75B (SEQ ID NO: 14 and SEQ ID NO: 19), and MER75A (SEQ ID NO: 15 and SEQ ID NO: 20). The results were compared to that of Myotis lucifugus helper RNA (SEQ ID NO: 10) in combination with left end sequence and right end sequence from Myotis lucifugus.

The results are shown in TABLE 1.

TABLE 1
Donor DNA integration efficiency after
transfection of HEK293 cells

| Helper RNA | Donor DNA ends | Integration efficiency %* |
|---|---|---|
| Myotis lucifugus | Myotis lucifugus | 3.5 |
| PBGD4 | MER75A | 0.9 |
| PBGD4 | MER75 | 0.8 |
| PBGD4 | MER75B | 0.7 |
| PBGD4 | Pteropus vampyrus | 0.6 |

*Integration efficiency—% GFP+ cells at day 21 divided by % GFP+ cells at day 2 post transfection

HEK293 were seeded at a density of 1.25×10⁶ cells in duplicate T25 flasks. Lipofectamine LTX (Invitrogen) was used to transfect DNA donor (CMV-GFP):RNA Helper (3.0 ug:1.5 ug). Cells were split twice a week and % GFP was measured by FACs at 48 hours and three weeks. Integration efficiency %=% GFP positive cells at 3 weeks—% GFP positive cells at 48 hours. Mock, RNA alone, and untreated cells showed no GFP fluorescence. Overall cell viability was high at 95.2%.

Additional experiments can be carried out to test the DNA integration efficiency of other helper enzymes with various donor DNA ends. For instance, helper RNA from PBGD4 hyperactive mutant (SEQ ID NO: 4), PBGD1 (SEQ ID NO: 6), PBGD2 (SEQ ID NO: 7), PBGD3 (SEQ ID NO: 8), PBGD5 (SEQ ID NO: 9) can be tested in combination with left end sequence and right end sequence from Pteropus vampyrus (SEQ ID NO: 11 and SEQ ID NO: 16), MER75 (SEQ ID NO: 13 and SEQ ID NO: 18), MER75B (SEQ ID NO: 14 and SEQ ID NO: 19), MER75A (SEQ ID NO: 15 and SEQ ID NO: 20), PGBD4 (SEQ ID NO: 12 and SEQ ID NO: 17), or Myotis lucifugus. The results can be compared to that of Myotis lucifugus helper RNA (SEQ ID NO: 10) in combination with left end sequence and right end sequence from Myotis lucifugus.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A composition comprising:
(a) a recombinant helper enzyme, or a nucleotide sequence encoding the same, having gene cleavage (Exc) and/or gene integration (Int) activity and at least about 90% identity to the amino acid sequence of SEQ ID NO: 2, and/or
(b) a gene transfer construct comprises a vector comprising a donor DNA comprising left and right end sequences recognized by the recombinant helper enzyme, the left and right end sequences having at least about 90% identity to the nucleotide sequences of SEQ ID NO: 11 and SEQ ID NO: 16.

2. The composition of claim 1, wherein the helper enzyme has at least about 95%, or at least about 96%, at least about 97%, at least about 98%, at least about 99% identity to the amino acid sequence SEQ ID NO: 2.

3. The composition of claim 1 or claim 2, wherein the helper enzyme has one or more mutations which confer hyperactivity.

4. The composition of any one of claims 1 to 3, wherein the helper enzyme has an amino acid sequence having mutations at positions which correspond to at least one of S8P and G17R mutations relative to the amino acid sequence of SEQ ID NO: 2 or a functional equivalent thereof.

5. The composition of claim 4, wherein the helper enzyme has the nucleotide sequence having at least about 90% identity to SEQ ID NO: 1 or a codon-optimized form thereof.

6. The composition of claim 5, wherein the helper enzyme has the nucleotide sequence having at least about 95%, or at least about 96%, at least about 97%, at least about 98%, at least about 99% identity to SEQ ID NO: 1, or a codon-optimized form thereof.

7. The composition of claim 5 or 6, wherein the nucleotide sequence comprises a thymine (T) at position 1933 of SEQ ID NO: 1, or a position corresponding thereto of SEQ ID NO: 1.

8. The composition of any one of the above claims, wherein the composition comprises a gene transfer construct.

9. The composition of claim 8, wherein the gene transfer construct comprises a vector comprising a donor DNA comprising left and right end sequences recognized by the helper enzyme.

10. The composition of claim 9, wherein the end sequences are selected from Pteropus vampyrus ends, MER75, MER75A, MER75B, and MER85.

11. The composition of claim 10, wherein the end sequences are selected from nucleotide sequences of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20, or a nucleotide sequence having at least about 90% identity thereto.

12. The composition of any one of claims 9 to 11, wherein one or more of the end sequences are optionally flanked by a TTAA sequence.

13. The composition of claim 11, wherein the end sequences include at least one repeat from a nucleotide sequence having at least about 90% identity to the nucleotide sequence of SEQ ID NO: 11, and wherein the at least one repeat from the nucleotide sequence having at least about 90% identity to the nucleotide sequence of SEQ ID NO: 11 is positioned at the 5′ end of the donor DNA.

14. The composition of claim 13, wherein the end sequences include at least one repeat from a nucleotide sequence having at least about 90% identity to the nucleotide sequence of SEQ ID NO: 16, and wherein the at least one repeat from the nucleotide sequence having at least about 90% identity to the nucleotide sequence of SEQ ID NO: 16 is positioned at the 3′ end of the donor DNA.

15. The composition of claim 13 or claim 14, wherein the end sequences are optionally flanked by a TTAA sequence.

16. The composition of claim 11, wherein the end sequences include at least one repeat from a nucleotide sequence having at least about 90% identity to the nucleotide sequence of SEQ ID NO: 12, and wherein the at least one repeat from the nucleotide sequence having at least about 90% identity to the nucleotide sequence of SEQ ID NO: 12 is positioned at the 5′ end of the donor DNA.

17. The composition of claim 16, wherein the end sequences include at least one repeat from a nucleotide sequence having at least about 90% identity to the nucleotide sequence of SEQ ID NO: 17, and wherein the at least one repeat from the nucleotide sequence having at least about 90% identity to the nucleotide sequence of SEQ ID NO: 17 is positioned at the 3′ end of the donor DNA.

18. The composition of claim 16 or claim 17, wherein the end sequences are optionally flanked by a TTAA sequence.

19. The composition of claim 11, wherein the end sequences include at least one repeat from a nucleotide sequence having at least about 90% identity to the nucleotide sequence of SEQ ID NO: 13, wherein the at least one repeat from the nucleotide sequence having at least about 90% identity to the nucleotide sequence of SEQ ID NO: 13 is positioned at the 5′ end of the donor DNA.

20. The composition of claim 19, wherein the end sequences include at least one repeat from a nucleotide sequence having at least about 90% identity to the nucleotide sequence of SEQ ID NO: 18, wherein the at least one repeat from the nucleotide sequence having at least about 90% identity to the nucleotide sequence of SEQ ID NO: 18 is positioned at the 3′ end of the donor DNA.

21. The composition of claim 19 or claim 20, wherein the end sequences are optionally flanked by a TTAA sequence.

22. The composition of claim 11, wherein the end sequences include at least one repeat from a nucleotide sequence having at least about 90% identity to the nucleotide sequence of SEQ ID NO: 14, wherein the at least one repeat from the nucleotide sequence having at least about 90% identity to the nucleotide sequence of SEQ ID NO: 14 is positioned at the 5′ end of the donor DNA.

23. The composition of claim 22, wherein the end sequences include at least one repeat from a nucleotide sequence having at least about 90% identity to the nucleotide sequence of SEQ ID NO: 19, wherein the at least one repeat from the nucleotide sequence having at least about 90% identity to the nucleotide sequence of SEQ ID NO: 19 is positioned at the 3′ end of the donor DNA.

24. The composition of claim 22 or claim 23, wherein the end sequences are optionally flanked by a TTAA sequence.

25. The composition of claim 11, wherein the end sequences include at least one repeat from a nucleotide sequence having at least about 90% identity to the nucleotide sequence of SEQ ID NO: 15, wherein the at least one repeat from the nucleotide sequence having at least about 90% identity to the nucleotide sequence of SEQ ID NO: 15 is positioned at the 5′ end of the donor DNA.

26. The composition of claim 25, wherein the end sequences include at least one repeat from a nucleotide sequence having at least about 90% identity to the nucleotide sequence of SEQ ID NO: 20, wherein the at least one repeat from the nucleotide sequence having at least about 90% identity to the nucleotide sequence of SEQ ID NO: 20 is positioned at the 3′ end of the donor DNA.

27. The composition of claim 25 or claim 26, wherein the end sequences are optionally flanked by a TTAA sequence.

28. A composition comprising:
(a) a recombinant helper enzyme, or a nucleotide sequence encoding the same, having gene cleavage (Exc) and/or gene integration (Int) activity and at least about 90% identity to the amino acid sequence of SEQ ID NO: 3, SEQ ID NO: 4, SEQ ID NO: 6, SEQ ID NO: 7, SEQ ID NO: 8, or SEQ ID NO: 9, and
(b) a gene transfer construct comprises a vector comprising a donor DNA comprising left and right end sequences recognized by the recombinant helper enzyme, the end sequences having at least about 90% identity to the nucleotide sequences of SEQ ID NO: 11 and SEQ ID NO: 16.

29. The composition of any one of claims 1-28, wherein the composition comprises a targeting element.

30. The composition of any one of claims 1-29, wherein the composition is capable of inserting a donor comprising a transgene in a genomic safe harbor site (GSHS).

31. The composition of claim 30, wherein the binding of a GSHS of a nucleic acid molecule in a mammalian cell is with high target specificity.

32. The composition of any one of claims 29-31, wherein the targeting element is able to direct a transposition machinery to the GSHS of a nucleic acid molecule in a mammalian cell.

33. The composition of any one of claims 30-32, wherein the GSHS is in an open chromatin location in a chromosome.

34. The composition of any one of claims 30-33, wherein the GSHS is selected from adeno-associated virus site 1 (AAVS1), chemokine (C—C motif) receptor 5 (CCR5) gene, HIV-1 coreceptor, and human Rosa26 locus.

35. The composition of any one of claims 30-34, wherein the GSHS is an adeno-associated virus site 1 (AAVS1).

36. The composition of any one of claims 30-35, wherein the GSHS is a human Rosa26 locus.

37. The composition of any one of claims 30-36, wherein the GSHS is located on human chromosome 2, 4, 6, 11, 17, 22, or X.

38. The composition of any one of claims 30-37, wherein the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

39. The composition of any one of claims 30-38, wherein the targeting element comprises one or more of a Cas enzyme, which is optionally catalytically inactive and which is optionally associated with a guide RNA (gRNA), transcription activator-like effector (TALE) DNA binding domain (DBD), catalytically inactive Zinc finger, catalytically inactive transcription factor, catalytically inactive nickase, a transcriptional activator, a transcriptional repressor, a recombinase, a DNA methyltransferase, a histone methyltransferase, and a paternally expressed gene 10 (PEG10).

40. The composition of claim 39, wherein the targeting element comprises a TALE DBD.

41. The composition of claim 40, wherein the TALE DBD comprises one or more repeat sequences.

42. The composition of claim 41, wherein the TALE DBD comprises about 14, or about 15, or about, 16, or about 17, or about 18, or about 18.5 repeat sequences.

43. The composition of claim 41 or claim 42, wherein the repeat sequences each independently comprises about 33 or 34 amino acids.

44. The composition of claim 43, wherein the repeat sequences each independently comprises a repeat variable di-residue (RVD) at residue 12 or 13 of the 33 or 34 amino acids, respectively.

45. The composition of claim 44, wherein the RVD recognizes one base pair in a target nucleic acid sequence.

46. The composition of claim 44 or claim 45, wherein the RVD recognizes a C residue in the target nucleic acid sequence and is selected from HD, N(gap), HA, ND, and HI.

47. The composition of claim 44 or claim 45, wherein the RVD recognizes a G residue in the target nucleic acid sequence and is selected from NN, NH, NK, HN, and NA.

48. The composition of claim 44 or claim 45, wherein the RVD recognizes an A residue in the target nucleic acid sequence and is selected from NI and NS.

49. The composition of claim 44 or claim 45, wherein the RVD recognizes a T residue in the target nucleic acid sequence and is selected from NG, HG, H(gap), and IG.

50. The composition of claim 39, wherein the targeting element comprises a Cas9 enzyme associated with a gRNA.

51. The composition of claim 50, wherein the Cas9 enzyme associated with a gRNA comprises a catalytically inactive dCas9 associated with a gRNA.

52. The composition of claim 51, wherein the catalytically inactive dCas9 comprises at least about 90%, or at least about 95%, or at least about 96%, or at least about 97%, or at least about 98%, or at least about 99% identity to an amino acid sequence of SEQ ID NO: 21 or a nucleic acid comprising a nucleotide sequence of SEQ ID NO: 22 or a codon-optimized form thereof.

53. The composition of any one of claims 29-52, wherein the targeting element comprises a Cas12 enzyme associated with a gRNA.

54. The composition of claim 53, wherein the targeting element comprises a catalytically inactive Cas12 associated with a gRNA, optionally wherein the catalytically inactive Cas12 is dCas12j or dCas12a.

55. The method of any one of claims 29-54, wherein the targeting element comprises: a gRNA of or comprising a sequence of TABLE 3A-3F, or a variant thereof; or a TALE DBD of or comprising a sequence of TABLE 4A-4F, or a variant thereof; or a ZNF of or comprising a sequence of TABLE 5A-5E, or a variant thereof.

56. The composition of any one of claims 29-55, wherein the targeting element comprises a nucleic acid binding component of a gene-editing system.

57. The composition of any one of claims 29-56, wherein the enzyme or variant thereof and the targeting element are connected.

58. The composition of claim 57, wherein the enzyme and the targeting element are fused to one another or linked via a linker to one another.

59. The composition of claim 58, wherein the linker is a flexible linker.

60. The composition of claim 59, wherein the flexible linker is substantially comprised of glycine and serine residues, optionally wherein the flexible linker comprises (Gly₄Ser)ₙ, where n is an integer from 1-12.

61. The composition of claim 60, wherein the flexible linker is of about 20, or about 30, or about 40, or about 50, or about 60 amino acid residues.

62. The composition of claim 61, wherein the enzyme is directly fused to the N-terminus of the dCas9 enzyme.

63. The composition of any one of claims 1-62, wherein the enzyme or variant thereof is able to directly or indirectly cause transposition of a target gene.

64. The composition of any one of claims 1-63, wherein the enzyme or variant thereof is able to directly or indirectly interact and/or form a complex with one or more proteins or nucleic acids.

65. The composition of any one of claims 1-64, further comprising a nucleic acid encoding a donor comprising a transgene to be integrated.

66. The composition of claim 65, wherein the transgene comprises a cargo nucleic acid sequence and a first and a second donor end sequences.

67. The composition of claim 66, wherein the cargo nucleic acid sequence is flanked by the first and the second donor end sequences.

68. The composition of any one of claims 1-67, wherein the enzyme or variant thereof is incorporated into a vector or a vector-like particle.

69. The composition of any one of claims 1-68, wherein the vector or a vector-like particle comprises one or more expression cassettes.

70. The composition of claim 69, wherein the vector or a vector-like particle comprises one expression cassette.

71. The composition of claim 70, wherein the expression cassette further comprises the enzyme or variant thereof, the transgene, the donor end sequences, or a combination thereof.

72. The composition of claim 71, wherein the enzyme or variant thereof, the transgene, the donor end sequences, or a combination thereof are incorporated into one or more vectors or vector-like particles.

73. The composition of claim 72, wherein the enzyme or variant thereof, the transgene, the donor end sequences, or combination thereof are incorporated into a same vector or vector-like particle.

74. The composition of claim 72, wherein the enzyme or variant thereof, the transgene, the donor end sequences, or combination thereof is incorporated into different vectors vector-like particles.

75. The composition of claim of any one of claims 68-74, wherein the vector or vector-like particle is nonviral.

76. The composition of any one of claims 1-75, wherein the composition comprises DNA, RNA, or both.

77. The composition of any one of claims 1-76, wherein the enzyme or variant thereof is in the form of RNA.

78. A host cell comprising the composition any one of claims 1-77.

79. The composition of any one of claims 1-77, wherein the composition is encapsulated in a lipid nanoparticle (LNP).

80. The composition of any one of claims 1-79, wherein the polynucleotide encoding the enzyme or variant thereof and the polynucleotide encoding the donor are in the form of the same LNP, optionally in a co-formulation.

81. The composition of claim 79 or claim 80, wherein the LNP comprises one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol −2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GaINAc).

82. A method for inserting a gene into the genome of a cell, comprising contacting a cell with the composition of any one of claim 1-77 or 79-81 or host cell of claim 78.

83. The method of claim 82, further comprising contacting the cell with a polynucleotide encoding a donor.

84. The method of claim 82 or claim 83, wherein the donor comprises a gene encoding a complete polypeptide.

85. The method of any one of claims 82-84, wherein the donor comprises a gene which is defective or substantially absent in a disease state.

86. A method for treating a disease or disorder ex vivo, comprising contacting a cell with the composition of any one of claim 1-77 or 79-85 or host cell of claim 78 and administering the cell to a subject in need thereof.

87. A method for treating a disease or disorder in vivo, comprising administering the composition of any one of claim 1-77 or 79-86 or host cell of claim 78 to a subject in need thereof.
